SEQUENCE LISTING (18 sequences)

SEQ ID NO: 1 - protein, 86 aa, Homo sapiens
  Human FcRy chain (NP_004097.1 (GI4758344))
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 2 - DNA, 21 bp, Artificial Sequence
  Primer
atatttacagaatggcacagg

SEQ ID NO: 3 - DNA, 20 bp, Artificial Sequence
  Primer
gacttggtacccaggttgaa

SEQ ID NO: 4 - DNA, 32 bp, Artificial Sequence
  Primer
atcagattcgatcctacttctgcagggggcat

SEQ ID NO: 5 - DNA, 32 bp, Artificial Sequence
  Primer
acgtgctgagcttgagtgatggtgatgttcac

SEQ ID NO: 6 - DNA, 24 bp, Artificial Sequence
  Primer
cccaactcaacttcccagtgtgat

SEQ ID NO: 7 - DNA, 30 bp, Artificial Sequence
  Primer
gaaatctaccttttcctctaatagggcaat

SEQ ID NO: 8 - DNA, 29 bp, Artificial Sequence
  Primer
gaaatctaccttttcctctaatagggcaa

SEQ ID NO: 9 - DNA, 28 bp, Artificial Sequence
  Primer
gaaatctaccttttcctctaatagggca

SEQ ID NO: 10 - DNA, 21 bp, Artificial Sequence
  Primer
ccaaaagccacactcaaagac

SEQ ID NO: 11 - DNA, 21 bp, Artificial Sequence
  Primer
acccaggtggaaagaatgatg

SEQ ID NO: 12 - DNA, 25 bp, Artificial Sequence
  Probe
aacatcaccatcactcaaggtttgg

SEQ ID NO: 13 - DNA, 25 bp, Artificial Sequence
  Primer
ctgaagacacatttttactcccaaa

SEQ ID NO: 14 - DNA, 22 bp, Artificial Sequence
  Primer
tccaaaagccacactcaaagac

SEQ ID NO: 15 - DNA, 25 bp, Artificial Sequence
  Primer
ctgaagacacatttttactcccaac

SEQ ID NO: 16 - protein, 238 aa, Artificial Sequence
  CD16 (F158)
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 17 - protein, 16 aa, Artificial Sequence
  Signal peptide
MWQLLLPTALLLLVSA

SEQ ID NO: 18 - protein, 238 aa, Artificial Sequence
  CD16 158V+ (VAR_003960)
GMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLVGSKNVSSETVNITITQGLAVSTISSFFPPGYQVSFCLVMVLLFAVDTGLYFSVKTNIRSSTRDWKDHKFKWRKDPQDK

(19) United States
(12) Patent Application Publication
(10) Pub. No.: US 2024/0197783 A1
(43) Pub. Date: Jun. 20, 2024

(54) METHODS OF TREATMENT AND DOSING OF NATURAL KILLER CELL COMPOSITIONS

(71) Applicant: Indapta Therapeutics, Inc., Houston, TX (US)

(72) Inventors: Guy DIPIERRO, Houston, TX (US); Austin BIGLEY, Houston, TX (US)

(73) Assignee: Indapta Therapeutics, Inc., Houston, TX (US)

(21) Appl. No.: 18/556,333

(22) PCT Filed: Apr. 20, 2022
(86) PCT No.: PCT/US2022/025651

§ 371 Date: Oct. 19, 2023

Related U.S. Application Data
(60) Provisional application No. 63/177,956, filed on Apr. 21, 2021.

(51) Int. Cl. A61K 35/17; A61K 31/675; A61K 31/7076; A61K 38/47; A61K 39/00; A61K 45/06; A61P 35/00

(52) U.S. Cl. CPC A61K 35/17; A61K 31/675; A61K 31/7076; A61K 38/47; A61K 45/06; A61P 35/00; A61K 2039/505; A61K 2039/545

(57) ABSTRACT

Provided herein are methods for treatment and uses involving dosing of compositions containing NK cells. Among the provided methods and uses are methods and uses for treating cancer, such as multiple myeloma or lymphoma, including in combination with an antibody therapeutic for the cancer.

### CROSS-REFERENCE TO RELATED APPLICATION

This application claims priority to U.S. provisional application No. 63/177,956, filed Apr. 21, 2021, entitled “METHODS OF TREATMENT AND DOSING OF NATURAL KILLER CELL COMPOSITIONS,” the contents of which are incorporated by reference in their entirety for all purposes.

### INCORPORATION BY REFERENCE OF SEQUENCE LISTING

The present application is being filed along with a Sequence Listing in electronic format. The Sequence Listing is provided as a file entitled 776032001240_SEQLIST.Txt, created Apr. 20, 2022, which is 7,852 bytes in size. The information in the electronic format of the Sequence Listing is incorporated by reference in its entirety.

### FIELD

The present disclosure provides methods for treatment and uses involving dosing of compositions containing NK cells. Among the provided methods and uses are methods and uses for treating cancer, such as multiple myeloma or lymphoma, including in combination with an antibody therapeutic for the cancer.

### BACKGROUND

Antibody-based therapy has become frequently used for treating cancers and other diseases. Responses to antibody therapy have typically focused on the direct inhibitory effects of these antibodies on the tumor cells (e.g. inhibition of growth factor receptors and the subsequent induction of apoptosis), but the in vivo effects of these antibodies may be more complex and may involve the host immune system. Natural killer (NK) cells are immune effector cells that mediate antibody-dependent cellular cytotoxicity when the Fc receptor (CD16; FcγRIII) binds to the Fc portion of antibodies bound to an antigen-bearing cell. NK cells, including specific specialized subsets thereof, can be used in therapeutic methods, including for improving responses to antibody therapy. Improved methods are needed for therapeutic uses involving NK cells, including in combination with antibodies. Provided herein are embodiments that meet such needs.

### SUMMARY

Provided herein is a method of treating multiple myeloma, where the method includes administering a composition of Natural Killer (NK) cells deficient in expression of FcRγ chain (g-NK cells) to a subject having multiple myeloma (MM), wherein the composition of g-NK cells may be administered once weekly for a predetermined number of doses. Provided herein is a composition of Natural Killer (NK) cells deficient in expression of FcRγ chain (g-NK cells) for use in a method of treating a subject having multiple myeloma (MM), wherein the composition of g-NK cells may be administered once weekly for a predetermined number of doses.

In some embodiments, the method may be a monotherapy without combined administration of an exogenous antibody for treating the multiple myeloma. In some embodiments, the method further comprises administering to the subject an antibody that is directed against a multiple myeloma antigen. In some embodiments, the multiple myeloma antigen comprises an antigen selected from the group consisting of CD38, SLAMF7, and BCMA. In some embodiments, the antibody is a full-length antibody. In some embodiments, the antibody is an anti-SLAMF7 antibody. In some embodiments, the antibody is an anti-BCMA antibody. In some embodiments, the antibody is an anti-CD38 antibody. In some embodiments the antibody is a bispecific antibody. In some embodiments, the bispecific antibody is directed against CD16 and BCMA. In some embodiments, the bispecific antibody is directed against CD16 and SLAMF7. In some embodiments, the bispecific antibody is directed against CD16 and CD38. In some embodiments, at least one dose of anti-CD38 antibody has been administered to the subject prior to administration of a dose of the composition of g-NK cells.

In some embodiments, the antibody is administered once every four weeks. In some embodiments, the antibody is administered once every three weeks. In some embodiments, the antibody is administered once every two weeks. In some embodiments, the antibody is administered once weekly. In some embodiments, the antibody is administered twice weekly. In some embodiments, the antibody is administered more than twice weekly. Also provided herein is a method of treating multiple myeloma, where the method includes administering a composition of Natural Killer (NK) cells deficient in expression of FcRγ chain (g-NK cells) to a subject having multiple myeloma (MM), wherein the composition of g-NK cells may be administered once weekly for a predetermined number of doses, and wherein the subject has received prior administration of at least one dose of an anti-CD38 antibody. Also provided herein is a composition of Natural Killer (NK) cells deficient in expression of FcRγ chain (g-NK cells) for use in a method of treating a subject having multiple myeloma (MM), wherein the composition of g-NK cells may be administered once weekly for a predetermined number of doses, and wherein the subject has received prior administration of at least one dose of an anti-CD38 antibody.

In some embodiments, the g-NK cell composition may be administered as two doses in a 14-day cycle, wherein the 14-day cycle may be repeated one to three times. In some embodiments, the g-NK cell composition may be administered as six total doses. In some embodiments, the anti-CD38 antibody may be daratumumab. In some embodiments, administration of the at least one dose of the anti-CD38 antibody may be initiated within one month prior to administration of the composition of g-NK cells. In some embodiments, administration of the at least one dose of the anti-CD38 antibody may be initiated within three weeks prior to administration of the composition of g-NK cells. In some embodiments, administration of the at least one dose of the anti-CD38 antibody may be initiated within two weeks prior to administration of the composition of g-NK cells.

In some embodiments, the anti-CD38 antibody may be administered intravenously. In some embodiments, the anti-CD38 antibody may be administered as a once weekly dose, optionally for one or two 28-day cycles. In some embodiments, each dose of the anti-CD38 antibody (e.g. daratumumab) may be administered in an amount that may be from or from about 8 mg/kg to about 32 mg/kg, optionally at or about 16 mg/kg. In some embodiments, the anti-CD38 antibody may be administered subcutaneously. In some embodiments, the anti-CD38 antibody (e.g. daratumumab) may be administered in an anti-CD38 antibody composition including a hyaluronidase, optionally wherein the anti-CD38 antibody composition includes daratumumab and recombinant human hyaluronidase PH20 (e.g. hyaluronidase-fihj).

In some embodiments, the anti-CD38 antibody composition may be administered as a once weekly dose, optionally for one or two 28-day cycles. In some embodiments, each dose of the anti-CD38 antibody composition includes from at or about 1200 mg to about 2400 mg anti-CD38 antibody (e.g. daratumumab) and from at or about 15,000 Units (U) to about 45,000 U hyaluronidase (e.g. hyaluronidase-fihj). In some embodiments, each dose of the anti-CD38 antibody composition includes about 1800 mg anti-CD38 antibody (e.g. daratumumab) and about 30,000 U hyaluronidase (e.g. hyaluronidase-fihj). In some embodiments, the method includes administering the anti-CD38 antibody, optionally the anti-CD38 antibody composition, once weekly for 8 total doses and administering the g-NK cell composition once weekly for 6 total doses, wherein one dose or two doses of the anti-CD38 antibody may be administered prior to administration of the composition including g-NK cells. In some embodiments, the multiple myeloma may be relapsed/refractory multiple myeloma.

In some embodiments, the g-NK cells have low or no expression of CD38, optionally wherein less than 25% of the cells in the g-NK cell composition are positive for surface CD38. In some embodiments, the cells in the g-NK cell composition are not engineered to reduce or eliminate CD38 expression. In some embodiments, the g-NK cell composition exhibits minimal anti-CD38-induced fratricide, optionally wherein less than 10% of cells in the g-NK cell composition exhibit anti-CD38 induced fratricide.

Provided herein is a method of treating lymphoma, where the method includes administering a composition of Natural Killer (NK) cells deficient in expression of FcRγ chain (g-NK cells) to a subject having lymphoma, wherein the composition of g-NK cells may be administered once weekly for a predetermined number of doses. Provided herein is a composition of Natural Killer (NK) cells deficiency in expression of FcRγ chain (g-NK cells) for use in a method of treating a subject having lymphoma, wherein the composition of g-NK cells may be administered once weekly for a predetermined number of doses. In some embodiments, the method may be a monotherapy without combined administration of an exogenous antibody for treating the lymphoma. In some embodiments, the method further comprises administering to the subject an antibody that is directed against a lymphoma antigen. In some embodiments, the lymphoma antigen comprises an antigen selected from the group consisting of CD19, CD20, and CD30. In some embodiments, the antibody is a full-length antibody. In some embodiments, the antibody is an anti-CD19 antibody. In some embodiments, the antibody is an anti-CD30 antibody. In some embodiments, the antibody is an anti-CD20 antibody. In some embodiments, the antibody is a bispecific antibody. In some embodiments, the bispecific antibody is directed to CD16 and a second antigen selected from the group consisting of CD19, CD20, and CD30. In some embodiments, the bispecific antibody is directed to CD16 and CD19. In some embodiments, the bispecific antibody is directed to CD16 and CD20. In some embodiments, the bispecific antibody is directed to CD16 and CD30. In some embodiments, at least one dose of anti-CD20 antibody has been administered to the subject prior to administration of a dose of the composition of g-NK cells.

In some embodiments, the antibody is administered once every four weeks. In some embodiments, the antibody is administered once every three weeks. In some embodiments, the antibody is administered once every two weeks. In some embodiments, the antibody is administered once weekly. In some embodiments, the antibody is administered twice weekly. In some embodiments, the antibody is administered more than twice weekly.

Also provided herein is a method of treating lymphoma, where the method includes administering a composition of Natural Killer (NK) cells deficient in expression of FcRγ chain (g-NK cells) to a subject having lymphoma, wherein the composition of g-NK cells may be administered once weekly for a predetermined number of doses, and wherein the subject has received prior administration of at least one dose of an anti-CD20 antibody. Also provided herein is a composition of Natural Killer (NK) cells deficient in expression of FcRγ chain (g-NK cells) for use in a method of treating a subject having lymphoma, wherein the composition of g-NK cells may be administered once weekly for a predetermined number of doses, and wherein the subject has received prior administration of at least one dose of an anti-CD20 antibody.

In some embodiments, the lymphoma may be a Non-Hodgkin's Lymphoma (NHL). In some embodiments, the g-NK cell composition may be administered as two doses in a 14-day cycle, wherein the 14-day cycle may be repeated one to three times. In some embodiments, the g-NK cell composition may be administered as six total doses. In some embodiments, the anti-CD20 antibody may be rituximab.

In some embodiments, administration of the at least one dose of the anti-CD20 antibody may be initiated within one month prior to administration of the composition of g-NK cells. In some embodiments, at least one dose of the anti-CD20 antibody may be initiated within three weeks prior to administration of the composition of g-NK cells. In some embodiments, administration of the at least one dose of the anti-CD20 antibody may be initiated within two weeks prior to administration of the composition of g-NK cells. In some embodiments, the anti-CD20 antibody may be administered intravenously. In some embodiments, the anti-CD20 antibody may be administered as a once weekly dose, optionally for 4 or 8 doses. In some embodiments, each dose of the anti-CD20 antibody may be administered in an amount that may be from or from about 250 mg/m2 to 500 mg/m2, optionally at or about 375 mg/m2. In some embodiments, the anti-CD20 antibody may be administered subcutaneously.

In some embodiments, the anti-CD20 antibody (e.g. rituximab) may be administered in an anti-CD20 antibody composition including a hyaluronidase, optionally wherein the anti-CD20 antibody composition includes rituximab and a human recombinant hyaluronidase PH20. In some embodiments, the anti-CD20 antibody composition may be administered as a once weekly dose, optionally for 4 or 8 doses or optionally for 3 or 7 doses following a once weekly dose of the anti-CD20 antibody intravenously. In some embodiments, each dose of the anti-CD20 antibody composition includes from at or about 1200 mg to about 2400 mg anti-CD20 antibody (e.g. rituximab) and from at or about 15,000 Units (U) to about 45,000 U hyaluronidase. In some embodiments, each dose of the anti-CD20 antibody composition includes about 1400 mg anti-CD20 antibody (e.g. rituximab) and about 23,400 U hyaluronidase. In some embodiments, each dose of the anti-CD20 antibody composition includes about 1600 mg anti-CD20 antibody (e.g. rituximab) and about 26,800 U hyaluronidase.

In some embodiments, the method includes administering the anti-CD20 antibody, optionally the anti-CD20 antibody composition, once weekly for 8 total doses and administering the g-NK cell composition once weekly for 6 total doses, wherein one dose or two doses of the anti-CD20 antibody may be administered prior to administration of the composition including g-NK cells. In some embodiments, among cells in the g-NK cell composition, greater than at or about 60% of the cells are g-NK cells, greater than at or about 70% of the cells are g-NK cells, greater than at or about 80% of the cells are g-NK cells, greater than at or about 90% of the cells are g-NK cells, or greater than at or about 95% of the cells are g-NK cells.

In some embodiments, at least at or about 50% of the cells in the g-NK cell composition are FcRγ-deficient (FcRγneg) NK cells (g-NK), wherein greater than at or about 70% of the g-NK cells are positive for perforin and greater than at or about 70% of the g-NK cells are positive for granzyme B. In some embodiments, (i) greater than at or about 80% of the g-NK cells are positive for perforin and greater than at or about 80% of the g-NK cells are positive for granzyme B, (ii) greater than at or about 90% of the g-NK cells are positive for perforin and greater than at or about 90% of the g-NK cells are positive for granzyme B, or (iii) greater than at or about 95% of the g-NK cells are positive for perforin and greater than at or about 95% of the g-NK cells are positive for granzyme B.

In some embodiments, among the cells positive for perforin, the cells express a mean level of perforin as measured by intracellular flow cytometry that is, based on mean fluorescence intensity (MFI), at least at or about two times the mean level of perforin expressed by cells that are FcRγpos; and/or among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγpos. In some embodiments, greater than 10% of the cells in the g-NK cell composition are capable of degranulation against tumor target cells, optionally as measured by CD107a expression, optionally wherein the degranulation may be measured in the absence of an antibody against the tumor target cells.

In some embodiments, among the cells in the g-NK cell composition, greater than at or about 15%, greater than at or about 20%, greater than at or about 30%, greater than at or about 40% or greater than at or about 50% exhibit degranulation, optionally as measured by CD107a expression, in the presence of cells expressing a target antigen (target cells) and an antibody directed against the target antigen (anti-target antibody).

In some embodiments, greater than 10% of the cells in the g-NK cell composition are capable of producing interferon-gamma or TNF-alpha against tumor target cells, optionally wherein the interferon-gamma or TNF-alpha may be measured in the absence of an antibody against the tumor target cells. In some embodiments, among the cells in the g-NK cell composition, greater than at or about 15%, greater than at or about 20%, greater than at or about 30%, greater than at or about 40% or greater than at or about 50% produce an effector cytokine in the presence of cells expressing a target antigen (target cells) and an antibody directed against the target antigen (anti-target antibody). In some embodiments, the effector cytokine may be IFN-gamma or TNF-alpha. In some embodiments, the effector cytokine may be IFN-gamma and TNF-alpha. In some embodiments, the g-NK cell composition has been produced by ex vivo expansion of CD3−/CD57+ cells cultured with irradiated HLA-E+ feeder cells, wherein the CD3−/CD57+ cells are enriched from a biological sample from a donor subject.

In some embodiments, the donor subject may be CMV-seropositive. In some embodiments, the donor subject has the CD16 158V/V NK cell genotype or the CD16 158V/F NK cell genotype, optionally wherein the biological sample may be from a human subject selected for the CD16 158V/V NK cell genotype or the CD16 158V/F NK cell genotype. In some embodiments, at least at or about 20% of natural killer (NK) cells in a peripheral blood sample from the donor subject are positive for NKG2C (NKG2Cpos) and at least 70% of NK cells in the peripheral blood sample are negative or low for NKG2A (NKG2Aneg). In some embodiments, the irradiated feeder cells are deficient in HLA class I and HLA class II. In some embodiments, the irradiated feeder cells are 221.AEH cells.

In some embodiments, the culturing may be performed in the presence of two or more recombinant cytokines, wherein at least one recombinant cytokine may be interleukin (IL)-2 and at least one recombinant cytokine may be IL-21. In some embodiments, the recombinant cytokines are IL-21 and IL-2. In some embodiments, the recombinant cytokines are IL-21, IL-2, and IL-15. In some embodiments, the g-NK cells in the composition are from a single donor subject that have been expanded from the same biological sample. In some embodiments, wherein the g-NK cell composition may be formulated in a serum-free cryopreservation medium including a cryoprotectant, optionally wherein the cryoprotectant may be DMSO and the cryopreservation medium may be 5% to 10% DMSO (v/v).

In some embodiments, the g-NK cells are not engineered with an antigen receptor, optionally wherein the antigen receptor may be a chimeric antigen receptor. In some embodiments, the g-NK cells are not engineered with a secretable cytokine, optionally a cytokine receptor fusion protein, such as IL-15 receptor fusion (IL-15RF). In some embodiments, the method does not include exogenous cytokine administration to the subject to support NK cell survival or expansion, wherein the exogenous cytokine may be one or more of IL-2, IL-7, IL-15 or IL-21.

In some embodiments, each dose of g-NK cells may be from at or about from at or about 1×108 cells to at or about 50×109 cells of the g-NK cell composition. In some embodiments, each dose of g-NK cells may be or may be about 5×108 cells of the g-NK cell composition. In some embodiments, each dose of g-NK cells may be or may be about 5×109 cells of the g-NK cell composition. In some embodiments, each dose of g-NK cells may be or may be about 10×109 cells of the g-NK cell composition. In some embodiments, prior to the administration of the dose of g-NK cells, the subject has received a lymphodepleting therapy. In some embodiments, the lymphodepleting therapy includes fludarabine and/or cyclophosphamide. In some embodiments, the lymphodepleting includes the administration of fludarabine at or about 20-40 mg/m2 body surface area of the subject, optionally at or about 30 mg/m2, daily, for 2-4 days, and/or cyclophosphamide at or about 200-400 mg/m2 body surface area of the subject, optionally at or about 300 mg/m2, daily, for 2-4 days.

In some embodiments, the lymphodepleting therapy includes fludarabine and cyclophosphamide. In some embodiments, the lymphodepleting therapy includes the administration of fludarabine at or about 30 mg/m2 body surface area of the subject, daily, and cyclophosphamide at or about 300 mg/m2 body surface area of the subject, daily, each for 2-4 days, optionally 3 days.

In some embodiments, administration of a dose of g-NK cells may be initiated within two weeks or at or about two weeks after initiation of the lymphodepleting therapy. In some embodiments, administration of a dose of g-NK cells may be initiated within 7 days or at or about 7 days after initiation of the lymphodepleting therapy. In some embodiments, the individual may be a human. In some embodiments, the NK cells in the composition are allogenic to the individual. In some embodiments, the method further includes administering exogenous cytokine support to facilitate expansion or persistence of the g-NK cells in vivo in the subject, optionally wherein the exogenous cytokine may be or includes IL-15.

### BRIEF DESCRIPTION OF THE DRAWINGS

FIG. 1A and FIG. 1B depict the expansion of g-NK cells expanded in the presence of 221.AEH or K562-mbIL15-41BBL feeder cells with or without IL-21 included in the NK cell media. FIG. 1A shows total NK cell counts. FIG. 1B shows g-NK cell counts after 21 days of expansion.

FIG. 2A and FIG. 2B depict daratumumab- and elotuzumab-mediated cytotoxic activity 21 days post-expansion of g-NK cells expanded in the presence of 221.AEH or K562-mbIL15-41BBL feeder cells with or without IL-21 included in the NK cell media. FIG. 2A shows g-NK cell cytotoxicity against the LP1 cell line. FIG. 2B shows g-NK cell cytotoxicity against the MM. 1S cell line.

FIG. 3A-3D depict daratumumab- and elotuzumab-mediated degranulation levels (CD107a^{pos}) of g-NK cells expanded in the presence of 221.AEH or K562-mbIL15-41BBL feeder cells with or without IL-21 included in the NK cell media. FIG. 3A shows g-NK cell degranulation levels 13 days post-expansion against the LP1 cell line. FIG. 3B shows g-NK cell degranulation levels 13 days post-expansion against the MM. 1S cell line. FIG. 3C shows g-NK cell degranulation levels 21 days post-expansion against the LP1 cell line. FIG. 3D shows g-NK cell degranulation levels 21 days post-expansion against the MM.1S cell line.

FIG. 4A-4D depict levels of perforin and granzyme B expression in g-NK cells expanded in the presence of 221.AEH or K562-mbIL15-41BBL feeder cells with or without IL-21 included in the NK cell media. FIG. 4A shows perforin and granzyme B expression 13 days post-expansion as percentages of g-NK cells. FIG. 4B shows total perforin and granzyme B expression 13 days post-expansion. FIG. 4C shows perforin and granzyme B expression 21 days post-expansion as percentages of g-NK cells. FIG. 4D shows total perforin and granzyme B expression 21 days post-expansion.

FIG. 5A-5D depict daratumumab- and elotuzumab-mediated Interferon-γ expression levels of g-NK cells expanded in the presence of 221.AEH or K562-mbIL15-41BBL feeder cells with or without IL-21 included in the NK cell media. FIG. 5A shows g-NK cell Interferon-γ expression levels 13 days post-expansion against the LP1 cell line. FIG. 5B shows g-NK cell Interferon-γ expression levels 13 days post-expansion against the MM.1S cell line. FIG. 5C shows g-NK cell Interferon-γ expression levels 21 days post-expansion against the LP1 cell line. FIG. 5D shows g-NK cell Interferon-γ expression levels 21 days post-expansion against the MM.1S cell line.

FIG. 6A-6D depict daratumumab- and elotuzumab-mediated TNF-α expression levels of g-NK cells expanded in the presence of 221.AEH or K562-mbIL15-41BBL feeder cells with or without IL-21 included in the NK cell media. FIG. 6A shows g-NK cell TNF-α expression levels 13 days post-expansion against the LP1 cell line. FIG. 6B shows g-NK cell TNF-α expression levels 13 days post-expansion against the MM.1S cell line. FIG. 6C shows g-NK cell TNF-α expression levels 21 days post-expansion against the LP1 cell line. FIG. 6D shows g-NK cell TNF-α expression levels 21 days post-expansion against the MM.1S cell line.

FIG. 7 depicts g-NK cell expansion of NK cells expanded for 15 days in the presence of various cytokine mixtures and concentrations.

FIG. 8A-8J show cell effector function of g-NK cells expanded in the presence of various cytokine mixtures and concentrations.

FIG. 8A and FIG. 8B depict daratumumab- and elotuzumab-mediated cytotoxic activity of g-NK cells expanded in the presence of various cytokine mixtures and concentrations. FIG. 8A shows g-NK cell cytotoxicity against the LP1 cell line. FIG. 8B shows g-NK cell cytotoxicity against the MM. 1S cell line.

FIG. 8C and FIG. 8D depict daratumumab- and elotuzumab-mediated degranulation levels (CD107a^{pos}) of g-NK cells expanded in the presence of various cytokine mixtures and concentrations. FIG. 8C shows g-NK cell degranulation levels against the LP1 cell line. FIG. 8D shows g-NK cell degranulation levels against the MM.1S cell line.

FIG. 8E and FIG. 8F depict levels of perforin and granzyme B expression in g-NK cells expanded in the presence of various cytokine mixtures and concentrations. FIG. 8E shows perforin and granzyme B expression as percentages of g-NK cells. FIG. 8F shows total perforin and granzyme B expression.

FIG. 8G and FIG. 8H depict daratumumab- and elotuzumab-mediated Interferon-γ expression levels of g-NK cells expanded in the presence of various cytokine mixtures and concentrations. FIG. 8G shows g-NK cell Interferon-γ expression levels against the LP1 cell line. FIG. 8H shows g-NK cell Interferon-γ expression levels against the MM.1S cell line.

FIG. 8I and FIG. 8J depict daratumumab- and elotuzumab-mediated TNF-α expression levels of g-NK cells expanded in the presence of various cytokine mixtures and concentrations. FIG. 8I shows g-NK cell TNF-α expression levels against the LP1 cell line. FIG. 34J shows g-NK cell TNF-α expression levels against the MM.1S cell line.

FIG. 9A-9L show expansion and cell effector function of g-NK cells expanded for 14 days in the presence of IL-21 compared to g-NK cells expanded without IL-21 (n=6).

FIG. 9A and FIG. 9B depict the expansion of g-NK cells expanded in the presence of IL-21 compared to g-NK cells expanded without IL-21. FIG. 9A shows g-NK cell percentage before and after expansion. FIG. 9B shows the number of g-NK cells expanded per 10 million NK cells. Values are mean±SE. #p<0.001 for comparisons of CD3^{neg}/CD57^{pos}+IL-21 expansions vs. CD3^{neg}/CD57^{pos} expansions without IL-21. \hat{ }p<0.05 for comparisons of CD3^{neg}/CD57^{pos} expansions vs. other CMV^{pos} expansions. *p<0.001 for comparisons of CMV^{pos} expansions vs. CMV^{neg} CD3^{neg} expansion.

FIG. 9C depicts comparison of the proportion of g-NK (% of total NK-cells from CMV+ (n=8) and CMV− donors (n=6) before and after expansion. FIG. 9D depicts comparison of the n-fold expansion rate of g-NK from CMV+ and CMV− donors. FIG. 9E provides representative flow plot of FcεR1γ vs. CD56 for a CMV+ donor. FIG. 9F provides representative histogram of FcεR1γ expression on CD3−/CD56+NK-cells for CMV+ and CMV− donors. Independent samples t-tests were used to determine the differences between CMV+ and CMV− donors before and after expansion (FIG. 9C and FIG. 9D). Values are mean±SE. *p<0.05, **p<0.01, and ***p<0.001.

FIG. 9G and FIG. 9H depict daratumumab- and elotuzumab-mediated cytotoxic activity 14 days post-expansion of g-NK cells expanded in the presence of IL-21 compared to g-NK cells expanded without IL-21. FIG. 9G shows g-NK cell cytotoxicity against the LP1 cell line. FIG. 9H shows g-NK cell cytotoxicity against the MM.1S cell line. Values are mean±SE. *p<0.05, **p<0.01, and ***p<0.001 for comparisons of CD3^{neg}/CD57^{pos}+IL-21 expansions vs. CD3^{neg}/CD57^{pos} expansions without IL-21.

FIG. 9I and FIG. 9J depict daratumumab- and elotuzumab-mediated degranulation levels (CD107a^{pos}) of g-NK cells expanded in the presence of IL-21 compared to g-NK cells expanded without IL-21. FIG. 9I shows g-NK cell degranulation levels 14 days post-expansion against the LP1 cell line. FIG. 9J shows g-NK cell degranulation levels 14 days post-expansion against the MM.1S cell line. Values are mean±SE. *p<0.05, **p<0.01, and ***p<0.001 for comparisons of CD3^{neg}/CD57^{pos}+IL-21 expansions vs. CD3^{neg}/CD57^{pos} expansions without IL-21.

FIG. 9K and FIG. 9L depict levels of perforin and granzyme B expression in g-NK cells expanded in the presence of IL-21 compared to g-NK cells expanded without IL-21. FIG. 9K shows perforin and granzyme B expression 14 days post-expansion as percentages of NK cells. FIG. 9L shows total perforin and granzyme B expression 14 days post-expansion. Values are mean±SE. *p<0.05, **p<0.01, and ***p<0.001 for comparisons of CD3^{neg}/CD57^{pos}+IL-21 expansions vs. CD3^{neg}/CD57^{pos} expansions without IL-21.

FIG. 9M depicts baseline expression of perforin (left) and granzyme B (right) in expanded g-NK cells than cNK cells (n=5). To compare effector perforin and granzyme B expression between g-NK and cNK, an independent sample 1-test was used. Values are mean±SE. Statistically significant differences from cNK cells are indicated by ***p<0.001.

FIG. 9N depicts representative histograms of perforin and granzyme B expression for g-NK and cNK cells.

FIG. 9O and FIG. 9P depict daratumumab- and elotuzumab-mediated Interferon-γ expression levels of g-NK cells expanded in the presence of IL-21 compared to g-NK cells expanded without IL-21. FIG. 9O shows g-NK cell Interferon-γ expression levels 14 days post-expansion against the LP1 cell line. FIG. 9P shows g-NK cell Interferon-γ expression levels 14 days post-expansion against the MM. 1S cell line. Values are mean±SE. *p<0.05, **p<0.01, and ***p<0.001 for comparisons of CD3^{neg}/CD57^{pos}+IL-21 expansions vs. CD3^{neg}/CD57^{pos} expansions without IL-21.

FIG. 9Q and FIG. 9R depict daratumumab- and elotuzumab-mediated TNF-α expression levels of g-NK cells expanded in the presence of IL-21 compared to g-NK cells expanded without IL-21. FIG. 9Q shows g-NK cell TNF-α expression levels 14 days post-expansion against the LP1 cell line. FIG. 9R shows g-NK cell TNF-α expression levels 14 days post-expansion against the MM.1S cell line. Values are mean±SE. *p<0.05, **p<0.01, and ***p<0.001 for comparisons of CD3^{neg}/CD57^{pos}+IL-21 expansions vs. CD3^{neg}/CD57^{pos} expansions without IL-21.

FIG. 9S depicts daratumumab- and elotuzumab-mediated interferon-γ expression levels of expanded g-NK cells compared to cNK cells against MM.1S cell line among different donors. FIG. 9T depicts daratumumab- and elotuzumab-mediated TNF-α expression levels of expanded g-NK cells compared to cNK cells against MM.1S cell line among different donors.

FIG. 10 depicts the expansion of g-NK expanded in the presence of an IL-21/anti-IL-21 complex (n=4). Values are mean±SE. #p<0.001 for comparisons of expansions with IL-21 vs. expansions with IL-21/anti-IL-21 complex.

FIG. 11A-11H show NK cell effector function of previously cryopreserved g-NK cells compared to that of freshly enriched g-NK cells (n=4). Values are mean±SE. #p<0.05 for comparisons of freshly enriched g-NK cells vs. previously cryopreserved g-NK cells.

FIG. 11A and FIG. 11B depict daratumumab- and elotuzumab-mediated degranulation levels (CD107a^{pos}) of previously cryopreserved g-NK cells compared to freshly enriched g-NK cells. FIG. 11A shows g-NK cell degranulation levels against the LP1 cell line. FIG. 11B shows g-NK cell degranulation levels against the MM.1S cell line.

FIG. 11C and FIG. 11D depict levels of perforin and granzyme B expression in previously cryopreserved g-NK cells compared to freshly enriched g-NK cells. FIG. 37C shows total perforin expression of g-NK cells. FIG. 11D shows total granzyme B expression of g-NK cells.

FIG. 11E and FIG. 11F depict daratumumab- and elotuzumab-mediated Interferon-γ expression levels of previously cryopreserved g-NK cells compared to freshly enriched g-NK cells. FIG. 11E shows g-NK cell Interferon-γ expression levels against the LP1 cell line. FIG. 11F shows g-NK cell Interferon-γ expression levels against the MM.1S cell line.

FIG. 11G and FIG. 11H depict daratumumab- and elotuzumab-mediated TNF-α expression levels of previously cryopreserved g-NK cells compared to freshly enriched g-NK cells. FIG. 37G shows g-NK cell TNF-α expression levels against the LP1 cell line. FIG. 11H shows g-NK cell TNF-α expression levels against the MM.1S cell line.

FIGS. 12A-C depict the persistence of cNK (cryopreserved) and g-NK (cryopreserved or fresh) cells in NSG mice after infusion of a single dose of 1×10⁷ expanded cells. FIG. 12A shows the number of cNK and g-NK cells in peripheral blood collected at days 6, 16, 26, and 31 post-infusion. FIG. 12B shows the number of NK cells present in the spleen at day 31 post-infusion, the time of sacrifice. FIG. 12C shows the number of NK cells present in the bone marrow the time of sacrifice. N=3 for all 3 arms. Values are mean±SE. *p<0.05 and ***p<0.001 for comparisons of cryopreserved cNK cells and fresh or cryopreserved g-NK cells.

FIGS. 13A-13D depict the expression of CD20 (the target for rituximab), CD38 (the target for daratumumab), and SLAMF7 (the target for elotuzumab) on g-NK and cNK. FIG. 13A shows the percentage of expanded g-NK cells, unexpanded NK-cells (CD3^{neg}/CD56^{pos}), and MM.1S cells expressing CD20. FIG. 13B shows the percentage of expanded g-NK cells, unexpanded NK-cells (CD3^{neg}/CD56^{pos}), and MM.1S cells expressing CD38. FIG. 13C shows the percentage of expanded g-NK cells, unexpanded NK-cells (CD3^{neg}/CD56^{pos}), and MM.1S cells expressing SLAMF7. FIG. 13D shows the percentage of cNK and g-NK expressing CD38 before and after expansion. N=3 for all arms.

FIG. 13E depicts the mean fluorescence intensity (MFI) for CD38^{pos} NK-cells before and after expansion (n=4).

FIG. 13F provides a representative histogram depicting the reduced CD38 expression of g-NK cells relative to cNK and MM.1S cells. Values are mean±SE. #p<0.001 for comparisons of g-NK cells vs. all other cells.

FIG. 13G depicts comparison of daratumumab-induced fratricide by expanded g-NK and cNK cells

FIGS. 14A-F show effect of treatment with cNK and daratumumab (cNK+Dara) or g-NK and daratumumab (g-NK+Dara) on tumor burden and survival in a mouse model of multiple myeloma. 5×10⁵ luciferase-labeled MM.1S human myeloma cells were injected intravenously (I.V.) into the tail veins of female NSG mice. Weekly, for a duration of five weeks, expanded NK cells were I.V. administered (6.0×10⁶ cells per mouse) and daratumumab was I.P. injected (10 μg per mouse) to NSG mice. FIG. 14A shows BLI imaging of mice twice per week at days 20, 27, 37, 41, 48, and 57 following tumor inoculation (left). Correspondent days post-treatment are shown on the right side of the figure. Colors indicate intensity of BLI (blue, lowest; red, highest). FIG. 14B shows tumor BLI (photons/second) over time in the g-NK+Dara group relative to the control and cNK+Dara groups. *p<0.05 for comparisons of g-NK and control or cNK groups. FIG. 14C shows percent survival over time, and arrows indicate administration of therapy with either cNK+Dara or g-NK+Dara. FIG. 14D presents the change in body weight over time of mice in the control, cNK+Dara, and g-NK+Dara groups. FIG. 14E depicts the number of CD138⁺ tumor cells present in bone marrow at the time of sacrifice in cNK+Dara- and g-NK+Dara-treated mice. ***p<0.001 for comparisons of g-NK and cNK cells. Values are mean±SE. FIG. 14F shows a representative flow plot using a gating strategy to resolve the presence of NK cells and tumor cells in the control group and in mice treated with either cNK+Dara or g-NK+Dara. N=8 for the control group, and N=7 for the g-NK or cNK group.

FIG. 14G presents all BLI images collected over the entire study for all control, cNK+Dara, and g-NK+Dara treated mice. Colors indicate intensity of BLI (blue, lowest; red, highest).

FIG. 14H depicts X-ray images obtained for all mice in the control, cNK+Dara, and g-NK+Dara groups prior to sacrifice. Arrows indicate bone fractures and deformities. The day of sacrifice is indicated under each mouse.

FIGS. 15A-C present comparative data of persistent NK cells in NSG mice following treatment with cNK+Dara or g-NK+Dara. All data present the amount of cells detected using flow cytometry at the time of sacrifice. FIG. 15A shows the number of cNK and g-NK cells in blood. FIG. 15B shows the number of NK cells present in the spleen. FIG. 15C shows the number of NK cells present in bone marrow. Values are mean±SE. ***p<0.001 for comparisons of g-NK and cNK cells.

FIGS. 16A-C show effect of treatment with cNK and rituximab or g-NK and rituximab on presence of Raji cells and survival in a mouse model of lymphoma. 5×10⁵ luciferase-labeled Raji lymphoma cells were injected intravenously (I.V.) into the tail veins of female NSG mice. Weekly, for a duration of seven weeks, expanded NK cells were I.V. administered (15×10⁶ cells per mouse) and rituximab was I.P. injected (200 μg per mouse) to NSG mice. FIG. 16A shows BLI imaging once per week at days 0, 7, 14, 21, 28, and 35 following tumor inoculation. FIG. 16B shows percent survival over time. FIG. 16C shows body weight change (%) over time.

### DETAILED DESCRIPTION

Provided herein is a method of treating multiple myeloma, where the method includes administering a composition of Natural Killer (NK) cells deficient in expression of FcRγ chain (g-NK cells) to a subject having a cancer. In some embodiments, the provided methods relate to methods and uses of compositions containing g-NK cells for treating multiple myeloma (MM). In some embodiments the provided methods relate to methods and uses of compositions containing g-NK cells for treating lymphoma. In some embodiments, the composition of g-NK cells may be administered once weekly for a predetermined number of doses. In some embodiments, the composition of g-NK cells may be administered in combination with an antibody therapeutic for treating the cancer, such as with an anti-CD38 antibody (e.g. daratumumab), with an anti-SLAMF7 antibody (e.g. elotuzumab), or with an anti-BCMA antibody (e.g., belantamab) for treating multiple myeloma or with an anti-CD20 antibody (e.g. rituximab), with an anti-CD19 antibody (e.g. tafasitamab or loncastuximab), or with an anti-CD30 antibody (e.g. brentuximab) for treating lymphoma.

Natural killer (NK) cells are innate lymphocytes important for mediating anti-viral and anti-cancer immunity through cytokine and chemokine secretion, and through the release of cytotoxic granules (Vivier et al. Science 331(6013):44-49 (2011); Caligiuri, Blood 112(3):461-469 (2008); Roda et al., Cancer Res. 66(1):517-526 (2006)). NK cells are effector cells that comprise the third largest population of lymphocytes and are important for host immuno-surveillance against tumor and pathogen-infected cells. However, unlike T and B lymphocytes, NK cells use germline-encoded activation receptors and are thought to have only a limited capacity for target recognition (Bottino et al., Curr Top Microbiol Immunol. 298:175-182 (2006); Stewart et al., Curr Top Microbiol Immunol. 298:1-21 (2006)).

Activation of NK cells can occur through the direct binding of NK cell receptors to ligands on the target cell, as seen with direct tumor cell killing, or through the crosslinking of the Fc receptor (CD16; also known as CD16a or FcγRIIIa) by binding to the Fc portion of antibodies bound to an antigen-bearing cell. Upon activation, NK cells produce cytokines and chemokines abundantly and at the same time exhibit potent cytolytic activity. NK cells are capable of killing tumor cells via antibody dependent cell mediated cytotoxicity (ADCC). In some cases, ADCC is triggered when receptors on the NK cell surface (such as CD16) recognize IgG1 or IgG3 antibodies bound to the surface of a cell. This triggers release of cytoplasmic granules containing perforin and granzymes, leading to target cell death. Because NK cells express the activating Fc receptor CD16, which recognizes IgG-coated target cells, target recognition is broadened (Ravetch & Bolland, Annu Rev Immunol. 19:275-290 (2001); Lanier Nat. Immunol. 9(5):495-502 (2008); Bryceson & Long, Curr Opin Immunol. 20(3):344-352 (2008)). ADCC and antibody-dependent cytokine/chemokine production are primarily mediated by NK cells.

CD16 also exists in a glycosylphosphatidylinositol-anchored form (also known as FcγRIIIB or CD16B). It is understood that reference to CD16 herein is with reference to the CD16a form that is expressed on NK cells and that is involved in antibody-dependent responses (such as NK cell-mediated ADCC), and it is not meant to refer to the glycosylphosphatidylinositol-anchored form.

The CD16 receptor is able to associate with adaptors, the ζ chain of the TCR-CD3 complex (CD3ζ) and/or the FcRγ chain, to transduce signals through immunoreceptor tyrosine-based activation motifs (ITAMs). In some aspects, CD16 engagement (CD16 crosslinking) initiates NK cell responses via intracellular signals that are generated through one, or both, of the CD16-associated adaptor chains, FcRγ or CD3ζ. Triggering of CD16 leads to phosphorylation of the γ or ζ chain, which in turn recruits tyrosine kinases, syk and ZAP-70, initiating a cascade of signal transduction leading to rapid and potent effector functions. The most well-known effector function is the release of cytoplasmic granules carrying toxic proteins to kill nearby target cells through the process of antibody-dependent cellular cytotoxicity. CD16 crosslinking also results in the production of cytokines and chemokines that, in turn, activate and orchestrate a series of immune responses.

This release of cytokines and chemokines can play a role in the anti-cancer activity of NK cells in vivo. NK cells also have small granules in their cytoplasm containing perforin and proteases (granzymes). Upon release from the NK cell, perforin forms pores in the cell membrane of targeted cells through which the granzymes and associated molecules can enter, inducing apoptosis. The fact that NK cells induce apoptosis rather than necrosis of target cells is significant-necrosis of a virus-infected cell would release the virions, whereas apoptosis leads to destruction of the virus inside the cells.

A specialized subset of NK cells lacking the FcRγ adaptor protein, also known as g-NK cells, are able to mediate robust ADCC responses (see e.g. published Patent Appl. No. US2013/0295044). The mechanism for increased responses may be due to changes in epigenetic modification that influence the expression of the FcRγ. The g-NK cells express the signaling adaptor ζ chain abundantly, but are deficient in the expression of the signaling adaptor γ chain. Compared to conventional NK cells, these γ-deficient g-NK cells exhibit dramatically enhanced activity when activated by antibodies. For example, the g-NK cells can be activated by antibody-mediated crosslinking of CD16 or by antibody-coated tumor cells. In some aspects, the g-NK cells produce greater amounts of cytokines (e.g. IFN-γ or TNF-α) and chemokines (e.g. MIP-1α, MIP-1β, and RANTES) and/or display higher degranulation responses than conventional NK cells expressing the γ chain. The g-NK cells provide high expression of Granzyme B, a component of natural killer cell cytotoxic machinery. Moreover, the g-NK cells have a prolonged lifespan, compared to conventional NK cells, and their presence is maintained long-term. In some embodiments, g-NK cells are functionally and phenotypically stable.

In some embodiments, g-NK cells are more effective in eliciting ADCC responses than conventional NK cells, e.g. NK cells that are not deficient in the γ chain. In some embodiments, g-NK cells are more effective in eliciting cell-mediated cytotoxicity than are conventional NK cells even in the absence of antibody. In some cases, ADCC is a mechanism of action of therapeutic antibodies, including anti-cancer antibodies. In some aspects, cell therapy by administering NK cells can be used in concert with antibodies for therapeutic and related purposes.

For instance, certain therapeutic monoclonal antibodies, such as daratumumab targeting CD38, elotuzumab targeting SLAMF7, belantamab targeting BCMA are FDA approved for treating disease, such as multiple myeloma (MM). Other therapeutic monoclonal antibodies, such as rituximab targeting CD20, tafasitamab or loncastuximab targeting CD19, and brentuximab targeting CD30 are FDA approved for treating disease, such as lymphoma. While clinical responses of therapeutic antibodies are promising, they are often not ideal. For example, while initial clinical responses have generally been encouraging, particularly for daratumumab, essentially all patients eventually develop progressive disease. Thus, there is a significant need for new strategies to either drive deeper remissions or overcome resistance to these agents. The provided embodiments, including compositions, address these needs.

Provided herein are methods involving combined administration of a composition containing g-NK cells, e.g. as produced by the provided methods, and an antibody, e.g. an anti-cancer antibody. In some embodiments, antibody-directed targeting of g-NK cells leads to improved outcomes for patients due to the improved affinity, cytotoxic and/or cytokine-mediated effect functions of the g-NK cell subset.

In some embodiments, a potential mechanism of action of monoclonal antibodies as therapeutics is by an anti-tumor action due to complement-dependent cytotoxicity, antibody-dependent cellular phagocytosis, and/or antibody-dependent cellular cytotoxicity. In some cases, it is contemplated that ADCC, mediated by NK-cells can potently eliminate antibody-bound tumors cells, particularly in the case of a multiple myeloma (MM) tumors.

NK-cells are activated when the Fc portion of an antibody binds their Fc receptor (FcγRIIIa or CD16a) and triggers activation and degranulation through a process involving the adapter proteins CD3ζ and FcεR1γ. Efforts to enhance the clinical ADCC response to antibodies, including MM antibodies, have been challenging because NK-cells also express CD38 and SLAMF7 (the targets for example of daratumumab and elotuzumab, respectively). High CD38 expression particularly results in rapid depletion of NK cells early in the daratumumab treatment course, largely eliminating this source of innate immune cells which could potentially drive even more complete tumor eradication.

The provided g-NK cells and compositions containing the same, such produced by the provided methods, exhibit a number of features that overcome these problems. g-NK cells are a relatively rare subset as g-NK cells are only detectable at levels of ˜3-10% of total NK-cells in only 25-30% of CMV seropositive individuals. The provided methods relate to methods that are particularly robust in the ability to expand and enrich g-NK cells, thus allowing sufficient expansion required for in vivo use.

In some embodiments, the g⁻NK cells produce significantly greater amounts of a cytokine than natural killer cells that do express FcRγ. In another embodiment, the cytokine is interferon-gamma (IFN-γ), tumor necrosis factor-α (TNF-α), or a combination thereof. In one embodiment, the g⁻NK cells produce significantly greater amounts of a chemokine. In one embodiment, the chemokine is MIP-1α, MIP-1β or a combination thereof. In another embodiment, the g⁻NK cells produce the cytokine or the chemokine upon stimulation through the Fc receptor CD16.

g-NK cells represent a relatively small percentage of NK cells in the peripheral blood, thereby limiting the ability to use these cells in therapeutic methods. In particular, to utilize g-NK cells in the clinic, a high preferential expansion rate is necessary because g-NK cells are generally a rare population. Other methods for expanding NK cells are able to achieve thousand-fold 14-day NK-cell expansion rates, but they yield low differentiation, NKG2C^{neg}, FceRIγ^{pos} (FcRγ^{pos}) NK-cells (Fujisaki et al. (2009) Cancer Res., 69:4010-4017; Shah et al. (2013) PLOS One, 8:e76781). Further, it is found herein that an expansion optimized for expanding NK cells that phenotypically overlap with g-NK cells does not preferentially expand g-NK cells to amounts that would support therapeutic use. In particular, it has been previously reported that NKG2C^{pos} NK-cells, which exhibit phenotypic overlap with g-NK cells, can be preferentially expanded using HLA-E transfected 221.AEH cells and the inclusion of IL-15 in the culture medium (Bigley et al. (2016) Clin. Exp. Immunol., 185:239-251). Culture with such HLA-expressing cells that constitutively expresses HLA-E pushes the NK-cells in the direction of an NKG2C^{pos}/NKG2A^{neg} phenotype (NKG2C is the activating receptor for HLA-E, while NKG2A is the inhibitory receptor for HLA-E). It was thought that because such cells include within it the g-NK, such methods would be sufficient to expand g-NK cells. However, this method does not achieve robust expansion of g-NK cells.

Methods described herein are able to produce NK cell compositions enriched in g-NK cells that overcome these limitations. The provided methods utilize a greater ratio of HLA-E+ feeder cells deficient in HLA class I and HLA class II, for instance 221.AEH cells, to NK− cells compared to previous methods. In particular, previous methods have used a lower ratio of 221.AEH cells, such as a ratio of 10:1 NK cell to 221.AEH ratio. It is found herein that a greater ratio of HLA-E-expressing feeder cells, such as 221.AEH cells, results in overall expansion that is greater and more skewed towards the g-NK phenotype. In some embodiments, the greater ratio of HLA-E+ feeder cells, for instance 221.AEH cells, is possible by irradiating the feeder cells. In some aspects, the use of irradiated feeder cell lines also is advantageous because it provides for a method that is GMP compatible. The inclusion of any of recombinant IL-2, IL-7, IL-15, IL-12, IL-18, IL-21, IL-27, or combinations thereof during the expansion also is found to support robust expansion. In particular embodiments of the provided methods at least one recombinant cytokine is IL-2. In some embodiments, there are two or more recombinant cytokines wherein at least one recombinant cytokine is IL-2 and at least one recombinant cytokine is IL-21.

Provided methods herein are based on the finding that culture of NK cells for expansion in the presence of IL-21 supercharges the NK cells to produce cytokines or effector molecules such as perforin and granzyme B. Compositions containing NK cells produced by the expanded processes herein are highly functional, exhibit robust proliferation, and work well even after they are cryofrozen without rescue. For example, the NK cells produced by the provided processes when expanded in the presence of IL-21 not only exhibit strong ADCC activity, but they also exhibit antibody-independent cytotoxic activities. For example, effector molecules (e.g. perforin and granzymes) are spontaneously present in NK cells expanded by the provided methods, thereby providing cells that exhibit high cytotoxic potential. As shown herein, NK cell composition produced by the provided processes that include IL-21 (e.g. IL-2, IL-15 and IL-21) not only exhibit a higher percentage of NK cells positive for perforin or granzyme B than NK cell compositions produced by a process that only includes IL-2 without addition of IL-21, but they also exhibit a higher average level or degree of expression of the molecules in the cells. Further, the NK cell composition produced by the method provided herein that includes IL-21 (e.g. IL-2, IL-15 and IL-12) also result in g-NK cell compositions that exhibit substantial effector activity, including degranulation and ability to express more IFN-gamma and TNF-alpha, in response to target cells when combined with an antibody (e.g. daratumumab) against the target antigen (e.g. CD38). This functional activity is highly preserved even after cryopreservation and thawing of expanded NK cells. The marked increases in cytolytic enzymes, as well as more robust activation phenotypes, underpin the enhanced capacity of expanded g-NK cells to induce apoptosis of tumor targets when engaged with antibody via CD16-crosslinking. The marked antibody-independent effector phenotype also supports potential utility of the g-NK cells as a monotherapy.

Further, findings herein also demonstrate the potential of the provided NK cells expanded in the presence of IL-21 to persist and proliferate well for an extended period of time, which is greater than cells expanded, for example, only in the presence of IL-2 without the addition of IL-21. Furthermore, results showed that cryopreserved g-NK cells persisted at comparable levels to fresh g-NK cells. This significantly improved persistence emphasizes the potential utility of fresh or cryopreserved g-NK as an off-the-shelf cellular therapy to enhance antibody-mediated ADCC. This finding of improved persistence is advantageous, since clinical utility of many NK cell therapies has been hampered by limited NK cell persistence.

Moreover, results herein demonstrate the surprising finding that g-NK cells express low levels of CD38, which is the target of therapeutic antibodies such as daratumumab. A problem with many existing NK cell therapies against certain target antigens, such as CD38, is that the NK cells may express the target antigen thereby resulting in “fratricide,” whereby ADCC activity leads to elimination of NK cells in addition to tumor. In fact, other reported NK cell compositions are reported to express a high percentage (e.g. >90%) of CD38high NK cells. In contrast, the findings herein demonstrate that the percentage of CD38pos cells was markedly lower on donor-isolated g-NK cells and on g-NK cells expanded therefrom, than on conventional NK cells or MM target cell line. The lower CD38 expression led to markedly reduced anti-CD38 (e.g. daratumumab)-mediated fratricide by the g-NK cells related to the conventional NK cell. These results support utility of the provided g-NK cell compositions to confer enhanced antibody anti-tumor activity in MM without suffering from fratricide-related depletion. The results further suggest that the g-NK cell composition could be optimal for daratumumab refractory patients as expanded g-NK cells are resistant to daratumumab-induced fratricide and enhance daratumumab-specific cell cytotoxicity against even dimly CD38 expressing myeloma cells.

Moreover, the above activities as demonstrated by the g-NK cells can be achieved without the need to further engineer cells to enhance antibody efficacy. For example, CD38-knockout NK cell lines have been created to avoid daratumumab fratricide and NK cell lines with non-cleavable CD16 have been developed to enhance anti-tumor ADCC. However, potential drawbacks for clinical use include need for genetic engineering and irradiation of immortalized cell lines.

The superiority of the provided g-NK cell compositions, including those produced by the provided methods, was further demonstrated in studies evaluating the in vivo activity of g-NK cells. Activity in an exemplary mouse model of MM showed that the g-NK cells in combination with antibody (e.g. daratumumab) eliminated myeloma tumor burden in a majority of the mice with sustained and significant tumor regression. These results underscore the superiority of g-NK cells, particularly compared to conventional NK cells that are FcεR1γ+, for enhancing antibody effects in vivo and support the therapeutic potential of this NK cell therapy. The high persistence and enhanced survival of the NK cells and their resistance to fratricide in this model may support the superior anti-tumor effects and persistence of the g-NK cells.

It also is found that enrichment of NK cells from a cell sample prior to the expansion method, such as by enrichment for CD16 or CD57 cells prior to expansion, further substantially increases the amount of g-NK cell expansion that can be achieved compared to methods that initially enrich NK cells based on CD3 depletion alone. In another embodiment, another enrichment that can be carried out prior to expansion is enriching for NK cells by positive selection for CD56 and negative selection or depletion for CD38. In a further embodiment, another enrichment that can be carried out prior to expansion is enriching for NK cells by positive selection for CD56 followed by negative selection or depletion for NKG2A^{neg} and negative selection or depletion for CD161^{neg}. In another embodiment, another enrichment that can be carried out prior to expansion is enriching for NK cells by positive selection for CD57 followed by negative selection or depletion for NKG2A and/or positive selection for NKG2C. In another embodiment, another enrichment that can be carried out prior to expansion is enriching for NK cells by positive selection for CD56 followed by negative selection or depletion for NKG2A and/or positive selection for NKG2C. In any of such embodiments, enrichment for NKG2C^{pos} and/or NKG2A^{neg} NK cells can be carried out after expansion.

In any of such embodiments, the enriched NK cells can be enriched from a cell sample containing NK cells, such as from peripheral blood mononuclear cells (PBMCs). In some embodiments, prior to the enrichment for NK cells from the cell sample, T cells can be removed by negative selection or depletion for CD3. In any of such embodiments, the enriched NK cells can be enriched from a biological sample from a human subject containing NK cells (e.g. PBMCs) with a relatively high proportion of g-NK cells, for instance from a human subject selected for having a high percentage of g-NK cells among NK cells. In any of such embodiments, the enriched NK cells can be enriched from a biological sample from a human subject containing NK cells, e.g. PBMCs, in which the sample contains a relatively high proportion of NKG2C^{pos} NK cells (e.g. at or about or greater than 20% NKG2C^{pos} NK cells) and/or NKG2A^{neg} NK cells (e.g. at or about or greater than 70% NKG2A^{neg} NK cells). In any of such embodiments, the enriched NK cells can be enriched from a biological sample from a human subject containing NK cells, e.g. PBMCs, in which the sample contains a relatively high proportion of NKG2C^{pos} NK cells (e.g. at or about or greater than 20% NKG2C^{pos} NK cells) and NKG2A^{neg} NK cells (e.g. at or about or greater than 70% NKG2A^{neg} NK cells).

Together, the provided approach for expanding g-NK cells can achieve expansion of NK cells that exceeds over 1 billion cells, and in some cases up to 8 billion or more, from an initial 10 million enriched NK cells at the initiation of culture. In particular, the provided methods can result in high-yield (>1000 fold) expansion rates with maintained or, in some cases, increased functionality of the g-NK cells after expansion. In some embodiments, the provided methods can result in a g-NK cell population expressing high levels of perforin and granzyme B. Further, it is found that the provided methods are sufficient to expand previously frozen NK cells, which is not commonly achieved by many existing methods that involve rescue of thawed NK cells. In some embodiments, this is achieved by increasing the duration of the expansion protocol. In some embodiments, this is achieved by decreasing the ratio of HLA-E+ feeder cells to NK cells, e.g. to about 1:1 221.AEH to NK cells. In some embodiments, this is achieved with the inclusion of any of recombinant IL-2, IL-7, IL-15, IL-12, IL-18, IL-21, IL-27, or combinations thereof during the expansion. In particular embodiments, at least one recombinant cytokine is IL-2. In some embodiments, expansion is carried out in the presence of two or more recombinant cytokines in which at least one is recombinant IL-21 and at least one is recombinant IL-2. As shown herein, the provided methods yield g-NK cells that exhibit potent antibody-dependent cell-mediated cytotoxicity (ADCC) as well as antibody-independent cell-mediated cytotoxicity, supporting the utility of such cells for therapeutic applications.

As shown here, the provided g-NK cells and compositions containing the same, such produced by the provided methods, can be used for cancer therapy. In some aspects, the provided studies demonstrate that g-NK cells have markedly enhanced ADCC/effector functions when combined with a target antibody against a tumor antigen (e.g. anti-myeloma), and adoptive transfer of expanded g-NK cells eliminates tumor burden in vivo when combined with a therapeutic antibody (e.g. daratumumab). Importantly, adoptive transfer of allogeneic NK-cells does not result in severe graft-versus-host (GVHD), and thus such a cell therapy, including in combination with an antibody as an antibody-directed NK-cell therapy, can be given in an “off-the-shelf” manner for clinical use.

All references cited herein, including patent applications, patent publications, and scientific literature and databases, are herein incorporated by reference in their entirety for all purposes to the same extent as if each individual reference were specifically and individually indicated to be incorporated by reference.

For clarity of disclosure, and not by way of limitation, the detailed description is divided into the subsections that follow. The section headings used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

As used herein, “antibody” refers to immunoglobulins and immunoglobulin fragments, whether natural or partially or wholly synthetically, such as recombinantly, produced, including any fragment thereof containing at least a portion of the variable heavy chain and/or light chain region of the immunoglobulin molecule that is sufficient to form an antigen binding site and, when assembled, to specifically bind antigen. Hence, an antibody includes any protein having a binding domain that is homologous or substantially homologous to an immunoglobulin antigen-binding domain (antibody combining site). Typically, antibodies minimally include all or at least a portion of the variable heavy (V_{H}) chain and/or the variable light (V_{L}) chain. In general, the pairing of a V_{H} and V_{L} together form the antigen-binding site, although, in some cases, a single V_{H} or V_{L} domain is sufficient for antigen-binding. The antibody also can include all or a portion of the constant region. Reference to an antibody herein includes full-length antibody and antigen-binding fragments. The term “immunoglobulin” (Ig) is used interchangeably with “antibody” herein.

The terms “full-length antibody,” “intact antibody” or “whole antibody” are used interchangeably to refer to an antibody in its substantially intact form, as opposed to an antibody fragment. A full-length antibody is an antibody typically having two full-length heavy chains (e.g., VH-CH1-CH2-CH3 or VH-CH1-CH2-CH3-CH4) and two full-length light chains (VL-CL) and hinge regions, such as antibodies produced from mammalian species (e.g. human, mouse, rat, rabbit, non-human primate, etc.) by antibody secreting B cells and antibodies with the same domains that are produced synthetically. Specifically whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, the antigen binding and/or the variable region of the intact antibody. Antibody fragments, include, but are not limited to, Fab fragments, Fab′ fragments, F(ab′)₂ fragments, Fv fragments, disulfide-linked Fvs (dsFv), Fd fragments, Fd′ fragments; diabodies; linear antibodies (see U.S. Pat. No. 5,641,870, Example 2; Zapata et al., Protein Eng. 8(10): 1057-1062 [1995]); single-chain antibody molecules, including single-chain Fvs (scFv) or single-chain Fabs (scFab); antigen-binding fragments of any of the above and multispecific antibodies from antibody fragments. For purposes herein, an antibody fragment typically includes one that is sufficient to engage or crosslink CD16 on the surface of an NK cell.

The term “autologous” refers to cells or tissues originating within or taken from an individual's own tissues. For example, in an autologous transfer or transplantation of NK cells, the donor and recipient are the same person.

The term “allogeneic” refers to cells or tissues that belong to or are obtained from the same species but that are genetically different, and which, in some cases, are therefore immunologically incompatible. Typically, the term “allogeneic” is used to define cells that are transplanted from a donor to a recipient of the same species.

The term “enriched” with reference to a cell composition refers to a composition in which there is an increase in the number or percentage of the cell type or population as compared to the number or percentage of the cell type in a starting composition of the same volume, such as a starting composition directly obtained or isolated from a subject. The term does not require complete removal of other cells, cell type, or populations from the composition and does not require that the cells so enriched be present at or even near 100% in the enriched composition.

The term “expression” refers to the process by which a polynucleotide is transcribed from a DNA template (such as into an mRNA or other RNA transcript) and/or the process by which a transcribed mRNA is subsequently translated into peptide, polypeptides or proteins. Transcripts and encoded polypeptides may be collectively referred to as “gene product.” If the polynucleotide is derived from genomic DNA, expression may include splicing of the mRNA in a eukaryotic cell.

The term “heterologous” with reference to a protein or nucleic acid refers to a protein or nucleic acid originating from a different genetic source. For example, a protein or nucleic acid that is heterologous to a cell originates from an organism or individual other than the cell in which it is expressed.

As used herein, the term “introducing” encompasses a variety of methods of introducing DNA into a cell, either in vitro or in vivo, such methods including transformation, transduction, transfection (e.g. electroporation), and infection. Vectors are useful for introducing DNA encoding molecules into cells. Possible vectors include plasmid vectors and viral vectors. Viral vectors include retroviral vectors, lentiviral vectors, or other vectors such as adenoviral vectors or adeno-associated vectors.

The term “composition” refers to any mixture of two or more products, substances, or compounds, including cells or antibodies. It may be a solution, a suspension, liquid, powder, a paste, aqueous, non-aqueous or any combination thereof. The preparation is generally in such form as to permit the biological activity of the active ingredient (e.g. antibody) to be effective.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, combination refers to any association between or among two or more items. The combination can be two or more separate items, such as two compositions or two collections, can be a mixture thereof, such as a single mixture of the two or more items, or any variation thereof. The elements of a combination are generally functionally associated or related.

As used herein, a kit is a packaged combination that optionally includes other elements, such as additional agents and instructions for use of the combination or elements thereof, for a purpose including, but not limited to, therapeutic uses.

As used herein, the term “treatment” or “treating” refers to clinical intervention designed to alter the natural course of the individual or cell being treated during the course of clinical pathology. Desirable effects of treatment include decreasing the rate of disease progression, ameliorating or palliating the disease state, and remission or improved prognosis. An individual is successfully “treated”, for example, if one or more symptoms associated with a disorder (e.g., an eosinophil-mediated disease) are mitigated or eliminated. For example, an individual is successfully “treated” if treatment results in increasing the quality of life of those suffering from a disease, decreasing the dose of other medications required for treating the disease, reducing the frequency of recurrence of the disease, lessening severity of the disease, delaying the development or progression of the disease, and/or prolonging survival of individuals.

An “effective amount” refers to at least an amount effective, at dosages and for periods of time necessary, to achieve the desired or indicated effect, including a therapeutic or prophylactic result. An effective amount can be provided in one or more administrations. A “therapeutically effective amount” is at least the minimum dose of cells required to effect a measurable improvement of a particular disorder. In some embodiments, a therapeutically effective amount is the amount of a composition that reduces the severity, the duration and/or the symptoms associated with cancer, viral infection, microbial infection, or septic shock in an animal. A therapeutically effective amount herein may vary according to factors such as the disease state, age, sex, and weight of the patient. A therapeutically effective amount may also be one in which any toxic or detrimental effects of the antibody are outweighed by the therapeutically beneficial effects. A “prophylactically effective amount” refers to an amount effective, at the dosages and for periods of time necessary, to achieve the desired prophylactic result. Typically but not necessarily, since a prophylactic dose is used in subjects prior to or at the earlier stage of disease, the prophylactically effective amount can be less than the therapeutically effective amount.

As used herein, an “individual” or a “subject” is a mammal. A “mammal” for purposes of treatment includes humans, domestic and farm animals, and zoo, sports, or pet animals, such as dogs, horses, rabbits, cattle, pigs, hamsters, gerbils, mice, ferrets, rats, cats, etc. In some embodiments, the individual or subject is human.

### II. Methods of Treatment

Provided herein are compositions and methods relating to the provided cell compositions comprising g-NK cells described herein for use in treating diseases or conditions in a subject. In some embodiments, provided herein is a method of treating a condition in an individual, comprising administering any of the provided compositions, such as compositions comprising g-NK cells, to an individual in need thereof. In particular embodiments, the composition is produced by the methods provided herein. Such methods and uses include therapeutic methods and uses, for example, involving administration of the therapeutic cells, or compositions containing the same, to a subject having a disease, condition, or disorder. In some cases, the disease or disorder is a tumor or cancer. In some embodiments, the disease or disorder is a virus infection. In some embodiments, the cells or pharmaceutical composition thereof is administered in an effective amount to effect treatment of the disease or disorder. Uses include uses of the cells or pharmaceutical compositions thereof in such methods and treatments, and in the preparation of a medicament in order to carry out such therapeutic methods. In some embodiments, the methods thereby treat the disease or condition or disorder in the subject.

In one aspect, disclosed herein is a method of treating multiple myeloma, where the method includes administering a composition of Natural Killer (NK) cells deficient in expression of FcRγ chain (g-NK cells) to a subject having multiple myeloma (MM), wherein the composition of g-NK cells may be administered once weekly for a predetermined number of doses.

In one aspect, disclosed herein is a method of treating lymphoma, where the method includes administering a composition of Natural Killer (NK) cells deficient in expression of FcRγ chain (g-NK cells) to a subject having lymphoma, wherein the composition of g-NK cells may be administered once weekly for a predetermined number of doses.

In some embodiments, the predetermined number of once weekly doses is one dose, two doses, three doses, four doses, five doses, six doses, seven doses, eight doses, nine doses, ten doses, eleven doses or twelve doses. In some embodiments, the once weekly doses are administered for 4 weeks, 6 weeks, 8 weeks, 10 weeks, 12 weeks, 16 weeks, 20 weeks, 24 weeks, 28 weeks, 32 weeks, 36 weeks or more. In some embodiments, six (6) once weekly doses of the g-NK cell composition is administered. In some embodiments, the once weekly doses are administered in consecutive weeks.

In some embodiments the once weekly dose is administered in a cycling regimen. In some embodiments, the cycling regimen is a 14 day cycle. In some embodiments, the once weekly dose is administered two times in the 14 day cycle. In some embodiments, the 14 day cycle is repeated twice. In some embodiments, the 14 day cycle is repeated three times.

In some embodiments, the methods of treatment or uses involve administration of an effective amount of a composition containing a composition of expanded NK cells produced by the provided method to an individual. In some embodiments, from at or about 10⁵ to at about 10¹², or from at or about 10⁵ and at or about 10⁸, or from at or about 10⁶ and at or about 10¹², or from at or about 10⁸ and at or about 10¹¹, or from at or about 10⁹ and at or about 10¹⁰ of such expanded NK cells is administered to an individual subject. In some embodiments, a dose of cells containing at or greater than at or about 10⁵, at or greater than at or about 10⁶, at or greater than at or about 10⁷, at or greater than at or about 10⁸, at or greater than at or about 10⁹, at or greater than at or about 10¹⁰, at or greater than at or about 10¹¹, or at or greater than at or about 10¹² of such expanded NK cells are administered to the individual. In some embodiments, from or from about 10⁶ to 10¹⁰ of such expanded NK cells per kg are administered to the subject.

In some embodiments, the methods of treatment or uses involve administration of an effective amount of any of the provided NK cell compositions, including any as described herein, to an individual. In some embodiments, from at or about 10⁵ to at about 10¹², or from at or about 10⁵ and at or about 10⁸, or from at or about 10⁶ and at or about 10¹², or from at or about 10⁸ and at or about 10¹¹, or from at or about 10⁹ and at or about 10¹⁰ of NK cells from any of the provided compositions is administered to an individual subject. In some embodiments, a dose of cells containing at or greater than at or about 10⁵, at or greater than at or about 10⁶, at or greater than at or about 10⁷, at or greater than at or about 10⁸, at or greater than at or about 10⁹, at or greater than at or about 10¹⁰, at or greater than at or about 10¹¹, or at or greater than at or about 10¹² of NK cells from any of the provided compositions are administered to the individual. In some embodiments, from or from about 10⁶ to 10¹⁰ of NK cells of any of the provided compositions per kg are administered to the subject.

In some embodiments, each dose of g-NK cells may be from at or about from at or about 1×10⁸ cells to at or about 50×10⁹ cells of the g-NK cell composition. In some embodiments, each dose of g-NK cells may be or may be about 5×10⁸ cells of the g-NK cell composition. In some embodiments, each dose of g-NK cells may be or may be about 5×10⁹ cells of the g-NK cell composition. In some embodiments, each dose of g-NK cells may be or may be about 10×10⁹ cells of the g-NK cell composition.

In some embodiments, the methods of treatment comprises administering an effective amount of a composition containing g-NK cells to an individual. In some embodiments, from at or about 10⁵ to at about 10¹² g-NK cells, or from at or about 10⁵ and at or about 10⁸ g-NK cells, or from at or about 10⁶ and at or about 10¹² g-NK cells, or from at or about 10⁸ and at or about 10¹¹ g-NK cells, or from at or about 10⁹ and at or about 10¹⁰ g-NK cells. In some embodiments, a dose of cells containing at or greater than at or about 10⁵ g-NK cells, at or greater than at or about 10⁶ g-NK cells, at or greater than at or about 10⁷ g-NK cells, at or greater than at or about 10⁸ g-NK cells, at or greater than at or about 10⁹ g-NK cells, at or greater than at or about 10¹⁰ g-NK cells, at or greater than at or about 10¹¹ g-NK cells, or at or greater than at or about 10¹² g-NK cells are administered to the individual. In some embodiments, from or from about 10⁶ to 10¹⁰ g-NK cells/kg are administered to the subject.

In some embodiments, the dose for administration in accord with any of the provided methods of treatment or uses is from at or about 1×10⁵ cells/kg to at or about 1×10⁷ cells/kg, such as from at or about 1×10⁵ cells/kg to at or about 7.5×10⁶ cells/kg, from at or about 1×10⁵ cells/kg to at or about 5×10⁶ cells/kg, from at or about 1×10⁵ cells/kg to at or about 2.5×10⁶ cells/kg, from at or about 1×10⁵ cells/kg to at or about 1×10⁶ cells/kg, from at or about 1×10⁵ cells/kg to at or about 7.5×10⁵ cells/kg, from at or about 1×10⁵ cells/kg to at or about 5×10⁵ cells/kg, from at or about 1×10⁵ cells/kg to at or about 2.5×10⁵ cells/kg, from at or about 2.5×10⁵ cells/kg to at or about 1×10⁷ cells/kg, from at or about 2.5×10⁵ cells/kg to at or about 7.5×10⁶ cells/kg, from at or about 2.5×10⁵ cells/kg to at or about 5×10⁶ cells/kg, from at or about 2.5×10⁵ cells/kg to at or about 2.5×10⁶ cells/kg, from at or about 2.5×10⁵ cells/kg to at or about 1×10⁶ cells/kg, from at or about 2.5×10⁵ cells/kg to at or about 7.5×10⁵ cells/kg, from at or about 2.5×10⁵ cells/kg to at or about 5×10⁵ cells/kg, from at or about 5×10⁵ cells/kg to at or about 1×10⁷ cells/kg, from at or about 5×10⁵ cells/kg to at or about 7.5×10⁶ cells/kg, from at or about 5×10⁵ cells/kg to at or about 5×10⁶ cells/kg, from at or about 5×10⁵ cells/kg to at or about 2.5×10⁶ cells/kg, from at or about 5×10⁵ cells/kg to at or about 1×10⁶ cells/kg, from at or about 5×10⁵ cells/kg to at or about 7.5×10⁵ cells/kg, from at or about 1×10⁶ cells/kg to at or about 1×10⁷ cells/kg, from at or about 1×10⁶ cells/kg to at or about 7.5×10⁶ cells/kg, from at or about 1×10⁶ cells/kg to at or about 5×10⁶ cells/kg, from at or about 1×10⁶ cells/kg to at or about 2.5×10⁶ cells/kg, from at or about 2.5×10⁶ cells/kg to at or about 1×10⁷ cells/kg, from at or about 2.5×10⁶ cells/kg to at or about 7.5×10⁶ cells/kg, from at or about 2.5×10⁶ cells/kg to at or about 5×10⁶ cells/kg, from at or about 5×10⁶ cells/kg to at or about 1×10⁷ cells/kg, from at or about 5×10⁶ cells/kg to at or about 7.5×10⁶ cells/kg, or from at or about 7.5×10⁶ cells/kg to at or about 1×10⁷ cells/kg. In some embodiments, the dose for administration is from at or about 1×10⁵ cells/kg to at or about 1×10⁸ cells/kg, such as from at or about 2.5×10⁵ cells/kg to at or about 1×10⁸ cells/kg, from at or about 5×10⁵ cells/kg to at or about 1×10⁸ cells/kg, from at or about 7.5×10⁵ cells/kg to at or about 1×10⁸ cells/kg, from at or about 1×10⁶ cells/kg to at or about 1×10⁸ cells/kg, from at or about 2.5×10⁶ cells/kg to at or about 1×10⁸ cells/kg, from at or about 5×10⁶ cells/kg to at or about 1×10⁸ cells/kg, from at or about 7.5×10⁶ cells/kg to at or about 1×10⁸ cells/kg, from at or about 1×10⁷ cells/kg to at or about 1×10⁸ cells/kg, from at or about 2.5×10⁷ cells/kg to at or about 1×10⁸ cells/kg, from at or about 5×10⁷ cells/kg to at or about 1×10⁸ cells/kg, or from at or about 7.5×10⁷ cells/kg to at or about 1×10⁸ cells/kg.

In some embodiments, the dose is given as the number of g-NK cells or an NK cell subset that is associated with or includes a surrogate marker for g-NK cells, such as any of the NK cell subsets described herein, or a number of viable cells of any of the foregoing. In any of the above embodiments, the dose is given as the number of cells in a composition of expanded cells produced by the provided method, or a number of viable cells of any of the foregoing.

In some embodiments, the dose for administration in accord with any of the methods of treatment or uses is from at or about 5×10⁷ to at or about 10×10⁹, such as from at or about 5×10⁷ to at or about 5×10⁹, from about or about 5×10⁷ to at or about 1×10⁹, from at or about 5×10⁷ to at or about 5×10⁸, from about or about 5×10⁷ to at or about 1×10⁸, 1×10⁸ to at or about 10×10⁹, from at or about 1×10⁸ to at or about 5×10⁹, from about or about 1×10⁸ to at or about 1×10⁹, from at or about 1×10⁸ to at or about 5×10⁸, from at or about 5×10⁸ to at or about 10×10⁹, from at or about 5×10⁸ to at or about 5×10⁹, from about or about 5×10⁸ to at or about 1×10⁹, from at or about 1×10⁹ to at or about 10×10⁹, from at or about 1×10⁹ to at or about 5×10⁹, or from at or about 5×10⁹ to at or about 10×10⁹. In some embodiments, the dose for administration is at or about 5×10⁸ cells. In some embodiments, the dose for administration is at or about 1×10⁹ cells. In some embodiments, the dose for administration is at or about 5×10⁹ cells. In some embodiments, the dose for administration is at or about 1×10¹⁰ cells. In some embodiments, the dose is given as the number of g-NK cells or an NK cell subset that is associated with or includes a surrogate marker for g-NK cells, such as any of the NK cell subsets described herein, or a number of viable cells of any of the foregoing. In any of the above embodiments, the dose is given as the number of cells in a composition of expanded cells produced by the provided method, or a number of viable cells of any of the foregoing.

In some embodiments, the composition containing expanded NK cells are administered to an individual soon after expansion according to the provided methods. In other embodiments, the expanded NK cells are stored or expanded by growth in culture prior to administration, such as by methods described above. For example, the NK cells can be stored for greater than 6, 12, 18, or 24 months prior to administration to the individual.

In some embodiments, the provided compositions containing NK cells and subsets thereof, such as g-NK cells, can be administered to a subject by any convenient route including parenteral routes such as subcutaneous, intramuscular, intravenous, and/or epidural routes of administration.

In particular embodiments, the provided compositions are administered by intravenous infusion. In some embodiments, at or about 10×10⁶ cells to 10×10⁹ cells are administered by intravenous infusion in a volume of 1 mL to 100 mL. In some embodiments, at or about 50×10⁶ cells are administered. In some embodiments, at or about 1×10⁹ cells are administered. In some embodiments, at or about 5×10⁹ cells are administered. In some embodiments, at or about 10×10⁹ cells are administered. It is within the level of a skilled artisan to determine the volume of cells for infusion to administer the number of cells. In one example, 0.5×10⁹ cells is administered by intravenous infusion of a volume of about 20 mL from a composition, such as a thawed cryopreserved composition, formulated at a concentration of at or about 2.5×10⁷ cells/mL (e.g. at or about 5×10⁹ cells in 200 mL).

In any of the preceding embodiments, the provided g-NK cells and compositions thereof can be used as a monotherapy for the treatment of the disease or disorder.

### A. Compositions and Pharmaceutical Formulations

In some embodiments, the compositions for use in the provided methods contain g-NK cells. In particular, among the provided compositions are compositions of cells that are enriched for g-NK cells. In some embodiments, the compositions for use in the provided methods contain g-NK cells that are expanded NK cells such as produced by any of the provided methods. In some embodiments, the compositions contain NKG2C^{pos} cells or a subset thereof. In some embodiments, the compositions contain NKG2A^{neg} cells or a subset thereof. In some embodiments, the compositions contain NKG2C^{pos}/NKG2A^{neg} cells or a subset thereof.

In some embodiments, the composition comprises about 5-99% NKG2C^{pos} cells or a subset thereof, or any percentage of NKG2C^{pos} cells or a subset thereof between 5 and 99% inclusive. In some embodiments, the composition can include an increased or greater percentages of NKG2C^{pos} cells or a subset thereof relative to total NK cells or total cells compared to the percentage of NKG2C^{pos} cells or the subset thereof relative to total NK cells or total cells naturally present in the subject from which the cells were isolated. In some embodiments, the percentage is increased at least or at least about 2-fold, 3-fold, 4-fold, 5-fold, 10-fold, 20-fold, 30-fold, 40-fold, 50-fold, 60-fold, 70-fold, 80-fold, 90-fold, 100-fold, 150-fold, 200-fold or more.

In some embodiments, the composition can include at least at or about 20%, at least at or about 30%, at least at or about 40%, at least at or about 50%, at least at or about 60%, at least at or about 65%, at least at or about 70%, at least at or about 75%, at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99%, or substantially 100% NKG2C^{pos} cells or a subset thereof. In some embodiments, the composition comprises more than 50% NKG2C^{pos} cells or a subset thereof. In another embodiment, the composition comprises more than 60% NKG2C^{pos} cells or a subset thereof. In another embodiment, the composition comprises more than 70% NKG2C^{pos} cells or a subset thereof. In another embodiment, the composition comprises more than 80% NKG2C^{pos} cells or a subset thereof. In some embodiments, the provided compositions include those in which the NKG2C^{pos} cells or a subset thereof make up at least at or about 60%, at least at or about 70%, at least at or about 80%, at least at or about 85%, at least at or about 90%, at least at or about 95% or more of the cells in the composition or of the NK cells in the composition.

In some embodiments, the composition comprises about 5-99% NKG2A^{neg} cells or a subset thereof, or any percentage of NKG2A^{neg} cells or a subset thereof between 5 and 99% inclusive. In some embodiments, the composition can include an increased or greater percentages of NKG2A^{neg} cells or a subset thereof relative to total NK cells or total cells compared to the percentage of NKG2A^{neg} cells or the subset thereof relative to total NK cells or total cells naturally present in the subject from which the cells were isolated. In some embodiments, the percentage is increased at least or at least about 2-fold, 3-fold, 4-fold, 5-fold, 10-fold, 20-fold, 30-fold, 40-fold, 50-fold, 60-fold, 70-fold, 80-fold, 90-fold, 100-fold, 150-fold, 200-fold or more.

In some embodiments, the composition can include at least at or about 20%, at least at or about 30%, at least at or about 40%, at least at or about 50%, at least at or about 60%, at least at or about 65%, at least at or about 70%, at least at or about 75%, at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99%, or substantially 100% NKG2A^{neg} cells or a subset thereof. In some embodiments, the composition comprises more than 50% NKG2A^{neg} cells or a subset thereof. In another embodiment, the composition comprises more than 60% NKG2A^{neg} cells or a subset thereof. In another embodiment, the composition comprises more than 70% NKG2A^{neg} cells or a subset thereof. In another embodiment, the composition comprises more than 80% NKG2A^{neg} cells or a subset thereof. In some embodiments, the provided compositions include those in which the NKG2A^{neg} cells or a subset thereof make up at least at or about 60%, at least at or about 70%, at least at or about 80%, at least at or about 85%, at least at or about 90%, at least at or about 95% or more of the cells in the composition or of the NK cells in the composition.

In some embodiments, the composition comprises about 5-99% NKG2C^{pos}NKG2A^{neg} cells or a subset thereof, or any percentage of NKG2C^{pos}NKG2A^{neg} cells or a subset thereof between 5 and 99% inclusive. In some embodiments, the composition can include an increased or greater percentages of NKG2C^{pos}NKG2A^{neg} cells or a subset thereof relative to total NK cells or total cells compared to the percentage of NKG2C^{pos}NKG2A^{neg} cells or the subset thereof relative to total NK cells or total cells naturally present in the subject from which the cells were isolated. In some embodiments, the percentage is increased at least or at least about 2-fold, 3-fold, 4-fold, 5-fold, 10-fold, 20-fold, 30-fold, 40-fold, 50-fold, 60-fold, 70-fold, 80-fold, 90-fold, 100-fold, 150-fold, 200-fold or more.

In some embodiments, the composition can include at least at or about 20%, at least at or about 30%, at least at or about 40%, at least at or about 50%, at least at or about 60%, at least at or about 65%, at least at or about 70%, at least at or about 75%, at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99%, or substantially 100% NKG2C^{pos}NKG2A^{neg} cells or a subset thereof. In some embodiments, the composition comprises more than 50% NKG2C^{pos}NKG2A^{neg} cells or a subset thereof. In another embodiment, the composition comprises more than 60% NKG2C^{pos}NKG2A^{neg} cells or a subset thereof. In another embodiment, the composition comprises more than 70% NKG2C^{pos}NKG2A^{neg} cells or a subset thereof. In another embodiment, the composition comprises more than 80% NKG2C^{pos}NKG2A^{neg} cells or a subset thereof. In some embodiments, the provided compositions include those in which the NKG2C^{pos}NKG2A^{neg} cells or a subset thereof make up at least at or about 60%, at least at or about 70%, at least at or about 80%, at least at or about 85%, at least at or about 90%, at least at or about 95% or more of the cells in the composition or of the NK cells in the composition.

In some embodiments, the composition comprises about 5-99% g-NK cells, or any percentage of g-NK cells between 5 and 99% inclusive. In some embodiments, the composition can include an increased or greater percentages of g-NK cells relative to total NK cells or total cells compared to the percentage of g-NK relative to total NK cells or total cells naturally present in the subject from which the cells were isolated. In some embodiments, the percentage is increased at least or at least about 2-fold, 3-fold, 4-fold, 5-fold, 10-fold, 20-fold, 30-fold, 40-fold, 50-fold, 60-fold, 70-fold, 80-fold, 90-fold, 100-fold, 150-fold, 200-fold or more.

In some embodiments, the composition can include at least at or about 20%, at least at or about 30%, at least at or about 40%, at least at or about 50%, at least at or about 60%, at least at or about 65%, at least at or about 70%, at least at or about 75%, at least at or about 80%, at least at or about 81%, at least at or about 82%, at least at or about 83%, at least at or about 84%, at least at or about 85%, at least at or about 86%, at least at or about 87%, at least at or about 88%, at least at or about 89%, at least at or about 90%, at least at or about 91%, at least at or about 92%, at least at or about 93%, at least at or about 94%, at least at or about 95%, at least at or about 96%, at least at or about 97%, at least at or about 98%, at least at or about 99%, or substantially 100% g-NK cells. In some embodiments, the composition comprises more than 50% g-NK cells. In another embodiment, the composition comprises more than 70% g-NK cells. In another embodiment, the composition comprises more than 80% g-NK cells. In some embodiments, the provided compositions include those in which the g-NK cells make up at least at or about 60%, at least at or about 70%, at least at or about 80%, at least at or about 85%, at least at or about 90%, at least at or about 95% or more of the cells in the composition or of the NK cells in the composition.

In some embodiments, the composition includes a population of a natural killer (NK) cell subset, wherein at least at or about 40%, at least at or about 50%, at least at or about 55%, at least at or about 60%, at least at or about 65%, at least at or about 70%, at least at or about 75%, at least at least at or about 80%, at least at or about 85%, at least at or about 90%, or at least at or about 95% of the cells in the composition have a g-NK cell surrogate marker profile that is CD57^{pos}. In some embodiments, from or from about 70% to at or about 90% of the cells in the composition have the phenotype CD57^{pos}. In some embodiments, at least at or about 72%, at least at or about 74%, at least at or about 76%, at least at or about 78%, at least at or about 80%, at least at or about 82%, at least at or about 84%, at least at or about 86%, at least at or about 88%, at least at or about 90%, at least at or about 92%, at least at or about 94%, at least at or about 96% or at least at or about 98% of cell in the composition have the phenotype CD57^{pos} In some of any of the provided embodiments, at least at or about 60% of the cells in the composition comprise the phenotype CD57^{pos}. In some of any of the provided embodiments, at least at or about 70% of the cells in the composition comprise the phenotype CD57^{pos}. In some embodiments, the phenotype further includes the surface phenotype CD3^{neg}. In some embodiments, the phenotype further includes the surface phenotype CD45^{pos}/CD3^{neg}/CD56^{pos}. In some of any of the provided embodiments, of the cells that have such a phenotype greater than 50% are FcRγ^{neg}, optionally between at or about 50% and 90% are FcRγneg. In some of any of the provided embodiments, of the cells that have such a phenotype greater than 70% are FcRγneg, optionally between at or about 70% and 90% are FcRγneg.

In some embodiments, the composition includes a population of a natural killer (NK) cell subset, wherein at least at or about 40%, at least at or about 50%, at least at or about 55%, at least at or about 60%, at least at or about 65%, at least at or about 70%, at least at or about 75%, at least at least at or about 80%, at least at or about 85%, at least at or about 90%, or at least at or about 95% of the cells in the composition have a g-NK cell surrogate marker profile that is CD16^{pos}/CD57^{pos}/CD7^{dim/neg}/CD161^{neg}. In some embodiments, from or from about 70% to at or about 90% of the cells in the composition have the phenotype CD16^{pos}/CD57^{pos}/CD7^{dim/neg}/CD161^{neg}. In some embodiments, at least at or about 72%, at least at or about 74%, at least at or about 76%, at least at or about 78%, at least at or about 80%, at least at or about 82%, at least at or about 84%, at least at or about 86%, at least at or about 88%, at least at or about 90%, at least at or about 92%, at least at or about 94%, at least at or about 96% or at least at or about 98% of cell in the composition have the phenotype CD16^{pos}/CD57^{pos}/CD7^{dim/neg}/CD161^{neg}. In some of any of the provided embodiments, at least at or about 60% of the cells in the composition comprise the phenotype CD16^{pos}/CD57^{pos}/CD7^{dim/neg}/CD161^{neg}. In some of any of the provided embodiments, at least at or about 70% of the cells in the composition comprise the phenotype CD16^{pos}/CD57^{pos}/CD7^{dim/neg}/CD161^{neg}. In some embodiments, the phenotype further includes the surface phenotype CD3^{neg}. In some embodiments, the phenotype further includes the surface phenotype CD45^{pos}/CD3^{neg}/CD56^{pos}. In some of any of the provided embodiments, of the cells that have such a phenotype greater than 50% are FcRγ^{neg}, optionally between at or about 50% and 90% are FcRγ^{neg}. In some of any of the provided embodiments, of the cells that have such a phenotype greater than 70% are FcRγneg, optionally between at or about 70% and 90% are FcRγ^{neg}.

In some embodiments, the composition includes a population of a natural killer (NK) cell subset, wherein at least at or about 40%, at least at or about 50%, at least at or about 55%, at least at or about 60%, at least at or about 65%, at least at or about 70%, at least at or about 75%, at least at least at or about 80%, at least at or about 85%, at least at or about 90%, or at least at or about 95% of the cells in the composition have a phenotype that is CD38^{neg}. In some embodiments, from or from about 70% to at or about 90% of the cells in the composition have the phenotype CD38^{neg}. In some embodiments, at least at or about 72%, at least at or about 74%, at least at or about 76%, at least at or about 78%, at least at or about 80%, at least at or about 82%, at least at or about 84%, at least at or about 86%, at least at or about 88%, at least at or about 90%, at least at or about 92%, at least at or about 94%, at least at or about 96% or at least at or about 98% of cell in the composition have the phenotype CD38^{neg}. In some of any of the provided embodiments, at least at or about 60% of the cells in the composition comprise the phenotype CD38^{neg}. In some of any of the provided embodiments, at least at or about 70% of the cells in the composition comprise the phenotype CD38^{neg}. In some embodiments, the phenotype further includes the surface phenotype CD3^{neg}. In some embodiments, the phenotype further includes the surface phenotype CD45^{pos}/CD3^{neg}/CD56^{pos}. In some of any of the provided embodiments, of the cells that have such a phenotype greater than 50% are FcRγneg, optionally between at or about 50% and 90% are FcRγneg. In some of any of the provided embodiments, of the cells that have such a phenotype greater than 70% are FcRγ^{neg}, optionally between at or about 70% and 90% are FcRγ^{neg}.

In some embodiments, the composition includes a population of a natural killer (NK) cell subset, wherein at least at or about 40%, at least at or about 50%, at least at or about 55%, at least at or about 60%, at least at or about 65%, at least at or about 70%, at least at or about 75%, at least at least at or about 80%, at least at or about 85%, at least at or about 90%, or at least at or about 95% of the cells in the composition have a phenotype that is CD16^{pos}. In some embodiments, from or from about 70% to at or about 90% of the cells in the composition have the phenotype CD16^{pos}. In some embodiments, at least at or about 72%, at least at or about 74%, at least at or about 76%, at least at or about 78%, at least at or about 80%, at least at or about 82%, at least at or about 84%, at least at or about 86%, at least at or about 88%, at least at or about 90%, at least at or about 92%, at least at or about 94%, at least at or about 96% or at least at or about 98% of cell in the composition have the phenotype CD16^{pos}. In some of any of the provided embodiments, at least at or about 60% of the cells in the composition comprise the phenotype CD16^{pos}. In some of any of the provided embodiments, at least at or about 70% of the cells in the composition comprise the phenotype CD16^{pos}. In some embodiments, the phenotype further includes the surface phenotype CD3^{neg}. In some embodiments, the phenotype further includes the surface phenotype CD45^{pos}/CD3^{neg}/CD56^{pos}. In some of any of the provided embodiments, of the cells that have such a phenotype greater than 50% are FcRγ^{neg}, optionally between at or about 50% and 90% are FcRγ^{neg}. In some of any of the provided embodiments, of the cells that have such a phenotype greater than 70% are FcRγ^{neg}, optionally between at or about 70% and 90% are FcRγ^{neg}.

In some embodiments, the composition includes a population of a natural killer (NK) cell subset, wherein at least at or about 40%, at least at or about 50%, at least at or about 55%, at least at or about 60%, at least at or about 65%, at least at or about 70%, at least at or about 75%, at least at least at or about 80%, at least at or about 85%, at least at or about 90%, or at least at or about 95% of the cells in the composition have a g-NK cell surrogate marker profile that is NKG2A^{neg}/CD161^{neg}. In some embodiments, from or from about 70% to at or about 90% of the cells in the composition have the phenotype NKG2A^{neg}/CD161^{neg}. In some embodiments, at least at or about 72%, at least at or about 74%, at least at or about 76%, at least at or about 78%, at least at or about 80%, at least at or about 82%, at least at or about 84%, at least at or about 86%, at least at or about 88%, at least at or about 90%, at least at or about 92%, at least at or about 94%, at least at or about 96% or at least at or about 98% of cell in the composition have the phenotype NKG2A^{neg}/CD161^{neg}. In some of any of the provided embodiments, at least at or about 60% of the cells in the composition comprise the phenotype NKG2A^{neg}/CD161^{neg}. In some of any of the provided embodiments, at least at or about 70% of the cells in the composition comprise the phenotype NKG2A^{neg}/CD161^{neg}. In some embodiments, the phenotype further includes the surface phenotype CD3^{neg}. In some embodiments, the phenotype further includes the surface phenotype CD45^{pos}/CD3^{neg}/CD56^{pos}. In some of any of the provided embodiments, of the cells that have such a phenotype greater than 50% are FcRγ^{neg}, optionally between at or about 50% and 90% are FcRγ^{neg}. In some of any of the provided embodiments, of the cells that have such a phenotype greater than 70% are FcRγ^{neg}, optionally between at or about 70% and 90% are FcRγ^{neg}.

In some embodiments, the composition includes a population of NK cells wherein greater than at or about 50% of the NK cells in the composition are g-NK cells (FcRγ^{neg}) or NK cells expressing a surrogate marker profile thereof. In some embodiments, the composition includes a population of NK cells wherein greater than at or about 55% of the NK cells in the composition are g-NK cells (FcRγ^{neg}) or NK cells expressing a surrogate marker profile thereof. In some embodiments, the composition includes a population of NK cells wherein greater than at or about 60% of the NK cells in the composition are g-NK cells (FcRγ^{neg}) or NK cells expressing a surrogate marker profile thereof. In some embodiments, the composition includes a population of NK cells wherein greater than at or about 65% of the NK cells in the composition are g-NK cells (FcRγ^{neg}) or NK cells expressing a surrogate marker profile thereof. In some embodiments, the composition includes a population of NK cells wherein greater than at or about 70% of the NK cells in the composition are g-NK cells (FcRγ^{neg}) or NK cells expressing a surrogate marker profile thereof. In some embodiments, the composition includes a population of NK cells wherein greater than at or about 75% of the NK cells in the composition are g-NK cells (FcRγ^{neg}) or NK cells expressing a surrogate marker profile thereof. In some embodiments, the composition includes a population of NK cells wherein greater than at or about 80% of the NK cells in the composition are g-NK cells (FcRγ^{neg}) or NK cells expressing a surrogate marker profile thereof. In some embodiments, the composition includes a population of NK cells wherein greater than at or about 85% of the NK cells in the composition are g-NK cells (FcRγ^{neg}) or NK cells expressing a surrogate marker profile thereof. In some embodiments, the composition includes a population of NK cells wherein greater than at or about 90% of the NK cells in the composition are g-NK cells (FcRγ^{neg}) or NK cells expressing a surrogate marker profile thereof. In some embodiments, the composition includes a population of NK cells wherein greater than at or about 95% of the NK cells in the composition are g-NK cells (FcRγ^{neg}) or NK cells expressing a surrogate marker profile thereof. The surrogate marker profile may be any as described herein. For example, the surrogate marker profile may be CD16^{pos}/CD57^{pos}/CD7^{dim/neg}/CD161^{neg}. In other examples, the surrogate marker profile may be NKG2A^{neg}/CD161^{neg}. In further example, the g-NK cell surrogate marker profile is CD38^{neg}. A surrogate surface marker profile may further include the phenotype CD45^{pos}/CD3^{neg}/CD56^{pos}.

In some embodiments, the g-NK cells of the composition, or a certain percentage thereof, e.g. greater than about 70%, are positive for perforin and/or granzyme B. Methods for measuring the number of cells positive for perforin or granzyme B are known to a skilled artisan. Methods include, for example, intracellular flow cytometry. In an example, the percentage or number of cells positive for perforin or granzyme B may be determined by the permeabilization of cells, for instance using the Inside Stain Kit from Miltenyi Biotec, prior to staining with antibodies against perforin and granzyme B. Cell staining can then be resolved for instance using flow cytometry.

In some embodiments, greater than at or about 70% of the g-NK cells of the composition are positive for perforin, and greater than at or about 70% of the g-NK cells of the composition are positive for granzyme B. In some embodiments, greater than at or about 75% of the g-NK cells of the composition are positive for perforin, and greater than at or about 75% of the g-NK cells of the composition are positive for granzyme B. In some embodiments, greater than at or about 80% of the g-NK cells of the composition are positive for perforin, and greater than at or about 80% of the g-NK cells of the composition are positive for granzyme B. In some embodiments, greater than at or about 85% of the g-NK cells of the composition are positive for perforin, and greater than at or about 85% of the g-NK cells of the composition are positive for granzyme B. In some embodiments, greater than at or about 90% of the g-NK cells of the composition are positive for perforin, and greater than at or about 90% of the g-NK cells of the composition are positive for granzyme B. In some embodiments, greater than at or about 95% of the g-NK cells of the composition are positive for perforin, and greater than at or about 95% of the g-NK cells of the composition are positive for granzyme B.

In some embodiments, perforin and granzyme B expression levels by NK cells, for instance g-NK cells, can be measured by intracellular flow cytometry and levels measured based on levels of mean fluorescence intensity (MFI). In some embodiments, perforin and granzyme B expression levels based on MFI will differ between g-NK cells and cells that are FcRγ^{pos}. In some embodiments, the g-NK cells of the composition that are positive for perforin express a mean level of perforin, based on MFI levels, at least at or about two times the mean level of perforin expressed by FcRγ^{pos} NK cells. In some embodiments, the g-NK cells of the composition that are positive for perforin express a mean level of perforin, based on MFI levels, at least at or about three times the mean level of perforin expressed by FcRγ^{pos} NK cells. In some embodiments, the g-NK cells of the composition that are positive for perforin express a mean level of perforin, based on MFI levels, at least at or about four times the mean level of perforin expressed by FcRγ^{pos} NK cells. In some embodiments, the g-NK cells of the composition that are positive for granzyme B express a mean level of granzyme B, based on MFI levels, at least at or about two times the mean level of granzyme B expressed by FcRγ^{pos} NK cells. In some embodiments, the g-NK cells of the composition that are positive for granzyme B express a mean level of granzyme B, based on MFI levels, at least at or about three times the mean level of granzyme B expressed by FcRγ^{pos} NK cells. In some embodiments, the g-NK cells of the composition that are positive for granzyme B express a mean level of granzyme B, based on MFI levels, at least at or about four times the mean level of granzyme B expressed by FcRγ^{pos} NK cells.

In some embodiments, at least at or about 50% of the cells in the composition are FcRγ-deficient NK cells (g-NK), wherein greater than at or about 70% of the g-NK cells are positive for perforin and greater than at or about 70% of the g-NK cells are positive for granzyme B. In some embodiments, greater than at or about 80% of the g-NK cells are positive for perforin and greater than at or about 80% of the g-NK cells are positive for granzyme B. In some embodiments, greater than at or about 90% of the g-NK cells are positive for perforin and greater than at or about 90% of the g-NK cells are positive for granzyme B. In some embodiments, greater than at or about 95% of the g-NK cells are positive for perforin and greater than at or about 95% of the g-NK cells are positive for granzyme B. In some embodiments, the g-NK cells are FcRγ^{neg}.

In some of any embodiments, among the cells positive for perforin, the cells express a mean level of perforin as measured by intracellular flow cytometry that is, based on mean fluorescence intensity (MFI), at least at or about two times the mean level of perforin expressed by cells that are FcRγ^{pos}. In some of any embodiments, among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγ^{pos}.

In some of any embodiments, greater than 10% of the cells in the composition are capable of degranulation against tumor target cells, optionally as measured by CD107a expression, optionally wherein the degranulation is measured in the absence of an antibody against the tumor target cells. In some of any embodiments, among the cells in the composition, greater than at or about 15%, greater than at or about 20%, greater than at or about 30%, greater than at or about 40% or greater than at or about 50% exhibit degranulation, optionally as measured by CD107a expression, in the presence of cells expressing a target antigen (target cells) and an antibody directed against the target antigen (anti-target antibody). In some of any such embodiments, greater than 10% of the cells in the composition are capable of producing interferon-gamma or TNF-alpha against tumor target cells, optionally wherein the interferon-gamma or TNF-alpha is measured in the absence of an antibody against the tumor target cells. In some embodiments, among the cells in the composition, greater than at or about 15%, greater than at or about 20%, greater than at or about 30%, greater than at or about 40% or greater than at or about 50% produce an effector cytokine in the presence of cells expressing a target antigen (target cells) and an antibody directed against the target antigen (anti-target antibody). In some embodiments, for instance, the target cells may be a tumor cell line expressing CD38 and the antibody is an anti-CD38 antibody (e.g. daratumumab). In some embodiments, for instance, the target cells may be a tumor cell line expressing SLAMF7 and the antibody is an anti-SLAMF7 antibody (e.g. eltouzumab). In some embodiments, for instance, the target cells may be a tumor cell line expressing BCMA and the antibody is an anti-BCMA antibody (e.g. belantamab). In some embodiments, for instance, the target cells may be a tumor cell line expressing CD20 and the antibody is an anti-CD20 antibody (e.g. rituximab). In some embodiments, for instance, the target cells may be a tumor cell line expressing CD19 and the antibody is an anti-CD19 antibody (e.g. tafasitamab or loncastuximab). In some embodiments, for instance, the target cells may be a tumor cell line expressing CD30 and the antibody is an anti-CD30 antibody (e.g. brentuximab).

In some embodiments, at least at or about 50% of the cells in the composition are FcRγ-deficient (FcRγ^{neg}) NK cells (g-NK), and wherein greater than at or about 15% of the cells in the composition produce an effector cytokine in the presence of cells expressing a target antigen (target cells) and an antibody directed against the target antigen (anti-target antibody). In some embodiments, greater than at or about 20%, greater than at or about 30%, greater than at or about 40% or greater than at or about 50% produce an effector cytokine in the presence of cells expressing a target antigen (target cells) and an antibody directed against the target antigen (anti-target antibody). In some embodiments, for instance, the target cells may be a tumor cell line expressing CD38 and the antibody is an anti-CD38 antibody (e.g. daratumumab). In some embodiments, for instance, the target cells may be a tumor cell line expressing SLAMF7 and the antibody is an anti-SLAMF7 antibody (e.g. eltouzumab). In some embodiments, for instance, the target cells may be a tumor cell line expressing BCMA and the antibody is an anti-BCMA antibody (e.g. belantamab). In some embodiments, for instance, the target cells may be a tumor cell line expressing CD20 and the antibody is an anti-CD20 antibody (e.g. rituximab). In some embodiments, for instance, the target cells may be a tumor cell line expressing CD19 and the antibody is an anti-CD19 antibody (e.g. tafasitamab or loncastuximab). In some embodiments, for instance, the target cells may be a tumor cell line expressing CD30 and the antibody is an anti-CD30 antibody (e.g. brentuximab).

In some of any embodiments, the effector cytokine is IFN-gamma or TNF-alpha. In some of any embodiments, the effector cytokine is IFN-gamma and TNF-alpha.

In some of any embodiments, among the cells in the composition, greater than at or about 15%, greater than at or about 20%, greater than at or about 30%, greater than at or about 40% or greater than at or about 50% exhibit degranulation, optionally as measured by CD107a expression, in the presence of cells expressing a target antigen (target cells) and an antibody directed against the target antigen (anti-target antibody). In some embodiments, for instance, the target cells may be a tumor cell line expressing CD38 and the antibody is an anti-CD38 antibody (e.g. daratumumab). In some embodiments, for instance, the target cells may be a tumor cell line expressing SLAMF7 and the antibody is an anti-SLAMF7 antibody (e.g. eltouzumab). In some embodiments, for instance, the target cells may be a tumor cell line expressing BCMA and the antibody is an anti-BCMA antibody (e.g. belantamab). In some embodiments, for instance, the target cells may be a tumor cell line expressing CD20 and the antibody is an anti-CD20 antibody (e.g. rituximab). In some embodiments, for instance, the target cells may be a tumor cell line expressing CD19 and the antibody is an anti-CD19 antibody (e.g. tafasitamab or loncastuximab). In some embodiments, for instance, the target cells may be a tumor cell line expressing CD30 and the antibody is an anti-CD30 antibody (e.g. brentuximab).

In some embodiments, at least at or about 50% of the cells in the composition are FcRγ-deficient (FcRγ^{neg}) NK cells (g-NK), and wherein greater than at or about 15% of the cells in the composition exhibit degranulation, optionally as measured by CD107a expression, in the presence of cells expressing a target antigen (target cells) and an antibody directed against the target antigen (anti-target antibody). In some embodiments, greater than at or about 20%, greater than at or about 30%, greater than at or about 40% or greater than at or about 50% exhibit degranulation, optionally as measured by CD107a expression, in the presence of cells expressing a target antigen (target cells) and an antibody directed against the target antigen (anti-target antibody). In some embodiments, for instance, the target cells may be a tumor cell line expressing CD38 and the antibody is an anti-CD38 antibody (e.g. daratumumab). In some embodiments, for instance, the target cells may be a tumor cell line expressing SLAMF7 and the antibody is an anti-SLAMF7 antibody (e.g. eltouzumab). In some embodiments, for instance, the target cells may be a tumor cell line expressing BCMA and the antibody is an anti-BCMA antibody (e.g. belantamab). In some embodiments, for instance, the target cells may be a tumor cell line expressing CD20 and the antibody is an anti-CD20 antibody (e.g. rituximab). In some embodiments, for instance, the target cells may be a tumor cell line expressing CD19 and the antibody is an anti-CD19 antibody (e.g. tafasitamab or loncastuximab). In some embodiments, for instance, the target cells may be a tumor cell line expressing CD30 and the antibody is an anti-CD30 antibody (e.g. brentuximab).

In some of any of the provided embodiments greater than at or about 60% of the cells in the composition are g-NK cells. In some of any of the provided embodiments, greater than at or about 70% of the cells in the composition are g-NK cells. In some of any of the provided embodiments, greater than at or about 80% of the cells in the composition are g-NK cells. In some of any of the provided embodiments, greater than at or about 90% of the cells in the composition are g-NK cells. In some of any of the provided embodiments, greater than at or about 95% of the cells in the composition are g-NK cells.

In some embodiments, the g-NK cells exhibit a g-NK cell surrogate marker profile. In some embodiments, the g-NK cell surrogate marker profile is CD16pos/CD57^{pos}/CD7^{dim/neg}/CD161^{neg}. In some embodiments, the g-NK cell surrogate marker profile is NKG2A^{neg}/CD161^{neg}. In some embodiments, the g-NK cell surrogate marker profile is CD38^{neg}. In some embodiments, the g-NK cell surrogate surface marker profile further is CD45^{pos}/CD3^{neg}/CD56^{pos}.

In some of any of the preceding embodiments, greater than at or about 60% of the cells are g-NK cells. In some of any of the preceding embodiments, greater than at or about 70% of the cells are g-NK cells. In some of any of the preceding embodiments, greater than at or about 80% of the cells are g-NK cells. In some of any of the preceding embodiments, greater than at or about 90% of the cells are g-NK cells. In some of any of the preceding embodiments, greater than at or about 95% of the cells are g-NK cells.

In some of any of the preceding embodiments, greater than at or at about 80% of the cells are positive for perforin. In some of any of the preceding embodiments, greater than at or at about 90% of the cells are positive for perforin. In some of any of the preceding embodiments, among the cells positive for perforin, the cells express a mean level of perforin as measured by intracellular flow cytometry that is, based on mean fluorescence intensity (MFI), at least at or about two times the mean level of perforin expressed by cells that are FcRγ^{pos}.

In some of any of the preceding embodiments, greater than at or at about 80% of the cells are positive for granzyme B. In some of any of the preceding embodiments, greater than at or at about 90% of the cells are positive for granzyme B. In some of any of the preceding embodiments, among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγ^{pos}.

In some of any of the provided embodiments, the composition comprises from at or about 10⁶ cells to at or about 10¹² cells. In some of any of the provided embodiments, the composition comprises from at or about 10⁶ to at or about 10¹¹ cells, from at or about 10⁶ to at or about 10¹⁰ cells, from at or about 10⁶ to at or about 10⁹ cells, from at or about 10⁶ to at or about 10⁸ cells, from at or about 10⁶ to at or about 10⁷ cells, from at or about 10⁷ to at or about 10¹² cells, from at or about 10⁷ to at or about 10¹¹ cells, from at or about 10⁷ to at or about 10¹⁰ cells, from at or about 10⁷ to at or about 10⁹ cells, or from at or about 10⁷ to at or about 10⁸ cells, from at or about 10⁸ to at or about 10¹² cells, from at or about 10⁸ to at or about 10¹¹ cells, from at or about 10⁸ to at or about 10¹⁰ cells, from at or about 10⁸ to at or about 10⁹ cells, from at or about 10⁹ to at or about 10¹² cells, from at or about 10⁹ to at or about 10¹¹ cells, from at or about 10⁹ to at or about 10¹⁰ cells, from at or about 10¹⁰ to at or about 10¹² cells, from at or about 10¹⁰ to at or about 10¹¹ cells, or from at or about 10¹¹ to at or about 10¹² cells.

In some of any of the provided embodiments, the composition comprises at least or about at least 10⁶ cells. In some of any of the provided embodiments, the composition comprises from at or about 10⁶ to at or about 10¹⁰ cells, from at or about 10⁶ to at or about 10⁹ cells, from at or about 10⁶ to at or about 10⁸ cells, from at or about 10⁶ to at or about 10⁷ cells, from at or about 10⁷ to at or about 10¹⁰ cells, from at or about 10⁷ to at or about 10⁹ cells, from at or about 10⁷ to at or about 10⁸ cells, from at or about 10⁸ to at or about 10¹⁰ cells, from at or about 10⁸ to at or about 10⁹ cells, or from at or about 10⁹ to at or about 10¹⁰ cells.

In some of any of the provided embodiments, the composition comprises at least or about at least 10⁸ cells. In some of any of the provided embodiments, the composition comprises at least at or about 10⁹ cells. In some of any of the provided embodiments, the composition comprises at least at or about 10¹⁰ cells. In some of any of the provided embodiments, the composition comprises at least at or about 10¹¹ cells. In some of any of the provided embodiments, the composition comprises from at or about 10⁸ to at or about 10¹¹ cells. In some of any of the provided embodiments, the composition comprises from at or about 10⁸ to at or about 10¹⁰ cells. In some of any of the provided embodiments, the composition comprises from at or about 10⁸ to at or about 10⁹ cells. In some of any of the provided embodiments, the composition comprises from at or about 10⁹ to at or about 10¹¹ cells. In some of any of the provided embodiments, the composition comprises from at or about 10⁹ to at or about 10¹⁰ cells. In some of any of the provided embodiments, the composition comprises from at or about 10¹⁰ to at or about 10¹¹ cells.

In some of any of the provided embodiments, the composition comprises at least at or about 10⁶ g-NK cells. In some of any of the provided embodiments, the composition comprises from at or about 10⁶ to at or about 10¹⁰ g-NK cells, from at or about 10⁶ to at or about 10⁹ g-NK cells, from at or about 10⁶ to at or about 10⁸ g-NK cells, from at or about 10⁶ to at or about 10⁷ g-NK cells, from at or about 10⁷ to at or about 10¹⁰ g-NK cells, from at or about 10⁷ to at or about 10⁹ g-NK cells, from at or about 10⁷ to at or about 10⁸ g-NK cells, from at or about 10⁸ to at or about 10¹⁰ g-NK cells, from at or about 10⁸ to at or about 10⁹ g-NK cells, or from at or about 10⁹ to at or about 10¹⁰ g-NK cells. In some of any of the provided embodiments, the g-NK cells are FcRγ^{neg}. In some of any of the provided embodiments, the g-NK cells are cells having a g-NK surrogate surface marker profile. In some embodiments, the g-NK cell surrogate surface marker profile is CD16^{pos}/CD57^{pos}/CD7^{dim/neg}/CD161^{neg}. In some embodiments, the g-NK cell surrogate surface marker profile is NKG2A^{neg}/CD161^{neg}. In some of any of the provided embodiments, the g-NK cells or cells having a g-NK surrogate marker profile further include the surface phenotype CD45^{pos}/CD3^{neg}/CD56^{pos}. In some of any of the provided embodiments, the g-NK cells or cells having a g-NK surrogate marker profile further include the surface phenotype CD38^{neg}.

In particular embodiments of any of the provided compositions, the cells in the composition are from the same donor. As such, the compositions do not include a mixed population of cells from one or more different donors. As provided here, the methods of expansion result in high yield expansion of at or greater than 500-fold, at or greater than 600-fold, at or greater than 700-fold, at or greater than 800-fold, at or greater than 900-fold, at or greater than 1000-fold or more of certain NK cell subsets, particularly the g-NK cell subset or an NK cell subset that is associated with or includes a surrogate marker for g-NK cells, such as any of the NK cell subsets described above. In some of any embodiments, the increase is at or about 1000-fold greater. In some of any embodiments, the increase is at or about 2000-fold greater. In some of any embodiments, the increase is at or about 2500-fold greater. In some of any embodiments, the increase is at or about 3000-fold greater. In some of any embodiments, the increase is at or about 5000-fold greater. In some of any embodiments, the increase is at or about 10000-fold greater. In some of any embodiments, the increase is at or about 15000-fold greater. In some of any embodiments, the increase is at or about 20000-fold greater. In some of any embodiments, the increase is at or about 25000-fold greater. In some of any embodiments, the increase is at or about 30000-fold greater. In some of any embodiments, the increase is at or about 35000-fold greater. In particular embodiments, expansion results in at or about 1,000 fold increase in number of certain NK cell subsets, particularly the g-NK cell subset or an NK cell subset that is associated with or includes a surrogate marker for g-NK cells, such as any of the NK cell subsets described above. In particular embodiments, expansion results in at or about 3,000 fold increase in number of certain NK cell subsets, particularly the g-NK cell subset or an NK cell subset that is associated with or includes a surrogate marker for g-NK cells, such as any of the NK cell subsets described above. In particular embodiments, expansion results in at or about 35,000 fold increase in number of certain NK cell subsets, particularly the g-NK cell subset or an NK cell subset that is associated with or includes a surrogate marker for g-NK cells, such as any of the NK cell subsets described above.

In some cases, expansion achieved by the provided methods from an initial source of NK cells obtained from a single donor can produce a composition of cells to provide a plurality of individual doses for administration to a subject in need. As such, the provided methods are particularly suitable for allogeneic methods. In some cases, a single expansion from a starting population of NK cells isolated from one donor in accord with the provided methods can result in greater than or greater than about 20 individual doses for administration to a subject in need, such as at or about 30 individual doses, 40 individual doses, 50 individual doses, 60 individual doses, 70 individual doses, 80 individual doses, 90 individual doses, 100 individual doses, or an individual dose that is a value between any of the foregoing. In some embodiments, the individual dose is from at or about 1×10⁵ cells/kg to at or about 1×10⁷ cells/kg, such as from at or about 1×10⁵ cells/kg to at or about 7.5×10⁶ cells/kg, from at or about 1×10⁵ cells/kg to at or about 5×10⁶ cells/kg, from at or about 1×10⁵ cells/kg to at or about 2.5×10⁶ cells/kg, from at or about 1×10⁵ cells/kg to at or about 1×10⁶ cells/kg, from at or about 1×10⁵ cells/kg to at or about 7.5×10⁵ cells/kg, from at or about 1×10⁵ cells/kg to at or about 5×10⁵ cells/kg, from at or about 1×10⁵ cells/kg to at or about 2.5×10⁵ cells/kg, from at or about 2.5×10⁵ cells/kg to at or about 1×10⁷ cells/kg, from at or about 2.5×10⁵ cells/kg to at or about 7.5×10⁶ cells/kg, from at or about 2.5×10⁵ cells/kg to at or about 5×10⁶ cells/kg, from at or about 2.5×10⁵ cells/kg to at or about 2.5×10⁶ cells/kg, from at or about 2.5×10⁵ cells/kg to at or about 1×10⁶ cells/kg, from at or about 2.5×10⁵ cells/kg to at or about 7.5×10⁵ cells/kg, from at or about 2.5×10⁵ cells/kg to at or about 5×10⁵ cells/kg, from at or about 5×10⁵ cells/kg to at or about 1×10⁷ cells/kg, from at or about 5×10⁵ cells/kg to at or about 7.5×10⁶ cells/kg, from at or about 5×10⁵ cells/kg to at or about 5×10⁶ cells/kg, from at or about 5×10⁵ cells/kg to at or about 2.5×10⁶ cells/kg, from at or about 5×10⁵ cells/kg to at or about 1×10⁶ cells/kg, from at or about 5×10⁵ cells/kg to at or about 7.5×10⁵ cells/kg, from at or about 1×10⁶ cells/kg to at or about 1×10⁷ cells/kg, from at or about 1×10⁶ cells/kg to at or about 7.5×10⁶ cells/kg, from at or about 1×10⁶ cells/kg to at or about 5×10⁶ cells/kg, from at or about 1×10⁶ cells/kg to at or about 2.5×10⁶ cells/kg, from at or about 2.5×10⁶ cells/kg to at or about 1×10⁷ cells/kg, from at or about 2.5×10⁶ cells/kg to at or about 7.5×10⁶ cells/kg, from at or about 2.5×10⁶ cells/kg to at or about 5×10⁶ cells/kg, from at or about 5×10⁶ cells/kg to at or about 1×10⁷ cells/kg, from at or about 5×10⁶ cells/kg to at or about 7.5×10⁶ cells/kg, or from at or about 7.5×10⁶ cells/kg to at or about 1×10⁷ cells/kg. In some embodiments, the individual dose is from at or about 1×10⁵ cells/kg to at or about 1×10⁸ cells/kg, such as from at or about 2.5×10⁵ cells/kg to at or about 1×10⁸ cells/kg, from at or about 5×10⁵ cells/kg to at or about 1×10⁸ cells/kg, from at or about 7.5×10⁵ cells/kg to at or about 1×10⁸ cells/kg, from at or about 1×10⁶ cells/kg to at or about 1×10⁸ cells/kg, from at or about 2.5×10⁶ cells/kg to at or about 1×10⁸ cells/kg, from at or about 5×10⁶ cells/kg to at or about 1×10⁸ cells/kg, from at or about 7.5×10⁶ cells/kg to at or about 1×10⁸ cells/kg, from at or about 1×10⁷ cells/kg to at or about 1×10⁸ cells/kg, from at or about 2.5×10⁷ cells/kg to at or about 1×10⁸ cells/kg, from at or about 5×10⁷ cells/kg to at or about 1×10⁸ cells/kg, or from at or about 7.5×10⁷ cells/kg to at or about 1×10⁸ cells/kg. In some embodiments, the individual dose is from at or about 5×10⁷ to at or about 10×10⁹, such as from at or about 5×10⁷ to at or about 5×10⁹, from about or about 5×10⁷ to at or about 1×10⁹, from at or about 5×10⁷ to at or about 5×10⁸, from about or about 5×10⁷ to at or about 1×10⁸, 1×10⁸ to at or about 10×10⁹, from at or about 1×10⁸ to at or about 5×10⁹, from about or about 1×10⁸ to at or about 1×10⁹, from at or about 1×10⁸ to at or about 5×10⁸, from at or about 5×10⁸ to at or about 10×10⁹, from at or about 5×10⁸ to at or about 5×10⁹, from about or about 5×10⁸ to at or about 1×10⁹, from at or about 1×10⁹ to at or about 10×10⁹, from at or about 1×10⁹ to at or about 5×10⁹, or from at or about 5×10⁹ to at or about 10×10⁹. In some embodiments, the individual dose is or is about 5×10⁸ cells. In some embodiments, the individual dose is or is about 1×10⁹ cells. In some embodiments, the individual dose is or is about 5×10⁹ cells. In some embodiments, the individual dose is or is about 1×10¹⁰ cells. In any of the above embodiments, the dose is given as the number of cells g-NK cells or an NK cell subset that is associated with or includes a surrogate marker for g-NK cells, such as any of the NK cell subsets described above, or a number of viable cells of any of the foregoing. In any of the above embodiments, the dose is given as the number of cells in a composition of expanded cells produced by the method, or a number of viable cells of any of the foregoing.

Among the compositions are pharmaceutical compositions and formulations for administration, such as for adoptive cell therapy. In some embodiments, the engineered cells are formulated with a pharmaceutically acceptable carrier.

A pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy, Lippincott, Williams & Wilkins, Philadelphia, PA). Examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for NK cells, such as a saline solution, a dextrose solution or a solution comprising human serum albumin.

In some embodiments, the pharmaceutically acceptable carrier or vehicle for such compositions is any non-toxic aqueous solution in which the NK cells can be maintained, or remain viable, for a time sufficient to allow administration of live NK cells. For example, the pharmaceutically acceptable carrier or vehicle can be a saline solution or buffered saline solution. The pharmaceutically acceptable carrier or vehicle can also include various bio materials that may increase the efficiency of NK cells. Cell vehicles and carriers can, for example, include polysaccharides such as methylcellulose (M. C. Tate, D. A. Shear, S. W. Hoffman, D. G. Stein, M. C. LaPlaca, Biomaterials 22, 1113, 2001, which is incorporated herein by reference in its entirety), chitosan (Suh J K F, Matthew H W T. Biomaterials, 21, 2589, 2000; Lahiji A, Sohrabi A, Hungerford D S, et al., J Biomed Mater Res, 51, 586, 2000, each of which is incorporated herein by reference in its entirety), N-isopropylacrylamide copolymer P(NIPAM-CO-AA) (Y. H. Bae, B. Vernon, C. K. Han, S. W. Kim, J. Control. Release 53, 249, 1998; H. Gappa, M. Baudys, J. J. Koh, S. W. Kim, Y. H. Bae, Tissue Eng. 7, 35, 2001, each of which is incorporated herein by reference in its entirety), as well as Poly(oxyethylene)/poly(D,L-lactic acid-co-glycolic acid) (B. Jeong, K. M. Lee, A. Gutowska, Y. H. An, Biomacromolecules 3, 865, 2002, which is incorporated herein by reference in its entirety), P(PF-co-EG) (Suggs L J, Mikos A G. Cell Trans, 8, 345, 1999, which is incorporated herein by reference in its entirety), PEO/PEG (Mann B K, Gobin A S, Tsai A T, Schmedlen R H, West J L., Biomaterials, 22, 3045, 2001; Bryant S J, Anseth K S. Biomaterials, 22, 619, 2001, each of which is incorporated herein by reference in its entirety), PVA (Chih-Ta Lee, Po-Han Kung and Yu-Der Lee, Carbohydrate Polymers, 61, 348, 2005, which is incorporated herein by reference in its entirety), collagen (Lee C R, Grodzinsky A J, Spector M., Biomaterials 22, 3145, 2001, which is incorporated herein by reference in its entirety), alginate (Bouhadir K H, Lee K Y, Alsberg E, Damm K L, Anderson K W, Mooney D J. Biotech Prog 17, 945, 2001; Smidsrd O, Skjak-Braek G., Trends Biotech, 8, 71, 1990, each of which is incorporated herein by reference in its entirety).

In some embodiments, the NK cells such as NKG2C^{pos} cells or a subset thereof can be present in the composition in an effective amount. In some embodiments, the composition contains an effective amount of g-NK cells, such as FcRγ^{neg} cells or cells having a g-NK surrogate marker profile thereof. An effective amount of cells can vary depending on the patient, as well as the type, severity and extent of disease. Thus, a physician can determine what an effective amount is after considering the health of the subject, the extent and severity of disease, and other variables.

In certain embodiments, the number of such cells in the composition is a therapeutically effective amount. In some embodiments, the amount is an amount that reduces the severity, the duration and/or the symptoms associated with cancer, viral infection, microbial infection, or septic shock in an animal. In some embodiments, a therapeutically effective amount is a dose of cells that results in a reduction of the growth or spread of cancer by at least 2.5%, at least 5%, at least 10%, at least 15%, at least 25%, at least 35%, at least 45%, at least 50%, at least 75%, at least 85%, by at least 90%, at least 95%, or at least 99% in a patient or an animal administered a composition described herein relative to the growth or spread of cancer in a patient (or an animal) or a group of patients (or animals) not administered the composition. In some embodiments, a therapeutically effective amount is an amount to result in cytotoxic activity resulting in activity to inhibit or reduce the growth of cancer, viral and microbial cells.

In some embodiments, the composition comprises an amount of NKG2C^{pos} cells or a subset thereof that is from at or about 10⁵ and at or about 10¹² NKG2C^{pos} cells or a subset thereof, or from at or about 10⁵ to at or about 10⁸ NKG2C^{pos} cells or a subset thereof, or from at or about 10⁶ and at or about 10¹² NKG2C^{pos} cells or a subset thereof, or from at or about 10⁸ and at or about 10¹¹ NKG2C^{pos} cells or a subset thereof, or from at or about 10⁹ and at or about 10¹⁰ NKG2C^{pos} cells or a subset thereof. In some embodiments, the composition comprises greater than or greater than at or about 10⁵ NKG2C^{pos} cells or a subset thereof, at or about 10⁶ NKG2C^{pos} cells or a subset thereof, at or about 10⁷ NKG2C^{pos} cells or a subset thereof, at or about 10⁸ NKG2C^{pos} cells or a subset thereof, at or about 10⁹ NKG2C^{pos} cells or a subset thereof, at or about 10¹⁰ NKG2C^{pos} cells or a subset thereof, at or about 10¹¹ NKG2C^{pos} cells or a subset thereof, or at or about 10¹² NKG2C^{pos} cells or a subset thereof. In some embodiments, such an amount can be administered to a subject having a disease or condition, such as to a cancer patient.

In some embodiments, the composition comprises an amount of g-NK cells that is from at or about 10⁵ and at or about 10¹² g-NK cells, or from at or about 10⁵ to at or about 10⁸ g-NK cells, or from at or about 10⁶ and at or about 10¹² g-NK cells, or from at or about 10⁸ and at or about 10¹¹ g-NK cells, or from at or about 10⁹ and at or about 10¹⁰ g-NK cells. In some embodiments, the composition comprises greater than or greater than at or about 10⁵ g-NK cells, at or about 10⁶ g-NK cells, at or about 10⁷ g-NK cells, at or about 10⁸ g-NK cells, at or about 10⁹ g-NK cells, at or about 10¹⁰ g-NK cells, at or about 10¹¹ g-NK cells, or at or about 10¹² g-NK cells. In some embodiments, such an amount can be administered to a subject having a disease or condition, such as to a cancer patient.

In some embodiments, the volume of the composition is at least or at least about 10 mL, 50 mL, 100 mL, 200 mL, 300 mL, 400 mL or 500 mL, such as is from or from about 10 mL to 500 mL, 10 mL to 200 mL, 10 mL to 100 mL, 10 mL to 50 mL, 50 mL to 500 mL, 50 mL to 200 mL, 50 mL to 100 mL, 100 mL to 500 mL, 100 mL to 200 mL or 200 mL to 500 mL, each inclusive. In some embodiments, the composition has a cell density of at least or at least about 1×10⁵ cells/mL, 5×10⁵ cells/mL, 1×10⁶ cells/mL, 5×10⁶ cells/mL, 1×10⁷ cells/mL, 5×10⁷ cells/mL or 1×10⁸ cells/mL. In some embodiments, the cell density of the composition is between or between about 1×10⁵ cells/mL to 1×10⁸ cells/mL, 1×10⁵ cells/mL to 1×10⁷ cells/mL, 1×10⁵ cells/mL to 1×10⁶ cells/mL, 1×10⁶ cells/mL to 1×10⁷ cells/mL, 1×10⁶ cells/mL to 1×10⁸ cells/mL, 1×10⁶ cells/mL to 1×10⁷ cells/mL or 1×10⁷ cells/mL to 1×10⁸ cells/mL, each inclusive.

In some embodiments, the composition, including pharmaceutical composition, is sterile. In some embodiments, isolation, enrichment, or culturing of the cells is carried out in a closed or sterile environment, for example and for instance in a sterile culture bag, to minimize error, user handling and/or contamination. In some embodiments, sterility may be readily accomplished, e.g., by filtration through sterile filtration membranes. In some embodiments, culturing is carried out using a gas permeable culture vessel. In some embodiments, culturing is carried out using a bioreactor.

Also provided herein are compositions that are suitable for cryopreserving the provided NK cells. In some embodiments, the NK cells are cryopreserved in a serum-free cryopreservation medium. In some embodiments, the composition comprises a cryoprotectant. In some embodiments, the cryoprotectant is or comprises DMSO and/or s glycerol. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10). CryoStor™ CS10 is a cryopreservation medium containing 10% dimethyl sulfoxide (DMSO). In some embodiments, compositions formulated for cryopreservation can be stored at low temperatures, such as ultra-low temperatures, for example, storage with temperature ranges from −40° C. to −150° C., such as or about 80° C.±6.0° C.

In some embodiments, the compositions can be preserved at ultra-low temperature before the administration to a patient. In some aspects, NK cell subsets, such as g-NK cells, can be isolated, processed and expanded, such as in accord with the provided methods, and then stored at ultra-low temperature prior to administration to a subject.

A typical method for the preservation at ultra-low temperature in small scale is described, for example, in U.S. Pat. No. 6,0168,991. For small-scale, cells can be preserved at ultra-low temperature by low density suspension (e.g., at a concentration of about 200×10⁶/ml) in 5% human albumin serum (HAS) which is previously cooled. An equivalent amount of 20% DMSO can be added into the HAS solution. Aliquots of the mixture can be placed into vials and frozen overnight inside an ultra-low temperature chamber at about −80° C.

In some embodiments, the cryopreserved NK cells are prepared for administration by thawing. In some cases, the NK cells can be administered to a subject immediately after thawing. In such an embodiment, the composition is ready-to-use without any further processing. In other cases, the NK cells are further processed after thawing, such as by resuspension with a pharmaceutically acceptable carrier, incubation with an activating or stimulating agent, or are activated washed and resuspended in a pharmaceutically acceptable buffer prior to administration to a subject.

### B. Combination Therapy

In some embodiments, compositions containing g-NK cells as provided herein can be administered in a combination therapy with one or more other agents for treating a disease or condition in a subject. In such embodiments, the composition containing g-NK cells as provided herein can be administered prior to, concurrently with or subsequent (after) the administration of one or more other agents. For example, the g-NK cells can be administered simultaneously or sequentially with anti-microbial, anti-viral and other therapeutic agents. Exemplary combination therapies are described in the following subsections.

In some embodiments, compositions containing g-NK cells as provided herein exhibit enhanced activity when activated by or contacted with antibodies or Fc-containing proteins, such as compared to conventional NK cells. For example, the g-NK cells can be activated by antibody-mediated crosslinking of CD16 or by antibody-coated tumor cells.

In some embodiments, provided herein is a method of treating a condition in an individual comprising administering g-NK cells or composition thereof and an antibody to a subject. One of ordinary skill in the art can select an appropriate therapeutic (e.g., anti-cancer) monoclonal antibody to administer to the subject with the provided g-NK cells and compositions described herein, such as depending on the particular disease or condition of the individual. Suitable antibodies may include polyclonal, monoclonal, fragments (such as Fab fragments), single chain antibodies and other forms of specific binding molecules.

In some embodiments, the antibody may further include humanized or human antibodies. Humanized forms of non-human antibodies are chimeric Igs, Ig chains or fragments (such as Fv, Fab, Fab′, F(ab′)₂ or other antigen-binding subsequences of an antibody) that contain minimal sequence derived from non-human Ig. In some embodiments, the antibody comprises an Fc domain.

Generally, a humanized antibody has one or more amino acid residues introduced from a non-human source. These non-human amino acid residues are often referred to as “import” residues, which are typically taken from an “import” variable domain. Humanization is accomplished by substituting rodent CDRs or CDR sequences for the corresponding sequences of a human antibody (Jones et al., 1986; Riechmann et al., 1988; Verhoeyen et al., 1988). Such “humanized” antibodies are chimeric antibodies (1989), wherein substantially less than an intact human variable domain has been substituted by the corresponding sequence from a non-human species. In practice, humanized antibodies are typically human antibodies in which some CDR residues and possibly some Fc residues are substituted by residues from analogous sites in rodent antibodies. Humanized antibodies include human antibodies (recipient antibody) in which residues from a complementary determining region (CDR) of the recipient are replaced by residues from a CDR of a non-human species (donor antibody) such as mouse, rat or rabbit, having the desired specificity, affinity and capacity. In some instances, corresponding non-human residues replace Fv framework residues of the human antibody. Humanized antibodies may comprise residues that are found neither in the recipient antibody nor in the imported CDR or framework sequences. In general, the humanized antibody comprises substantially all of at least one, and typically two, variable domains, in which most if not all of the CDR regions correspond to those of a non-human Ig and most if not all of the FR regions are those of a human antibody consensus sequence. The humanized antibody optimally also comprises at least a portion of an antibody constant region (Fc), typically that of a human antibody (Jones et al., 1986; Presta, 1992; Riechmann et al., 1988).

Human antibodies can also be produced using various techniques, including phage display libraries (Hoogenboom et al., 1991; Marks et al., 1991) and the preparation of human mAbs (Boerner et al., 1991; Reisfeld and Sell, 1985). Similarly, introducing human Ig genes into transgenic animals in which the endogenous antibody genes have been partially or completely inactivated can be exploited to synthesize human Abs. Upon challenge, human antibody production is observed, which closely resembles that seen in humans in all respects, including gene rearrangement, assembly, and antibody repertoire (1997a; 1997b; 1997c; 1997d; 1997; 1997; Fishwild et al., 1996; 1997; 1997; 2001; 1996; 1997; 1997; 1997; Lonberg and Huszar, 1995; Lonberg et al., 1994; Marks et al., 1992; 1997; 1997; 1997).

### 1. Multiple Myeloma

a. Anti-CD38 Antibody

In some embodiments, the cells of the present invention can be targeted to tumors by administration with an antibody that recognizes a tumor associated antigen that is CD38. In some embodiments, the method further includes administering to the subject an anti-CD38 antibody. In some embodiments, the methods are for treating multiple myeloma. In some embodiments, the antibody is Daratumumab (e.g. Darzalex™).

The g-NK cells and the additional agent can be administered sequentially or simultaneously. In some embodiments, the additional agent can be administered before administration of the g-NK cells. In some embodiments, the additional agent can be administered after administration of the g-NK cells. For example, the g-NK cells can be administered simultaneously with antibodies specific for a selected cancer type. Alternatively, the g-NK cells can be administered at selected times that are distinct from the times when antibodies specific for a selected cancer type are administered.

In some embodiments, at least one dose of anti-CD38 antibody has been administered to the subject prior to administration of a dose of the composition of g-NK cells. In one aspect, disclosed herein is a method of treating multiple myeloma, where the method includes administering a composition of Natural Killer (NK) cells deficient in expression of FcRγ chain (g-NK cells) to a subject having multiple myeloma (MM), wherein the composition of g-NK cells may be administered once weekly for a predetermined number of doses, and wherein the subject has received prior administration of at least one dose of an anti-CD38 antibody.

In some embodiments, the anti-CD38 antibody may be daratumumab. In some embodiments, administration of the at least one dose of the anti-CD38 antibody may be initiated within one month prior to administration of the composition of g-NK cells. In some embodiments, administration of the at least one dose of the anti-CD38 antibody may be initiated within three weeks prior to administration of the composition of g-NK cells. In some embodiments, administration of the at least one dose of the anti-CD38 antibody may be initiated within two weeks prior to administration of the composition of g-NK cells

In particular examples, the subject is administered an effective dose of an antibody before, after, or substantially simultaneously with the population of g-NK cells. An effective amount of the antibody can be selected by a skilled clinician, taking into consideration the particular antibody, the particular disease or conditions (e.g. tumor or other disorder), the general condition of the subject, any additional treatments the subject is receiving or has previously received, and other relevant factors. The subject is also administered a population of g-NK cells described herein. Both the antibody and the population of g-NK cells are typically administered parenterally, for example intravenously; however, injection or infusion to a tumor or close to a tumor (local administration) or administration to the peritoneal cavity can also be used. One of skill in the art can determine appropriate routes of administration.

In some embodiments, the anti-CD38 antibody may be administered as a once weekly dose. In some embodiments, the anti-CD38 antibody may be administered in a cycling regimen. In some embodiments, the antibody is administered in a 28-day cycle. In some embodiments, the antibody is administered for one or two 28-day cycles. In some embodiments, the antibody is administered once weekly in at least one cycle, such as each cycle. In some embodiments, the antibody is administered once weekly for 4 weeks, 6 weeks, 8 weeks, 10 weeks, 12 weeks, 16 weeks, 20 weeks, 24 weeks, 28 weeks, 32 weeks, 36 weeks or more. In some embodiments, eight (8) once weekly doses of the antibody is administered. In some embodiments, the once weekly doses are administered in consecutive weeks.

In some embodiments, the anti-CD38 antibody may be administered intravenously.

In some embodiments, each dose of the anti-CD38 antibody (e.g. daratumumab) may be administered in an amount that may be from or from about 8 mg/kg to about 32 mg/kg. In some embodiments, each dose is at or about 16 mg/kg.

In some embodiments, the anti-CD38 antibody may be administered subcutaneously. In some embodiments, the anti-CD38 antibody (e.g. daratumumab) may be administered in an anti-CD38 antibody composition including a hyaluronidase. For instance, the antibody may be administered as an anti-CD38 antibody composition includes daratumumab and recombinant human hyaluronidase PH20 (e.g. hyaluronidase-fihj). Exemplary of such compositions are described in published U.S. patent publication No. US20170121414. In some embodiments, each dose of the anti-CD38 antibody composition includes from at or about 1200 mg to about 2400 mg anti-CD38 antibody (e.g. daratumumab) and from at or about 15,000 Units (U) to about 45,000 U hyaluronidase (e.g. hyaluronidase-fihj). In some embodiments, each dose of the anti-CD38 antibody composition includes about 1800 mg anti-CD38 antibody (e.g. daratumumab) and about 30,000 U hyaluronidase (e.g. hyaluronidase-fihj).

In some embodiments, the method includes administering the anti-CD38 antibody, once weekly for 8 total doses and administering the g-NK cell composition once weekly for 6 total doses, wherein one dose or two doses of the anti-CD38 antibody may be administered prior to administration of the composition including g-NK cells.

In some embodiments, the multiple myeloma may be relapsed/refractory multiple myeloma.

In some embodiments, the g-NK cells have low or no expression of CD38, such as wherein less than 25% of the cells in the g-NK cell composition are positive for surface CD38. In some embodiments, the cells in the g-NK cell composition are not engineered to reduce or eliminate CD38 expression. In some embodiments, the g-NK cell composition exhibits minimal anti-CD38-induced fratricide, optionally wherein less than 10% of cells in the g-NK cell composition exhibit anti-CD38 induced fratricide.

b. Anti-SLAMF7 Antibody

In some embodiments, the cells of the present invention can be targeted to tumors by administration with an antibody that recognizes a tumor associated antigen that is SLAMF7. In some embodiments, the method further includes administering to the subject an anti-SLAMF7 antibody. In some embodiments, the methods are for treating multiple myeloma. In some embodiments, the antibody is elotuzumab (e.g. EMPLICITI®).

The g-NK cells and the additional agent can be administered sequentially or simultaneously. In some embodiments, the additional agent can be administered before administration of the g-NK cells. For example, the g-NK cells can be administered simultaneously with antibodies specific for a selected cancer type. Alternatively, the g-NK cells can be administered at selected times that are distinct from the times when antibodies specific for a selected cancer type are administered.

In some embodiments, at least one dose of anti-SLAMF7 antibody has been administered to the subject prior to administration of a dose of the composition of g-NK cells. In one aspect, disclosed herein is a method of treating multiple myeloma, wherein the method includes administering a composition of Natural Killer (NK) cells deficient in expression of FcRγ chain (g-NK cells) to a subject having multiple myeloma (MM), wherein the composition of g-NK cells may be administered once weekly for a predetermined number of doses, and wherein the subject has received prior administration of at least one dose of an anti-SLAMF7 antibody.

In some embodiments, the anti-SLAMF7 antibody may be elotuzumab. In some embodiments, administration of the at least one dose of the anti-SLAMF7 antibody may be initiated within one month prior to administration of the composition of g-NK cells. In some embodiments, administration of the at least one dose of the anti-SLAMF7 antibody may be initiated within three weeks prior to administration of the composition of g-NK cells. In some embodiments, administration of the at least one dose of the anti-SLAMF7 antibody may be initiated within two weeks prior to administration of the composition of g-NK cells.

In particular, examples, the subject is administered an effective dose of an antibody before, after, or substantially simultaneously with the population of g-NK cells. An effective amount of the antibody can be selected by a skilled clinician, taking into consideration the particular antibody, the particular disease or conditions (e.g. tumor or other disorder), the general condition of the subject, any additional treatments the subject is receiving or has previously received, and other relevant factors. The subject is also administered a population of g-NK cells described herein. Both the antibody and the population of g-NK cells are typically administered parenterally, for example intravenously; however, injection or infusion to a tumor or close to a tumor (local administration) or administration to the peritoneal cavity can also be used. One of skill in the art can determine appropriate routes of administration.

In some embodiments, the anti-SLAMF7 antibody may be administered as a once weekly dose. In some embodiments the anti-SLAMF7 antibody may be administered in a cycling regimen. In some embodiments, the antibody is administered in a 28-day cycle. In some embodiments, the antibody is administered for one or two 28-day cycles. In some embodiments, the antibody is administered once weekly in at least one cycle, such as each cycle. In some embodiments, the antibody is administered once weekly for 4 week, 6 weeks, 8 weeks, 10 weeks, 12 weeks, 16 weeks, 20 weeks, 24 weeks, 28 weeks, 32 weeks, 36 weeks or more. In some embodiments, eight (8) once weekly doses of the antibody is administered. In some embodiments, the once weekly doses are administered in consecutive weeks.

In some embodiments, the anti-SLAMF7 antibody may be administered intravenously. In some embodiments, the anti-SLAMF7 antibody may be administered subcutaneously.

In some embodiments, each dose of the anti-SLAMF7 antibody (e.g. elotuzumab) may be administered in an amount that may be at or about 10 mg/kg weekly for two cycles and every 2 weeks thereafter. In some embodiments, the anti-SLAMF7 antibody is administered with lenalidomide and dexamethasone. In some embodiments, the anti-SLAMF7 antibody is administered after dexamethasone, diphenhydramine, rantidine, and acetaminophen.

In some embodiments, the method includes administering the anti-SLAMF7 antibody, once weekly for 8 total doses and administering the g-NK cell composition once weekly for 6 total doses, wherein one dose or two doses of the anti-SLAMF7 antibody may be administered prior to administration of the composition including g-NK cells.

In some embodiments, the multiple myeloma may be relapsed/refractory multiple myeloma.

In some embodiments, the g-NK cells have low or no expression of SLAMF7, such as wherein less than 25% of the cells in the g-NK cell composition are positive for surface SLAMF7. In some embodiments, the cells in the g-NK cell composition are not engineered to reduce or eliminate SLAMF7 expression. In some embodiments, the g-NK cell composition exhibits minimal anti-SLAMF7-induced fratricide, optionally wherein less than 10% of cells in the g-NK cell composition exhibit anti-SLAMF7 induced fratricide.

c. Anti-BCMA Antibody

In some embodiments, the cells of the present invention can be targeted to tumors by administration with an antibody that recognizes a tumor associated antigen that is BCMA. In some embodiments, the method further includes administering to the subject an anti-BCMA antibody. In some embodiments, the methods are for treating multiple myeloma. In some embodiments, the antibody is belantamab (e.g. Blenrep).

The g-NK cells and the additional agent can be administered sequentially or simultaneously. In some embodiments, the additional agent can be administered before administration of the g-NK cells. For example, the g-NK cells can be administered simultaneously with antibodies specific for a selected cancer type. Alternatively, the g-NK cells can be administered at selected times that are distinct from the times when antibodies specific for a selected cancer type are administered.

In some embodiments, at least one dose of anti-BCMA antibody has been administered to the subject prior to administration of a dose of the composition of g-NK cells. In one aspect, disclosed herein is a method of treating multiple myeloma, wherein the method includes administering a composition of Natural Killer (NK) cells deficient in expression of FcRγ chain (g-NK cells) to a subject having multiple myeloma (MM), wherein the composition of g-NK cells may be administered once weekly for a predetermined number of doses, and wherein the subject has received prior administration of at least one dose of an anti-BCMA antibody.

In some embodiments, the anti-BCMA antibody may be belantamab. In some embodiments, administration of the at least one dose of the anti-BCMA antibody may be initiated within one month prior to administration of the composition of g-NK cells. In some embodiments, administration of the at least one dose of the anti-BCMA antibody may be initiated within three weeks prior to administration of the composition of g-NK cells. In some embodiments, administration of the at least one dose of the anti-BCMA antibody may be initiated within two weeks prior to administration of the composition of g-NK cells.

In particular, examples, the subject is administered an effective dose of an antibody before, after, or substantially simultaneously with the population of g-NK cells. An effective amount of the antibody can be selected by a skilled clinician, taking into consideration the particular antibody, the particular disease or conditions (e.g. tumor or other disorder), the general condition of the subject, any additional treatments the subject is receiving or has previously received, and other relevant factors. The subject is also administered a population of g-NK cells described herein. Both the antibody and the population of g-NK cells are typically administered parenterally, for example intravenously; however, injection or infusion to a tumor or close to a tumor (local administration) or administration to the peritoneal cavity can also be used. One of skill in the art can determine appropriate routes of administration.

In some embodiments, the anti-BCMA antibody may be administered as a once weekly dose. In some embodiments the anti-BCMA antibody may be administered in a cycling regimen. In some embodiments, the antibody is administered in a 28-day cycle. In some embodiments, the antibody is administered for one or two 28-day cycles. In some embodiments, the antibody is administered once weekly in at least one cycle, such as each cycle. In some embodiments, the antibody is administered once weekly for 4 week, 6 weeks, 8 weeks, 10 weeks, 12 weeks, 16 weeks, 20 weeks, 24 weeks, 28 weeks, 32 weeks, 36 weeks or more. In some embodiments, eight (8) once weekly doses of the antibody is administered. In some embodiments, the once weekly doses are administered in consecutive weeks.

In some embodiments, the anti-BCMA antibody may be administered intravenously. In some embodiments, the anti-BCMA antibody may be administered subcutaneously. In some embodiments, the anti-BCMA antibody (e.g., Blenrep) may be administered at or about 2.5 mg/kg as an intravenous infusion over at or about 30 minutes. In some embodiments, the anti-BCMA antibody (e.g., Blenrep) is administered once every three weeks.

In some embodiments, the method includes administering the anti-BCMA antibody, once weekly for 8 total doses and administering the g-NK cell composition once weekly for 6 total doses, wherein one dose or two doses of the anti-BCMA antibody may be administered prior to administration of the composition including g-NK cells.

In some embodiments, the multiple myeloma may be relapsed/refractory multiple myeloma.

In some embodiments, the g-NK cells have low or no expression of BCMA, such as wherein less than 25% of the cells in the g-NK cell composition are positive for surface BCMA. In some embodiments, the cells in the g-NK cell composition are not engineered to reduce or eliminate BCMA expression. In some embodiments, the g-NK cell composition exhibits minimal anti-BCMA-induced fratricide, optionally wherein less than 10% of cells in the g-NK cell composition exhibit anti-BCMA induced fratricide.

### 2. Lymphoma

a. Anti-CD20 Antibody

In some embodiments, the cells of the present invention can be targeted to tumors by administration with an antibody that recognizes a tumor associated antigen that is CD20. In some embodiments, the method further includes administering to the subject an anti-CD20 antibody. In some embodiments, the methods are for treating a lymphoma, such as a Non-Hodgkin's lymphoma. In some embodiments, the antibody is rituximab (e.g. Rituxan®).

The g-NK cells and the additional agent can be administered sequentially or simultaneously. In some embodiments, the additional agent can be administered before administration of the g-NK cells. In some embodiments, the additional agent can be administered after administration of the g-NK cells. For example, the g-NK cells can be administered simultaneously with antibodies specific for a selected cancer type. Alternatively, the g-NK cells can be administered at selected times that are distinct from the times when antibodies specific for a selected cancer type are administered.

In some embodiments, at least one dose of anti-CD20 antibody has been administered to the subject prior to administration of a dose of the composition of g-NK cells. In one aspect, disclosed herein is a method of treating lymphoma, where the method includes administering a composition of Natural Killer (NK) cells deficient in expression of FcRγ chain (g-NK cells) to a subject having lymphoma, wherein the composition of g-NK cells may be administered once weekly for a predetermined number of doses, and wherein the subject has received prior administration of at least one dose of an anti-CD20 antibody.

In some embodiments, the anti-CD20 antibody may be rituximab.

In some embodiments, administration of the at least one dose of the anti-CD20 antibody may be initiated within one month prior to administration of the composition of g-NK cells. In some embodiments, at least one dose of the anti-CD20 antibody may be initiated within three weeks prior to administration of the composition of g-NK cells. In some embodiments, administration of the at least one dose of the anti-CD20 antibody may be initiated within two weeks prior to administration of the composition of g-NK cells.

In particular examples, the subject is administered an effective dose of an antibody before, after, or substantially simultaneously with the population of g-NK cells. An effective amount of the antibody can be selected by a skilled clinician, taking into consideration the particular antibody, the particular disease or conditions (e.g. tumor or other disorder), the general condition of the subject, any additional treatments the subject is receiving or has previously received, and other relevant factors. The subject is also administered a population of g-NK cells described herein. Both the antibody and the population of g-NK cells are typically administered parenterally, for example intravenously; however, injection or infusion to a tumor or close to a tumor (local administration) or administration to the peritoneal cavity can also be used. One of skill in the art can determine appropriate routes of administration.

In some embodiments, the anti-CD20 antibody may be administered as a once weekly dose. In some embodiments, the anti-CD20 antibody may be administered in a cycling regimen. In some embodiments, the antibody is administered in a 28-day cycle. In some embodiments, the antibody is administered for one or two 28-day cycles. In some embodiments, the antibody is administered once weekly in at least one cycle, such as each cycle. In some embodiments, the antibody is administered once weekly for 4 weeks, 6 weeks, 8 weeks, 10 weeks, 12 weeks, 16 weeks, 20 weeks, 24 weeks, 28 weeks, 32 weeks, 36 weeks or more. In some embodiments, eight (8) once weekly doses of the antibody is administered. In some embodiments, the once weekly doses are administered in consecutive weeks.

In some embodiments, the anti-CD20 antibody may be administered intravenously.

In some embodiments, each dose of the anti-CD20 antibody may be administered in an amount that may be from or from about 250 mg/m2 to 500 mg/m². In some embodiments, each does is administered at or about 375 mg/m².

In some embodiments, the anti-CD20 antibody may be administered subcutaneously. In some embodiments, the anti-CD20 antibody (e.g. rituximab) may be administered in an anti-CD20 antibody composition including a hyaluronidase. For instance, the antibody may be administered as an anti-CD20 antibody composition includes rituximab and recombinant human hyaluronidase PH20. Exemplary examples of such compositions are described in published PCT publication No. WO2011029892.

In some embodiments, each dose of the anti-CD20 antibody composition includes from at or about 1200 mg to about 2400 mg anti-CD20 antibody (e.g. rituximab) and from at or about 15,000 Units (U) to about 45,000 U hyaluronidase. In some embodiments, each dose of the anti-CD20 antibody composition includes about 1400 mg anti-CD20 antibody (e.g. rituximab) and about 23,400 U hyaluronidase. In some embodiments, each dose of the anti-CD20 antibody composition includes about 1600 mg anti-CD20 antibody (e.g. rituximab) and about 26,800 U hyaluronidase.

In some embodiments, the anti-CD20 antibody composition may be administered as a once weekly dose. In some embodiments, the anti-CD20 antibody is administered as 4 or 8 doses. In some embodiments, the antibody is administered for 3 or 7 doses subcutaneously following a once weekly dose of the anti-CD20 antibody intravenously. In some embodiments, the method includes administering the anti-CD20 antibody once weekly for 8 total doses and administering the g-NK cell composition once weekly for 6 total doses, wherein one dose or two doses of the anti-CD20 antibody may be administered prior to administration of the composition including g-NK cells.

b. Anti-CD19 Antibody

In some embodiments, the cells of the present invention can be targeted to tumors by administration with an antibody that recognizes a tumor associated antigen that is CD19. In some embodiments, the method further includes administering to the subject an anti-CD19 antibody. In some embodiments, the methods are for treating a lymphoma, such as a Non-Hodgkin's lymphoma. In some embodiments, the antibody is tafasitamab (e.g. MONJUVI®). In other embodiments, the antibody is loncastuximab (e.g. ZYNLONTA®).

The g-NK cells and the additional agent can be administered sequentially or simultaneously. In some embodiments, the additional agent can be administered before administration of the g-NK cells. In some embodiments, the additional agent can be administered after administration of the g-NK cells. For example, the g-NK cells can be administered simultaneously with the antibodies specific for a selected cancer type. Alternatively, the g-NK cells can be administered at selected times that are distinct from the times when antibodies specific for a selected cancer type are administered.

In some embodiments, at least one dose of anti-CD19 antibody has been administered to the subject prior to administration of a dose of the composition of g-NK cells. In one aspect, disclosed here is a method of treating lymphoma, wherein the method includes administering a composition of Natural Killer (NK) cells deficient in expression of FcRγ chain (g-NK cells) to a subject having lymphoma, wherein the composition of g-NK cells may be administered once weekly for a predetermined number of doses, and wherein the subject has received prior administration of an anti-CD19 antibody.

In some embodiments, the CD19 antibody may be tafasitamab. In other embodiments, the CD19 antibody may be loncastuximab.

In some embodiments, the administration of the at least one dose of the anti-CD19 antibody may be initiated within one month prior to administration of the composition of g-NK cells. In some embodiments, at least one dose of the anti-CD19 antibody may be initiated within three weeks prior to administration of the composition of g-NK cells. In some embodiments, administration of the at least one dose of the anti-CD19 antibody may be initiated within two weeks prior to administration of the composition of g-NK cells.

In one particular example, the subject is administered an effective dose of an antibody before, after, or substantially simultaneously with the population of g-NK cells. An effective amount of the antibody can be selected by a skilled clinician, taking into consideration the particular antibody, the particular disease or condition (e.g. tumor or other disorder), the general condition of the subject, any additional treatments the subject is receiving or has previously received, and other relevant factors. The subject is also administered a population of g-NK cells described herein. Both the antibody and the population of g-NK cells are typically administered parenterally, for example intravenously; however, injection or infusion to a tumor or close to a tumor (local administration) or administration to the peritoneal cavity can also be used. One of skill in the art can determine appropriate routes of administration.

In some embodiments, the anti-CD19 antibody may be administered as a once weekly dose. In some embodiments, the anti-CD19 antibody may be administered in a cycling regiment. In some embodiments, the antibody is administered in a 28-day cycle. In some embodiments, the antibody is administered once weekly in at least one cycle, such as each cycle. In some embodiments, the antibody is administered once weekly for 4 weeks, 6 weeks, 8 weeks, 10 weeks, 12 weeks, 16 weeks, 20 weeks, 24 weeks, 28 weeks, 32 weeks, 36 weeks or more. In some embodiments, eight (8) once weekly doses of the antibody is administered. In some embodiments, the once weekly doses are administered in consecutive weeks.

In some embodiments, the anti-CD19 antibody may be administered intravenously. In some embodiments, the anti-CD19 antibody may be administered subcutaneously. In some embodiments, the anti-CD19 antibody (e.g., tafasitamab) is administered at or about 12 mg/kg. In some embodiments, the anti-CD19 antibody (e.g., tafasitamab) is administered over four cycles. In some embodiments, the first cycle comprises administration on days 1, 4, 8, 15, and 22 of a 28-day cycle. In some embodiments, the second and third cycles comprise administration on days 1, 8, 15, and 22 of a 28-day cycle. In some embodiments, the fourth cycle and beyond comprises administration on days 1 and 15 of a 28-day cycle. In some embodiments, the anti-CD19 antibody (e.g., tafasitamab) is administered for 1, 2, 3, 4, 5, 6, 7, 8, 9, 10, 11, or 12 cycles.

In some embodiments, the anti-CD19 antibody (e.g., loncastuximab) is administered at or about 0.15 mg/kg every 3 weeks for 2 cycles. In some embodiments, the anti-CD19 antibody (e.g., loncastuximab) is administered at or about 0.075 mg/kg every 3 weeks for subsequent cycles. In some embodiments, dexamethasone is administered prior to administration of the anti-CD19 antibody (e.g., loncastuximab).

In some embodiments, the anti-CD19 antibody composition may be administered as a once weekly dose. In some embodiments, the anti-CD19 antibody is administered as 4 or 8 doses. In some embodiments, the antibody is administered for 3 or 7 doses subcutaneously following a once weekly dose of the anti-CD19 antibody intravenously. In some embodiments, the method includes administering the anti-CD19 antibody once weekly for 8 total doses and administering the g-NK cell composition once weekly for 6 total doses, wherein one dose or two doses of the anti-CD19 antibody may be administered prior to administration of the composition including g-NK cells.

Exemplary examples are described in WO2020249528A1 and U.S. Pat. No. 8,524,867.

c. Anti-CD30 Antibody

In some embodiments, the cells of the present invention can be targeted to tumors by administration with an antibody that recognizes a tumor associated antigen that is CD30. In some embodiments, the method further includes administering to the subject an anti-CD30 antibody. In some embodiments, the methods are for treating a lymphoma, such as a Non-Hodgkin's lymphoma. In some embodiments, the antibody is brentuximab (ADCETRIS®).

The g-NK cells and the additional agent can be administered sequentially or simultaneously. In some embodiments, the additional agent can be administered before administration of the g-NK cells. In some embodiments, the additional agent can be administered after administration of the g-NK cells. For example, the g-NK cells can be administered simultaneously with the antibodies specific for a selected cancer type. Alternatively, the g-NK cells can be administered at selected times that are distinct from the times when antibodies specific for a selected cancer type are administered.

In some embodiments, at least one dose of anti-CD30 antibody has been administered to the subject prior to administration of a dose of the composition of g-NK cells. In one aspect, disclosed here is a method of treating lymphoma, wherein the method includes administering a composition of Natural Killer (NK) cells deficient in expression of FcRγ chain (g-NK cells) to a subject having lymphoma, wherein the composition of g-NK cells may be administered once weekly for a predetermined number of doses, and wherein the subject has received prior administration of an anti-CD30 antibody.

In some embodiments, the CD30 antibody may be brentuximab.

In some embodiments, the administration of the at least one dose of the anti-CD30 antibody may be initiated within one month prior to administration of the composition of g-NK cells. In some embodiments, at least one dose of the anti-CD30 antibody may be initiated within three weeks prior to administration of the composition of g-NK cells. In some embodiments, administration of the at least one dose of the anti-CD30 antibody may be initiated within two weeks prior to administration of the composition of g-NK cells.

In one particular example, the subject is administered an effective dose of an antibody before, after, or substantially simultaneously with the population of g-NK cells. An effective amount of the antibody can be selected by a skilled clinician, taking into consideration the particular antibody, the particular disease or condition (e.g. tumor or other disorder), the general condition of the subject, any additional treatments the subject is receiving or has previously received, and other relevant factors. The subject is also administered a population of g-NK cells described herein. Both the antibody and the population of g-NK cells are typically administered parenterally, for example intravenously; however, injection or infusion to a tumor or close to a tumor (local administration) or administration to the peritoneal cavity can also be used. One of skill in the art can determine appropriate routes of administration.

In some embodiments, the anti-CD30 antibody may be administered as a once weekly dose. In some embodiments, the anti-CD30 antibody may be administered in a cycling regiment. In some embodiments, the antibody is administered in a 28-day cycle. In some embodiments, the antibody is administered once weekly in at least one cycle, such as each cycle. In some embodiments, the antibody is administered once weekly for 4 weeks, 6 weeks, 8 weeks, 10 weeks, 12 weeks, 16 weeks, 20 weeks, 24 weeks, 28 weeks, 32 weeks, 36 weeks or more. In some embodiments, eight (8) once weekly doses of the antibody is administered. In some embodiments, the once weekly doses are administered in consecutive weeks.

In some embodiments, the anti-CD30 antibody may be administered intravenously. In some embodiments, the anti-CD30 antibody may be administered subcutaneously. In some embodiments, the anti-CD30 antibody (e.g. brentuximab) may be administered at or about 1.8 mg/kg. In some embodiments the anti-CD30 antibody (e.g., brentuximab) may be administered up to a maximum of 180 mg. In some embodiments, the anti-CD30 (e.g., brentuximab) may be administered every three weeks.

In some embodiments, the anti-CD30 antibody composition may be administered as a once weekly dose. In some embodiments, the anti-CD30 antibody is administered as 4 or 8 doses. In some embodiments, the antibody is administered for 3 or 7 doses subcutaneously following a once weekly dose of the anti-CD30 antibody intravenously. In some embodiments, the method includes administering the anti-CD30 antibody once weekly for 8 total doses and administering the g-NK cell composition once weekly for 6 total doses, wherein one dose or two doses of the anti-CD30 antibody may be administered prior to administration of the composition including g-NK cells.

Exemplary examples are described in U.S. Pat. No. 7,659,241.

### 3. Bi-Specific Antibody (BsAb)

In some embodiments provided herein, the g-NK cells can be administered to an individual in combination with a bispecific antibody (BsAb). BsAbs are designed to recognize and bind to two different antigens or epitopes. Examples of BsAbs are bispecific T cell engagers (BiTEs) and bispecific Natural Killer cell engagers (BiKEs). BiKEs have been generated to engage CD16 on a Natural Killer cell and a second tumor antigen, and various examples of BiKEs targeting CD16 and a second tumor antigen have been described in the literature (Felices, et al. (2018) Methods Mol. Bio., 1441:333-346). For example, BiKEs have been developed for CD16 with CD19 or CD20 in B cell Non-Hodgkin's lymphomas (Glorius, et al. (2013) Leukemia, 27:190-201; Kipriyanov, et al. (2002), J. Immunol, 169:137-144, Portner, et al. (2012) Cancer immunology, immunotherapy: CII 61:1869-1875); for CD16 with CD19 or CD33 for mixed lineage leukemia (Schubert, et al. (2011) MAbs, 3:21-30); for CD16 with CD19/CD22 for B cell Non-Hodgkin's lymphomas (Gleason, et al. (2012), Mol. Cancer Ther., 11:2674-2684); for CD16 with CD30 for Hodgkin's lymphoma (Hombach, et al. (1993), Int. J. Cancer, 55:830-836); and for CD16 with BCMA for multiple myeloma (Kakiuchi-Kiyota, et al. (2022), Leukemia, 36:1006-1014).

In some embodiments, the bispecific antibody is a bispecific T cell enhancer. In some embodiments, the bispecific antibody is a bispecific NK cell enhancer. In some embodiments, the first tumor target of the bispecific NK cell enhancer (BiKE) is CD16 and the second tumor target of the BiKE is directed to a tumor antigen. In some embodiments, the first tumor target of the BiKE is CD16 and the second tumor target of the BiKE is CD19. In some embodiments, the first tumor target of the BiKE is CD16 and the second tumor target of the BiKE is CD20. In some embodiments, the first tumor target of the BiKE is CD16 and the second tumor target of the BiKE is CD30. In some embodiments, the first tumor target of the BiKE is CD16 and the second tumor target of the BiKE is CD38. In some embodiments, the first tumor target of the BiKE is CD16 and the second tumor target of the BiKE is SLAMF7. In some embodiments, the first tumor target of the BiKE is CD16 and the second tumor target of the BiKE is BCMA.

### 4. Cytokines or Growth Factors

In some embodiments provided herein, the g-NK cells can be administered to an individual in combination with cytokines and/or growth factors. According to some embodiments, the at least one growth factor comprises a growth factor selected from the group consisting of SCF, FLT3, IL-2, IL-7, IL-15, IL-12, IL-21, and IL-27. In particular embodiments, recombinant IL-2 is administered to the subject. In other particular embodiments, recombinant IL-15 is administered to the subject. In other particular embodiments, recombinant IL-21 is administered to the subject. In some embodiments, the g-NK cells and the cytokines or growth factors are administered sequentially. For example, the g-NK cells may be administered first, followed by administration of the cytokines and/or growth factors. In some embodiments, the g-NK cells are administered simultaneously with the cytokines or growth factors.

In some embodiments, the subject is administered one or more cytokines (such as IL-2, IL-15, IL-21, IL-27, and/or IL-12) to support survival and/or growth of NK cells. The cytokine(s) can be administered before, after, or substantially simultaneously with the NK cells. In some examples, the cytokine(s) can be administered after the NK cells. In one specific example, the cytokine(s) is administered to the subject within about 1-8 hours (such as within about 1-4 hours, about 2-6 hours, about 4-6 hours, or about 5-8 hours) of the administration of the NK cells.

### 5. Lymphodepleting Therapy

In some embodiments, the provided methods also can include administering g-NK cells with another treatment, such as with a chemotherapeutic agent or cytotoxic agent or other treatment.

In some aspects, the provided methods can further include administering one or more lymphodepleting therapies, such as prior to or simultaneous with initiation of administration of the g-NK cell composition. In some embodiments, the lymphodepleting therapy comprises administration of a phosphamide, such as cyclophosphamide. In some embodiments, the lymphodepleting therapy can include administration of fludarabine.

In some aspects, preconditioning subjects with immunodepleting (e.g., lymphodepleting) therapies can improve the effects of adoptive cell therapy (ACT). In some embodiments, the lymphodepleting therapy includes combinations of cyclosporine and fludarabine.

Such preconditioning can be carried out with the goal of reducing the risk of one or more of various outcomes that could dampen efficacy of the therapy. These include the phenomenon known as “cytokine sink,” by which T cells, B cells, NK cells compete with TILs for homeostatic and activating cytokines, such as IL-2, IL-7, and/or IL-15; suppression of TILs by regulatory T cells, NK cells, or other cells of the immune system; impact of negative regulators in the tumor microenvironment. Muranski et al., Nat Clin Pract Oncol. December; 3(12): 668-681 (2006).

Thus in some embodiments, the provided method further involves administering a lymphodepleting therapy to the subject. In some embodiments, the method involves administering the lymphodepleting therapy to the subject prior to the administration of the dose of cells. In some embodiments, the lymphodepleting therapy contains a chemotherapeutic agent such as fludarabine and/or cyclophosphamide. In some embodiments, the administration of the cells and/or the lymphodepleting therapy is carried out via outpatient delivery.

In some embodiments, the methods include administering a preconditioning agent, such as a lymphodepleting or chemotherapeutic agent, such as cyclophosphamide, fludarabine, or combinations thereof, to a subject prior to the administration of the dose of cells. For example, the subject may be administered a preconditioning agent, such as a lymphodepleting or chemotherapeutic agent, such as cyclophosphamide, fludarabine, or combinations thereof, at least 2 days prior, such as at least 3, 4, 5, 6, or 7 days prior, to the first or subsequent dose. In some embodiments, the subject is administered a preconditioning agent, such as a lymphodepleting or chemotherapeutic agent, such as cyclophosphamide, fludarabine, or combinations thereof, no more than 7 days prior, such as no more than 6, 5, 4, 3, or 2 days prior, to the administration of the dose of cells. In some embodiments, the subject is administered a preconditioning agent, such as a lymphodepleting or chemotherapeutic agent, such as cyclophosphamide, fludarabine, or combinations thereof, no more than 14 days prior, such as no more than 13, 12, 11, 10, 9 or 8 days prior, to the administration of the dose of cells.

In some embodiments, the subject is preconditioned with cyclophosphamide at a dose between or between about 20 mg/kg and 100 mg/kg, such as between or between about 40 mg/kg and 80 mg/kg. In some aspects, the subject is preconditioned with or with about 60 mg/kg of cyclophosphamide. In some embodiments, the fludarabine can be administered in a single dose or can be administered in a plurality of doses, such as given daily, every other day or every three days. In some embodiments, the cyclophosphamide is administered once daily for one or two days.

In some embodiments, where the lymphodepleting agent comprises fludarabine, the subject is administered fludarabine at a dose between or between about 1 mg/m² and 100 mg/m², such as between or between about 10 mg/m² and 75 mg/m², 15 mg/m² and 50 mg/m², 20 mg/m² and 30 mg/m², or 24 mg/m² and 26 mg/m². In some instances, the subject is administered 25 mg/m² of fludarabine. In some embodiments, the fludarabine can be administered in a single dose or can be administered in a plurality of doses, such as given daily, every other day or every three days. In some embodiments, fludarabine is administered daily, such as for 1-5 days, for example, for 3 to 5 days.

In some embodiments, the lymphodepleting agent comprises a combination of agents, such as a combination of cyclophosphamide and fludarabine. Thus, the combination of agents may include cyclophosphamide at any dose or administration schedule, such as those described above, and fludarabine at any dose or administration schedule, such as those described above. For example, in some aspects, the subject is administered 60 mg/kg (˜2 g/m²) of cyclophosphamide and 3 to 5 doses of 25 mg/m² fludarabine prior to the dose of cells.

In some embodiments, prior to the administration of the dose of g-NK cells, the subject has received a lymphodepleting therapy. In some embodiments, the lymphodepleting therapy includes fludarabine and/or cyclophosphamide. In some embodiments, the lymphodepleting includes the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.

In some embodiments, the lymphodepleting therapy includes fludarabine and cyclophosphamide. In some embodiments, the lymphodepleting therapy includes the administration of fludarabine at or about 30 mg/m² body surface area of the subject, daily, and cyclophosphamide at or about 300 mg/m² body surface area of the subject, daily, each for 2-4 days, optionally 3 days.

In some embodiments, the administration of the preconditioning agent prior to infusion of the dose of cells improves an outcome of the treatment. For example, in some aspects, preconditioning, such as a lymphodepleting or chemotherapeutic agent, such as cyclophosphamide, fludarabine, or combinations thereof, improves the efficacy of treatment with the dose or increases the persistence of the NK cells in the subject. In some embodiments, preconditioning treatment increases disease-free survival, such as the percent of subjects that are alive and exhibit no minimal residual or molecularly detectable disease after a given period of time following the dose of cells. In some embodiments, the time to median disease-free survival is increased.

Once the cells are administered to the subject (e.g., human), the biological activity of the engineered cell populations in some aspects is measured by any of a number of known methods. Parameters to assess include specific binding of an engineered or natural T cell or other immune cell to antigen, in vivo, e.g., by imaging, or ex vivo, e.g., by ELISA or flow cytometry. In certain embodiments, the ability of the NK cells to destroy target cells can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the cells also can be measured by assaying expression and/or secretion of certain cytokines or other effector molecules, such as CD107a, IFNγ, and TNF. In some aspects the biological activity is measured by assessing clinical outcome, such as reduction in tumor burden or load. In some aspects, toxic outcomes, persistence and/or expansion of the cells, and/or presence or absence of a host immune response, are assessed.

### III. Methods for Expanding Natural Killer Cell Subsets

In some embodiments, the g-NK cell compositions for use in the provided methods are expanded ex vivo from a subset of NK cells from a biological sample from a human subject. In some embodiments, the methods for expanding and producing a g-NK cell composition can include expanding a subset of cells that are FcRγ-deficient NK cells (g NK) from a biological sample from a human subject. In some embodiments, the methods can include expanding a subset of NK cells that are NKG2C^{pos} from a biological sample from a human subject. In some embodiments, the methods can include expanding a subset of NK cells that are NKG2A^{neg} from a biological sample from a human subject. In some embodiments, the method includes isolating a population of cells enriched for natural killer (NK) cells from a biological sample from a human subject and culturing the cells under conditions in which preferential growth and/or expansion of the g-NK cell subject and/or an NK cell subset that overlaps or shares extracellular surface markers with the g-NK cell subset. For example, the NK cells may be cultured using feeder cells, or in the presence of cytokines to enhance the growth and/or expansion of g-NK cell subject and/or an NK cell subset that overlaps or shares extracellular surface markers with the g-NK cell subset. In some aspects, the provided methods also can expand other subsets of NK cells, such as any NK cell that is NKG2C^{pos} and/or NKG2A^{neg}.

In some embodiments, the sample, e.g. biological sample, is one containing a plurality of cell populations that includes an NK cell population. In some embodiments, the biological sample is or comprises blood cells, e.g. peripheral blood mononuclear cells. In some aspects, the biological sample is a whole blood sample, an apheresis product or a leukapheresis product. In some embodiments, the sample is a sample of peripheral blood mononuclear cells (PBMCs). Thus, in some embodiments of the provided methods, a population of peripheral blood mononuclear cells (PBMCs) can be obtained. The sample containing a plurality of cell populations that includes an NK cell population can be used as the cells for enriching or selecting an NK cell subset for expansion in accord with the provided methods.

In some embodiments, the biological sample is from a subject that is a healthy subject. In some embodiments, the biological sample is from a subject that has a disease of conditions, e.g. a cancer.

In some embodiments, the cells are isolated or selected from a sample, such as a biological sample, e.g., one obtained from or derived from a subject, such as one having a particular disease or condition or in need of a cell therapy or to which cell therapy will be administered. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom. In some aspects, the sample is blood or a blood-derived sample, or is or is derived from an apheresis or leukapheresis product.

In some examples, cells from the circulating blood of a subject are obtained. The samples, in some aspects, contain lymphocytes, including NK cells, T cells, monocytes, granulocytes, B cells, other nucleated white blood cells, red blood cells, and/or platelets, and in some aspects contains cells other than red blood cells and platelets. In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient, such as by using a Histopaque® density centrifugation.

In some embodiments, the biological sample is from an enriched leukapheresis product collected from normal peripheral blood. In some embodiments, the enriched leukapheresis product can contain fresh cells. In some embodiments, the enriched leukapheresis product is a cryopreserved sample that is thawed for use in the provided methods.

In some embodiments, the source of biological cells contains from at or about 5×10⁵ to at or about 5×10⁸ NK cells or a g-NK cell subset or an NK cell subset that is associated with or includes a surrogate marker for g-NK cells. In some embodiments, the number of NK cells, or a g-NK cell subset or an NK cell subset that is associated with or includes a surrogate marker for g-NK cells, in the biological sample is from at or about 5×10⁵ to at or about 1×10⁸, from at or about 5×10⁵ to at or about 5×10⁷, from at or about 5×10⁵ to at or about 1×10⁷, from at or about 5×10⁵ to at or about 5×10⁶, from at or about 5×10⁵ to at or about 1×10⁶, from at or about 1×10⁶ to at or about 1×10⁸, from at or about 1×10⁶ to at or about 5×10⁷, from at or about 1×10⁶ to at or about 1×10⁷, from at or about 1×10⁶ to at or about 5×10⁶, from at or about 5×10⁶ to at or about 1×10⁸, from at or about 5×10⁶ to at or about 5×10⁷, from at or about 5×10⁶ to at or about 1×10⁷, from at or about 1×10⁷ to at or about 1×10⁸, from at or about 1×10⁷ to at or about 5×10⁷, or from at or about 5×10⁷ to at or about 1×10⁸.

In some embodiments, the percentage of g-NK cells, or of an NK cell subset that is associated with or includes a surrogate marker for g-NK cells, among NK cells in the biological sample is greater than at or about 3%. In some embodiments, the percentage of g-NK cells, or of an NK cell subset that is associated with or includes a surrogate marker for g-NK cells, among NK cells in the biological sample is greater than at or about 5%. In some embodiments, the percentage of g-NK cells, or of an NK cell subset that is associated with or includes a surrogate marker for g-NK cells, among NK cells in the biological sample is greater than at or about 10%. In some embodiments, the percentage of g-NK cells, or of an NK cell subset that is associated with or includes a surrogate marker for g-NK cells, among NK cells in the biological sample is greater than at or about 12%. In some embodiments, the percentage of g-NK cells, or of an NK cell subset that is associated with or includes a surrogate marker for g-NK cells, among NK cells in the biological sample is greater than at or about 14%. In some embodiments, the percentage of g-NK cells, or of an NK cell subset that is associated with or includes a surrogate marker for g-NK cells, among NK cells in the biological sample is greater than at or about 16%. In some embodiments, the percentage of g-NK cells, or of an NK cell subset that is associated with or includes a surrogate marker for g-NK cells, among NK cells in the biological sample is greater than at or about 18%. In some embodiments, the percentage of g-NK cells, or of an NK cell subset that is associated with or includes a surrogate marker for g-NK cells, among NK cells in the biological sample is greater than at or about 20%. In some embodiments, the percentage of g-NK cells, or of an NK cell subset that is associated with or includes a surrogate marker for g-NK cells, among NK cells in the biological sample is greater than at or about 22%. In some embodiments, the percentage of g-NK cells, or of an NK cell subset that is associated with or includes a surrogate marker for g-NK cells, among NK cells in the biological sample is greater than at or about 24%. In some embodiments, the percentage of g-NK cells, or of an NK cell subset that is associated with or includes a surrogate marker for g-NK cells, among NK cells in the biological sample is greater than at or about 26%. In some embodiments, the percentage of g-NK cells, or of an NK cell subset that is associated with or includes a surrogate marker for g-NK cells, among NK cells in the biological sample is greater than at or about 28%. In some embodiments, the percentage of g-NK cells, or of an NK cell subset that is associated with or includes a surrogate marker for g-NK cells, among NK cells in the biological sample is greater than at or about 30%.

In some embodiments, a subject is selected if the percentage of g-NK cells, or of an NK cell subset that is associated with or includes a surrogate marker for g-NK cells, among NK cells in the biological sample is greater than at or about 3%. In some embodiments, a subject is selected if the percentage of g-NK cells, or of an NK cell subset that is associated with or includes a surrogate marker for g-NK cells, among NK cells in the biological sample is greater than at or about 5%. In some embodiments, a subject is selected if the percentage of g-NK cells, or of an NK cell subset that is associated with or includes a surrogate marker for g-NK cells, among NK cells in the biological sample is greater than at or about 10%. In some embodiments, a subject is selected if the percentage of g-NK cells, or of an NK cell subset that is associated with or includes a surrogate marker for g-NK cells, among NK cells in the biological sample is greater than at or about 12%. In some embodiments, a subject is selected if the percentage of g-NK cells, or of an NK cell subset that is associated with or includes a surrogate marker for g-NK cells, among NK cells in the biological sample is greater than at or about 14%. In some embodiments, a subject is selected if the percentage of g-NK cells, or of an NK cell subset that is associated with or includes a surrogate marker for g-NK cells, among NK cells in the biological sample is greater than at or about 16%. In some embodiments, a subject is selected if the percentage of g-NK cells, or of an NK cell subset that is associated with or includes a surrogate marker for g-NK cells, among NK cells in the biological sample is greater than at or about 18%. In some embodiments, a subject is selected if the percentage of g-NK cells, or of an NK cell subset that is associated with or includes a surrogate marker for g-NK cells, among NK cells in the biological sample is greater than at or about 20%. In some embodiments, a subject is selected if the percentage of g-NK cells, or of an NK cell subset that is associated with or includes a surrogate marker for g-NK cells, among NK cells in the biological sample is greater than at or about 22%. In some embodiments, a subject is selected if the percentage of g-NK cells, or of an NK cell subset that is associated with or includes a surrogate marker for g-NK cells, among NK cells in the biological sample is greater than at or about 24%. In some embodiments, a subject is selected if the percentage of g-NK cells, or of an NK cell subset that is associated with or includes a surrogate marker for g-NK cells, among NK cells in the biological sample is greater than at or about 26%. In some embodiments, a subject is selected if the percentage of g-NK cells, or of an NK cell subset that is associated with or includes a surrogate marker for g-NK cells, among NK cells in the biological sample is greater than at or about 28%. In some embodiments, a subject is selected if the percentage of g-NK cells, or of an NK cell subset that is associated with or includes a surrogate marker for g-NK cells, among NK cells in the biological sample is greater than at or about 30%.

In some embodiments, the biological sample is from a subject that is CMV seropositive. CMV infection can result in phenotypic and functional differentiation of NK cells, including development of high fractions of NK cells expressing NKG2C that exhibit enhanced antiviral activity. CMV-associated NK cells expressing NKG2C display altered DNA methylation patterns and reduced expression of signaling molecules, such as FcRγ (Schlums et al., Immunity (2015) 42:443-56). These NK cells are linked to more potent antibody-dependent activation, expansion, and function relative to conventional NK-cell subsets. In some cases, the biological sample can be from a subject that is CMV seronegative as NK cells with reduced expression of FcRγ can also be detected in CMV seronegative individuals, albeit generally at lower levels. In some cases, the biological sample can be from CMV seropositive individuals.

In some embodiments, a subject is selected based on the percentage of NK cells in a peripheral blood sample that are positive for NKG2C. In some embodiments, the subject is selected if at least at or about 20% of NK cells in the peripheral blood sample are positive for NKG2C. In some embodiments, the subject is selected if at least at or about 25% of NK cells in the peripheral blood sample are positive for NKG2C. In some embodiments, the subject is selected if at least at or about 30% of NK cells in the peripheral blood sample are positive for NKG2C. In some embodiments, the subject is selected if at least at or about 35% of NK cells in the peripheral blood sample are positive for NKG2C. In some embodiments, the subject is selected if at least at or about 40% of NK cells in the peripheral blood sample are positive for NKG2C. In some embodiments, the subject is selected if at least at or about 45% of NK cells in the peripheral blood sample are positive for NKG2C. In some embodiments, the subject is selected if at least at or about 50% of NK cells in the peripheral blood sample are positive for NKG2C. In some embodiments, the subject is selected if at least at or about 55% of NK cells in the peripheral blood sample are positive for NKG2C. In some embodiments, the subject is selected if at least at or about 60% of NK cells in the peripheral blood sample are positive for NKG2C.

In some embodiments, a subject is selected based on the percentage of NK cells in a peripheral blood sample that are negative or low for NKG2A. In some embodiments, a subject is selected if at least at or about 70% of NK cells in the peripheral blood sample are negative or low for NKG2A. In some embodiments, a subject is selected if at least at or about 75% of NK cells in the peripheral blood sample are negative or low for NKG2A. In some embodiments, a subject is selected if at least at or about 80% of NK cells in the peripheral blood sample are negative or low for NKG2A. In some embodiments, a subject is selected if at least at or about 85% of NK cells in the peripheral blood sample are negative or low for NKG2A. In some embodiments, a subject is selected if at least at or about 90% of NK cells in the peripheral blood sample are negative or low for NKG2A.

In some embodiments, a subject is selected based on both the percentage of NK cells in a peripheral blood sample that are positive for NKG2C and the percentage of NK cells in the peripheral blood sample that are negative or low for NKG2A. In some embodiments, the subject is selected if at least at or about 20% of NK cells in the peripheral blood sample are positive for NKG2C and at least at or about 70% of NK cells in the peripheral blood sample are negative or low for NKG2A. In some embodiments, the subject is selected if at least at or about 30% of NK cells in the peripheral blood sample are positive for NKG2C and at least at or about 75% of NK cells in the peripheral blood sample are negative or low for NKG2A. In some embodiments, the subject is selected if at least at or about 40% of NK cells in the peripheral blood sample are positive for NKG2C and at least at or about 80% of NK cells in the peripheral blood sample are negative or low for NKG2A. In some embodiments, the subject is selected if at least at or about 50% of NK cells in the peripheral blood sample are positive for NKG2C and at least at or about 85% of NK cells in the peripheral blood sample are negative or low for NKG2A. In some embodiments, the subject is selected if at least at or about 60% of NK cells in the peripheral blood sample are positive for NKG2C and at least at or about 90% of NK cells in the peripheral blood sample are negative or low for NKG2A. In some embodiments, the subject is selected if at least at or about 60% of NK cells in the peripheral blood sample are positive for NKG2C and at least at or about 95% of NK cells in the peripheral blood sample are negative or low for NKG2A.

In some embodiments, a subject is selected for expansion of cells in accord with the provided methods if the subject is CMV seropositive, and if among NK cells in a peripheral blood sample from the subject, the percentage of g-NK cells is greater than at or about 30%, the percentage of NKG2C^{pos} cells is greater than at or about 20%, and the percentage of NKG2A^{neg} cells is greater than at or about 70%.

In some embodiments, NK cells from the subject bear a single nucleotide polymorphism (SNP rs396991) in the CD16 gene, nucleotide 526 [thymidine (T)→guanine (G)] resulting in an amino acid (aa) substitution of valine (V) for phenylalanine (F) at position 158 in the mature (processed) form of the protein (F158V). In some embodiments, NK cells bear the CD16 158V polymorphism in both alleles (called 158V/V herein). In some embodiments, NK cells bear the CD16 158V polymorphism in a single allele (called 158V/F herein). It is understood that reference to a 158V+ genotype herein refers to both the 158V/V genotype and the 158V/F genotype. It has been found that the CD16 F158V polymorphism is associated with substantially higher affinity for IgG1 antibodies and have the ability to mount more robust NK cell-mediated ADCC responses (Mellor et al. (2013) Journal of Hematology & Oncology, 6:1; Musolino et al. (2008) Journal of Clinical Oncology, 26:1789-1796 and Hatjiharissi et al. (2007) Blood, 110:2561-2564). In some embodiments, antibody-directed targeting of CD16 158V+/g-NK cells leads to improved outcomes for patients due to the improved affinity, cytotoxic and/or cytokine-mediated effect functions of the CD16 158V+/g-NK cell subset.

In some embodiments, the provided methods include enriching or isolating NK cells or a subset thereof from a biological sample of a subject identified as having the CD16 158V+NK cell genotype. In some embodiments, the method includes screening subjects for the presence of the CD16 158V+NK cell genotype. In some embodiments, genomic DNA is extracted from a sample from a subject that is or includes NK cells, such as blood sample or bone marrow sample. In some embodiments, the sample is or comprises blood cells, e.g. peripheral blood mononuclear cells. In some embodiments, the sample is or comprises isolated NK cells. In some embodiments, the sample is a sample from a healthy donor subject. Any method for extracting DNA from the sample can be employed. For instance, nucleic acids can be readily isolated from a sample, e.g. cells, using standard techniques such as guanidium thiocyanate-phenol-chloroform extraction (Chomocyznski et al. (1987) Anal. Biochem. 162: 156). Commercially available kits also are readily available for extracting genomic DNA, such as the Wizard genomic DNA purification kit (Promega, Madison, WI).

Genotyping can be performed on any suitable sample. In any of the embodiments described herein, the genotyping reaction can be, for example, a pyrosequencing reaction, DNA sequencing reaction, MassARRAY MALDI-TOF, RFLP, allele-specific PCR, real-time allelic discrimination, or microarray. In some embodiments, a PCR-based technique, such as RT-PCR, of genomic DNA is carried out using allele-specific primers for the polymorphism. The PCR method for amplifying target nucleic acid sequences in a sample is well known in the art and has been described in, e.g., Innis et al. (eds.) PCR Protocols (Academic Press, N Y 1990); Taylor (1991) Polymerase chain reaction: basic principles and automation, in PCR: A Practical Approach, McPherson et al. (eds.) IRL Press, Oxford; Saiki et al. (1986) Nature 324: 163; as well as in U.S. Pat. Nos. 4,683,195, 4,683,202 and 4,889,818, all incorporated herein by reference in their entireties.

Primers for detecting the 158V+ polymorphism are known or can be easily designed by a skilled artisan, See. e.g. International published PCT Appl. No. WO2012/061814; Kim et al. (2006) Blood, 108:2720-2725; Cartron et al. (2002) Blood, 99:754-758; Koene et al. (1997) Blood, 90:1109-1114; Hatijiharissi et al. (2007) Blood, 110:2561-2564; Somboonyosdech et al. (2012) Asian Biomedicine, 6:883-889). In some embodiments, PCR can be carried out using nested primers followed by allele-specific restriction enzyme digestion. In some embodiments, the first PCR primers comprise nucleic acid sequences 5′-ATA TTT ACA GAA TGG CAC AGG-3′ (SEQ ID NO:2) and 5′-GAC TTG GTA CCC AGG TTG AA-3′ (SEQ ID NO:3), while the second PCR primers are 5′-ATC AGA TTC GAT CCT ACT TCT GCA GGG GGC AT-3′ (SEQ ID NO:4) and 5′-ACG TGC TGA GCT TGA GTG ATG GTG ATG TTC AC-3′ (SEQ ID NO:5), which, in some cases, generates a 94-bp fragment depending on the nature of allele. In some embodiments, the primer pair comprises the nucleic acid sequences set forth in SEQ ID NO:6 (CCCAACTCAA CTTCCCAGTG TGAT) and SEQ ID NO:7 (GAAATCTACC TTTTCCTCTA ATAGGGCAAT). In some embodiments, the primer pair comprises the nucleic acid sequences set forth in SEQ ID NO:6 (CCCAACTCAA CTTCCCAGTG TGAT) and SEQ ID NO:8 (GAAATCTACC TTTTCCTCTA ATAGGGCAA). In some embodiments, the primer pair comprises the nucleic acid sequences set forth in SEQ ID NO:6 (CCCAACTCAA CTTCCCAGTG TGAT) and SEQ ID NO:9 (GAAATCTACC TTTTCCTCTA ATAGGGCA).

In some embodiments, genotyping can be carried out by quantitative real-time RT-PCR following extraction of RNA using primer sequences as follows: CD16 sense set forth in SEQ ID NO:10 (5′-CCAAAAGCCACACTCAAAGAC-3′) and antisense set forth in SEQ ID NO:11 (5′-ACCCAGGTGGAAAGAATGATG-3′) and TaqMan probe set forth in SEQ ID NO: 12 (5′-AACATCACCATCACTCAAGGTTTGG-3′).

To confirm the genotyping, allele specific amplification can be used with a set of V allele specific primers (e.g. forward primer set forth in SEQ ID NO:13, 5′-CTG AAG ACA CAT TTT TAC TCC CAAA-3′; and reverse primer set forth in SEQ ID NO:14, 5′-TCC AAA AGC CAC ACT CAA AGA C-3′) or a set of F allele specific primers (e.g., forward primer set forth in SEQ ID NO:15, 5′-CTG AAG ACA CAT TTT TAC TCC CAAC-3′; and reverse primer set forth in SEQ ID NO:14, 5′-TCC AAA AGC CAC ACT CAA AGA C-3′).

The genomic sequence for CD16a is available in the NCBI database at NG_009066.1. The gene ID for CD16A is 2214. Sequence information for CD16, including gene polymorphisms, is available at UniProt Acc. No. P08637. The sequence of CD16 (F158) is set forth in SEQ ID NO:16 (residue F158 is bold and underlined). In some embodiments, CD16 (F158) further comprises a signal peptide set forth as MWQLLLPTALLLLVSA (SEQ ID NO:17).

(SEQ ID NO: 16)
GMRTEDLPKAVVFLEPQWYRVLEKDSVTLKCQGAYSPEDNSTQWFHNES
LISSQASSYFIDAATVDDSGEYRCQTNLSTLSDPVQLEVHIGWLLLQAP
RWVFKEEDPIHLRCHSWKNTALHKVTYLQNGKGRKYFHHNSDFYIPKAT
LKDSGSYFCRGLFGSKNVSSETVNITITQGLAVSTISSFFPPGYQVSFC
LVMVLLFAVDTGLYFSVKTNIRSSTRDWKDHKFKWRKDPQDK

The sequence of CD16 158V+(polymorphism resulting in F158V) is known as VAR_003960 and has the sequence set forth in SEQ ID NO: 18 (158V+ polymorphism is in bold and underline). In some embodiments, CD16 (158V+) further comprises a signal peptide set forth as MWQLLLPTALLLLVSA (SEQ ID NO:17).

(SEQ ID NO: 18)
GMRTEDLPKAVVFLEPQWYRVLEKDSVTLKCQGAYSPEDNSTQWFHNES
LISSQASSYFIDAATVDDSGEYRCQTNLSTLSDPVQLEVHIGWLLLQAP
RWVFKEEDPIHLRCHSWKNTALHKVTYLQNGKGRKYFHHNSDFYIPKAT
LKDSGSYFCRGLVGSKNVSSETVNITITQGLAVSTISSFFPPGYQVSFC
LVMVLLFAVDTGLYFSVKTNIRSSTRDWKDHKFKWRKDPQDK

In some embodiments, single nucleotide polymorphism (SNP) analysis is employed on genomic deoxyribonucleic acid (DNA) samples using allele-specific probes containing a fluorescent dye label (e.g. FAM or VIC) on the 5′ end and a minor groove binder (MGB) and nonfluorescent quencher (NFQ) on the 3′ end and an unlabeled PCR primers to detect a specific SNP targets. In some embodiments, the assay measures or detects the presence of an SNP by a change in fluorescence of the dyes associated with the probe. In such embodiments, probes hybridize to the target DNA between the two unlabeled primers and signal from the fluorescent dye on the 5′ end is quenched by the NFQ on its 3′ end by fluorescence resonance energy transfer (FRET). During PCR, Taq polymerase extends the unlabeled primers using the template as a guide and when the polymerase reaches the labeled probe, it cleaves the molecule separating the dye from the quencher. In some aspects, a qPCR instrument can detect fluorescence from the unquenched label. Exemplary reagents are commercially available SNP Assays, e.g. code C_25815666_10 for rs396991 (Applied Biosystems, Cat No. 4351379 for SNP genotyping of F158V in CD16).

In some embodiments, subjects heterozygous or homozygous for the CD16 158V (F158V) polymorphism are identified. In some embodiments, subjects homozygous for the CD16 158V (F158V) polymorphism are identified. In some embodiments, NK cells or an NK cell subset are isolated or enriched from a biological sample from a subject identified as being heterozygous or homozygous for the CD16 158V polymorphism. In some embodiments, NK cells or an NK cell subset are isolated or enriched from a biological sample from a subject identified as being homozygous for the CD16 158V polymorphism.

In some embodiments, the method includes enriching NK cells from the biological sample, such as from a population PBMCs isolated or obtained from the subject. In some embodiments, the population of cells enriched for NK cells is enriched by isolation or selection based on one or more natural killer cell-specific markers. It is within the level of a skilled artisan to choose particular markers or combinations of surface markers. In some embodiments, the surface marker(s) is any one or more of the from the following surface antigens CD11a, CD3, CD7, CD14, CD16, CD19, CD25, CD27, CD56, CD57, CD161, CD226, NKB1, CD62L; CD244, NKG2D, NKp30, NKp44, NKp46, NKG2A, NKG2C, KIR2DL1 and/or KIR2DL3. In some embodiments, the surface marker(s) is any one or more of the from the following surface antigens CD11a, CD3, CD7, CD14, CD16, CD19, CD25, CD27, CD38, CD56, CD57, CD161, CD226, NKB1, CD62L; CD244, NKG2D, NKp30, NKp44, NKp46, NKG2A, NKG2C, SLAMF7 (CD319), KIR2DL1 and/or KIR2DL3. In particular embodiments, the one or more surface antigen includes CD3 and one or more of the following surface antigens CD16, CD56 or CD57. In some embodiments, the one or more surface antigen is CD3 and CD57. In some embodiments, the one or more surface antigen is CD3, CD56 and CD16. In other embodiments, the one or more surface antigen is CD3, CD56 and CD38. In further embodiments, the one or more surface antigen is CD3, CD56, NKG2A and CD161. In some embodiments, the one or more surface antigen is CD3, CD57, and NKG2C. In some embodiments, the one or more surface antigen is CD3, CD57, and NKG2A. In some embodiments, the one or more surface antigen is CD3, CD57, NKG2C, and NKG2A. In some embodiments, the one or more surface antigen is CD3 and CD56. In some embodiments, the one or more surface antigen is CD3, CD56, and NKG2C. In some embodiments, the one or more surface antigen is CD3, CD56, and NKG2A. In some embodiments, the one or more surface antigen is CD3, CD56, NKG2C, and NKG2A. Reagents, including fluorochrome-conjugated antibodies, for detecting such surface antigens are well known and available to a skilled artisan.

In some embodiments, the NK cell population is enriched, such as by isolation or selection, from a sample by the provided methods are cells that are positive for (marker+ or marker^{pos}) or express high levels (marker^{high}) of one or more particular markers, such as surface markers, or that are negative for or express relatively low levels (marker− or marker^{neg}) of one or more markers. Hence, it is understood that the terms positive, pos or + with reference to a marker or protein expressed on or in a cell are used interchangeably herein. Likewise, it is understood that the terms negative, neg or − with reference to a marker or protein expressed on or in a cell are used interchangeably herein. Further, it is understood that reference to cells that are marker^{neg} herein may refer to cells that are negative for the marker as well as cells expressing relatively low levels of the marker, such as a low level that would not be readily detectable compared to control or background levels. In some cases, such markers are those that are absent or expressed at relatively low levels on certain populations of NK cells but are present or expressed at relatively higher levels on certain other populations of lymphocytes (such as T cells). In some cases, such markers are those that are present or expressed at relatively higher levels on certain populations of NK cells but are absent or expressed at relatively low levels on certain other populations of lymphocytes (such as T cells or subsets thereof).

In some embodiments, any known method for separation based on such markers may be used. In some embodiments, the separation is affinity- or immunoaffinity-based separation. For example, the isolation in some aspects includes separation of cells and cell populations based on the expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. In some embodiments, incubation is static (without mixing). In some embodiments, incubation is dynamic (with mixing).

Such separation steps can be based on positive selection, in which the cells having bound the reagents are retained for further use, and/or negative selection, in which the cells having not bound to the antibody or binding partner are retained. In some examples, both fractions are retained for further use. The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells. For example, in some aspects, a negative selection for CD3 enriches for a population of cells that are CD3^{neg}, but also can contain some residual or small percentage of other non-selected cells, which can, in some cases, include a small percentage of cells still being present in the enriched population that are CD3^{pos}. In some examples, a positive selection of one of the CD57^{pos} or CD16^{pos} population enriches for said population, either the CD57^{pos} or CD16^{pos} population, but also can contain some residual or small percentage of other non-selected cells, which can, in some cases, include the other of the CD57 or CD16 population still being present in the enriched population.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In some aspects, the selection includes positive and/or negative selection steps based on expression of one or more of the surface antigens, such as in cells from a PBMC sample. In some embodiments, the isolation includes positive selection for cells expressing CD56, cells expressing CD16 or cells expressing CD57 and/or negative selection for cells expressing CD38 and/or negative selection for cells expressing non-NK cell markers, such as T cell markers, for example, negative selection for cells expressing CD3 (CD3^{neg}). For example, in some embodiments, the isolation includes positive selection for cells expressing CD56, cells expressing CD16 or cells expressing CD57 and/or negative selection for cells expressing non-NK cell markers, such as T cell markers, for example, negative selection for cells expressing CD3 (CD3^{neg}). In some embodiments, the isolation includes positive selection for cells expressing CD56, cells expressing CD16 or cells expressing CD57, and/or negative selection for cells expressing CD38 (CD38^{neg}), CD161 (CD161^{neg}), NKG2A (NKG2A^{neg}), and/or negative selection for cells expressing CD3 (CD3^{neg}). In some embodiments, the selection includes isolation of cells negative for CD3 (CD3^{neg}).

In some embodiments, the isolation includes negative selection for cells expressing CD3 (CD3^{neg}) and positive selection for cells expressing CD56 (CD56^{pos}). In some embodiments, the selection can further include negative selection for cells expressing CD38 (CD38^{neg}). In specific embodiments, the isolated or selected cells are CD3^{neg}CD56^{pos}CD38^{neg}.

In some embodiments, the selection includes negative selection for cells expressing CD3 (CD3^{neg}), positive selection for cells expressing CD56 (CD56^{pos}), followed by negative selection for cells expressing NKG2A (NKG2A^{neg}) and CD161 (CD161^{neg}). In specific embodiments, the isolated or selected cells are CD3^{neg}CD56^{pos}NKG2A^{neg} CD161^{neg}.

In some embodiments, the selection includes negative selection for cells expressing CD3 (CD3^{neg}) and positive selection for cells expressing CD57 (CD57^{pos}). In specific embodiments, the isolated or selected cells are CD3^{neg}CD57^{pos}.

In some embodiments, the selection includes negative selection for cells expressing CD3 (CD3^{neg}) and positive for cells expressing CD16 (CD16^{pos}). In specific embodiments, the isolated or selected cells are CD3^{neg}CD16^{pos}.

In some embodiments, the selection includes negative selection for cells expressing CD3 (CD3^{neg}) and positive selection for cells expressing CD57 (CD57^{pos}). In specific embodiments, the isolated or selected cells are CD3^{neg}CD57^{pos}. For example, the NK cells may be enriched by depletion of CD3^{pos} cells (negative selection for CD3^{pos} cells) followed by CD57^{pos} cell selection, thereby isolating and enriching CD57^{pos} NK cells. The separation can be carried out by immunoaffinity-based methods, such as using MACS™ Microbeads. For example, CD3 microbeads can be used to deplete CD3^{pos} cells in a negative selection for CD3^{neg} cells. Subsequently, CD57 MicroBeads can be used for CD57 enrichment of CD3 cell-depleted PBMCs. The CD3^{neg}/CD57^{pos} enriched NK cells can then be used in expansion in the provided methods.

In some embodiments, the selection may further include positive selection for cells expressing NKG2C (NKG2C^{pos}) and/or negative selection for cells NKG2A (NKG2A^{neg}). In some embodiments, the selection includes negative selection for cells expressing CD3 (CD3^{neg}), positive selection for cells expressing CD57 (CD57^{pos}), and positive selection for cells expressing NKG2C (NKG2C^{pos}). In specific embodiments, the isolated or selected cells are CD3^{neg}CD57^{pos}NKG2C^{pos}. In some embodiments, the selection includes negative selection for cells expressing CD3 (CD3^{neg}), positive selection for cells expressing CD57 (CD57^{pos}), and negative selection for cells expressing NKG2A (NKG2A^{neg}). In specific embodiments, the isolated or selected cells are CD3^{neg}CD57^{pos}NKG2A^{neg}. In some embodiments, the selection includes negative selection for cells expressing CD3 (CD3^{neg}), positive selection for cells expressing CD57 (CD57^{pos}), positive selection for cells expressing NKG2C (NKG2C^{pos}), and negative selection for cells expressing NKG2A (NKG2A^{neg}). In specific embodiments, the isolated or selected cells are CD3^{neg}CD57^{pos}NKG2C^{pos}NKG2A^{neg}.

In some of any of the provided embodiments, the selection can further include negative selection for cells expressing CD38 (CD38^{neg}). In specific embodiments, the isolated or selected cells are CD3^{neg}CD57^{pos}CD38^{neg}. In specific embodiments, the isolated or selected cells are CD3^{neg}CD57^{pos}CD38^{neg}NKG2C^{pos}. In specific embodiments, the isolated or selected cells are CD3^{neg}CD57^{pos}CD38^{neg}NKG2A^{neg} In specific embodiments, the isolated or selected cells are CD3^{neg}CD57^{pos}CD38^{neg}NKG2C^{pos}NKG2A^{neg}.

In some embodiments, the selection includes negative selection for cells expressing CD3 (CD3^{neg}) and positive selection for cells expressing CD56 (CD56^{pos}). In specific embodiments, the isolated or selected cells are CD3^{neg}CD56^{pos}. In some embodiments, the selection includes negative selection for cells expressing CD3 (CD3^{neg}), positive selection for cells expressing CD56 (CD56^{pos}), and positive selection for cells expressing NKG2C (NKG2C^{pos}). In specific embodiments, the isolated or selected cells are CD3^{neg}CD56^{pos}NKG2C^{pos}. In some embodiments, the selection includes negative selection for cells expressing CD3 (CD3^{neg}), positive selection for cells expressing CD56 (CD56^{pos}), and negative selection for cells expressing NKG2A (NKG2A^{neg}). In specific embodiments, the isolated or selected cells are CD3^{neg}CD56^{pos}NKG2A^{neg}. In some embodiments, the selection includes negative selection for cells expressing CD3 (CD3^{neg}), positive selection for cells expressing CD56 (CD56^{pos}), positive selection for cells expressing NKG2C (NKG2C^{pos}), and negative selection for cells expressing NKG2A (NKG2A^{neg}). In specific embodiments, the isolated or selected cells are CD3^{neg}CD56^{pos}NKG2C^{pos}NKG2A^{neg}.

In some of any of the provided embodiments, the selection can further include negative selection for cells expressing CD38 (CD38^{neg}). In specific embodiments, the isolated or selected cells are CD3^{neg}CD56^{pos}CD38^{neg}. In specific embodiments, the isolated or selected cells are CD3^{neg}CD56^{pos}CD38^{neg}NKG2C^{pos}. In specific embodiments, the isolated or selected cells are CD3^{neg}CD56^{pos}CD38^{neg}NKG2A^{neg}. In specific embodiments, the isolated or selected cells are CD3^{neg}CD56^{pos}CD38^{neg}NKG2C^{pos}NKG2A^{neg}.

In some of any of the provided embodiments, the g-NK cells are cells having a g-NK surrogate surface marker profile. In some embodiments, the g-NK cell surrogate surface marker profile is CD16^{pos}/CD57^{pos}/CD7^{dim/neg}/CD161^{neg}. In some embodiments, the g-NK cell surrogate surface marker profile is NKG2A^{neg}/CD161^{neg}. In some of any such embodiments, the g-NK cell surrogate surface marker profile is CD38^{neg}. In some of any such embodiments, CD45^{pos}/CD3^{neg}/CD56^{pos} is used as a surrogate surface marker profile for NK cells. In some of any such embodiments, the g-NK cell surrogate surface marker profile further includes an NK cell surrogate surface marker profile. In some of any such embodiments, the g-NK cell surrogate surface marker profile further includes CD45^{pos}/CD3^{neg}/CD56^{pos}. In particular embodiments the g-NK cell surrogate surface marker profile includes embodiments, the g-NK cell surrogate surface marker profile includes CD45^{pos}/CD3^{neg}/CD56^{pos}/NKG2A^{neg}/CD161^{neg}. In other particular embodiments, the g-NK cell surrogate surface marker profile includes CD45^{pos}/CD3^{neg}/CD56^{pos}/CD38^{neg}

In some embodiments, the methods of isolating, selecting and/or enriching for cells, such as by positive or negative selection based on the expression of a cell surface marker or markers, can include immunoaffinity-based selections. In some embodiments, the immunoaffinity-based selections include contacting a sample containing cells, such as PBMCs, with an antibody or binding partner that specifically binds to the cell surface marker or markers. In some embodiments, the antibody or binding partner is bound to a solid support or matrix, such as a sphere or bead, for example microbeads, nanobeads, including agarose, magnetic bead or paramagnetic beads, to allow for separation of cells for positive and/or negative selection. In some embodiments, the spheres or beads can be packed into a column to effect immunoaffinity chromatography, in which a sample containing cells, such as PBMCs, is contacted with the matrix of the column and subsequently eluted or released therefrom.

The incubation generally is carried out under conditions whereby the antibodies or binding partners, which specifically bind to such antibodies or binding partners, which are attached to the magnetic particle or bead, specifically bind to cell surface molecules if present on cells within the sample.

In some aspects, the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some embodiments, the magnetically responsive particles are left attached to the cells that are to be subsequently incubated and/or cultured; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

In some embodiments, the affinity-based selection is via magnetic-activated cell sorting (MACS) (Miltenyi Biotech, Auburn, CA). Magnetic Activated Cell Sorting (MACS) systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some of any of such embodiments, the method comprises administering IL-12, IL-15, IL-18, IL-2 and/or CCL5 to the subject prior to enriching, such as selecting and/or isolating, the NK cells or subset thereof.

In embodiments of the provided methods, the enriched NK cells are incubated or cultured in the presence of feeder cells, such as under conditions to support the proliferation and expansion of NK cell subsets, and in particular the g-NK cell subset.

In particular aspects, the feeder cells include cells that stimulate or promote expansion of NKG2C^{pos} and/or inhibit expansion of NKG2A^{pos} cells. In some embodiments, the feeder cells are cells that express or are transfected with HLA-E or a hybrid HLA-E containing the HLA-A2 signal sequence. For example, exemplary of such a hybrid is an AEH hybrid gene containing an MHC class I, such as HLA-A2, promoter and signal sequence and the HLA-E mature protein sequence, which, in some cases, can result in a mature protein identical to that encoded by the HLA-E gene but that can be stably expressed on the cell surface (see e.g. Lee et al. (1998) Journal of Immunology, 160:4951-4960). In some embodiments, the cell is an LCL 721.221, K562 cell or RMA-S cell that is transfected to express an MHC-E molecule stabilized in the presence of an MHC class I, such as HLA-A2, leader sequence. Cells lines that are engineered to express cell surface HLA-E stabilized in the presence of an MHC class I, such as HLA-A2, leader sequence peptide are known in the art (Lee et al. (1998) Journal of Immunology, 160:4951-4960; Zhongguo et al. (2005) 13:464-467; Garcia et al. (2002) Eur J. Immunol., 32:936-944). In some embodiments, 221.AEH cells, such as irradiated 221.AEH cells, can be used as feeder cells, or any other HLA-E-expressing cell line or irradiated HLA-E-expressing cell line that is otherwise HLA negative, such as K562. In some embodiments, the cell line can be transfected to express HLA-E. In some embodiments, K562 cells expressing membrane-bound IL-15 (K562-mb15) or membrane-bound IL-21 (K562-mb21) can be used as feeder cells. Exemplary of such a cell line for use in the methods provided herein are 221-AEH cells.

In embodiments, the HLA-expressing feeder cells are cryopreserved and thawed before use. In some embodiments, if the cells have been transfected to express HLA-E such as 221.AEH cells, the cells can be grown in the presence of appropriate nutrients, e.g. including serum or other appropriate serum replacement, and a selection agent prior to their use in the method. For example, in the case of 221.AEH cells, the cells can be cultured in cell culture media supplemented with Hygromycin B (e.g. 0.1% to 10%, such as at or about 1%) to maintain selective pressure on the cells to maintain the high level of plasmid HLA-E. The cells can be maintained at a density of 1×10⁵ cells/mL to 1×10⁶ cells/mL until use.

In particular embodiments, the HLA-E-expressing feeder cells, e.g. 221.AEH cells, added to the culture are non-dividing, such as by X-ray irradiation or gamma irradiation. The HLA-E-expressing feeder cells, e.g. 221.AEH, can be irradiated on the day of or just prior to their use in the provided methods. In some embodiments, the HLA-E-expressing feeder cells are irradiated with gamma rays in the range of about 1000 to 10000 rad, such as 1000-5000, rads to prevent cell division. In some embodiments, the HLA-E-expressing feeder cells are irradiated with gamma rays in the range of about 10 Gy to 100 Gy, such as 10-50 Gy to prevent cell division. In some embodiments, the cells are irradiated at 100 Gy. In other embodiments, irradiation is carried out by x-ray irradiation. In some embodiments, the HLA-E-expressing feeder cells are irradiated with x rays in the range of about 10 Gy to 100 Gy, such as 10-50 Gy to prevent cell division. In some embodiments, the A Rad-Sure™ blood irradiation indicator can be used to provide positive visual verification of irradiation. In aspects of the provided methods, the feeder cells are never removed; as a result of the irradiation the NK cells will be directly cytotoxic to the feeder cells and the feeder cells will die during the culture.

In some embodiments, the enriched, selected and/or isolated NK cells are incubated or cultured in the presence of HLA-E-expressing feeder cells (e.g. 221.AEH cells), such as an irradiated population thereof, at a ratio of feeder cells to enriched NK cells that is greater than or about 1:10 HLA-E feeder cells (e.g. 221.AEH cells), such as an irradiated population thereof, to enriched NK cells, such as from at or about 1:10 and at or about 10:1 of such feeder cells to enriched NK cells.

In some embodiments, the ratio of HLA-E-expressing feeder cells (e.g. 221.AEH cells), such as an irradiated population thereof, is at a ratio of such feeder cells to enriched NK cells that is between at or about 1:10 and at or about 10:1, between at or about 1:10 and at or about 5:1, between at or about 1:10 and at or about 2.5:1, between at or about 1:10 and at or about 1:1, between at or about 1:10 and at or about 1:2.5, between at or about 1:10 and at or about 1:5, between at or about 1:5 and at or about 10:1, between at or about 1:5 and at or about 5:1, between at or about 1:5 and at or about 2.5:1, between at or about 1:5 and at or about 1:1, between at or about 1:5 and at or about 1:2.5, between at or about 1:2.5 and at or about 10:1, between at or about 1:2.5 and at or about 5:1, between at or about 1:2.5 and at or about 2.5:1, between at or about 1:2.5 and at or about 1:1, between at or about 1:1 and at or about 10:1, between at or about 1:1 and at or about 5:1, between at or about 1:1 and at or about 3:1, between at or about 1:1 and at or about 2.5:1, between at or about 2.5:1 and at or about 10:1, between at or about 2.5:1 and at or about 5:1 or between at or about 5:1 and at or about 10:1, each inclusive.

In some embodiments, the ratio of HLA-expressing feeder cells (e.g. 221.AEH cells), such as an irradiated population thereof, is at a ratio of such feeder cells to enriched NK cells that is at or about 1.25:1, 1.5:1, 1.75:1, 2.0:1, 2.25:1, 2:5:1, 2.75:1, 3.0:1, 3.25:1, 3.5: 1, 3.75:1, 4.0:1, 4.25: 1, 4.5:1, 4.75:1 or 5:1, or any value between any of the foregoing. In some embodiments, the ratio of HLA-expressing feeder cells (e.g. 221.AEH cells), such as an irradiated population thereof, is at a ratio of such feeder cells to enriched cells that is less than or less than about 5:1. In some embodiments, the ratio of HLA-expressing feeder cells (e.g. 221.AEH cells), such as an irradiated population thereof, is at a ratio between at or about 1:1 and 2.5:1, inclusive. In some embodiments, the ratio of HLA-expressing feeder cells (e.g. 221.AEH cells), such as an irradiated population thereof, is at a ratio of at or about 2.5:1. In some embodiments, the ratio of HLA-expressing feeder cells (e.g. 221.AEH cells), such as an irradiated population thereof, is at a ratio of at or about 2:1.

In some cases if the starting NK cell population has been cryopreserved prior to expansion, i.e. subject to freeze/thaw, a lower 221.AEH to NK-cell ratio can be employed than for methods using fresh NK cells. It is found here that a ratio of 1:1 221.AEH to freeze/thaw NK-cell resulted in comparable expansion in a culture containing a ratio of 2.5:1 221.AEH to fresh NK cells. In some aspects, the lower ratio ensures a higher number of NK cells in the culture to permit more cell-to-cell contact, which may play a role in promoting initial growth and expansion. In some embodiments, if initial enriched population of NK cells from a sample has been subject to freeze/thaw, a ratio of at or about 2:1 to 1:2 221.AEH to freeze/thaw NK-cells is used. In particular embodiments, the ratio is 1:1. It is understood that higher ratio, such as 2.5:1 221.AEH to freeze/thaw NK-cells can be used, but this may require a longer culture, e.g. at or about 21 days, to reach a desired threshold density or number.

In some embodiments, the NK cells are expanded by further adding to the culture non-dividing peripheral blood mononuclear cells (PBMC). In some aspects, the non-dividing feeder cells can comprise X-ray-irradiated PBMC feeder cells. In some aspects, the non-dividing feeder cells can comprise gamma-irradiated PBMC feeder cells. In some embodiments, the PBMC are irradiated with gamma rays in the range of about 1000 to 10000 rad, such as 1000-5000, rads to prevent cell division. In some embodiments, the PBMC are irradiated with gamma rays in the range of about 10 Gy to 100 Gy, such as 10-50 Gy to prevent cell division. In some aspects, during at least a portion of the incubation, the irradiated feeder cells are present in the culture medium at the same time as the non-dividing (e.g. irradiated) HLA-E-expressing feeder cells. In some aspects, the non-dividing (e.g. irradiated) PBMC feeder cell, HLA-E-expressing feeder cells and enriched NK cells are added to the culture on the same day, such as on the day of the initiation of the incubation, e.g. at or about or near the same time.

In some embodiments, the incubation or culture is further carried out in the presence of irradiated PBMCs as feeder cells. In some embodiments, the irradiated PBMC feeder cells are autologous to, or from the same subject as, the enriched NK cells were isolated or selected. In particular embodiments, the PBMCs are obtained from the same biological sample, e.g. whole blood or leukapheresis or apheresis product, as used to enrich the NK cells. Once obtained, a portion of the PBMCs are reserved for irradiation prior to enrichment of NK cells as described above.

In some embodiments, irradiated PBMCs are present as feeder cells at a ratio of such feeder cells to enriched NK cells that is from at or about 1:10 to at or about 10:1, from at or about 1:10 to at or about 5:1, from at or about 1:10 to at or about 2.5:1, from at or about 1:10 to at or about 1:1, from at or about 1:10 to at or about 1:2.5, from at or about 1:10 to at or about 1:5, from at or about 1:5 to at or about 10:1, from at or about 1:5 to at or about 5:1, from at or about 1:5 to at or about 2.5:1, from at or about 1:5 to at or about 1:1, from at or about 1:5 to at or about 1:2.5, from at or about 1:2.5 to at or about 10:1, from at or about 1:2.5 to at or about 5:1, from at or about 1:2.5 to at or about 2.5:1, from at or about 1:2.5 to at or about 1:1, from at or about 1:1 to at or about 10:1, from at or about 1:1 to at or about 5:1, from at or about 1:1 to at or about 2.5:1, from at or about 2.5:1 to at or about 10:1, from at or about 2.5:1 to at or about 5:1 or from at or about 5:1 to at or about 10:1.

In some embodiments, the irradiated PBMCs are present as feeder cells at a ratio of such feeder cells to enriched NK cells that is between at or about 1:1 and at or about 5:1, such as at or about 1.25:1, 1.5:1, 1.75:1, 2.0:1, 2.25:1, 2:5:1, 2.75:1, 3.0:1, 3.25:1, 3.5: 1, 3.75: 1, 4.0:1, 4.25:1, 4.5:1, 4.75:1 or 5:1, or any value between any of the foregoing. In some embodiments, the irradiated PBMCs are present at a ratio of such feeder cells to enriched cells that is or is about 5:1.

In particular embodiments, during at least a portion of the incubation or culture one or more cells or cell types, such as T cells, of the irradiated PBMCs are activated and/or the incubation or culture is carried out in the presence of at least one stimulatory agent that is capable of stimulating the activation of one or more T cells of the PBMC feeder cells. In some embodiments, at least one stimulatory agent specifically binds to a member of a TCR complex. In some embodiments, the at least one stimulatory agent specifically binds to a CD3, optionally a CD3epsilon. In some aspects, the at least one stimulatory agent is an anti-CD3 antibody or antigen binding fragment. An exemplary anti-CD3 antibody includes mouse anti-human CD3 (OKT3).

In some embodiments, the anti-CD3 antibody or antigen-binding fragment is present during at least a portion of the incubation that includes irradiated PBMC feeder cells. In some embodiments, the anti-CD3 antibody or antigen-binding fragment is added to the culture or incubation at or about the same time as the irradiated PBMCs. For example, the anti-CD3 antibody or antigen-binding fragment is added at or about at the initiation of the incubation or culture. In particular aspects, the anti-CD3 antibody or antigen-binding fragment may be removed, or its concentration reduced, during the course of the culture or incubation, such as by exchanging or washing out the culture medium. In particular embodiments, after exchanging or washing, the methods do not include adding back or replenishing the culture media with the anti-CD3 antibody or antigen-binding fragment.

In some embodiments, the anti-CD3 antibody or antigen-binding fragment is added, or is present during at least a portion of the culture or incubation, at a concentration that is between at or about 10 ng/ml and at or about 5 μg/mL, such as between at or about 10 ng/ml and at or about 2 μg/mL, between at or about 10 ng/mL and at or about 1 μg/mL, between at or about 10 ng/ml and at or about 500 ng/ml, between at or about 10 ng/ml and at or about 100 ng/ml, between at or about 10 ng/ml and at or about 50 ng/ml, between at or about 50 ng/ml and at or about 5 μg/mL, such as between at or about 50 ng/mL and at or about 2 μg/mL, between at or about 50 ng/mL and at or about 1 μg/mL, between at or about 50 ng/ml and at or about 500 ng/ml, between at or about 50 ng/mL and at or about 100 ng/mL, between at or about 100 ng/ml and at or about 5 μg/mL, between at or about 100 ng/ml and at or about 2 μg/mL, between at or about 100 ng/ml and at or about 1 μg/mL, between at or about 100 ng/mL and at or about 500 ng/mL, between at or about 500 ng/mL and at or about 5 μg/mL, between at or about 500 ng/mL and at or about 2 μg/mL, between at or about 500 ng/ml and at or about 1 μg/mL, between at or about 1 μg/mL and at or about 5 μg/mL, between at or about 1 μg/mL and at or about 2 μg/mL, or between at or about 2 μg/mL and at or about 5 μg/mL, each inclusive. In some embodiments, the concentration of the anti-CD3 antibody or antigen-binding fragment is at or about 10 ng/ml, 20 ng/ml, 30 ng/mL, 40 ng/mL, 50 ng/mL, 60 ng/ml, 70 ng/mL, 80 ng/mL, 90 ng/mL or 100 ng/mL, or any value between any of the foregoing. In some embodiments, the concentration of the anti-CD3 antibody or antigen-binding fragment is or is about 50 ng/mL.

In some embodiments, the term “antibody” refers to immunoglobulin molecules and antigen-binding portions or fragments of immunoglobulin (Ig) molecules, i.e., molecules that contain an antigen binding site that specifically binds (immunoreacts with) an antigen. The term antibody encompasses not only intact polyclonal or monoclonal antibodies, but also fragments thereof, such as dAb, Fab, Fab′, F(ab′)₂, Fv), single chain (scFv) or single domain antibody (sdAb). Typically, an “antigen-binding fragment” contains at least one CDR of an immunoglobulin heavy and/or light chain that binds to at least one epitope of the antigen of interest. In this regard, an antigen-binding fragment may comprise 1, 2, 3, 4, 5, or all 6 CDRs of a variable heavy chain (VH) and variable light chain (VL) sequence from antibodies that bind the antigen, such as generally six CDRs for an antibody containing a VH and a VL (“CDR1,” “CDR2” and “CDR3” for each of a heavy and light chain), or three CDRs for an antibody containing a single variable domain.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; variable heavy chain (VH) regions, single-chain antibody molecules such as scFvs and single-domain VH single antibodies; and multispecific antibodies formed from antibody fragments. In particular embodiments, the antibodies are single-chain antibody fragments comprising a variable heavy chain region and/or a variable light chain region, such as scFvs.

In some embodiments, the incubation or culture is initiated in the presence of such enriched NK cells, such as selected and/or isolated NK cells, at a concentration that is at or about, or at least at or about, 0.05×10⁶ enriched NK cells/mL, at or about 0.1×10⁶ enriched NK cells/mL, at or about 0.2×10⁶ enriched NK cells/mL, at or about 0.5×10⁶ enriched NK cells/mL or at or about 1.0×10⁶ enriched NK cells/mL. In embodiments of the provided methods, the incubation or culture is initiated in the presence of such enriched NK cells, such as selected and/or isolated NK cells, at a concentration that is between at or about 0.05×10⁶ enriched NK cells/mL and at or about 1.0×10⁶ enriched NK cells/mL, such as between at or about 0.05×10⁶ enriched NK cells/mL and at or about 0.75×10⁶, between at or about 0.05×10⁶ enriched NK cells/mL and at or about 0.5×10⁶, between at or about 0.05×10⁶ enriched NK cells/mL and at or about 0.20×10⁶ enriched NK cells/mL, between at or about 0.05×10⁶ enriched NK cells/mL and at or about 0.1×10⁶ enriched NK cells/mL, between at or about 0.1×10⁶ enriched NK cells/mL and at or about 1.0×10⁶ enriched NK cells/mL, between at or about 0.1×10⁶ enriched NK cells/mL and at or about 0.75×10⁶, between at or about 0.1×10⁶ enriched NK cells/mL and at or about 0.5×10⁶, between at or about 0.1×10⁶ enriched NK cells/mL and at or about 0.20×10⁶ enriched NK cells/mL, between at or about 0.20×10⁶ enriched NK cells/mL and at or about 1.0×10⁶ enriched NK cells/mL, between at or about 0.20×10⁶ enriched NK cells/mL and at or about 0.75×10⁶, between at or about 0.20×10⁶ enriched NK cells/mL and at or about 0.5×10⁶, between at or about 0.5×10⁶ enriched NK cells/mL and at or about 1.0×10⁶ enriched NK cells/mL, between at or about 0.5×10⁶ enriched NK cells/mL and at or about 0.75×10⁶, between at or about 0.75×10⁶ enriched NK cells/mL and at or about 1.0×10⁶ enriched NK cells/mL, each inclusive. In some embodiments, the incubation or culture is initiated in the presence of such enriched NK cells, such as selected and/or isolated NK cells, at a concentration that is at or about 0.2×10⁶ enriched NK cells/mL.

In some of any such embodiments, the amount of enriched NK cells, such as selected or isolated from PBMCs as described above, added or present at the initiation of the incubation or culture is at least or at least about 1×10⁵ cells, at least or at least about 2×10⁵ cells, at least or at least about 3×10⁵ cells, at least or at least about 4×10⁵ cells, at least or at least about 5×10⁵ cells, at least or at least about 6×10⁵ cells, at least or at least about 7×10⁵ cells, at least or at least about 8×10⁵ cells, at least or at least about 9×10⁵ cells, at least or at least about 1×10⁶ cells or more. In particular embodiments, the amount of enriched NK cells, such as selected or isolated from PBMCs as described above, is at least or about at least or is or is about 1×10⁶ cells.

In some embodiments, the population of enriched NK cells comprises at least at or about 2.0×10⁶ enriched NK cells, at least at or about 3.0×10⁶ enriched NK cells, at least at or about 4.0×10⁶ enriched NK cells, at least at or about 5.0×10⁶ enriched NK cells, at least at or about 6.0×10⁶ enriched NK cells, at least at or about 7.0×10⁶ enriched NK cells, at least at or about 8.0×10⁶ enriched NK cells, at least at or about 9.0×10⁶ enriched NK cells, at least at or about 1.0×10⁷ enriched NK cells, at least at or about 5.0×10⁷ enriched NK cells, at least at or about 1.0×10⁸ enriched NK cells, at least at or about 5.0×10⁸ enriched NK cells, or at least at or about 1.0×10⁹ enriched NK cells. In some embodiments, the population of enriched NK cells comprises at least at or about 2.0×10⁵ enriched NK cells. In some embodiments, the population of enriched NK cells comprises at least at or about 1.0×10⁶ enriched NK cells. In some embodiments, the population of enriched NK cells comprises at least at or about 1.0×10⁷ enriched NK cells.

In some embodiments, the population of enriched NK cells comprises between at or about 2.0×10⁵ enriched NK cells and at or about 1.0×10⁹ enriched NK cells, between at or about 2.0×10⁵ enriched NK cells and at or about 5.0×10⁸ enriched NK cells, between at or about 2.0×10⁵ enriched NK cells and at or about 1.0×10⁸ enriched NK cells, between at or about 2.0×10⁵ enriched NK cells and at or about 5.0×10⁷ enriched NK cells, between at or about 2.0×10⁵ enriched NK cells and at or about 1.0×10⁷ enriched NK cells, between at or about 2.0×10⁵ enriched NK cells and at or about 5.0×10⁶ enriched NK cells, between at or about 2.0×10⁵ enriched NK cells and at or about 1.0×10⁶ enriched NK cells, between at or about 1.0×10⁶ enriched NK cells and at or about 1.0×10⁹ enriched NK cells, between at or about 1.0×10⁶ enriched NK cells and at or about 5.0×10⁸ enriched NK cells, between at or about 1.0×10⁶ enriched NK cells and at or about 1.0×10⁸ enriched NK cells, between at or about 1.0×10⁶ enriched NK cells and at or about 5.0×10⁷ enriched NK cells, between at or about 1.0×10⁶ enriched NK cells and at or about 1.0×10⁷ enriched NK cells, between at or about 1.0×10⁶ enriched NK cells and at or about 5.0×10⁶ enriched NK cells, between at or about 5.0×10⁶ enriched NK cells and at or about 1.0×10⁹ enriched NK cells, between at or about 5.0×10⁶ enriched NK cells and at or about 5.0×10⁸ enriched NK cells, between at or about 5.0×10⁶ enriched NK cells and at or about 1.0×10⁸ enriched NK cells, between at or about 5.0×10⁶ enriched NK cells and at or about 5.0×10⁷ enriched NK cells, between at or about 5.0×10⁶ enriched NK cells and at or about 1.0×10⁷ enriched NK cells, between at or about 1.0×10⁷ enriched NK cells and at or about 1.0×10⁹ enriched NK cells, between at or about 1.0×10⁷ enriched NK cells and at or about 5.0×10⁸ enriched NK cells, between at or about 1.0×10⁷ enriched NK cells and at or about 1.0×10⁸ enriched NK cells, between at or about 1.0×10⁷ enriched NK cells and at or about 5.0×10⁷ enriched NK cells, between at or about 5.0×10⁷ enriched NK cells and at or about 1.0×10⁹ enriched NK cells, between at or about 5.0×10⁷ enriched NK cells and at or about 5.0×10⁸ enriched NK cells, between at or about 5.0×10⁷ enriched NK cells and at or about 1.0×10⁸ enriched NK cells, between at or about 1.0×10⁸ enriched NK cells and at or about 1.0×10⁹ enriched NK cells, between at or about 1.0×10⁸ enriched NK cells and at or about 5.0×10⁸ enriched NK cells, or between at or about 5.0×10⁸ enriched NK cells and at or about 1.0×10⁹ enriched NK cells. In some embodiments, the population of enriched NK cells comprises between at or about 2.0×10⁵ enriched NK cells and at or about 5.0×10⁷ enriched NK cells. In some embodiments, the population of enriched NK cells comprises between at or about 1.0×10⁶ enriched NK cells and at or about 1.0×10⁸ enriched NK cells. In some embodiments, the population of enriched NK cells comprises between at or about 1.0×10⁷ enriched NK cells and at or about 5.0×10⁸ enriched NK cells. In some embodiments, the population of enriched NK cells comprises between at or about 1.0×10⁷ enriched NK cells and at or about 1.0×10⁹ enriched NK cells.

In some embodiments, the percentage of g-NK cells among the population of enriched NK cells is between at or about 20% and at or about 90%, between at or about 20% and at or about 80%, between at or about 20% and at or about 70%, between at or about 20% and at or about 60%, between at or about 20% and at or about 50%, between at or about 20% and at or about 40%, between at or about 20% and at or about 30%, between at or about 30% and at or about 90%, between at or about 30% and at or about 80%, between at or about 30% and at or about 70%, between at or about 30% and at or about 60%, between at or about 30% and at or about 50%, between at or about 30% and at or about 40%, between at or about 40% and at or about 90%, between at or about 40% and at or about 80%, between at or about 40% and at or about 70%, between at or about 40% and at or about 60%, between at or about 40% and at or about 50%, between at or about 50% and at or about 90%, between at or about 50% and at or about 80%, between at or about 50% and at or about 70%, between at or about 50% and at or about 60%, between at or about 60% and at or about 90%, between at or about 60% and at or about 80%, between at or about 60% and at or about 70%, between at or about 70% and at or about 90%, between at or about 70% and at or about 80%, or between at or about 80% and at or about 90%. In some embodiments, the percentage of g-NK cells among the population of enriched NK cells is between at or about 20% and at or about 90%. In some embodiments, the percentage of g-NK cells among the population of enriched NK cells is between at or about 40% and at or about 90%. In some embodiments, the percentage of g-NK cells among the population of enriched NK cells is between at or about 60% and at or about 90%.

In some of these embodiments, the NK cells can be cultured with a growth factor. According to some embodiments, the at least one growth factor comprises a growth factor selected from the group consisting of SCF, GSK3i, FLT3, IL-2, IL-6, IL-7, IL-15, IL-12, IL-18 and IL-21. According to some embodiments, the at least one growth factor is IL-2 or IL-7 and IL-15. According to some embodiments, the at least one growth factor is IL-2, IL-21 or IL-7 and IL-15. In some embodiments, the growth factor is a recombinant cytokine, such as a recombinant IL-2, recombinant IL-7, recombinant IL-21 or recombinant IL-15.

In some embodiments, the NK cells are cultured in the presence of one or more recombinant cytokines. In some embodiments, the one or more recombinant cytokines comprise any of SCF, GSK3i, FLT3, IL-2, IL-6, IL-7, IL-15, IL-12, IL-18, IL-21, IL-27, or combinations thereof. In some embodiments, the one or more recombinant cytokines comprise any of IL-2, IL-7, IL-15, IL-12, IL-18, IL-21, IL-27, or combinations thereof. In some embodiments, at least one of the one or more recombinant cytokines is IL-21. In some embodiments, the one or more recombinant cytokines further comprises IL-2, IL-7, IL-15, IL-12, IL-18, or IL-27, or combinations thereof. In some embodiments, at least one of the one or more recombinant cytokines is IL-2. In some embodiments, the one or more recombinant cytokines is at least IL-2 and IL-21. In some embodiments, the one or more recombinant cytokines are IL-21 and IL-2. In some embodiments, the one or more recombinant cytokines are IL-21, IL-2, and IL-15. In some embodiments, the one or more recombinant cytokines are IL-21, IL-12, IL-15, and IL-18. In some embodiments, the one or more recombinant cytokines are IL-21, IL-2, Il-12, IL-15, and IL-18. In some embodiments, the one or more recombinant cytokines are IL-21, IL-15, IL-18, and IL-27. In some embodiments, the one or more recombinant cytokines are IL-21, IL-2, IL-15, IL-18, and IL-27. In some embodiments, the one or more recombinant cytokines are IL-2 and IL-15.

In particular embodiments, the provided methods include incubation or culture of the enriched NK cells and feeder cells in the presence of recombinant IL-2. In some embodiments, during at least a portion of the incubation, e.g. added at the initiation of the culturing and optionally one or more times during the culturing, the recombinant IL-2 is present at a concentration of between at or about 1 IU/mL and at or about 500 IU/mL, such as between at or about 1 IU/mL and at or about 250 IU/mL, between at or about 1 IU/mL and at or about 100 IU/mL, between at or about 1 IU/mL and at or about 50 IU/mL, between at or about 50 IU/mL and at or about 500 IU/mL, between at or about 50 IU/mL and at or about 250 IU/mL, between at or about 50 IU/mL and at or about 100 IU/mL, between at or about 100 IU/mL and at or about 500 IU/mL, between at or about 100 IU/mL and at or about 250 IU/mL or between at or about 250 IU/mL and at or about 500 IU/mL, each inclusive. In some embodiments, during at least a portion of the incubation, e.g. added at the initiation of the culturing and optionally one or more times during the culturing, the concentration of the IL-2 is at or about 50 IU/mL, 60 IU/mL, 70 IU/mL, 80 IU/mL, 90 IU/mL, 100 IU/mL, 125 IU/mL, 150 IU/mL, 200 IU/mL, or any value between any of the foregoing. In particular embodiments, the concentration of the recombinant IL-2 added at the initiation of the culturing and optionally one or more times during the culturing is or is about 100 IU/mL. In particular embodiments, the concentration of the recombinant IL-2 added at the initiation of the culturing and optionally one or more times during the culturing is or is about 500 IU/mL.

In particular embodiments, the provided methods include incubation or culture of the enriched NK cells and feeder cells in the presence of recombinant IL-21. In some embodiments, during at least a portion of the incubation, e.g. added at the initiation of the culturing and optionally one or more times during the culturing, the recombinant IL-21 is present at a concentration of between at or about 1 IU/mL and at or about 500 IU/mL, such as between at or about 1 IU/mL and at or about 250 IU/mL, between at or about 1 IU/mL and at or about 100 IU/mL, between at or about 1 IU/mL and at or about 50 IU/mL, between at or about 50 IU/mL and at or about 500 IU/mL, between at or about 50 IU/mL and at or about 250 IU/mL, between at or about 50 IU/mL and at or about 100 IU/mL, between at or about 100 IU/mL and at or about 500 IU/mL, between at or about 100 IU/mL and at or about 250 IU/mL or between at or about 250 IU/mL and at or about 500 IU/mL, each inclusive. In some embodiments, during at least a portion of the incubation, e.g. added at the initiation of the culturing and optionally one or more times during the culturing, the concentration of the IL-21 is at or about 50 IU/mL, 60 IU/mL, 70 IU/mL, 80 IU/mL, 90 IU/mL, 100 IU/mL, 125 IU/mL, 150 IU/mL, 200 IU/mL, or any value between any of the foregoing. In particular embodiments, the concentration of the recombinant IL-21 added at the initiation of the culturing and optionally one or more times during the culturing, is or is about 100 IU/mL.

In particular embodiments, the provided methods include incubation or culture of the enriched NK cells and feeder cells in the presence of recombinant IL-21. In particular embodiments, the concentration of recombinant IL-21 during at least a portion of the culturing, e.g. added at the initiation of the culturing and optionally one or more times during the culturing, is between about 10 ng/ml and about 100 ng/mL, between about 10 ng/mL and about 90 ng/mL, between about 10 ng/ml and about 80 ng/mL, between about 10 ng/mL and about 70 ng/ml, between about 10 ng/mL and about 60 ng/mL, between about 10 ng/ml and about 50 ng/ml, between about 10 ng/mL and about 40 ng/mL, between about 10 ng/ml and about 30 ng/ml, between about 10 ng/mL and about 20 ng/ml, between about 20 ng/ml and about 100 ng/ml, between about 20 ng/ml and about 90 ng/mL, between about 20 ng/ml and about 80 ng/ml, between about 20 ng/ml and about 70 ng/ml, between about 20 ng/ml and about 60 ng/ml, between about 20 ng/ml and about 50 ng/ml, between about 20 ng/ml and about 40 ng/ml, between about 20 ng/ml and about 30 ng/ml, between about 30 ng/mL and about 100 ng/ml, between about 30 ng/mL and about 90 ng/mL, between about 30 ng/ml and about 80 ng/ml, between about 30 ng/ml and about 70 ng/ml, between about 30 ng/ml and about 60 ng/ml, between about 30 ng/ml and about 50 ng/mL, between about 30 ng/mL and about 40 ng/ml, between about 40 ng/ml and about 100 ng/mL, between about 40 ng/ml and about 90 ng/ml, between about 40 ng/mL and about 80 ng/mL, between about 40 ng/ml and about 70 ng/ml, between about 40 ng/mL and about 60 ng/mL, between about 40 ng/mL and about 50 ng/ml, between about 50 ng/ml and about 100 ng/ml, between about 50 ng/ml and about 90 ng/mL, between about 50 ng/ml and about 80 ng/mL, between about 50 ng/ml and about 70 ng/mL, between about 50 ng/mL and about 60 ng/mL, between about 60 ng/ml and about 100 ng/mL, between about 60 ng/mL and about 90 ng/mL, between about 60 ng/ml and about 80 ng/mL, between about 60 ng/ml and about 70 ng/mL, between about 70 ng/ml and about 100 ng/ml, between about 70 ng/mL and about 90 ng/mL, between about 70 ng/ml and about 80 ng/ml, between about 80 ng/mL and about 100 ng/mL, between about 80 ng/ml and about 90 ng/ml, or between about 90 ng/mL and about 100 ng/mL, inclusive. In particular embodiments, the concentration of recombinant IL-21 during at least a portion of the culturing, e.g. added at the initiation of the culturing and optionally one or more times during the culturing, is between about 10 ng/mL and about 100 ng/mL, inclusive. In particular embodiments, the concentration of recombinant IL-21 during at least a portion of the culturing, e.g. added at the initiation of the culturing and optionally one or more times during the culturing, is at or about 25 ng/ml.

In particular embodiments, the concentration of recombinant IL-15 during at least a portion of the culturing, e.g. added at the initiation of the culturing and optionally one or more times during the culturing, is between about 1 ng/ml and about 50 ng/mL, between about 1 ng/mL and about 40 ng/mL, between about 1 ng/mL and about 30 ng/mL, between about 1 ng/mL and about 20 ng/mL, between about 1 ng/mL and about 10 ng/mL, between about 1 ng/ml and about 5 ng/mL, between about 5 ng/mL and about 50 ng/ml, between about 5 ng/ml and about 40 ng/ml, between about 5 ng/mL and about 30 ng/mL, between about 5 ng/ml and about 20 ng/mL, between about 5 ng/mL and about 10 ng/mL, between about 10 ng/ml and about 50 ng/mL, between about 10 ng/mL and about 40 ng/mL, between about 10 ng/ml and about 30 ng/mL, between about 10 ng/mL and about 20 ng/mL, between about 20 ng/ml and about 50 ng/mL, between about 20 ng/mL and about 40 ng/mL, between about 20 ng/ml and about 30 ng/mL, between about 30 ng/mL and about 50 ng/mL, between about 30 ng/ml and about 40 ng/ml, or between about 40 ng/mL and about 50 ng/mL. In particular embodiments, the concentration of recombinant IL-15 during at least a portion of the culturing, e.g. added at the initiation of the culturing and optionally one or more times during the culturing, is between about 1 ng/mL and about 50 ng/mL. In particular embodiments, the concentration of recombinant IL-15 during at least a portion of the culturing, e.g. added at the initiation of the culturing and optionally one or more times during the culturing, is at or about 10 ng/mL.

In particular embodiments, the methods include culture in the presence of IL-2, IL-15 and IL-21. In embodiments of the provided methods, the concentration of recombinant cytokines, e.g. added to the culture at the initiation of the culturing and optionally one or more times during the culturing, is at between at or about 50 IU/mL and at or about 500 IU/mL IL-2, such as at or about 100 IU/mL or 500 IU/mL IL-2; between at or about 1 ng/mL and 50 ng/ml IL-15, such as at or about 10 ng/ml; and between at or about 10 ng/ml and at or about 100 ng/ml IL-21, such as at or about 25 ng/mL. In particular embodiments, 500 IU/mL of IL-2, 10 ng/ml of IL-15, and 25 ng/ml of IL-21 are added during at least a portion of the culturing, such as added at the initiation of the culturing and optionally one or more times during the culturing. In particular embodiments, 100 IU/mL of IL-2, 10 ng/ml of IL-15, and 25 ng/ml of IL-21 are added during at least a portion of the culturing, such as added at the initiation of the culturing and optionally one or more times during the culturing.

In some embodiments, the provided methods include incubation or culture of the enriched NK cells and feeder cells in the presence of recombinant IL-21 and the recombinant IL-21 is added as a complex with an anti-IL-21 antibody. In some embodiments, prior to the culturing, anti-IL-21 antibody is contacted with the recombinant IL-21, thereby forming an IL-21/anti-IL-21 complex, and the IL-21/anti-IL-21 complex is added to the culture medium. In some embodiments, contacting the recombinant IL-21 and the anti-IL-21 antibody to form an IL-21/anti-IL-21 complex is carried out under conditions that include temperature and time suitable for the formation of the complex. In some embodiments, the culturing is carried out at 37° C.±2 for 30 minutes.

In some embodiments, anti-IL-21 antibody is added at a concentration between at or about 100 ng/ml and at or about 500 ng/mL, between at or about 100 ng/ml and at or about 400 ng/mL, between at or about 100 ng/ml and at or about 300 ng/mL, between at or about 100 ng/mL and at or about 200 ng/mL, between at or about 200 ng/ml and at or about 500 ng/ml, between at or about 200 ng/ml and at or about 400 ng/mL, between at or about 200 ng/ml and at or about 300 ng/mL, between at or about 300 ng/ml and at or about 500 ng/mL, between at or about 300 ng/ml and at or about 400 ng/mL, or between at or about 400 ng/ml and at or about 500 ng/mL. In some embodiments, anti-IL-21 antibody is added at a concentration between at or about 100 ng/ml and at or about 500 ng/mL. In some embodiments, anti-IL-21 antibody is added at a concentration of 250 ng/ml.

In particular embodiments, the concentration of recombinant IL-21 used to form a complex with the anti-IL-21 antibody is between about 10 ng/ml and about 100 ng/ml, between about 10 ng/mL and about 90 ng/ml, between about 10 ng/ml and about 80 ng/ml, between about 10 ng/mL and about 70 ng/mL, between about 10 ng/ml and about 60 ng/ml, between about 10 ng/mL and about 50 ng/ml, between about 10 ng/ml and about 40 ng/ml, between about 10 ng/mL and about 30 ng/mL, between about 10 ng/ml and about 20 ng/ml, between about 20 ng/mL and about 100 ng/mL, between about 20 ng/ml and about 90 ng/ml, between about 20 ng/ml and about 80 ng/mL, between about 20 ng/ml and about 70 ng/ml, between about 20 ng/ml and about 60 ng/mL, between about 20 ng/mL and about 50 ng/mL, between about 20 ng/mL and about 40 ng/mL, between about 20 ng/ml and about 30 ng/ml, between about 30 ng/ml and about 100 ng/mL, between about 30 ng/ml and about 90 ng/ml, between about 30 ng/ml and about 80 ng/mL, between about 30 ng/mL and about 70 ng/mL, between about 30 ng/ml and about 60 ng/mL, between about 30 ng/ml and about 50 ng/ml, between about 30 ng/ml and about 40 ng/mL, between about 40 ng/ml and about 100 ng/ml, between about 40 ng/ml and about 90 ng/mL, between about 40 ng/ml and about 80 ng/ml, between about 40 ng/mL and about 70 ng/mL, between about 40 ng/ml and about 60 ng/ml, between about 40 ng/ml and about 50 ng/mL, between about 50 ng/ml and about 100 ng/ml, between about 50 ng/ml and about 90 ng/mL, between about 50 ng/mL and about 80 ng/ml, between about 50 ng/mL and about 70 ng/mL, between about 50 ng/mL and about 60 ng/ml, between about 60 ng/ml and about 100 ng/mL, between about 60 ng/ml and about 90 ng/ml, between about 60 ng/mL and about 80 ng/mL, between about 60 ng/ml and about 70 ng/ml, between about 70 ng/ml and about 100 ng/ml, between about 70 ng/ml and about 90 ng/mL, between about 70 ng/ml and about 80 ng/mL, between about 80 ng/ml and about 100 ng/ml, between about 80 ng/mL and about 90 ng/mL, or between about 90 ng/mL and about 100 ng/mL, inclusive. In particular embodiments, the concentration of recombinant IL-21 used to form a complex with the anti-IL-21 antibody is between about 10 ng/ml and about 100 ng/ml, inclusive. In particular embodiments, the concentration of recombinant IL-21 used to form a complex with the anti-IL-21 antibody is at or about 25 ng/ml.

In particular embodiments, the concentration of recombinant IL-12 during at least a portion of the culturing, e.g. added at the initiation of the culturing and optionally one or more times during the culturing, is between about 1 ng/mL and about 50 ng/ml, between about 1 ng/ml and about 40 ng/mL, between about 1 ng/mL and about 30 ng/mL, between about 1 ng/mL and about 20 ng/mL, between about 1 ng/mL and about 10 ng/ml, between about 1 ng/mL and about 5 ng/mL, between about 5 ng/ml and about 50 ng/ml, between about 5 ng/ml and about 40 ng/ml, between about 5 ng/mL and about 30 ng/ml, between about 5 ng/ml and about 20 ng/mL, between about 5 ng/mL and about 10 ng/mL, between about 10 ng/ml and about 50 ng/mL, between about 10 ng/mL and about 40 ng/mL, between about 10 ng/ml and about 30 ng/mL, between about 10 ng/ml and about 20 ng/mL, between about 20 ng/ml and about 50 ng/ml, between about 20 ng/mL and about 40 ng/mL, between about 20 ng/ml and about 30 ng/ml, between about 30 ng/ml and about 50 ng/mL, between about 30 ng/ml and about 40 ng/ml, or between about 40 ng/mL and about 50 ng/mL. In particular embodiments, the concentration of recombinant IL-12 during at least a portion of the culturing, e.g. added at the initiation of the culturing and optionally one or more times during the culturing, is between about 1 ng/mL and about 50 ng/mL. In particular embodiments, the concentration of recombinant IL-12 during at least a portion of the culturing, e.g. added at the initiation of the culturing and optionally one or more times during the culturing, is at or about 10 ng/mL.

In particular embodiments, the concentration of recombinant IL-18 during at least a portion of the culturing, e.g. added at the initiation of the culturing and optionally one or more times during the culturing, is between about 1 ng/mL and about 50 ng/ml, between about 1 ng/mL and about 40 ng/mL, between about 1 ng/mL and about 30 ng/mL, between about 1 ng/mL and about 20 ng/mL, between about 1 ng/ml and about 10 ng/ml, between about 1 ng/mL and about 5 ng/mL, between about 5 ng/ml and about 50 ng/ml, between about 5 ng/ml and about 40 ng/mL, between about 5 ng/mL and about 30 ng/mL, between about 5 ng/mL and about 20 ng/ml, between about 5 ng/mL and about 10 ng/ml, between about 10 ng/ml and about 50 ng/mL, between about 10 ng/mL and about 40 ng/mL, between about 10 ng/ml and about 30 ng/ml, between about 10 ng/ml and about 20 ng/mL, between about 20 ng/ml and about 50 ng/mL, between about 20 ng/mL and about 40 ng/mL, between about 20 ng/ml and about 30 ng/mL, between about 30 ng/ml and about 50 ng/mL, between about 30 ng/ml and about 40 ng/ml, or between about 40 ng/mL and about 50 ng/mL. In particular embodiments, the concentration of recombinant IL-18 during at least a portion of the culturing, e.g. added at the initiation of the culturing and optionally one or more times during the culturing, is between about 1 ng/mL and about 50 ng/mL. In particular embodiments, the concentration of recombinant IL-18 during at least a portion of the culturing, e.g. added at the initiation of the culturing and optionally one or more times during the culturing, is at or about 10 ng/mL.

In particular embodiments, the concentration of recombinant IL-27 during at least a portion of the culturing, e.g. added at the initiation of the culturing and optionally one or more times during the culturing, is between about 1 ng/mL and about 50 ng/ml, between about 1 ng/ml and about 40 ng/mL, between about 1 ng/mL and about 30 ng/ml, between about 1 ng/ml and about 20 ng/mL, between about 1 ng/mL and about 10 ng/mL, between about 1 ng/mL and about 5 ng/mL, between about 5 ng/mL and about 50 ng/mL, between about 5 ng/ml and about 40 ng/ml, between about 5 ng/ml and about 30 ng/mL, between about 5 ng/ml and about 20 ng/mL, between about 5 ng/mL and about 10 ng/ml, between about 10 ng/ml and about 50 ng/mL, between about 10 ng/ml and about 40 ng/mL, between about 10 ng/ml and about 30 ng/mL, between about 10 ng/mL and about 20 ng/mL, between about 20 ng/ml and about 50 ng/mL, between about 20 ng/ml and about 40 ng/mL, between about 20 ng/ml and about 30 ng/mL, between about 30 ng/mL and about 50 ng/ml, between about 30 ng/ml and about 40 ng/mL, or between about 40 ng/ml and about 50 ng/mL. In particular embodiments, the concentration of recombinant IL-27 during at least a portion of the culturing, e.g. added at the initiation of the culturing and optionally one or more times during the culturing, is between about 1 ng/mL and about 50 ng/mL. In particular embodiments, the concentration of recombinant IL-27 during at least a portion of the culturing, e.g. added at the initiation of the culturing and optionally one or more times during the culturing, is at or about 10 ng/mL.

In some embodiments, the methods include exchanging the culture medium, which, in some aspects includes washing the cells. For example, during at least a portion of the culture or incubation the culture medium can be exchanged or washed out intermittently, such as daily, every other day, every three days, or once a week. In particular embodiments, the culture medium is exchanged or washed out beginning within or within about 3 days to 7 days after initiation of the culture, such as at or about at day 3, day 4, day 5, day 6 or day 7. In particular embodiments, the culture medium is exchanged or washed out at or about beginning at day 5. For example, media is exchanged on day 5 and every 2-3 days afterwards.

Once the culture medium is removed or washed out, it is replenished. In some embodiments, the replenished culture medium includes the one or more growth factors or cytokines, such as any as described above. Hence, in some embodiments, the one or more growth factor or cytokine, such as recombinant IL-2, IL-15 and/or IL-21, is added intermittently during the incubation or culture. In some such aspects, the one or more growth factor or cytokine, such as recombinant IL-2, IL-15 and/or IL-21, is added at or about at the initiation of the culture or incubation, and then is added intermittently during the culture or incubation, such as each time the culture medium is exchanged or washed out. In some embodiments, the one or more growth factor or cytokine, such as recombinant IL-2, IL-15 and/or IL-21, is added to the culture or incubation beginning at day 0 (initiation of the incubation) and, at each exchange or wash out of the culture medium, it is further added to replenish the culture or incubation with the one or more growth factor or cytokine, such as recombinant IL-2, IL-15 and/or IL-21. In some embodiments, the methods include adding the one or more growth factor or cytokine, e.g. recombinant IL-2, IL-15 and/or IL-21, at the initiation of the incubation (day 0), and every two or three days at each wash or exchange of the culture medium for the duration of the incubation, e.g. at or about at day 5, day 7, day 9, day 11, and day 14 of the culture or incubation.

In particular embodiments, the culturing is carried out in the presence of at least one of IL-2, IL-15 and IL-21 and the culture medium is replenished to include at least one of IL-2, IL-15 and IL-21. In some embodiments, the culturing is carried out in the presence of IL-2 and IL-21 and the culture medium is replenished to include IL-2 and IL-21. In some embodiments, the culturing is carried out in the presence of IL-2 and IL-15 and the culture medium is replenished to include IL-2 and IL-15. In some embodiments, the culturing is carried out in the presence of IL-15 and IL-21 and the culture medium is replenished to include IL-15 and IL21. In some embodiments, the culturing is carried out in the presence of IL-2, IL-15 and IL-21 and the culture medium is replenished to include IL-2, IL-15 and IL-21. In some embodiments, one or more additional cytokines can be utilized in the expansion of the NK cells, including but not limited to recombinant IL-18, recombinant IL-7, and/or recombinant IL-12.

In some embodiments, the replenished culture medium includes the one or more growth factors or cytokines, such as recombinant IL-2. Hence, in some embodiments, the growth factor or cytokine, such as recombinant IL-2, is added intermittently during the incubation or culture. In some such aspects, the growth factor or cytokine, such as recombinant IL-2, is added at or about at the initiation of the culture or incubation, and then is added intermittently during the culture or incubation, such as each time the culture medium is exchanged or washed out. In some embodiments, the growth factor or cytokine, such as recombinant IL-2, is added to the culture or incubation beginning at day 0 (initiation of the incubation) and, at each exchange or wash out of the culture medium, it is further added to replenish the culture or incubation with the growth factor or cytokine, such as recombinant IL-2. In some embodiments, the methods include adding recombinant IL-2 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-2 is added to the culture or incubation at a concentration of between at or about 1 IU/mL and at or about 500 IU/mL, such as between at or about 1 IU/mL and at or about 250 IU/mL, between at or about 1 IU/mL and at or about 100 IU/mL, between at or about 1 IU/mL and at or about 50 IU/mL, between at or about 50 IU/mL and at or about 500 IU/mL, between at or about 50 IU/mL and at or about 250 IU/mL, between at or about 50 IU/mL and at or about 100 IU/mL, between at or about 100 IU/mL and at or about 500 IU/mL, between at or about 100 IU/mL and at or about 250 IU/mL or between at or about 250 IU/mL and at or about 500 IU/mL, each inclusive. In some embodiments, the recombinant IL-2 is added to the culture or incubation at a concentration that is at or about 50 IU/mL, 60 IU/mL, 70 IU/mL, 80 IU/mL, 90 IU/mL, 100 IU/mL, 125 IU/mL, 150 IU/mL, 200 IU/mL, or any value between any of the foregoing. In particular embodiments, the concentration of the recombinant IL-2 is or is about 100 IU/mL. In particular embodiments, the concentration of the recombinant IL-2 is or is about 500 IU/mL.

In some embodiments, the replenished culture medium includes the one or more growth factors or cytokines, such as recombinant IL-21. Hence, in some embodiments, the growth factor or cytokine, such as recombinant IL-21, is added intermittently during the incubation or culture. In some such aspects, the growth factor or cytokine, such as recombinant IL-21, is added at or about at the initiation of the culture or incubation, and then is added intermittently during the culture or incubation, such as each time the culture medium is exchanged or washed out. In some embodiments, the growth factor or cytokine, such as recombinant IL-21, is added to the culture or incubation beginning at day 0 (initiation of the incubation) and, at each exchange or wash out of the culture medium, it is further added to replenish the culture or incubation with the growth factor or cytokine, such as recombinant IL-21. In some embodiments, the methods include adding recombinant IL-21 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-21 is added to the culture or incubation at a concentration of between about 10 ng/ml and about 100 ng/ml, between about 10 ng/mL and about 90 ng/mL, between about 10 ng/mL and about 80 ng/ml, between about 10 ng/ml and about 70 ng/mL, between about 10 ng/ml and about 60 ng/ml, between about 10 ng/ml and about 50 ng/mL, between about 10 ng/ml and about 40 ng/ml, between about 10 ng/ml and about 30 ng/ml, between about 10 ng/mL and about 20 ng/ml, between about 20 ng/mL and about 100 ng/mL, between about 20 ng/mL and about 90 ng/ml, between about 20 ng/mL and about 80 ng/mL, between about 20 ng/mL and about 70 ng/ml, between about 20 ng/mL and about 60 ng/mL, between about 20 ng/ml and about 50 ng/ml, between about 20 ng/mL and about 40 ng/mL, between about 20 ng/ml and about 30 ng/ml, between about 30 ng/ml and about 100 ng/ml, between about 30 ng/mL and about 90 ng/ml, between about 30 ng/ml and about 80 ng/ml, between about 30 ng/ml and about 70 ng/ml, between about 30 ng/mL and about 60 ng/mL, between about 30 ng/ml and about 50 ng/ml, between about 30 ng/ml and about 40 ng/mL, between about 40 ng/mL and about 100 ng/mL, between about 40 ng/ml and about 90 ng/mL, between about 40 ng/ml and about 80 ng/mL, between about 40 ng/mL and about 70 ng/mL, between about 40 ng/mL and about 60 ng/mL, between about 40 ng/ml and about 50 ng/ml, between about 50 ng/mL and about 100 ng/ml, between about 50 ng/mL and about 90 ng/mL, between about 50 ng/mL and about 80 ng/ml, between about 50 ng/mL and about 70 ng/mL, between about 50 ng/ml and about 60 ng/mL, between about 60 ng/ml and about 100 ng/mL, between about 60 ng/ml and about 90 ng/ml, between about 60 ng/ml and about 80 ng/mL, between about 60 ng/mL and about 70 ng/ml, between about 70 ng/ml and about 100 ng/mL, between about 70 ng/mL and about 90 ng/ml, between about 70 ng/ml and about 80 ng/mL, between about 80 ng/mL and about 100 ng/ml, between about 80 ng/mL and about 90 ng/mL, or between about 90 ng/ml and about 100 ng/mL, inclusive. In particular embodiments, the recombinant IL-21 is added to the culture or incubation at a concentration of between about 10 ng/ml and about 100 ng/mL, inclusive. The recombinant IL-21 is added to the culture or incubation at a concentration of at or about 25 ng/mL.

In some embodiments, the replenished culture medium includes the one or more growth factors or cytokines, such as recombinant IL-21, added as a complex with an antibody, such as an anti-IL-21 antibody. Hence, in some embodiments, the complex, such as an IL-21/anti-IL-21 antibody complex, is added intermittently during the incubation or culture. In some such aspects, the complex, such as an IL-21/anti-IL-21 antibody complex, is added at or about at the initiation of the culture or incubation, and then is added intermittently during the culture or incubation, such as each time the culture medium is exchanged or washed out. In some embodiments, the complex, such as an IL-21/anti-IL-21 antibody complex, is added to the culture or incubation beginning at day 0 (initiation of the incubation) and, at each exchange or wash out of the culture medium, it is further added to replenish the culture or incubation with the complex, such as an IL-21/anti-IL-21 antibody complex. In some embodiments, the methods include adding the complex, such as an IL-21/anti-IL-21 antibody complex, at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the anti-IL-21 antibody is contacted with the recombinant IL-21, thereby forming an IL-21/anti-IL-21 complex, and the IL-21/anti-IL-21 complex is added to the culture medium. In any of such embodiments, contacting the recombinant IL-21 and the anti-IL-21 antibody to form an IL-21/anti-IL-21 complex is carried out under conditions that include temperature and time suitable for the formation of the complex. In any of such embodiments, the culturing is carried out at 37° C.±2 for 30 minutes. In any of such embodiments, anti-IL-21 antibody is added at a concentration between at or about 100 ng/ml and at or about 500 ng/mL, between at or about 100 ng/ml and at or about 400 ng/mL, between at or about 100 ng/ml and at or about 300 ng/ml, between at or about 100 ng/ml and at or about 200 ng/ml, between at or about 200 ng/ml and at or about 500 ng/mL, between at or about 200 ng/mL and at or about 400 ng/ml, between at or about 200 ng/ml and at or about 300 ng/mL, between at or about 300 ng/mL and at or about 500 ng/ml, between at or about 300 ng/mL and at or about 400 ng/mL, or between at or about 400 ng/ml and at or about 500 ng/mL. In some embodiments, anti-IL-21 antibody is added at a concentration between at or about 100 ng/ml and at or about 500 ng/mL. In some embodiments, anti-IL-21 antibody is added at a concentration of 250 ng/mL. In any of such embodiments, the concentration of recombinant IL-21 used to form a complex with the anti-IL-21 antibody is between about 10 ng/ml and about 100 ng/mL, between about 10 ng/ml and about 90 ng/mL, between about 10 ng/mL and about 80 ng/mL, between about 10 ng/ml and about 70 ng/ml, between about 10 ng/mL and about 60 ng/mL, between about 10 ng/ml and about 50 ng/mL, between about 10 ng/mL and about 40 ng/mL, between about 10 ng/ml and about 30 ng/mL, between about 10 ng/ml and about 20 ng/mL, between about 20 ng/ml and about 100 ng/mL, between about 20 ng/mL and about 90 ng/mL, between about 20 ng/ml and about 80 ng/mL, between about 20 ng/mL and about 70 ng/mL, between about 20 ng/ml and about 60 ng/mL, between about 20 ng/mL and about 50 ng/mL, between about 20 ng/ml and about 40 ng/mL, between about 20 ng/ml and about 30 ng/mL, between about 30 ng/ml and about 100 ng/ml, between about 30 ng/mL and about 90 ng/mL, between about 30 ng/ml and about 80 ng/mL, between about 30 ng/ml and about 70 ng/mL, between about 30 ng/ml and about 60 ng/mL, between about 30 ng/ml and about 50 ng/mL, between about 30 ng/ml and about 40 ng/mL, between about 40 ng/ml and about 100 ng/ml, between about 40 ng/ml and about 90 ng/mL, between about 40 ng/ml and about 80 ng/mL, between about 40 ng/ml and about 70 ng/mL, between about 40 ng/mL and about 60 ng/mL, between about 40 ng/ml and about 50 ng/ml, between about 50 ng/mL and about 100 ng/mL, between about 50 ng/ml and about 90 ng/mL, between about 50 ng/mL and about 80 ng/mL, between about 50 ng/ml and about 70 ng/mL, between about 50 ng/ml and about 60 ng/mL, between about 60 ng/mL and about 100 ng/ml, between about 60 ng/mL and about 90 ng/mL, between about 60 ng/ml and about 80 ng/mL, between about 60 ng/mL and about 70 ng/mL, between about 70 ng/ml and about 100 ng/mL, between about 70 ng/mL and about 90 ng/mL, between about 70 ng/ml and about 80 ng/mL, between about 80 ng/ml and about 100 ng/ml, between about 80 ng/ml and about 90 ng/mL, or between about 90 ng/ml and about 100 ng/mL, inclusive. In particular embodiments, the concentration of recombinant IL-21 used to form a complex with the anti-IL-21 antibody is between about 10 ng/ml and about 100 ng/mL, inclusive. In particular embodiments, the concentration of recombinant IL-21 used to form a complex with the anti-IL-21 antibody is at or about 25 ng/ml.

In some embodiments, the replenished culture medium includes the one or more growth factors or cytokines, such as recombinant IL-15. Hence, in some embodiments, the growth factor or cytokine, such as recombinant IL-15, is added intermittently during the incubation or culture. In some such aspects, the growth factor or cytokine, such as recombinant IL-15, is added at or about at the initiation of the culture or incubation, and then is added intermittently during the culture or incubation, such as each time the culture medium is exchanged or washed out. In some embodiments, the growth factor or cytokine, such as recombinant IL-15, is added to the culture or incubation beginning at day 0 (initiation of the incubation) and, at each exchange or wash out of the culture medium, it is further added to replenish the culture or incubation with the growth factor or cytokine, such as recombinant IL-15. In some embodiments, the methods include adding recombinant IL-15 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-15 is added to the culture or incubation at a concentration of between about 1 ng/ml and about 50 ng/mL, between about 1 ng/ml and about 40 ng/ml, between about 1 ng/mL and about 30 ng/ml, between about 1 ng/ml and about 20 ng/mL, between about 1 ng/mL and about 10 ng/mL, between about 1 ng/mL and about 5 ng/mL, between about 5 ng/ml and about 50 ng/mL, between about 5 ng/ml and about 40 ng/mL, between about 5 ng/ml and about 30 ng/mL, between about 5 ng/ml and about 20 ng/mL, between about 5 ng/mL and about 10 ng/ml, between about 10 ng/ml and about 50 ng/mL, between about 10 ng/mL and about 40 ng/mL, between about 10 ng/ml and about 30 ng/mL, between about 10 ng/ml and about 20 ng/ml, between about 20 ng/ml and about 50 ng/ml, between about 20 ng/ml and about 40 ng/ml, between about 20 ng/ml and about 30 ng/mL, between about 30 ng/ml and about 50 ng/ml, between about 30 ng/ml and about 40 ng/mL, or between about 40 ng/ml and about 50 ng/mL. In any of such embodiments, the recombinant IL-15 is added to the culture or incubation at a concentration of between about 1 ng/mL and about 50 ng/mL. In any of such embodiments, the recombinant IL-15 is added to the culture or incubation at a concentration of at or about 10 ng/ml. In particular embodiments, 500 IU/mL of IL-2, 10 ng/ml of IL-15, and 25 ng/ml of IL-21 are added to the culture or incubation.

In some embodiments, the replenished culture medium includes the one or more growth factors or cytokines, such as recombinant IL-12. Hence, in some embodiments, the growth factor or cytokine, such as recombinant IL-12, is added intermittently during the incubation or culture. In some such aspects, the growth factor or cytokine, such as recombinant IL-12, is added at or about at the initiation of the culture or incubation, and then is added intermittently during the culture or incubation, such as each time the culture medium is exchanged or washed out. In some embodiments, the growth factor or cytokine, such as recombinant IL-12, is added to the culture or incubation beginning at day 0 (initiation of the incubation) and, at each exchange or wash out of the culture medium, it is further added to replenish the culture or incubation with the growth factor or cytokine, such as recombinant IL-12. In some embodiments, the methods include adding recombinant IL-12 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-12 is added to the culture or incubation at a concentration of between about 1 ng/mL and about 50 ng/mL, between about 1 ng/mL and about 40 ng/mL, between about 1 ng/mL and about 30 ng/mL, between about 1 ng/mL and about 20 ng/mL, between about 1 ng/ml and about 10 ng/mL, between about 1 ng/mL and about 5 ng/mL, between about 5 ng/ml and about 50 ng/ml, between about 5 ng/ml and about 40 ng/mL, between about 5 ng/mL and about 30 ng/ml, between about 5 ng/mL and about 20 ng/mL, between about 5 ng/mL and about 10 ng/ml, between about 10 ng/mL and about 50 ng/mL, between about 10 ng/ml and about 40 ng/mL, between about 10 ng/mL and about 30 ng/mL, between about 10 ng/mL and about 20 ng/ml, between about 20 ng/ml and about 50 ng/ml, between about 20 ng/ml and about 40 ng/ml, between about 20 ng/ml and about 30 ng/mL, between about 30 ng/ml and about 50 ng/ml, between about 30 ng/ml and about 40 ng/mL, or between about 40 ng/ml and about 50 ng/mL. In any of such embodiments, the recombinant IL-12 is added to the culture or incubation at a concentration of between about 1 ng/mL and about 50 ng/mL. In any of such embodiments, the recombinant IL-12 is added to the culture or incubation at a concentration of at or about 10 ng/mL.

In some embodiments, the replenished culture medium includes the one or more growth factors or cytokines, such as recombinant IL-18. Hence, in some embodiments, the growth factor or cytokine, such as recombinant IL-18, is added intermittently during the incubation or culture. In some such aspects, the growth factor or cytokine, such as recombinant IL-18, is added at or about at the initiation of the culture or incubation, and then is added intermittently during the culture or incubation, such as each time the culture medium is exchanged or washed out. In some embodiments, the growth factor or cytokine, such as recombinant IL-18, is added to the culture or incubation beginning at day 0 (initiation of the incubation) and, at each exchange or wash out of the culture medium, it is further added to replenish the culture or incubation with the growth factor or cytokine, such as recombinant IL-18. In some embodiments, the methods include adding recombinant IL-18 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-18 is added to the culture or incubation at a concentration of between about 1 ng/mL and about 50 ng/mL, between about 1 ng/ml and about 40 ng/mL, between about 1 ng/mL and about 30 ng/mL, between about 1 ng/mL and about 20 ng/ml, between about 1 ng/mL and about 10 ng/ml, between about 1 ng/mL and about 5 ng/mL, between about 5 ng/mL and about 50 ng/ml, between about 5 ng/ml and about 40 ng/mL, between about 5 ng/mL and about 30 ng/mL, between about 5 ng/mL and about 20 ng/mL, between about 5 ng/ml and about 10 ng/mL, between about 10 ng/mL and about 50 ng/mL, between about 10 ng/mL and about 40 ng/mL, between about 10 ng/ml and about 30 ng/ml, between about 10 ng/ml and about 20 ng/mL, between about 20 ng/ml and about 50 ng/mL, between about 20 ng/ml and about 40 ng/ml, between about 20 ng/ml and about 30 ng/mL, between about 30 ng/mL and about 50 ng/mL, between about 30 ng/ml and about 40 ng/mL, or between about 40 ng/mL and about 50 ng/mL. In any of such embodiments, the recombinant IL-18 is added to the culture or incubation at a concentration of between about 1 ng/mL and about 50 ng/mL. In any of such embodiments, the recombinant IL-18 is added to the culture or incubation at a concentration of at or about 10 ng/ml.

In some embodiments, the replenished culture medium includes the one or more growth factors or cytokines, such as recombinant IL-27. Hence, in some embodiments, the growth factor or cytokine, such as recombinant IL-27, is added intermittently during the incubation or culture. In some such aspects, the growth factor or cytokine, such as recombinant IL-27, is added at or about at the initiation of the culture or incubation, and then is added intermittently during the culture or incubation, such as each time the culture medium is exchanged or washed out. In some embodiments, the growth factor or cytokine, such as recombinant IL-27, is added to the culture or incubation beginning at day 0 (initiation of the incubation) and, at each exchange or wash out of the culture medium, it is further added to replenish the culture or incubation with the growth factor or cytokine, such as recombinant IL-27. In some embodiments, the methods include adding recombinant IL-27 at the initiation of the incubation (day 0), and every two or three days at each wash or exchange of the culture medium for the duration of the incubation, e.g. at or about at day 5, day 7, day 9, day 11, and day 14 of the culture or incubation. In any of such embodiments, the recombinant IL-27 is added to the culture or incubation at a concentration of between about 1 ng/mL and about 50 ng/mL, between about 1 ng/ml and about 40 ng/mL, between about 1 ng/mL and about 30 ng/mL, between about 1 ng/mL and about 20 ng/mL, between about 1 ng/mL and about 10 ng/mL, between about 1 ng/ml and about 5 ng/mL, between about 5 ng/ml and about 50 ng/mL, between about 5 ng/ml and about 40 ng/mL, between about 5 ng/ml and about 30 ng/ml, between about 5 ng/ml and about 20 ng/mL, between about 5 ng/mL and about 10 ng/ml, between about 10 ng/ml and about 50 ng/ml, between about 10 ng/ml and about 40 ng/mL, between about 10 ng/ml and about 30 ng/mL, between about 10 ng/ml and about 20 ng/ml, between about 20 ng/ml and about 50 ng/mL, between about 20 ng/mL and about 40 ng/ml, between about 20 ng/mL and about 30 ng/mL, between about 30 ng/mL and about 50 ng/mL, between about 30 ng/ml and about 40 ng/mL, or between about 40 ng/mL and about 50 ng/mL. In any of such embodiments, the recombinant IL-27 is added to the culture or incubation at a concentration of between about 1 ng/mL and about 50 ng/mL. In any of such embodiments, the recombinant IL-27 is added to the culture or incubation at a concentration of at or about 10 ng/mL.

In embodiments of the provided methods, culturing or incubating includes providing the chemical and physical conditions (e.g., temperature, gas) which are required or useful for NK cell maintenance. Examples of chemical conditions which may support NK cell proliferation or expansion include but are not limited to buffers, nutrients, serum, vitamins and antibiotics which are typically provided in the growth (i.e., culture) medium. In one embodiment, the NK culture medium includes MEMα comprising 10% FCS or CellGro SCGM (Cell Genix) comprising 5% Human Serum/LiforCell® FBS Replacement (Lifeblood Products). Other media suitable for use with the invention include, but are not limited to Glascow's medium (Gibco Carlsbad Calif.), RPMI medium (Sigma-Aldrich, St Louis Mo.) or DMEM (Sigma-Aldrich, St Louis Mo.). It will be noted that many of the culture media contain nicotinamide as a vitamin supplement for example, MEMα (8.19 μM nicotinamide), RPMI (8.19 μM nicotinamide), DMEM (32.78 μM nicotinamide) and Glascow's medium (16.39 μM nicotinamide).

In some embodiments, such as for applications in which cells are introduced (or reintroduced) into a human subject, culturing is carried out using serum-free formulations, such as AIM V™ serum free medium for lymphocyte culture, MARROWMAX™ bone marrow medium or serum-free stem cell growth medium (SCGM) (e.g. CellGenix® GMP SCGM). Such medium formulations and supplements are available from commercial sources. The cultures can be supplemented with amino acids, antibiotics, and/or with other growth factors cytokines as described to promote optimal viability, proliferation, functionality and/or and survival. In some embodiments, the serum-free media also may be supplemented with a low percentage of human serum, such as 0.5% to 10% human serum, such as at or about 5% human serum. In such embodiments, the human serum can be human serum from human AB plasma (human AB serum) or autologous serum.

In some embodiments, the culturing with feeder cells, and optionally cytokines (e.g. recombinant IL-2 or IL-21) is carried out under conditions that include temperature suitable for the growth or expansion of human NK cells, for example, at least about 25 degrees Celsius, generally at least about 30 degrees, and generally at or about 37 degrees Celsius. In some embodiments, the culturing is carried out at 37° C.±2 in 5% CO₂.

In embodiments of the provided methods, the culturing includes incubation that is carried out under GMP conditions. In some embodiments, the incubation is in a closed system, which in some aspects may be a closed automated system. In some embodiments, the culture media containing the one or more recombinant cytokines or growth factors is a serum-free media. In some embodiments, the incubation is carried out in a closed automated system and with serum-free media.

In some embodiments, the expansion of the NK cells is carried out in a culture vessel suitable for cell expansion. In some embodiments, the culture vessel is a gas permeable culture vessel, such as a G-Rex system (e.g. G-Rex 10, G-Rex 10M, G-Rex 100 M/100M-CS or G-Rex 500 M/500M-CS). In some embodiments the culture vessel is a microplate, flask, bag or other culture vessel suitable for expansion of cells in a closed system. In some embodiments, expansion can be carried out in a bioreactor. In some embodiments, the expansion is carried out using a cell expansion system by transfer of the cells to gas permeable bags, such as in connection with a bioreactor (e.g. Xuri Cell Expansion System W25 (GE Healthcare)). In an embodiment, the cell expansion system includes a culture vessel, such as a bag, e.g. gas permeable cell bag, with a volume that is about 50 mL, about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, and about 10 L, or any value between any of the foregoing. In some embodiments, the process is automated or semi-automated. In some aspects, the expansion culture is carried out under static conditions. In some embodiments, the expansion culture is carried out under rocking conditions. The medium can be added in bolus or can be added on a perfusion schedule. In some embodiments, the bioreactor maintains the temperature at or near 37° C. and CO2 levels at or near 5% with a steady air flow at, at about, or at least 0.01 L/min, 0.05 L/min, 0.1 L/min, 0.2 L/min, 0.3 L/min, 0.4 L/min, 0.5 L/min, 1.0 L/min, 1.5 L/min, or 2.0 L/min or greater than 2.0 L/min. In certain embodiments, at least a portion of the culturing is performed with perfusion, such as with a rate of 290 ml/day, 580 ml/day, and/or 1160 ml/day.

In some aspects, cells are expanded in an automated closed expansion system that is perfusion enabled. Perfusions can continuously add media to the cells to ensure an optimal growth rate is achieved.

The expansion methods can be carried out under GMP conditions, including in a closed automated system and using serum free medium. In some embodiments, any one or more of the steps of the method can be carried out in a closed system or under GMP conditions. In certain embodiments, all process operations are performed in a GMP suite. In some embodiments, a closed system is used for carrying out one or more of the other processing steps of a method for manufacturing, generating or producing a cell therapy. In some embodiments, one or more or all of the processing steps, e.g., isolation, selection and/or enrichment, processing, culturing steps including incubation in connection with expansion of the cells, and formulation steps is carried out using a system, device, or apparatus in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps.

In some of any of the provided embodiments, the culturing is carried out until a time at which the method achieves expansion of at least or at least about 2.50×10⁸ g-NK cells. In some of any of the provided embodiments, the culturing is carried out until a time at which the method achieves expansion of at least or at least about 5.0×10⁸ g-NK cells. In some of any of the provided embodiments, the culturing is carried out until the method achieves expansion of at least or at least about 1.0×10⁹ g-NK cells. In some of any of the provided embodiments, the culturing is carried out until a time at which the method achieves expansion of at least or at least about 5.0×10⁹ g-NK cells.

In some of any of the provided embodiments, the culturing is carried out for at or about or at least at or at least about 5 days, 6 days, 7 days, 8 days, 9 days, 10 days, 11 days, 12 days, 13 days, 14 days, 15 days, 16 days, 17 days, 18 days, 19 days, 20 day, 21 days, 22 days, 23 days, 24 days or 25 days. In some embodiments, the culturing is carried out for at or about or at least at or about 14 days. In some embodiments the culturing is carried out for at or about or at least at or about 21 days.

In some of any of the provided embodiments, the culturing or incubation in accord with any of the provided methods is carried out for at or about or at least at or at least about 5 days, 6 days, 7 days, 8 days, 9 days, 10 days, 11 days, 12 days, 13 days, 14 days, 15 days, 16 days, 17 days, 18 days, 19 days, 20 day, 21 days, 22 days, 23 days, 24 days or 25 days. In some embodiments, the culturing is carried out for at or about or at least at or about 14 days. In some embodiments, the culturing is carried out for at or about or at least at or about 21 days. In certain embodiments, a longer duration of culturing is typically necessary if the enriched NK cells at the initiation of the culturing have been thawed after having been previously frozen or cryopreserved. It is within the level of a skilled artisan to empirically determine the optimal number of days to culture the cells depending on factors such as the state of the cells at the initiation of the culture, the health or viability of the cells that the initiation of the culture or during the culturing and/or the desired number of threshold cells at the end of the culturing depending, for example, on the desired application of the cells, such as the dose of cells to be administered to a subject for therapeutic purposes.

At the end of the culturing, the cells are harvested. Collection or harvesting of the cells can be achieved by centrifugation of the cells from the culture vessel after the end of the culturing. For example, cells are harvested by centrifugation after approximately 14 days of culture. After harvesting of the cells, the cells are washed. A sample of the cells can be collected for functional or phenotypic testing. Any other cells not used for functional or phenotypic testing can be separately formulated. In some cases, the cells are formulated with a cryoprotectant for cryopreservation of cells.

In some embodiments, the provided methods include steps for freezing, e.g., cryopreserving, the cells, either before or after isolation, selection and/or enrichment. In some embodiments, the provided methods include steps for freezing, e.g., cryopreserving, the cells, either before or after incubation and/or culturing. In some embodiments, the method includes cryopreserving the cells in the presence of a cryoprotectant, thereby producing a cryopreserved composition. In some aspects, prior to the incubating and/or prior to administering to a subject, the method includes washing the cryopreserved composition under conditions to reduce or remove the cyroprotectant. Any of a variety of known freezing solutions and parameters in some aspects may be used. In some embodiments, the cells are frozen, e.g., cryofrozen or cryopreserved, in media and/or solution with a final concentration of or of about 12.5%, 12.0%, 11.5%, 11.0%, 10.5%, 10.0%, 9.5%, 9.0%, 8.5%, 8.0%, 7.5%, 7.0%, 6.5%, 6.0%, 5.5%, or 5.0% DMSO, or between 1% and 15%, between 6% and 12%, between 5% and 10%, or between 6% and 8% DMSO. In particular embodiments, the cells are frozen, e.g., cryofrozen or cryopreserved, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to or to about −80° C. at a rate of or of about 1º per minute and stored in the vapor phase of a liquid nitrogen storage tank. In some embodiments, the cells are frozen in a serum-free cryopreservation medium comprising a cryoprotectant. In some embodiments, the cryoprotectant is DMSO. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10 or CS5). CryoStor™ CS10 is a cryopreservation medium containing 10% dimethyl sulfoxide (DMSO). CryoStor™ CS5 is a cryopreservation medium containing 5% dimethyl sulfoxide (DMSO). In some embodiments, the cryopreservation media contains one or more additional excipients, such as plasmalyte A or human serum albumin (HSA).

In some embodiments, the cells are cryopreserved at a density of 5×10⁶ to ×1×10⁸ cells/mL. For example, the cells are cryopreserved at a density of at or about 5×10⁶ cells/mL, at or about 10×10⁶ cells/mL, at or about 15×10⁶ cells/mL, at or about 20×10⁶ cells/mL, at or about 25×10⁶ cells/mL, at or about 30×10⁶ cells/mL, at or about 40×10⁶ cells/mL, at or about 50×10⁶ cells/mL, at or about 60×10⁶ cells/mL, at or about 70×10⁶ cells/mL, at or about 80×10⁶ cells/mL or at or about 90×10⁶ cells/mL, or any value between any of the foregoing. The cells can be cryopreserved in any volume as suitable for the cryopreservation vessel. In some embodiments, the cells are cryopreserved in a vial. The volume of the cryopreservation media may be between at or about 1 mL and at or about 50 mL, such as at or about 1 mL and 5 mL. In some embodiments, the cells are cryopreserved in a bag. The volume of the cryopreservation media may between at or about 10 mL and at or about 500 mL, such as between at or about 100 mL or at or about 200 mL. The harvested and expanded cells can be cryopreserved at low temperature environments, such as temperatures of −80° C. to −196° ° C. In some of any of the provided methods, the method produces an increased number of NKG2C^{pos} cells at the end of the culturing compared to at the initiation of the culturing. For example, the increase in NKG2C^{pos} cells at the end of culturing compared to at the initiation of the culturing can be greater than or greater than about 100-fold, greater than or greater than about 200-fold, greater than or greater than about 300-fold, greater than or greater than about 400-fold, greater than or greater than about 500-fold, greater than or greater than about 600-fold, greater than or greater than about 700-fold or greater than or greater than about 800-fold. In some of any embodiments, the increase is at or about 1000-fold greater. In some of any embodiments, the increase is at or about 2000-fold greater. In some of any embodiments, the increase is at or about 2500-fold greater. In some of any embodiments, the increase is at or about 3000-fold greater. In some of any embodiments, the increase is at or about 5000-fold greater. In some of any embodiments, the increase is at or about 10000-fold greater. In some of any embodiments, the increase is at or about 15000-fold greater. In some of any embodiments, the increase is at or about 20000-fold greater. In some of any embodiments, the increase is at or about 25000-fold greater. In some of any embodiments, the increase is at or about 30000-fold greater. In some of any embodiments, the increase is at or about 35000-fold greater. In some embodiments, the culturing or incubation in accord with any of the provided methods is carried out until a time at which the method achieves expansion of at least at or about 2.50×10⁸ NKG2C^{pos} cells, at least at or about 3.0×10⁸ NKG2C^{pos} cells, at least at or about 4.0×10⁸ NKG2C^{pos} cells, at least at or about 5.0×10⁸ NKG2C^{pos} cells, at least at or about 6.0×10⁸ NKG2C^{pos} cells, at least at or about 7.0×10⁸ NKG2C^{pos} cells, at least at or about 8.0×10⁸ NKG2C^{pos} cells, at least at or about 9.0×10⁸ NKG2C^{pos} cells, at least at or about 1.0×10⁹ NKG2C^{pos} cells, at least at or about 1.5×10⁹ NKG2C^{pos} cells, at least at or about 2.0×10⁹ NKG2C^{pos} cells, at least at or about 3.0×10⁹ NKG2C^{pos} cells, at least at or about 4.0×10⁹ NKG2C^{pos} cells, at least at or about 5.0×10⁹ NKG2C^{pos} cells, at least at or about 1.0×10¹⁰ NKG2C^{pos} cells, at least at or about 1.5×10¹⁰ NKG2C^{pos} cells, at least at or about 2.0×10¹⁰ NKG2C^{pos} cells, at least at or about 2.5×10¹⁰ NKG2C^{pos} cells or more.

In some of any of the provided methods, the method produces an increased number of NKG2A^{neg} cells at the end of the culturing compared to at the initiation of the culturing. For example, the increase in NKG2A^{neg} cells at the end of culturing compared to at the initiation of the culturing can be greater than or greater than about 100-fold, greater than or greater than about 200-fold, greater than or greater than about 300-fold, greater than or greater than about 400-fold, greater than or greater than about 500-fold, greater than or greater than about 600-fold, greater than or greater than about 700-fold or greater than or greater than about 800-fold. In some of any embodiments, the increase is at or about 1000-fold greater. In some of any embodiments, the increase is at or about 2000-fold greater. In some of any embodiments, the increase is at or about 3000-fold greater. In some of any embodiments, the increase is at or about 2500-fold greater. In some of any embodiments, the increase is at or about 5000-fold greater. In some of any embodiments, the increase is at or about 10000-fold greater. In some of any embodiments, the increase is at or about 15000-fold greater. In some of any embodiments, the increase is at or about 20000-fold greater. In some of any embodiments, the increase is at or about 25000-fold greater. In some of any embodiments, the increase is at or about 30000-fold greater. In some of any embodiments, the increase is at or about 35000-fold greater. In some embodiments, the culturing or incubation in accord with any of the provided methods is carried out until a time at which the method achieves expansion of at least at or about 2.50×10⁸ NKG2A^{neg} cells, at least at or about 3.0×10⁸ NKG2A^{neg} cells, at least at or about 4.0×10⁸ NKG2A^{neg} cells, at least at or about 5.0×10⁸ NKG2A^{neg} cells, at least at or about 6.0×10⁸ NKG2A^{neg} cells, at least at or about 7.0×10⁸ NKG2A^{neg} cells, at least at or about 8.0×10⁸ NKG2A^{neg} cells, at least at or about 9.0×10⁸ NKG2A^{neg} cells, at least at or about 1.0×10⁹ NKG2A^{neg} cells, at least at or about 1.5×10⁹ NKG2A^{neg} cells, at least at or about 2.0×10⁹ NKG2A^{neg} cells, at least at or about 3.0×10⁹ NKG2A^{neg} cells, at least at or about 4.0×10⁹ NKG2A^{neg} cells, at least at or about 5.0×10⁹ NKG2A^{neg} cells, at least at or about 1.0×10¹⁰ NKG2A^{neg} cells, at least at or about 1.5×10¹⁰ NKG2A^{neg} cells, at least at or about 2.0×10¹⁰ NKG2A^{neg} cells, at least at or about 2.5×10¹⁰ NKG2A^{neg} cells or more.

In some of any of the provided methods, the method produces an increased number of NKG2C^{pos}NKG2A^{neg} cells at the end of the culturing compared to at the initiation of the culturing. For example, the increase in NKG2C^{pos}NKG2A^{neg} cells at the end of culturing compared to at the initiation of the culturing can be greater than or greater than about 100-fold, greater than or greater than about 200-fold, greater than or greater than about 300-fold, greater than or greater than about 400-fold, greater than or greater than about 500-fold, greater than or greater than about 600-fold, greater than or greater than about 700-fold or greater than or greater than about 800-fold. In some of any embodiments, the increase is at or about 1000-fold greater. In some of any embodiments, the increase is at or about 2000-fold greater. In some of any embodiments, the increase is at or about 2500-fold greater. In some of any embodiments, the increase is at or about 3000-fold greater. In some of any embodiments, the increase is at or about 5000-fold greater. In some of any embodiments, the increase is at or about 10000-fold greater. In some of any embodiments, the increase is at or about 15000-fold greater. In some of any embodiments, the increase is at or about 20000-fold greater. In some of any embodiments, the increase is at or about 25000-fold greater. In some of any embodiments, the increase is at or about 30000-fold greater. In some of any embodiments, the increase is at or about 35000-fold greater. In some embodiments, the culturing or incubation in accord with any of the provided methods is carried out until a time at which the method achieves expansion of at least at or about 2.50×10⁸ NKG2C^{pos}NKG2A^{neg} cells, at least at or about 3.0×10⁸ NKG2C^{pos}NKG2A^{neg} cells, at least at or about 4.0×10⁸ NKG2C^{pos}NKG2A^{neg} cells, at least at or about 5.0×10⁸ NKG2C^{pos}NKG2A^{neg} cells, at least at or about 6.0×10⁸ NKG2C^{pos}NKG2A^{neg} cells, at least at or about 7.0×10⁸ NKG2C^{pos}NKG2A^{neg} cells, at least at or about 8.0×10⁸ NKG2C^{pos}NKG2A^{neg} cells, at least at or about 9.0×10⁸ NKG2C^{pos}NKG2A^{neg} cells, at least at or about 1.0×10⁹ NKG2C^{pos}NKG2A^{neg} cells, at least at or about 1.5×10⁹ NKG2C^{pos}NKG2A^{neg} cells, at least at or about 2.0×10⁹ NKG2C^{pos}NKG2A^{neg} cells, at least at or about 3.0×10⁹ NKG2C^{pos}NKG2A^{neg} cells, at least at or about 4.0×10⁹ NKG2C^{pos}NKG2A^{neg} cells, at least at or about 5.0×10⁹ NKG2C^{pos}NKG2A^{neg} cells, at least at or about 1.0×10¹⁰ NKG2C^{pos}NKG2A^{neg} cells, at least at or about 1.5×10¹⁰ NKG2C^{pos}NKG2A^{neg} cells, at least at or about 2.0×10¹⁰ NKG2C^{pos}NKG2A^{neg} cells, at least at or about 2.5×10¹⁰ NKG2C^{pos}NKG2A^{neg} cells or more.

In some of any of the provided methods, the method produces an increased number of g-NK cells at the end of the culturing compared to at the initiation of the culturing. For example, the increase in g-NK cells at the end of culturing compared to at the initiation of the culturing can be greater than or greater than about 100-fold, greater than or greater than about 200-fold, greater than or greater than about 300-fold, greater than or greater than about 400-fold, greater than or greater than about 500-fold, greater than or greater than about 600-fold, greater than or greater than about 700-fold or greater than or greater than about 800-fold. In some of any embodiments, the increase is at or about 1000-fold greater. In some of any embodiments, the increase is at or about 2000-fold greater. In some of any embodiments, the increase is at or about 2500-fold greater. In some of any embodiments, the increase is at or about 3000-fold greater. In some of any embodiments, the increase is at or about 5000-fold greater. In some of any embodiments, the increase is at or about 10000-fold greater. In some of any embodiments, the increase is at or about 15000-fold greater. In some of any embodiments, the increase is at or about 20000-fold greater. In some of any embodiments, the increase is at or about 25000-fold greater. In some of any embodiments, the increase is at or about 30000-fold greater. In some of any embodiments, the increase is at or about 35000-fold greater. In some embodiments, the culturing or incubation in accord with any of the provided methods is carried out until a time at which the method achieves expansion of at least at or about 2.50×10⁸ g-NK cells, at least at or about 3.0×10⁸ g-NK cells, at least at or about 4.0×10⁸ g-NK cells, at least at or about 5.0×10⁸ g-NK cells, at least at or about 6.0×10⁸ g-NK cells, at least at or about 7.0×10⁸ g-NK cells, at least at or about 8.0×10⁸ g-NK cells, at least at or about 9.0×10⁸ g-NK cells, at least at or about 1.0×10⁹ g-NK cells, at least at or about 1.5×10⁹ g-NK cells, at least at or about 2.0×10⁹ g-NK cells, at least at or about 3.0×10⁹ g-NK cells, at least at or about 4.0×10⁹ g-NK cells, at least at or about 5.0×10⁹ g-NK cells or more, at least at or about 1.0×10¹⁰ g-NK cells or more, at least at or about 1.5×10¹⁰ g-NK cells or more, at least at or about 2.0×10¹⁰ g-NK cells or more, or at least at or about 2.5×10¹⁰ g-NK cells or more.

In some embodiments, the provided methods result in the preferential expansion of g-NK cells. In some aspects, g-NK cells are identified by the presence, absence or level of surface expression of one or more various marker that distinguishes NK cells from other lymphocytes or immune cells and that distinguishes g-NK cells from conventional NK cells. In embodiments, surface expression can be determined by flow cytometry, for example, by staining with an antibody that specifically bind to the marker and detecting the binding of the antibody to the marker. Similar methods can be carried out to assess expression of intracellular markers, except that such methods typically include methods for fixation and permeabilization before staining to detect intracellular proteins by flow cytometry. In some embodiments, fixation is achieved using formaldehyde (e.g. 0.01%) followed by disruption of membranes using a detergent (e.g. 0.1% to 1% detergent, for example at or about 0.5%), such as Triton, NP-50, Tween 20, Saponin, Digitonin or Leucoperm.

Antibodies and other binding entities can be used to detect expression levels of marker proteins to identify, detect, enrich and/or isolate the g NK cells. Suitable antibodies may include polyclonal, monoclonal, fragments (such as Fab fragments), single chain antibodies and other forms of specific binding molecules.

In some embodiments, a cell (e.g. NK cell subset) is positive (pos) for a particular marker if there is detectable presence on or in the cell of a particular marker, which can be an intracellular marker or a surface marker. In embodiments, surface expression is positive if staining is detectable at a level substantially above the staining detected carrying out the same procedures with an isotype-matched control under otherwise identical conditions and/or at a level substantially similar to, or in some cases higher than, a cell known to be positive for the marker and/or at a level higher than that for a cell known to be negative for the marker.

In some embodiments, a cell (e.g. NK cell subset) is negative (neg) for a particular marker if there is an absence of detectable presence on or in the cell of a particular marker, which can be an intracellular marker or a surface marker. In embodiments, surface expression is negative if staining is not detectable at a level substantially above the staining detected carrying out the same procedures with an isotype-matched control under otherwise identical conditions and/or at a level substantially lower than a cell known to be positive for the marker and/or at a level substantially similar to a cell known to be negative for the marker.

In some embodiments, a cell (e.g. NK cell subset) is low (lo or min) for a particular marker if there is a lower level of detectable presence on or in the cell of a particular marker compared to a cell known to be positive for the marker. In embodiments, surface expression can be determined by flow cytometry, for example, by staining with an antibody that specifically bind to the marker and detecting the binding of the antibody to the marker, wherein expression, either surface or intracellular depending on the method used, is low if staining is at a level lower than a cell known to be positive for the marker.

In some embodiments, g-NK cells are cells having a phenotype of NK cells (e.g. CD45^{pos}, CD3^{neg} and/or CD56^{pos}) and express one or more markers that identify or that are associated with a g-NK cell subset.

In some embodiments, g-NK cells are identified as described in published Patent Appl. No. US2013/0295044 or Zhang et al. (2013) J. Immunol., 190:1402-1406.

In some embodiments, g-NK cells are cells that do not express substantial FcRγ but do express at least one marker for natural killer cells. An amino acid sequence for FcRγ chain (Homo sapiens, also called the High affinity immunoglobulin gamma Fc receptor I) is available in the NCBI database as accession number NP_004097.1 (GI:4758344), and is reproduced below as SEQ ID NO:1.

(SEQ ID NO: 1)
MIPAVVLLLLLLVEQAAALGEPQLCYILDAILFLYGIVLT
LLYCRLKIQVRKAAITSYEK SDGVYTGLSTRNQETYETLKHEKPPQ

In some embodiments, the g-NK cell subset of NK cells can be detected by observing whether FcRγ is expressed by a population of NK cells or a subpopulation of NK cells. In some cases, g-NK cells are identified as cells that do not express FcRγ. FcRγ protein is an intracellular protein. Thus, in some aspects, the presence or absence of FcRγ can be detected after treatment of cells, for example, by fixation and permeabilization, to allow intracellular proteins to be detected. In some embodiments, cells are further assessed for one or more surface markers (CD45, CD3 and/or CD56) prior to the intracellular detection, such as prior to fixation of cells. In some embodiments, g-NK cells are identified, detected, enriched and/or isolated as cells that are CD45^{pos}/CD3^{neg}/CD56^{pos}/FcRγ^{neg}.

In some embodiments, greater than at or about 50% of NK cells in the expanded population are FcRγ^{neg}. In some embodiments, greater than at or about 60% of NK cells in the expanded population are FcRγ^{neg}. In some embodiments, greater than at or about 70% of NK cells in the expanded population are FcRγ^{neg}. In some embodiments, greater than at or about 80% of NK cells in the expanded population are FcRγ^{neg}. In some embodiments, greater than at or about 90% of NK cells in the expanded population are FcRγ^{neg}. In some embodiments, greater than at or about 95% of NK cells in the expanded population are FcRγ^{neg}. For example, the methods herein generally result in a highly pure, e.g. 70-90%, g-NK cell product.

In some embodiments, it may be useful to detect expression of g-NK cells without employing intracellular staining, such as, for example, if cells of the sample are to be subjected to cell sorting or a functional assay. While treatments, e.g. fixation and permeabilization, to permit intracellular staining of FcRγ can be used to confirm the identity of a substantially pure population of cells, in many cases cell-surface markers can be employed that can be detected without injuring the cells when identifying, detecting or isolating g⁻NK cells. Thus, in some embodiments, g-NK cells are identified using a surrogate marker profile that correlates with the lack of FcRγ among a subset of NK cells. In some embodiments, a surrogate marker profile is of particular use when the presence or absence of an intracellular protein, such as FcRγ, is difficult or not possible to assess depending on the particular application of the cells.

It is found herein that certain combinations of cell surface marker correlate with the g-NK cell phenotype, i.e. cells that lack or are deficient in intracellular expression of FcRγ, thereby providing a surrogate marker profile to identify or detect g-NK cells in a manner that does not injure the cells. In some embodiments, a surrogate marker profile for g-NK cells provided herein is based on positive surface expression of one or more markers CD16 (CD16^{pos}), NKG2C (NKG2C^{pos}), or CD57 (CD57^{pos}) and/or based on low or negative surface expression of one or more markers CD7 (CD7^{dim/neg}), CD161 (CD16^{neg}) and/or NKG2A (NKG2A^{neg}). In some embodiments, cells are further assessed for one or more surface markers of NK cells, such as CD45, CD3 and/or CD56. In some embodiments, g-NK cells can be identified, detected, enriched and/or isolated with the surrogate marker profile CD45^{pos}/CD3^{neg}/CD56^{pos}/CD16^{pos}/CD57^{pos}/CD7^{dim/neg}/CD161^{neg}. In some embodiments, g-NK cells are identified, detected, enriched and/or isolated with the surrogate marker profile CD45^{pos}/CD3^{neg}/CD56^{pos}/NKG2A^{neg}/CD161^{neg}. In some embodiments, g-NK cells that are NKG2C^{pos} and/or NKG2A^{neg} are identified, detected, enriched for, and/or isolated.

In some embodiments, greater than at or about 30% of NK cells in the expanded population are positive for NKG2C and/or greater than at or about 50% of NK cells in the expanded population are negative or low for NKG2A. In some embodiments, greater than at or about 35% of NK cells in the expanded population are positive for NKG2C and/or greater than at or about 60% of NK cells in the expanded population are negative or low for NKG2A. In some embodiments, greater than at or about 40% of NK cells in the expanded population are positive for NKG2C and/or greater than at or about 70% of NK cells in the expanded population are negative or low for NKG2A. In some embodiments, greater than at or about 45% of NK cells in the expanded population are positive for NKG2C and/or greater than at or about 80% of NK cells in the expanded population are negative or low for NKG2A. In some embodiments, greater than at or about 50% of NK cells in the expanded population are positive for NKG2C and/or greater than at or about 85% of NK cells in the expanded population are negative or low for NKG2A. In some embodiments, greater than at or about 55% of NK cells in the expanded population are positive for NKG2C and/or greater than at or about 90% of NK cells in the expanded population are negative or low for NKG2A. In some embodiments, greater than at or about 60% of NK cells in the expanded population are positive for NKG2C and/or greater than at or about 95% of NK cells in the expanded population are negative or low for NKG2A.

In some embodiments, greater than at or about 70% of the g-NK cells in the expanded population are positive for perforin, and greater than at or about 70% of the g-NK cells in the expanded population are positive for granzyme B. In some embodiments, greater than at or about 75% of the g-NK cells in the expanded population are positive for perforin, and greater than at or about 75% of the g-NK cells in the expanded population are positive for granzyme B. In some embodiments, greater than at or about 80% of the g-NK cells in the expanded population are positive for perforin, and greater than at or about 80% of the g-NK cells in the expanded population are positive for granzyme B. In some embodiments, greater than at or about 85% of the g-NK cells in the expanded population are positive for perforin, and greater than at or about 85% of the g-NK cells in the expanded population are positive for granzyme B. In some embodiments, greater than at or about 90% of the g-NK cells in the expanded population are positive for perforin, and greater than at or about 90% of the g-NK cells in the expanded population are positive for granzyme B. In some embodiments, greater than at or about 95% of the g-NK cells in the expanded population are positive for perforin, and greater than at or about 95% of the g-NK cells in the expanded population are positive for granzyme B.

Cells expanded by the provided methods can be assessed for any of a number of functional or phenotypic activities, including but not limited to cytotoxic activity, degranulation, ability to produce or secrete cytokines, and expression of one or more intracellular or surface phenotypic markers. Methods to assess such activities are known and are exemplified herein and in working examples.

In some embodiments, antibody-dependent cell cytotoxicity (ADCC) cytotoxic activity against target cells can be used as a measure of functionality. For the ADCC cytotoxicity assays, cells from expansions can be co-cultured with appropriate targets cells in the presence or absence of an antibody specific to a target antigen on the target cells. For example, for anti-myeloma cytotoxicity any of a number of multiple myeloma (MM) target cells can be used (e.g. AM01, KMS11, KMS18, KMS34, LP1 or MM.1S) can be used and the assay performed with an anti-CD38 (e.g. Daratumumab) or anti-CD319 antibody (e.g. Elotuzumab). Cell killing can be determined by any number of methods. For example, cells can be stained with Propidium iodide (PI) and the number of NK-cells, live target cells, and dead target cells can be resolved, such as by flow cytometry.

In some embodiments, greater than at or about 10% of g-NK cells in the expanded population are capable of degranulation against tumor cells. Degranulation can be measured by assessing expression of CD107A. For example, in some embodiments, greater than at or about 20% of g-NK cells in the expanded population are capable of degranulation against tumor cells. In some embodiments, greater than at or about 30% of g-NK cells in the expanded population are capable of degranulation against tumor cells. In some embodiments, greater than at or about 40% of g-NK cells in the expanded population are capable of degranulation against tumor cells. In some embodiments, capacity for degranulation is measured in the absence of an antibody against the tumor cells.

In some embodiments, greater than at or about 10% of g-NK cells in the expanded population are capable of producing an effector cytokine, such as interferon-gamma or TNF-alpha, against tumor cells. In some embodiments, greater than at or about 20% of g-NK cells in the expanded population are capable of producing an effector cytokine, e.g. interferon-gamma or TNF-alpha, against tumor cells. In some embodiments, greater than at or about 30% of g-NK cells in the expanded population are capable of producing an effector cytokine, e.g. interferon-gamma or TNF-alpha, against tumor cells. In some embodiments, greater than at or about 40% of g-NK cells in the expanded population are capable of producing an effector cytokine, e.g. interferon-gamma or TNF-alpha, against tumor cells. In some embodiments, capacity for producing interferon-gamma or TNF-alpha is measured in the absence of an antibody against the tumor cells.

Provided herein are methods for identifying or detecting g-NK cells in a sample containing a population of cells by employing a surrogate marker profile of g-NK cells. In some embodiments, the methods include contacting a sample of cells with a binding molecule, such as an antibody or antigen-binding fragment that is specific for one or more markers CD16, CD57, CD7, CD161, NKG2C, and/or NKG2A. In some embodiments, the methods further include contacting the sample of cells with a binding molecule, such as an antibody or antigen-binding fragment that is specific for CD45, CD3 and/or CD56. In some embodiments of the methods, the one or more binding molecules can be contacted with the sample simultaneously. In some embodiments of the methods, the one or more binding molecules can be contacted with the sample sequentially. In some embodiments, following the contact, the methods can include one or more washing under conditions to retain cells that have bound to the one or more binding molecule and/or to separate away unbound binding molecules from the sample.

In some embodiments, each of the one or more binding molecules, e.g. antibody, may be attached directly or indirectly to a label for detection of cells positive or negative for the marker. For example, the binding molecule, e.g. antibody, may be conjugated, coupled or linked to the label. Labels are well known by one of skill in the art. Labels contemplated herein include, but are not limited to, fluorescent dyes, fluorescent proteins, radioisotopes, chromophores, metal ions, gold particles (e.g., colloidal gold particles), silver particles, particles with strong light scattering properties, magnetic particles (e.g., magnetic bead particles such as Dynabeads® magnetic beads), polypeptides (e.g., FLAG™ tag, human influenza hemagglutinin (HA) tag, etc.), enzymes such as peroxidase (e.g., horseradish peroxidase) or a phosphatase (e.g., alkaline phosphatase), streptavidin, biotin, luminescent compounds (e.g., chemiluminescent substrates), oligonucleotides, members of a specific binding pair (e.g., a ligands and its receptor) and other labels well known in the art that are used for visualizing or detecting a binding molecule, e.g. an antibody, when directly or indirectly attached to said antibody.

A number of well-known methods for assessing expression level of surface markers or proteins may be used, such as detection by affinity-based methods, e.g., immunoaffinity-based methods, e.g., in the context of surface markers, such as by flow cytometry. In some embodiments, the label is a fluorophore and the methods for detection or identification of g-NK cells is by flow cytometry. In some embodiments, different labels are used for each of the different markers by multicolor flow cytometry.

In some embodiments, the methods include contacting a sample with a binding molecule specific to CD45, CD3, CD56, CD57, CD7 and CD161. In some such embodiments, g-NK cells are identified or detected as cells having the g-NK cell surrogate marker profile CD45^{pos}/CD3^{neg}/CD56^{pos}/CD16^{pos}/CD57^{pos}/CD7^{dim/neg}/CD16 1^{neg}.

In some embodiments, the methods include contacting a sample with a binding molecule specific to CD45, CD3, CD56, NKG2A and CD161. In some such embodiments, g-NK cells are identified or detected as cells having the g-NK cell surrogate marker profile CD45^{pos}/CD3^{neg}/CD56^{pos}/NKG2A^{neg}/CD161^{neg}.

In some embodiments, the provided methods also can include isolating or enriching g-NK, such as g-NK cells preferentially expanded in accord with any of the provided methods. In some such embodiments, a substantially pure population of g-NK cells can be obtained, such as a cell population containing greater than or greater than about 90%, 91%, 92%, 93%, 94%, 95%, 96%, 97%, 98%, 99% or more g-NK cells, such as determined using any of the described panel or combinations of markers. Antibodies and other binding molecules can be used to detect the presence or absence of expression levels of marker proteins, for use in isolating or enriching g NK cells. In some embodiments, isolation or enrichment is carried out by fluorescence activated cell sorting (FACs). In examples of such methods, g-NK cells are identified or detected by flow cytometry using the methods as described above for staining cells for multiple cell surface markers and stained cells are carried in a fluidic stream for collection of cells that are positive or negative for markers associated with g-NK cells.

### IV. Kits and Articles of Manufacture

Provided herein are articles of manufacture and kits comprising the provided compositions containing NK cells enriched for particular subsets, such as g-NK cells. In some embodiments, the compositions are produced by any of the provided methods. In some embodiments, the kit comprises any of the provided compositions and instructions for administering the composition as a monotherapy. In some embodiments, provided herein is a kit comprising any of the provided compositions and an additional agent. In some embodiments, the additional agent is an antibody. In some embodiments, the additional agent is a human, humanized, or chimeric antibody. In some of these embodiments, the additional agent is a full length antibody. Exemplary antibodies included any as described.

Kits can optionally include one or more components such as instructions for use, devices and additional reagents (e.g., sterilized water or saline solutions for dilution of the compositions and/or reconstitution of lyophilized protein), and components, such as tubes, containers and syringes for practice of the methods. In some embodiments, the kits can further contain reagents for collection of samples, preparation and processing of samples, and/or reagents for quantitating the amount of one or more surface markers in a sample, such as, but not limited to, detection reagents, such as antibodies, buffers, substrates for enzymatic staining, chromagens or other materials, such as slides, containers, microtiter plates, and optionally, instructions for performing the methods. Those of skill in the art will recognize many other possible containers and plates and reagents that can be used in accord with the provided methods.

In some embodiments, the kits can be provided as articles of manufacture that include packing materials for the packaging of the cells, antibodies or reagents, or compositions thereof, or one or more other components. For example, the kits can contain containers, bottles, tubes, vial and any packaging material suitable for separating or organizing the components of the kit. The one or more containers may be formed from a variety of materials such as glass or plastic. In some embodiments, the one or more containers hold a composition comprising cells or an antibody or other reagents for use in the methods. The article of manufacture or kit herein may comprise the cells, antibodies or reagents in separate containers or in the same container.

In some embodiments, the one or more containers holding the composition may be a single-use vial or a multi-use vial, which, in some cases, may allow for repeat use of the composition. In some embodiments, the article of manufacture or kit may further comprise a second container comprising a suitable diluent. The article of manufacture or kit may further include other materials desirable from a commercial, therapeutic, and user standpoint, including other buffers, diluents, filters, needles, syringes, therapeutic agents and/or package inserts with instructions for use.

In some embodiments, the kit can, optionally, include instructions. Instructions typically include a tangible expression describing the cell composition, reagents and/or antibodies and, optionally, other components included in the kit, and methods for using such. In some embodiments, the instructions indicate methods for using the cell compositions and antibodies for administration to a subject for treating a disease or condition, such as in accord with any of the provided embodiments. In some embodiments, the instructions are provided as a label or a package insert, which is on or associated with the container. In some embodiments, the instructions may indicate directions for reconstitution and/or use of the composition.

### V. Exemplary Embodiments

Among the provided embodiments are:
1. A method of treating multiple myeloma, the method comprising administering a composition of Natural Killer (NK) cells deficient in expression of FcRγ chain (g-NK cells) to a subject having multiple myeloma (MM), wherein the composition of g-NK cells is administered once weekly for a predetermined number of doses.
2. The method of embodiment 1, wherein the method is a monotherapy without combined administration of an exogenous antibody for treating the multiple myeloma.
3. The method of embodiment 1, wherein the method further comprises administering to the subject an antibody that is directed against a multiple myeloma antigen.
4. The method of embodiment 3, wherein the multiple myeloma antigen comprises an antigen selected from the group consisting of CD38, SLAMF7, and BCMA.
5. The method of embodiment 3 or 4, wherein the antibody is a full-length antibody.
6. The method of any one of embodiments 3-5, wherein the antibody is an anti-SLAMF7 antibody.
7. The method of any one of embodiments 3-5, wherein the antibody is an anti-BCMA antibody.
8. The method of any one of embodiments 3-5, wherein the antibody is an anti-CD38 antibody.
9. The method of embodiment 3, wherein the antibody is a bispecific antibody.
10. The method of embodiment 9, wherein the bispecific antibody is directed against CD16 and a second multiple myeloma antigen selected from the group consisting of BCMA, SLAMF7, and CD38.
11. The method of embodiment 9 or 10, wherein the bispecific antibody is directed against CD16 and CD38.
12. The method of any one of embodiments 3-11, wherein the antibody is administered once every four weeks, once every three weeks, once every two weeks, once weekly, or twice weekly.
13. The method of embodiment 8, wherein at least one dose of anti-CD38 antibody has been administered to the subject prior to administration of a dose of the composition of g-NK cells.
14. A method of treating multiple myeloma, the method comprising administering a composition of Natural Killer (NK) cells deficient in expression of FcRγ chain (g-NK cells) to a subject having multiple myeloma (MM), wherein the composition of g-NK cells is administered once weekly for a predetermined number of doses, and wherein the subject has received prior administration of at least one dose of an anti-CD38 antibody.
15. The method of any of embodiments 1-14, wherein the g-NK cell composition is administered as two doses in a 14-day cycle, wherein the 14-day cycle is repeated one to three times.
16. The method of any of embodiments 1-15, wherein the g-NK cell composition is administered as six total doses.
17. The method of any of embodiments 8 and 13-16, wherein the anti-CD38 antibody is daratumumab.
18. The method of any of embodiments 13-17, wherein administration of the at least one dose of the anti-CD38 antibody is initiated within one month prior to administration of the composition of g-NK cells.
19. The method of any of embodiments 13-17, wherein administration of the at least one dose of the anti-CD38 antibody is initiated within three weeks prior to administration of the composition of g-NK cells.
20. The method of any of embodiments 13-17, wherein administration of the at least one dose of the anti-CD38 antibody is initiated within two weeks prior to administration of the composition of g-NK cells.
21. The method of any of embodiments 8 and 13-20, wherein the anti-CD38 antibody is administered intravenously.
22. The method of any of embodiments 8 and 13-21, wherein the anti-CD38 antibody is administered as a once weekly dose, optionally for one or two 28-day cycles.
23. The method of any of embodiments 8 and 13-22, wherein each dose of the anti-CD38 antibody (e.g. daratumumab) is administered in an amount that is from or from about 8 mg/kg to about 32 mg/kg, optionally at or about 16 mg/kg.
24. The method of any of embodiments 8 and 13-20, wherein the anti-CD38 antibody is administered subcutaneously.
25. The method of any of embodiments 8, 13-20, and 24, wherein the anti-CD38 antibody (e.g. daratumumab) is administered in an anti-CD38 antibody composition comprising a hyaluronidase, optionally wherein the anti-CD38 antibody composition comprises daratumumab and recombinant human hyaluronidase PH20 (e.g. hyaluronidase-fihj).
26. The method of embodiment 25, wherein the anti-CD38 antibody composition is administered as a once weekly dose, optionally for one or two 28-day cycles.
27. The method of embodiment 25 or embodiment 26, wherein each dose of the anti-CD38 antibody composition comprises from at or about 1200 mg to about 2400 mg anti-CD38 antibody (e.g. daratumumab) and from at or about 15,000 Units (U) to about 45,000 U hyaluronidase (e.g. hyaluronidase-fihj).
28. The method of any of embodiments 24-28, wherein each dose of the anti-CD38 antibody composition comprises about 1800 mg anti-CD38 antibody (e.g. daratumumab) and about 30,000 U hyaluronidase (e.g. hyaluronidase-fihj).
29. The method of any of embodiments 8 and 13-28, wherein the method comprises administering the anti-CD38 antibody, optionally the anti-CD38 antibody composition, once weekly for 8 total doses and administering the g-NK cell composition once weekly for 6 total doses, wherein one dose or two doses of the anti-CD38 antibody is administered prior to administration of the composition comprising g-NK cells.
30. The method of any of embodiments 1-29, wherein the multiple myeloma is relapsed/refractory multiple myeloma.
31. The method of any of embodiments 1-30, wherein the g-NK cells have low or no expression of CD38, optionally wherein less than 25% of the cells in the g-NK cell composition are positive for surface CD38.
32. The method of any of embodiments 1-31, wherein the cells in the g-NK cell composition are not engineered to reduce or eliminate CD38 expression.
33. The method of any of embodiments 1-32, wherein the g-NK cell composition exhibits minimal anti-CD38-induced fratricide, optionally wherein less than 10% of cells in the g-NK cell composition exhibit anti-CD38 induced fratricide.
34. A method of treating lymphoma, the method comprising administering a composition of Natural Killer (NK) cells deficient in expression of FcRγ chain (g-NK cells) to a subject having lymphoma, wherein the composition of g-NK cells is administered once weekly for a predetermined number of doses.
35. The method of embodiment 34, wherein the method is a monotherapy without combined administration of an exogenous antibody for treating the lymphoma.
36. The method of embodiment 34, wherein the method further comprises administering to the subject an antibody that is directed against a lymphoma antigen.
37. The method of embodiment 36, wherein the lymphoma antigen comprises an antigen selected from the group consisting of CD19, CD20, and CD30.
38. The method of embodiments 36 or 37, wherein the antibody is a full-length antibody.
39. The method of any one of embodiments 36-38, wherein the antibody is an anti-CD19 antibody.
40. The method of any one of embodiments 36-38, wherein the antibody is an anti-CD30 antibody.
41. The method of any one of embodiments 36-38, wherein the antibody is an anti-CD20 antibody.
42. The method of embodiment 36, wherein the antibody is a bispecific antibody.
43. The method of embodiment 42 wherein the bispecific antibody is directed against CD16 and a second antigen selected from the group consisting of CD19, CD20, and CD30.
44. The method of embodiment 43, wherein the bispecific antibody is directed against CD16 and CD20.
45. The method of embodiment 36-45, wherein the antibody is administered once every four weeks, once every three weeks, once every two weeks, once weekly, or twice weekly.
46. The method of embodiment 41, wherein at least one dose of anti-CD20 antibody has been administered to the subject prior to administration of a dose of the composition of g-NK cells.
47. A method of treating lymphoma, the method comprising administering a composition of Natural Killer (NK) cells deficient in expression of FcRγ chain (g-NK cells) to a subject having lymphoma, wherein the composition of g-NK cells is administered once weekly for a predetermined number of doses, and wherein the subject has received prior administration of at least one dose of an anti-CD20 antibody.
48. The method of any of embodiments 34-47, wherein the lymphoma is a Non-Hodgkin's Lymphoma (NHL).
49. The method of any of embodiments 34-48, wherein the g-NK cell composition is administered as two doses in a 14-day cycle, wherein the 14-day cycle is repeated one to three times.
50. The method of any of embodiments 34-49, wherein the g-NK cell composition is administered as six total doses.
51. The method of any of embodiments 41 and 45-50, wherein the anti-CD20 antibody is rituximab.
52. The method of any of embodiments 41 and 45-51, wherein administration of the at least one dose of the anti-CD20 antibody is initiated within one month prior to administration of the composition of g-NK cells.
53. The method of any of embodiments 41 and 45-52, wherein administration of the at least one dose of the anti-CD20 antibody is initiated within three weeks prior to administration of the composition of g-NK cells.
54. The method of any of embodiments 41 and 45-53, wherein administration of the at least one dose of the anti-CD20 antibody is initiated within two weeks prior to administration of the composition of g-NK cells.
55. The method of any of embodiments 41 and 45-54, wherein the anti-CD20 antibody is administered intravenously.
56. The method of any of embodiments 41 and 45-55, wherein the anti-CD20 antibody is administered as a once weekly dose, optionally for 4 or 8 doses.
57. The method of any of embodiments 41 and 45-56, wherein each dose of the anti-CD20 antibody is administered in an amount that is from or from about 250 mg/m² to 500 mg/m², optionally at or about 375 mg/m².
58. The method of any of embodiments 41 and 45-54, wherein the anti-CD20 antibody is administered subcutaneously.
59. The method of any of embodiments 41, 45-54, and 58, wherein the anti-CD20 antibody (e.g. rituximab) is administered in an anti-CD20 antibody composition comprising a hyaluronidase, optionally wherein the anti-CD20 antibody composition comprises rituximab and a human recombinant hyaluronidase PH20.
60. The method of embodiment 59, wherein the anti-CD20 antibody composition is administered as a once weekly dose, optionally for 4 or 8 doses or optionally for 3 or 7 doses following a once weekly dose of the anti-CD20 antibody intravenously.
61. The method of embodiment 59 or embodiment 60, wherein each dose of the anti-CD20 antibody composition comprises from at or about 1200 mg to about 2400 mg anti-CD20 antibody (e.g. rituximab) and from at or about 15,000 Units (U) to about 45,000 U hyaluronidase.
62. The method of any of embodiments 59-61, wherein each dose of the anti-CD20 antibody composition comprises about 1400 mg anti-CD20 antibody (e.g. rituximab) and about 23,400 U hyaluronidase.
63. The method of any of embodiments 59-61, wherein each dose of the anti-CD20 antibody composition comprises about 1600 mg anti-CD20 antibody (e.g. rituximab) and about 26,800 U hyaluronidase.
64. The method of any of embodiments 41 and 45-63, wherein the method comprises administering the anti-CD20 antibody, optionally the anti-CD20 antibody composition, once weekly for 8 total doses and administering the g-NK cell composition once weekly for 6 total doses, wherein one dose or two doses of the anti-CD20 antibody is administered prior to administration of the composition comprising g-NK cells.
65. The method of any of embodiments 1-64, wherein, among cells in the g-NK cell composition, greater than at or about 60% of the cells are g-NK cells, greater than at or about 70% of the cells are g-NK cells, greater than at or about 80% of the cells are g-NK cells, greater than at or about 90% of the cells are g-NK cells, or greater than at or about 95% of the cells are g-NK cells.
66. The method of any of embodiments 1-64, wherein at least at or about 50% of the cells in the g-NK cell composition are FcRγ-deficient (FcRγ^{neg}) NK cells (g-NK), wherein greater than at or about 70% of the g-NK cells are positive for perforin and greater than at or about 70% of the g-NK cells are positive for granzyme B.
67. The method of embodiment 65 or embodiment 66, wherein (i) greater than at or about 80% of the g-NK cells are positive for perforin and greater than at or about 80% of the g-NK cells are positive for granzyme B, (ii) greater than at or about 90% of the g-NK cells are positive for perforin and greater than at or about 90% of the g-NK cells are positive for granzyme B, or (iii) greater than at or about 95% of the g-NK cells are positive for perforin and greater than at or about 95% of the g-NK cells are positive for granzyme B.
68. The method of embodiment 66 or embodiment 67, wherein:
among the cells positive for perforin, the cells express a mean level of perforin as measured by intracellular flow cytometry that is, based on mean fluorescence intensity (MFI), at least at or about two times the mean level of perforin expressed by cells that are FcRγ^{pos}; and/or.
among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγ^{pos}.
69. The method of any of embodiments 1-68, wherein greater than 10% of the cells in the g-NK cell composition are capable of degranulation against tumor target cells, optionally as measured by CD107a expression, optionally wherein the degranulation is measured in the absence of an antibody against the tumor target cells.
70. The method of any of embodiments 1-69, wherein, among the cells in the g-NK cell composition, greater than at or about 15%, greater than at or about 20%, greater than at or about 30%, greater than at or about 40% or greater than at or about 50% exhibit degranulation, optionally as measured by CD107a expression, in the presence of cells expressing a target antigen (target cells) and an antibody directed against the target antigen (anti-target antibody).
71. The method of any of embodiments 1-70, wherein greater than 10% of the cells in the g-NK cell composition are capable of producing interferon-gamma or TNF-alpha against tumor target cells, optionally wherein the interferon-gamma or TNF-alpha is measured in the absence of an antibody against the tumor target cells.
72. The method of any of embodiments 1-71, wherein, among the cells in the g-NK cell composition, greater than at or about 15%, greater than at or about 20%, greater than at or about 30%, greater than at or about 40% or greater than at or about 50% produce an effector cytokine in the presence of cells expressing a target antigen (target cells) and an antibody directed against the target antigen (anti-target antibody).
73. The method of embodiment 72, wherein the effector cytokine is IFN-gamma or TNF-alpha.
74. The method of embodiment 72 or embodiment 73, wherein the effector cytokine is IFN-gamma and TNF-alpha.
75. The method of any of embodiments 1-74, wherein the g-NK cell composition has been produced by ex vivo expansion of CD3−/CD57+ cells cultured with irradiated HLA-E+ feeder cells, wherein the CD3−/CD57+ cells are enriched from a biological sample from a donor subject.
76. The method of embodiment 75, wherein the donor subject is CMV-seropositive.
77. The method of embodiment 75 or embodiment 76, wherein the donor subject has the CD16 158V/V NK cell genotype or the CD16 158V/F NK cell genotype, optionally wherein the biological sample is from a human subject selected for the CD16 158V/V NK cell genotype or the CD16 158V/F NK cell genotype.
78. The method of any of embodiments 75-77, wherein at least at or about 20% of natural killer (NK) cells in a peripheral blood sample from the donor subject are positive for NKG2C (NKG2Cpos) and at least 70% of NK cells in the peripheral blood sample are negative or low for NKG2A (NKG2Aneg).
79. The method of any of embodiments 75-77, wherein the irradiated feeder cells are deficient in HLA class I and HLA class II.
80. The method of any of embodiments 78-79, wherein the irradiated feeder cells are 221.AEH cells.
81. The method of any of embodiments 79-80, wherein the culturing is performed in the presence of two or more recombinant cytokines, wherein at least one recombinant cytokine is interleukin (IL)-2 and at least one recombinant cytokine is IL-21.
82. The method of embodiment 81, wherein the recombinant cytokines are IL-21 and IL-2.
83. The method of embodiment 81, wherein the recombinant cytokines are IL-21, IL-2, and IL-15.
84. The method of any of embodiments 1-83, wherein the g-NK cells in the composition are from a single donor subject that have been expanded from the same biological sample.
85. The method of any of embodiments 1-84, wherein the g-NK cell composition is formulated in a serum-free cryopreservation medium comprising a cryoprotectant, optionally wherein the cyroprotectant is DMSO and the cryopreservation medium is 5% to 10% DMSO (v/v).
86. The method of any of embodiments 1-85, wherein the g-NK cells are not engineered with an antigen receptor, optionally wherein the antigen receptor is a chimeric antigen receptor.
87. The method of any of embodiments 1-86, wherein the g-NK cells are not engineered with a secretable cytokine, optionally a cytokine receptor fusion protein, such as IL-15 receptor fusion (IL-15RF)
88. The method of any of embodiments 1-87, wherein the method does not include exogenous cytokine administration to the subject to support NK cell survival or expansion, wherein the exogenous cytokine is one or more of IL-2, IL-7, IL-15 or IL-21.
89. The method of any of embodiments 1-88, each dose of g-NK cells is from at or about from at or about 1×10⁸ cells to at or about 50×10⁹ cells of the g-NK cell composition.
90. The method of any of embodiments 1-89, wherein each dose of g-NK cells is or is about 5×10⁸ cells of the g-NK cell composition.
91. The method of any of embodiments 1-89, wherein each dose of g-NK cells is or is about 5×10⁹ cells of the g-NK cell composition.
92. The method of any of embodiments 1-89, wherein each dose of g-NK cells is or is about 10×10⁹ cells of the g-NK cell composition.
93. The method of any of embodiments 1-92, wherein prior to the administration of the dose of g-NK cells, the subject has received a lymphodepleting therapy.
94. The method of embodiment 93, wherein the lymphodepleting therapy comprises fludarabine and/or cyclophosphamide.
95. The method of embodiment 93 or embodiment 94, wherein the lymphodepleting comprises the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.
96. The method of embodiment 94 or embodiment 95, wherein the lymphodepleting therapy comprises fludarabine and cyclophosphamide.
97. The method of any of embodiments 1-96, wherein the lymphodepleting therapy comprises the administration of fludarabine at or about 30 mg/m² body surface area of the subject, daily, and cyclophosphamide at or about 300 mg/m² body surface area of the subject, daily, each for 2-4 days, optionally 3 days.
98. The method of any of embodiments 1-97, wherein administration of a dose of g-NK cells is initiated within two weeks or at or about two weeks after initiation of the lymphodepleting therapy.
99. The method of any of embodiments 1-97, wherein administration of a dose of g-NK cells is initiated within 7 days or at or about 7 days after initiation of the lymphodepleting therapy.
100. The method of any one of embodiments 1-99, wherein the individual is a human.
101. The method of any one of embodiments 1-100, wherein the NK cells in the composition are allogenic to the individual.
102. The method of any one of embodiments 1-101, further comprising administering exogenous cytokine support to facilitate expansion or persistence of the g-NK cells in vivo in the subject, optionally wherein the exogenous cytokine is or comprises IL-15.

### VI. Examples

The following examples are included for illustrative purposes only and are not intended to limit the scope of the invention.

### Example 1: Expansion of g-NK Cells in the Presence of Different Cytokines

Fifty mL of fresh whole blood from a CMV-seropositive donor (NKG2C^{pos} and NKG2A^{neg} NK-cell percentages of 56.24% and 11.68%, respectively) was collected into ACD vacutainer tubes and diluted 1:1 with PBS. PBMCs were isolated by Histopaque® density centrifugation as per manufacturer's instructions. After harvesting the PBMC-containing buffy coat, the PBMCs were washed with PBS and counted. Following the cell count, a magnetic bead separation was conducted to increase the frequency of g-NK cells. The magnetic bead separation was a CD3 depletion followed by CD57 enrichment in order to isolate CD57^{pos} NK cells.

The transgenic lymphoma cell line 221.AEH (Lee et al. (1998) Journal of Immunology, 160:4951-4960) and the transgenic leukemia cell line K562-mb15-41BBL (Fujisaki et al. (2009) Cancer Research, 69(9): 4010-4017) were prepared as feeder cells for the NK cell expansion. Feeder cells were taken from fresh culture (i.e., not cryopreserved stock) and were irradiated prior to use. 221.AEH and K562-mb15-41BBL cells were expanded with a seeding density of 5×10⁵ cells per mL and a subculture density of 2×10⁵ cells per mL. The media used to grow the 221.AEH feeder cells was RPMI-1640 with 10% FBS and 200 μg/mL of Hygromycin B. The media used to grow the K562-mb15-41BBL feeder cells was RPMI-1640 with 10% FBS.

The non-cryopreserved NK cells were expanded under four different conditions: at a 2:1 AEH to NK cell ratio with 500 IU/mL IL-2; at a 2:1 K562-mb15-41BBL to NK cell ratio with 500 IU/mL IL-2; at a 1:1:1 AEH to K562-mb15-41BBL to NK cell ratio with 500 IU/mL IL-2; and at a 2:1 AEH to NK cell ratio with 500 IU/mL IL-2, 10 ng/ml IL-15, and 25 ng/ml IL-21. All expansions were carried out in CellGenix GMP SCGM media supplemented with 5% human AB Serum and with the respective cytokines. The co-cultured cells were cultivated for 21 days at 37° C. and 5% CO2. Cells were counted every time the media was changed or replenished (day 5, 7, 10, 13, 16, 19, and 21), and the percentage of g-NK was assessed by flow cytometry at day 0, day 13, and day 21.

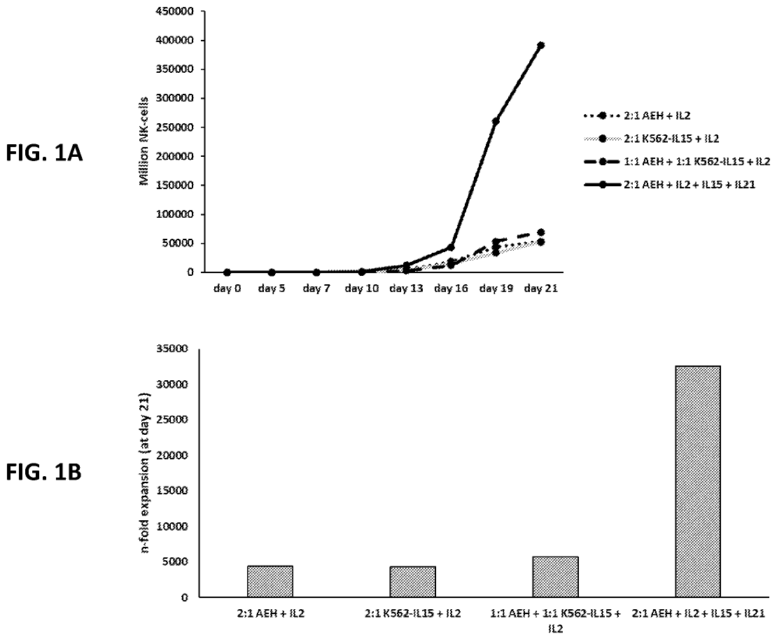

As shown in FIG. 1A-1B, the addition of IL-21 to the expansion media led to a marked increase in g-NK cell expansion. Total g-NK cell count was highest for g-NK cells expanded in the presence of IL-21 (FIG. 1A). Fold-expansion of g-NK cells by day 21 was also highest for g-NK cells expanded in the presence of IL-21 (FIG. 1B).

Together, these results show that the presence of IL-21 improves g-NK cell expansion.

### Example 2: Cell Effector Function of g-NK Cells Expanded in the Presence of Different Cytokines

In this study, NK cell effector function was measured in g-NK cells expanded in the presence of different feeder cells and cytokines, including in the presence of IL-21, as described in Example 1. Assays were performed as described below using target cell lines LP1 and MM.1S at a 0.5:1 NK to MM cell ratio and with antibodies daratumumab and elotuzumab.

### A. Cell Mediated Cytotoxicity

Upon thawing of expanded NK cells, 10⁴ NK cells were co-cultured with MM target cells at a 1:1 NK cell to MM cell ratio and in the presence of one μg/mL daratumumab (anti-CD38) or one μg/mL elotuzumab (anti-CD319). After a four-hour incubation at 37° C. in a CO₂ incubator, the cells were washed and stained with anti-CD3 and CD56 antibodies to quantify the number of NK cells. After a final wash, propidium iodide (PI) was added, and the number of NK cells, live target cells, and dead target cells were resolved using 4-color flow cytometry (Bigley et al. (2016), Clin. Exp. Immunol., 185:239-251).

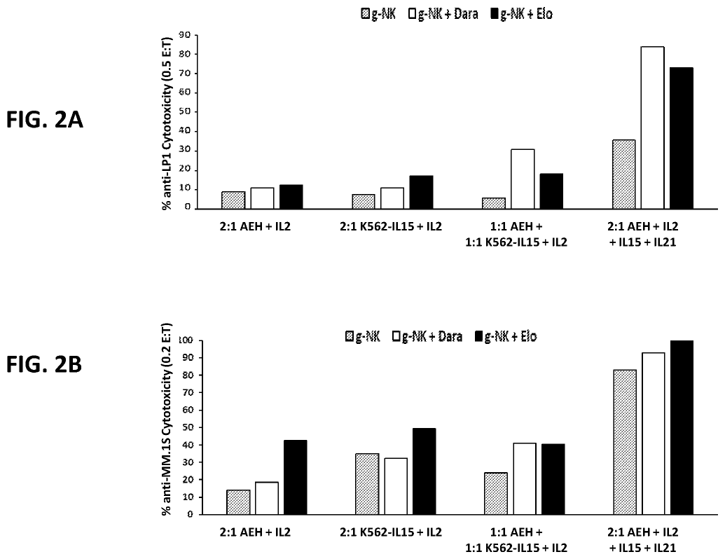

As shown in FIG. 2A-2B, g-NK cells expanded for 21 days in the presence of IL-21 had greater cell-mediated cytotoxicity against the CD38^{high} MM cell line LP1 (FIG. 2A) and the SLAMF7^{high} MM cell line MM.1S (FIG. 2B) than did g-NK cells expanded without IL-21. Greater cell-mediated cytotoxicity for IL-21 expanded g-NK cells was observed in the absence of antibody as well as in the presence of either daratumumab or elotuzumab.

Together, these results show that g-NK cells expanded in the presence of IL-21 have enhanced cell-mediated cytotoxicity against tumor cells compared to g-NK cells expanded without IL-21.

### B. Degranulation

Upon thawing of expanded NK cells, 2.0×10⁵ NK cells were co-cultured MM target cells at a 1:1 NK cell to MM cell ratio and in the presence of one μg/mL daratumumab or one μg/mL elotuzumab. For the degranulation assay, two μL of VioGreen-conjugated anti-CD107a was added to the co-culture for a one-hour incubation at 37° C. in a CO₂ incubator, after which four μL of BD GolgiStop containing monensin was added. For cytokine expression assays, six μL of BD GolgiStop containing brefeldin A was added instead. The cells were then incubated for an additional five hours at 37° C. in a CO2 incubator. Following incubation, the cells were harvested, washed, and stained with 0.5 μL of anti-CD45 antibody, 0.5 μL of anti-CD3 antibody, and one μL of anti-CD56 antibody (all antibodies purchased from Miltenyi Biotec). The cells were then fixed and permeabilized using the Inside Stain Kit from Miltenyi Biotec as per the manufacturer's instructions. The cells were then stained with one μL of anti-FcRγ, two μL of anti-perforin, two μL of anti-granzyme B, two μL of Interferon-gamma, and two μL of TNF-alpha antibodies, as described in Table E1. After a final wash, the cells were resolved using eight-color flow cytometry.

TABLE E1
Antibody Panel for Functional Assays.

| | | | | | | | | |
|---|---|---|---|---|---|---|---|---|
| Tubes | V1 | V2 | B1 | B2 | B3 | B4 | R1 | R2 |

Degranulation (release of cytotoxic granules)

| | | | | | | | |
|---|---|---|---|---|---|---|---|
| 1 | CD45 | CD107a | FcRγ | Perforin | CD3 | Granz B | CD56 |

Cytokine expression

| | | | | | | | |
|---|---|---|---|---|---|---|---|
| 2 | CD45 | CD107a | FcRγ | IFN-γ | CD3 | TNF-α | CD56 |

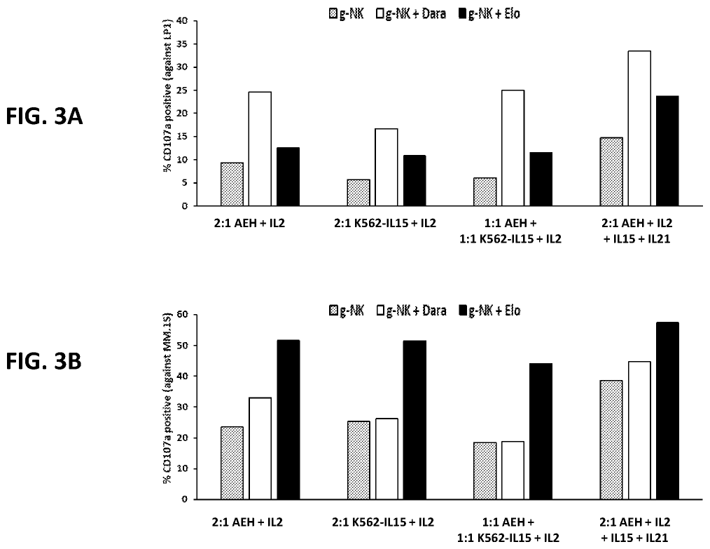

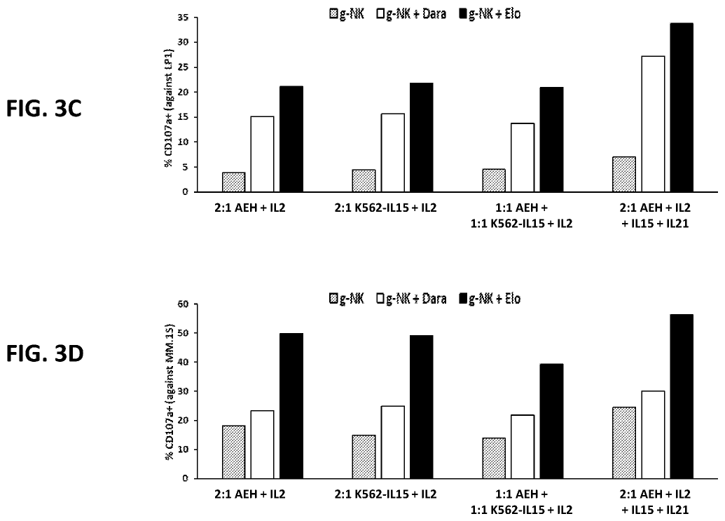

As shown in FIG. 3A-3D, after both 13 days (FIGS. 3A-3B) and 21 days (FIG. 3C-3D) of expansion, g-NK cells expanded in the presence of IL-21 degranulated more against the CD38^{high} MM cell line LP1 (FIG. 3A and FIG. 3C) and the SLAMF7^{high} MM cell line MM.1S (FIG. 3B and FIG. 3D) than did g-NK cells expanded without IL-21. Greater degranulation for IL-21 expanded g-NK cells was observed in the absence of antibody as well as in the presence of either daratumumab or elotuzumab.

Together, these results show that g-NK cells expanded in the presence of IL-21 have enhanced degranulation against tumor cells compared to g-NK cells expanded without IL-21.

### C. Perforin and Granzyme B Expression

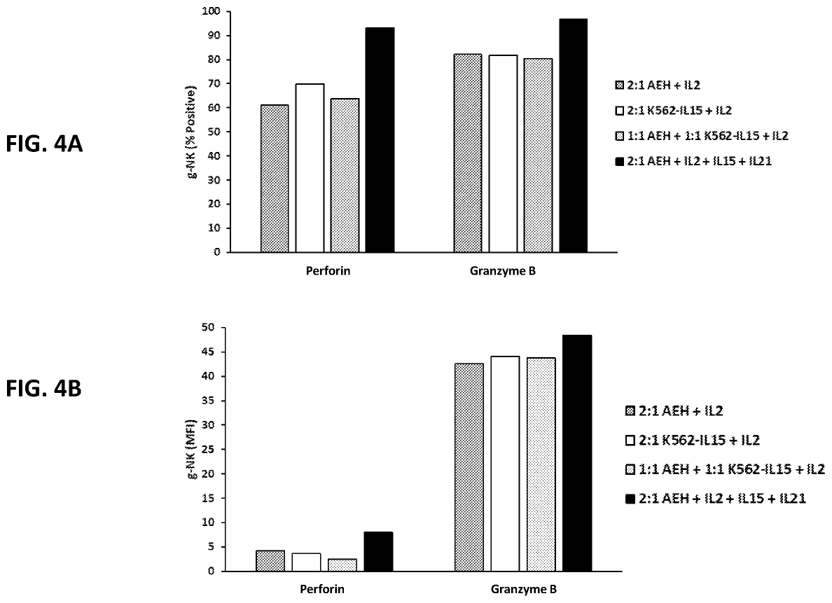

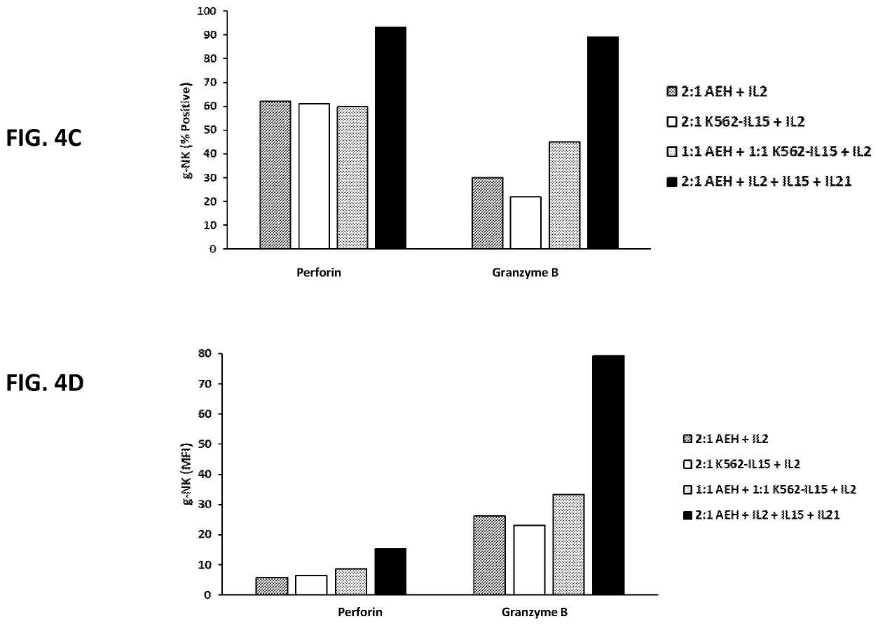

As shown in FIG. 4A-4D, after both 13 days (FIGS. 4A-4B) and 21 days (FIG. 4C-4D) of expansion, g-NK cells expanded in the presence of IL-21 expressed more of the cytolytic protein perforin than did g-NK cells expanded without IL-21, as measured by both the percentage of perforin positive cells (FIG. 4A and FIG. 4C) and the total perforin expression (MFI) (FIG. 4B and FIG. 4D). In addition, after both 13 days and 21 days of expansion, g-NK cells expanded in the presence of IL-21 expressed more of the pro-apoptotic protein granzyme B than did g-NK cells expanded without IL-21, as measured by both the percentage of granzyme B positive cells (FIG. 4A and FIG. 4C) and the total granzyme B expression (MFI) (FIG. 4B and FIG. 4D).

Together, these results show that g-NK cells expanded in the presence of IL-21 have enhanced expression of perforin and granzyme B compared to g-NK cells expanded without IL-21.

### D. Interferon-γ Expression

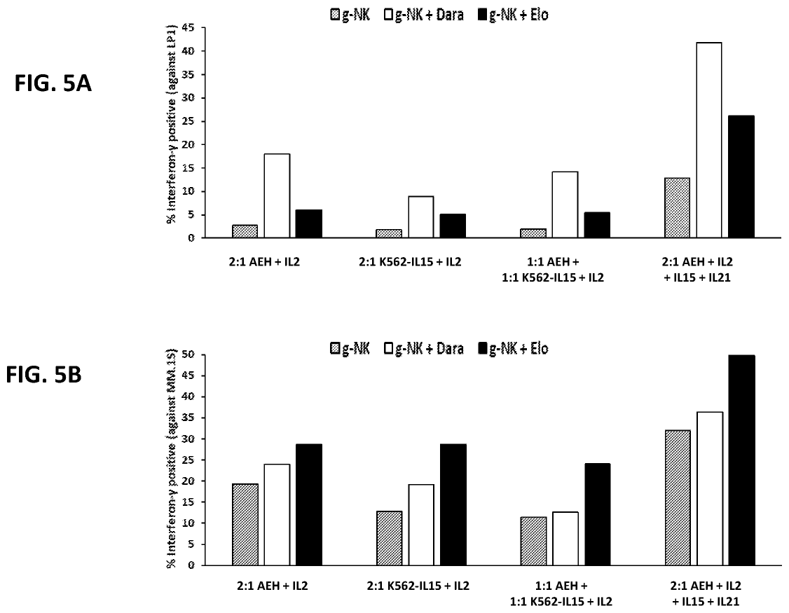

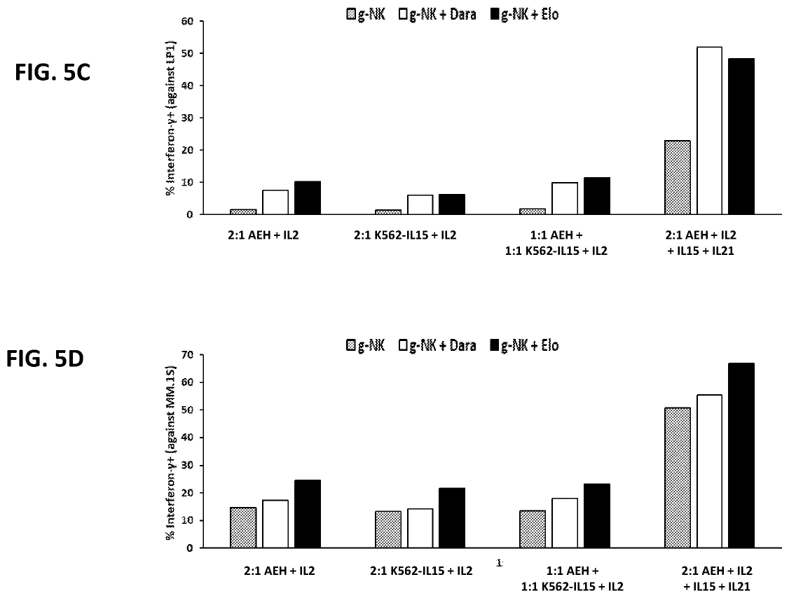

As shown in FIG. 5A-5D, after both 13 days (FIGS. 5A-5B) and 21 days (FIG. 5C-5D) of expansion, g-NK cells expanded in the presence of IL-21 expressed more Interferon-γ against the CD38^{high} MM cell line LP1 (FIG. 5A and FIG. 5C) and the SLAMF7^{high} MM cell line MM.1S (FIG. 5B and FIG. 5D) than did g-NK cells expanded without IL-21. Greater Interferon-γ expression for IL-21 expanded g-NK cells was observed in the absence of antibody as well as in the presence of either daratumumab or elotuzumab.

Together, these results show that g-NK cells expanded in the presence of IL-21 have enhanced Interferon-γ expression against tumor cells compared to g-NK cells expanded without IL-21.

### E. TNF-α Expression

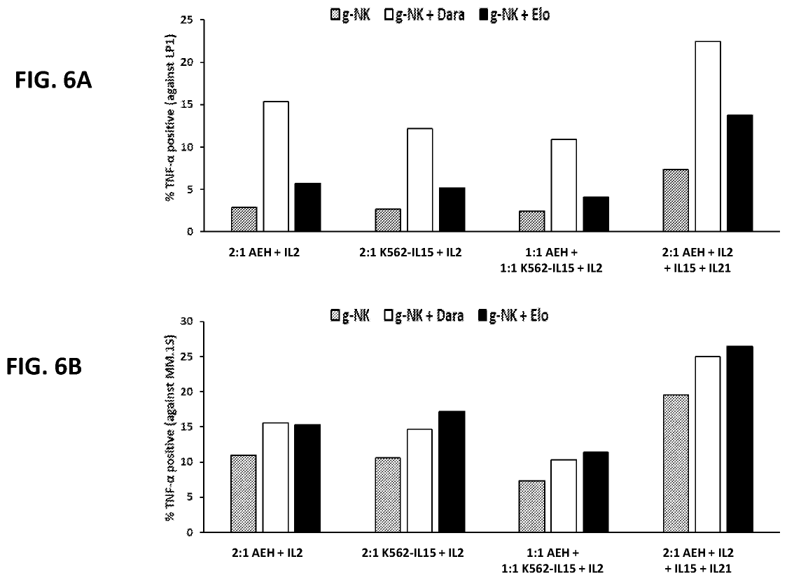

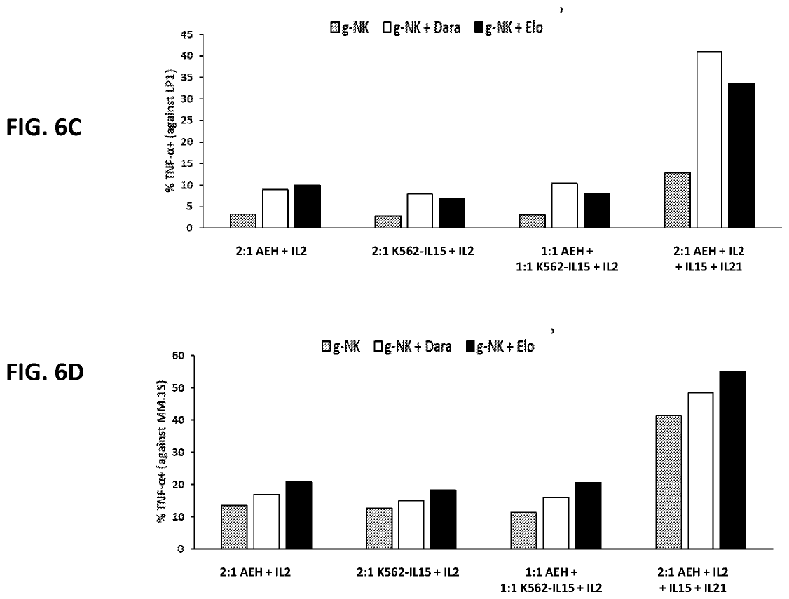

As shown in FIG. 6A-6D, after both 13 days (FIGS. 6A-6B) and 21 days (FIG. 6C-6D) of expansion, g-NK cells expanded in the presence of IL-21 expressed more TNF-α against the CD38^{high} MM cell line LP1 (FIG. 6A and FIG. 6C) and the SLAMF7^{high} MM cell line MM.1S (FIG. 6B and FIG. 6D) than did g-NK cells expanded without IL-21. Greater TNF-α expression for IL-21 expanded g-NK cells was observed in the absence of antibody as well as in the presence of either daratumumab or elotuzumab.

Together, these results show that g-NK cells expanded in the presence of IL-21 have enhanced TNF-α expression against tumor cells compared to g-NK cells expanded without IL-21.

### Example 3: Expansion of g-NK Cells in the Presence of Additional Cytokines

In another study, the expansion rates of NK cells expanded in the presence of various combinations of cytokine mixtures and concentrations were compared. NK cells were harvested from the same donor as in Example 1 and as described above. NK cells were seeded at both a density and a subculture density of 2×10⁵ cells per mL, and they were co-cultured with irradiated 221.AEH feeder cells at a 2:1 221.AEH to NK cell ratio. For the NK cell expansions, cytokines were added at the following concentrations: IL-2 at 100 IU/mL (low IL-2) or 500 IU/mL (IL-2); IL-15 at 10 ng/ml; IL-21 at 25 ng/ml; IL-12 at 10 ng/ml; IL-18 at 10 ng/ML; and/or IL-27 at 10 ng/mL. All expansions were carried out in CellGenix GMP SCGM media supplemented with 5% human AB Serum and with the respective cytokines.

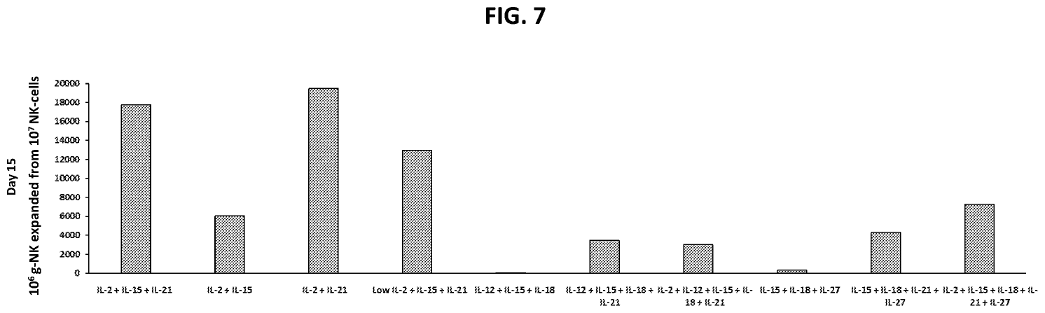

As shown in FIG. 7, NK cells expanded in the presence of IL-21 had a higher g-NK cell expansion rate than did NK cells expanded in the presence of IL-2 and IL-15; IL-12, IL-15, and IL-18; and IL-15, IL-18, and IL-27 by themselves. The combination of cytokines leading to the highest g-NK cell expansion rate was IL-2 and IL-21, either in the presence or absence of IL-15.

Together, these results show that the presence of IL-21 improves g-NK cell expansion rate more so than does other cytokine mixtures.

### Example 4: Cell Effector Function of g-NK Cells Expanded in the Presence of Additional Cytokines

NK cell effector function was measured in g-NK cells expanded for 15 days in the presence of cytokines, including in the presence of IL-21, as described in Example 3. Assays were performed as described in Example 2 using target cell lines LP1 and MM.1S at a 0.5:1 NK to MM cell ratio and with antibodies daratumumab and elotuzumab.

### A. Cell Mediated Cytotoxicity

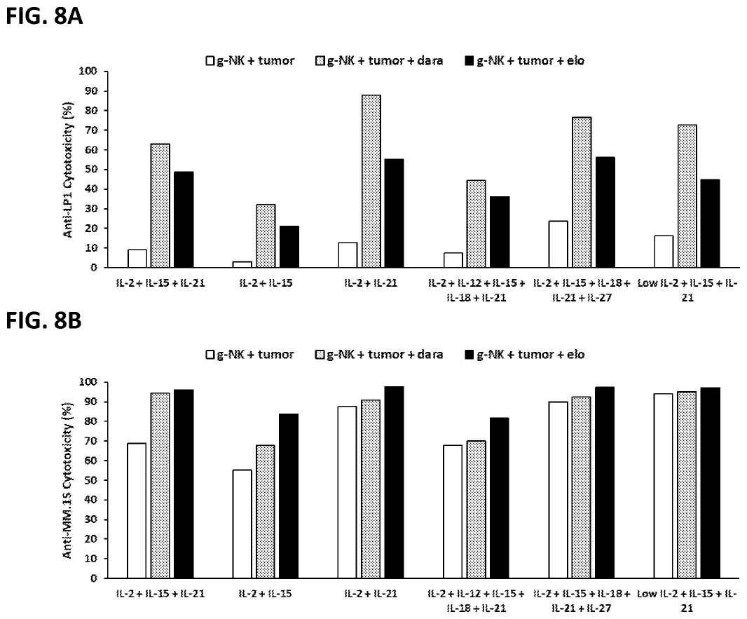

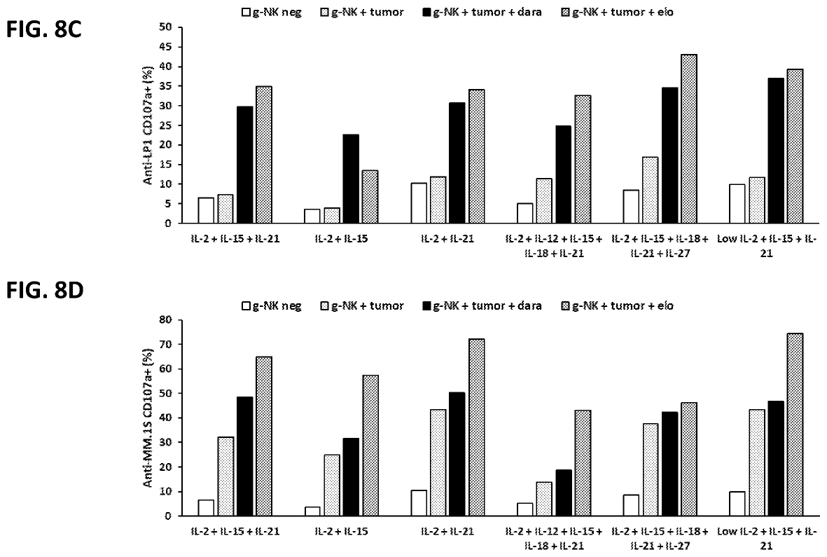

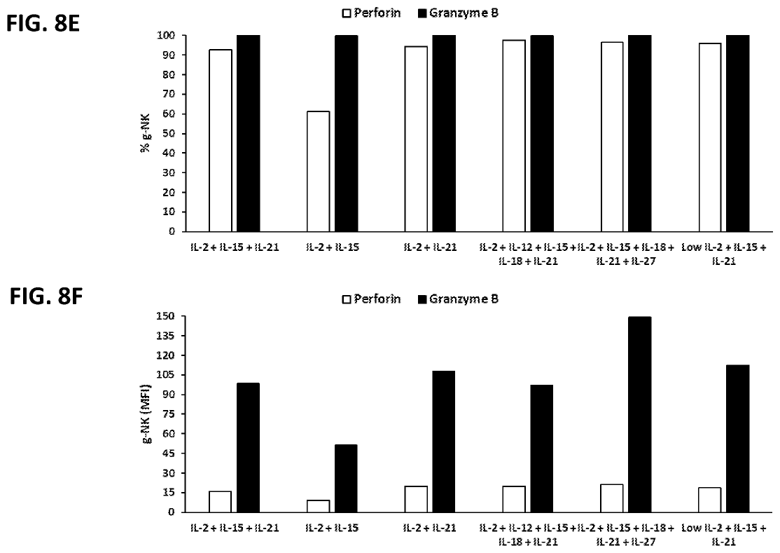

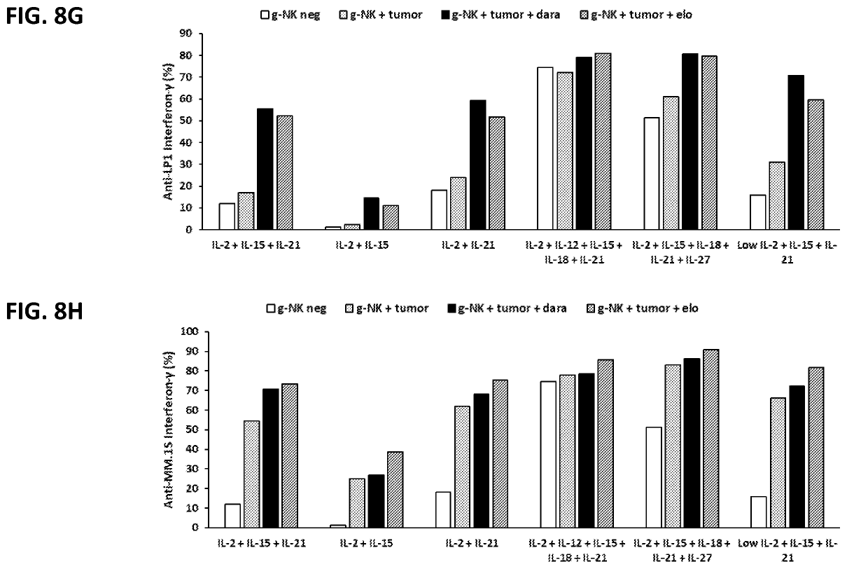

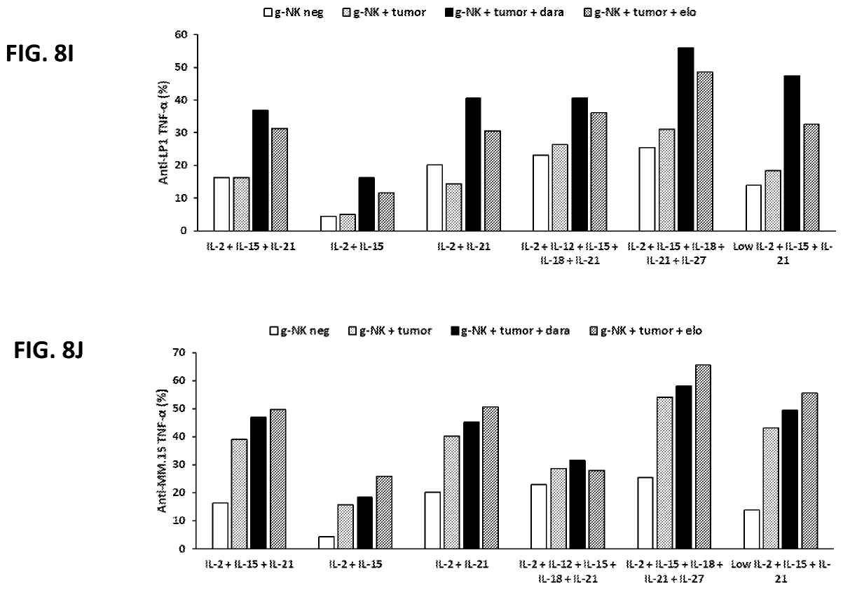

As shown in FIG. 8A and FIG. 8B, g-NK cells expanded in the presence of IL-2, IL-15, and IL-21 had greater cell-mediated cytotoxicity against the CD38^{high} MM cell line LP1 (FIG. 8A) and the SLAMF7^{high} MM cell line MM.1S (FIG. 8B) than did g-NK cells expanded in the presence of IL-2 and IL-15. Greater cell-mediated cytotoxicity for g-NK cells expanded in the presence of IL-2, IL-15, and IL-21 was observed in the absence of antibody as well as in the presence of either daratumumab or elotuzumab.

Together, these results show that g-NK cells expanded in the presence of IL-2, IL-15, and IL-21 have enhanced cell-mediated cytotoxicity against tumor cells compared to g-NK cells expanded in the presence of IL-2 and IL-15.

### B. Degranulation

As shown in FIG. 8C and FIG. 8D, g-NK cells expanded in the presence of IL-2, IL-15, and IL-21 degranulated more against the CD38^{high} MM cell line LP1 (FIG. 8C) and the SLAMF7^{high} MM cell line MM.1S (FIG. 8D) than did g-NK cells expanded in the presence of IL-2 and IL-15. Greater degranulation for g-NK cells expanded in the presence of IL-2, IL-15, and IL-21 was observed under all conditions, including in the absence of antibody.

Together, these results show that g-NK cells expanded in the presence of IL-2, IL-15, and IL-21 have enhanced degranulation against tumor cells compared to g-NK cells expanded in the presence of IL-2 and IL-15.

### C. Perforin and Granzyme B Expression

As shown in FIG. 8E and FIG. 8F, g-NK cells expanded in the presence of IL-2, IL-15, and IL-21 expressed more of the cytolytic protein perforin than did g-NK cells expanded in the presence of IL-2 and IL-15, as measured by both the percentage of perforin positive cells (FIG. 8E) and the total perforin expression (MFI) (FIG. 8F). In addition, g-NK cells expanded in the presence of IL-2, IL-15, and IL-21 expressed more of the pro-apoptotic protein granzyme B than did g-NK cells expanded in the presence of IL-2 and IL-15, as measured by both the percentage of granzyme B positive cells (FIG. 8E) and the total granzyme B expression (MFI) (FIG. 8F). Addition of IL-2, IL-15, IL-18, IL-21, and IL-27 to expansion media enhanced granzyme B expression by g-NK cells.

Together, these results show that g-NK cells expanded in the presence of IL-2, IL-15, and IL-21 have enhanced expression of perforin and granzyme B compared to g-NK cells expanded in the presence of IL-2 and IL-15.

### D. Interferon-γ Expression

As shown in FIG. 8G-8H, g-NK cells expanded in the presence of IL-2, IL-15, and IL-21 expressed more Interferon-γ against the CD38^{high} MM cell line LP1 (FIG. 8G) and the SLAMF7^{high} MM cell line MM.1S (FIG. 8H) than did g-NK cells expanded in the presence of IL-2 and IL-15. Greater Interferon-γ expression for g-NK cells expanded in the presence of IL-2, IL-15, and IL-21 was observed under all conditions, including in the absence of antibody. Addition of IL-2, IL-12, IL-15, IL-18, and IL-21 to expansion media enhanced interferon-y expression by g-NK cells under all conditions, including in the absence of antibody. Addition of IL-2, IL-15, IL-18, IL-21, and IL-27 to expansion media enhanced interferon-y expression by g-NK cells under all conditions, including in the absence of antibody.

Together, these results show that g-NK cells expanded in the presence of IL-2, IL-15, and IL-21 have enhanced Interferon-γ expression against tumor cells compared to g-NK cells expanded in the presence of IL-2 and IL-15.

### E. TNF-α Expression

As shown in FIG. 8I-8J, g-NK cells expanded in the presence of IL-2, IL-15, and IL-21 expressed more TNF-α against the CD38^{high} MM cell line LP1 (FIG. 8I) and the SLAMF7^{high} MM cell line MM.1S (FIG. 8J) than did g-NK cells expanded in the presence of IL-2 and IL-15. Greater TNF-α expression for g-NK cells expanded in the presence of IL-2, IL-15, and IL-21 was observed under all conditions, including in the absence of antibody. Addition of IL-2, IL-15, IL-18, IL-21, and IL-27 to expansion media enhanced antibody-induced TNF-α expression by g-NK cells under all conditions, including in the absence of antibody.

Together, these results show that g-NK cells expanded in the presence of IL-2, IL-15, and IL-21 have enhanced TNF-α expression against tumor cells compared to g-NK cells expanded in the presence of IL-2 and IL-15.

### Example 5: Expansion and Cell Effector Function of g-NK Cells Expanded in the Presence of IL-21

In this study, the expansion rate and NK cell effector function of NK cells expanded in the presence of IL-21 were compared to that of NK cells expanded in the absence of IL-21. Human peripheral blood mononuclear cells (PBMC) were isolated by Histopaque® density centrifugation from whole blood from a CMV-positive human donor, or for comparison a CMV-seronegative donor, as per manufacturer's instructions. Donors were CMV-seropositive (n=8) and CMV seronegative (n=6) (Age 37.8±10.6 yrs; 8 males and 6 females).

PBMCs were harvested from buffy coat, washed, and assessed by flow cytometry for viable CD45^{pos} cells. NK cells were enriched by immunoaffinity-based magnetic bead separation using Miltenyi MACS™ Microbeads either by depletion of CD3^{pos} cells to remove T cells (CD3 depletion, CD3^{neg}) or by CD3 depletion followed by positive selection for CD57 to enrich CD57^{pos} NK cells (CD3^{neg}CD57^{pos}). The latter method of initially enriching for CD3^{neg}/CD57^{pos} cells prior to expansion was used in subsequent experiments for expanding g-NK cells. As a further comparison, NK cells were enriched by CD3 depletion followed by positive selection for CD16 (enrich CD16^{pos} NK cells and monocytes (CD3negCD57pos). NK cells were seeded at a density of 2×10⁵ cells per mL and a subculture density of 2×10⁵ cells per mL. The NK cells were co-cultured with gamma irradiated (100 Gy) 221.AEH feeder cells at a 2:1 221.AEH to NK cell ratio and expanded in the presence of IL-2 (500 IU/mL), IL-15 (10 ng/mL), and IL-21 (25 ng/mL); or IL-2 alone (500 IU/mL). A ratio of 1:1 irradiated 221.AEH feeder cells to NK cells was used if the PBMCs had been cryopreserved prior to enrichment of NK cells, as further described in Example 6. All expansions were carried out in CellGenix GMP SCGM media supplemented with 5% human AB Serum and with the respective cytokines. NK cells were expanded for 2 weeks and media was changed every 2-5 days. Expanded NK-cells were cryopreserved using 90% FBS and 10% DMSO for later use in functional assays.

Expansion and cell effector function were assessed after 14 days of expansion. Assays were performed as described in Example 2 using target cell lines LP1 and MM.1S at a 0.5:1 NK to MM cell ratio and with antibodies daratumumab and elotuzumab.

In some studies described in subsequent examples, phenotypic and functional activities of g-NK cells were compared to cNK cells. Due to insufficient yield of cNK cells from CMV-seronegative donors and preferential expansion of g-NK cells from CMV-seropositive donors using the above described method (results described in section A below), an alternative method was used to expand cNK cells for in vitro functional and in vivo studies. This expansion method used K652-mbIL15-41BBL feeder cells and 500 IU/mL IL-2 to expand cNK cells 180±89 fold (n=5 CMV^{neg}) over 2 weeks (Fujisaki et al., 2009 Cancer Res., 68(9):4010-4017). The proportion of g-NK cells in the 5 CMVneg donors (Age 38.9±9.8 yrs; 3 males and 2 females) was 1.5±0.5% before and 1.6±0.4% after expansion.

A. Expansion Rate of g-NK Cells

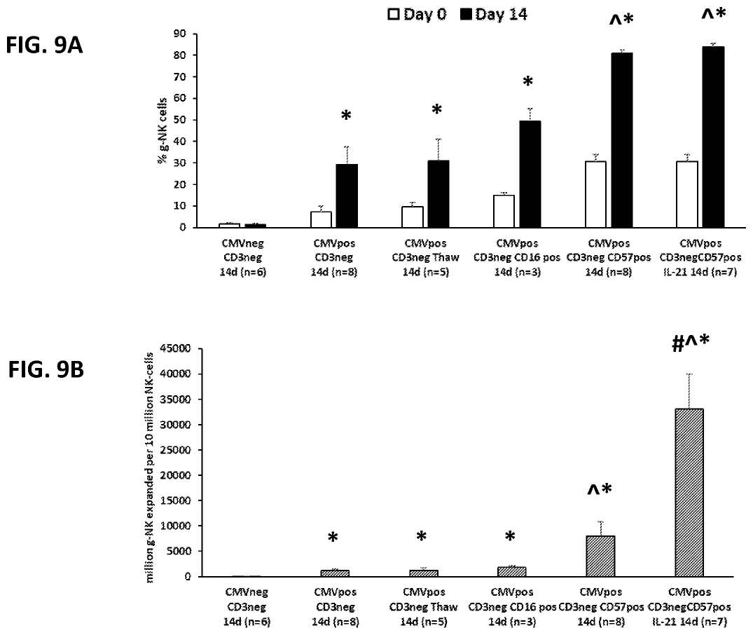

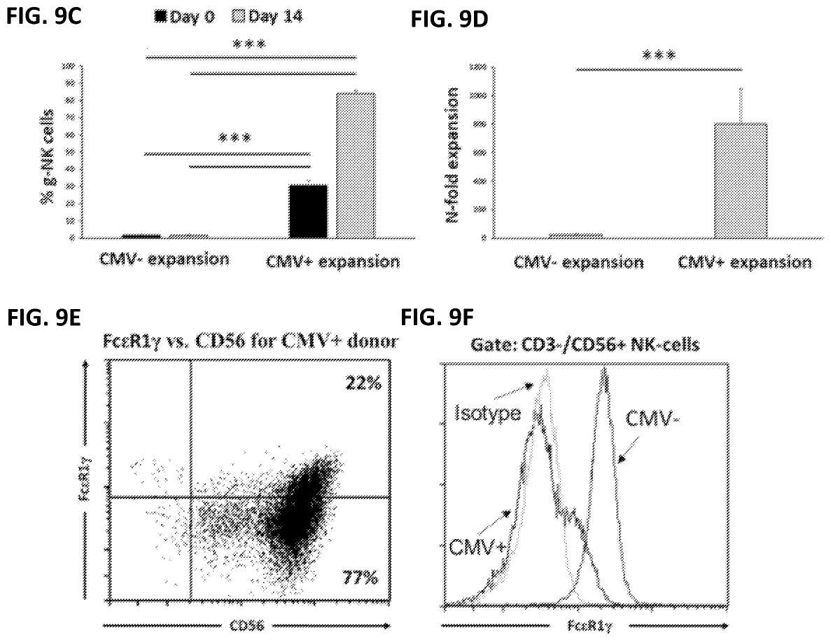

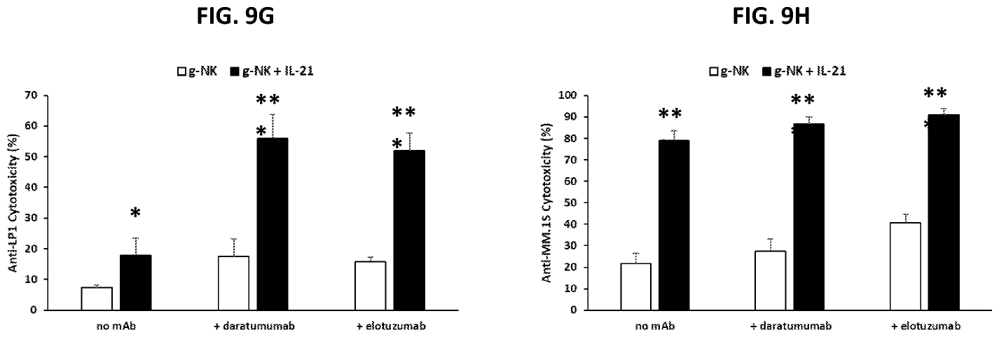

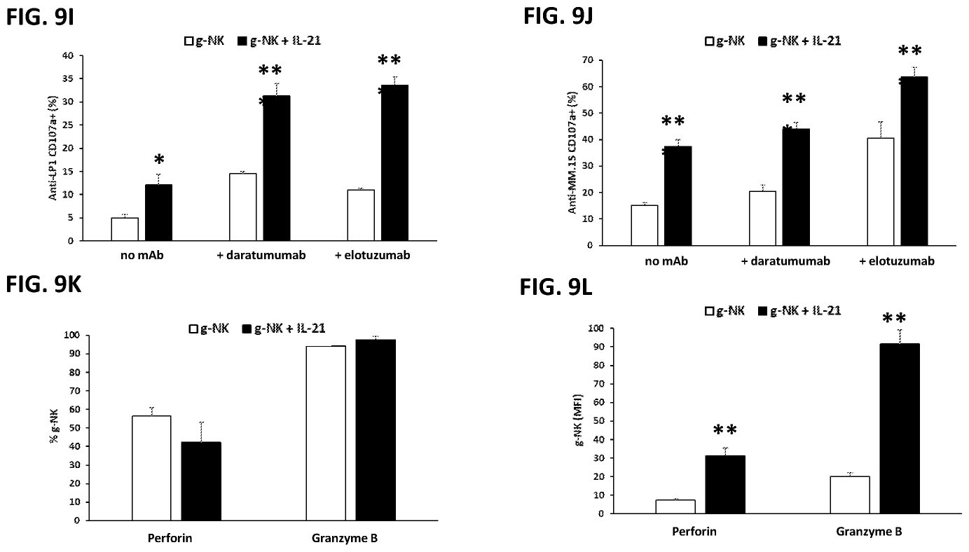

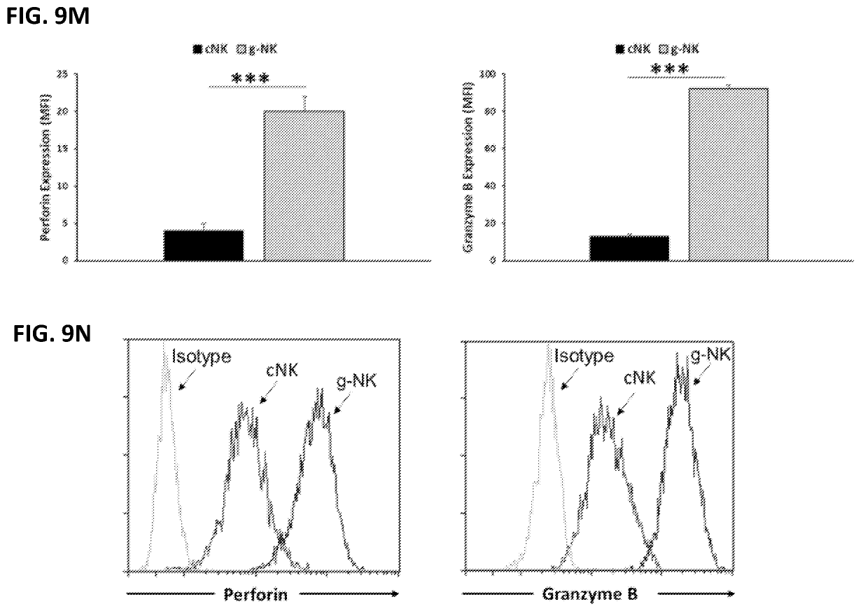

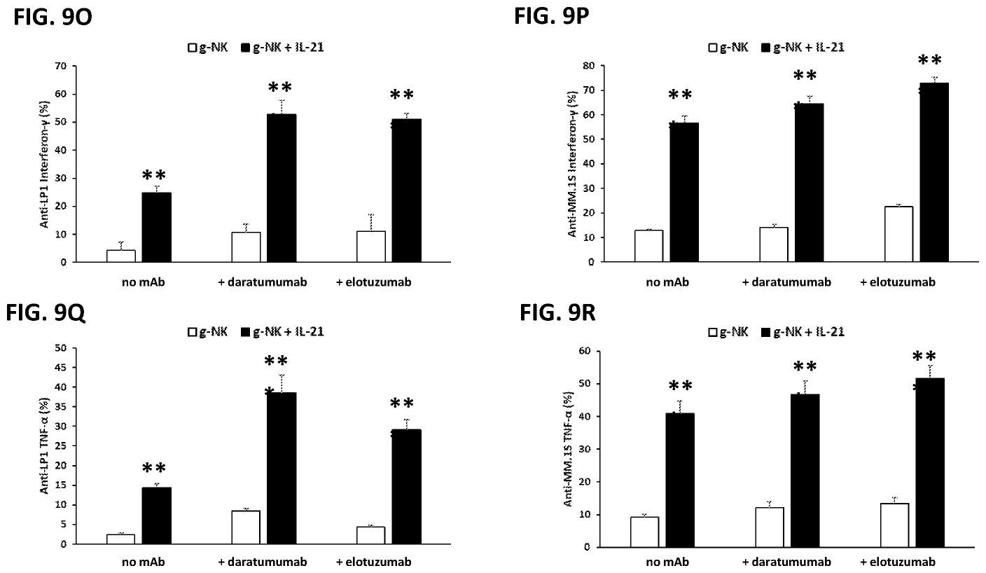

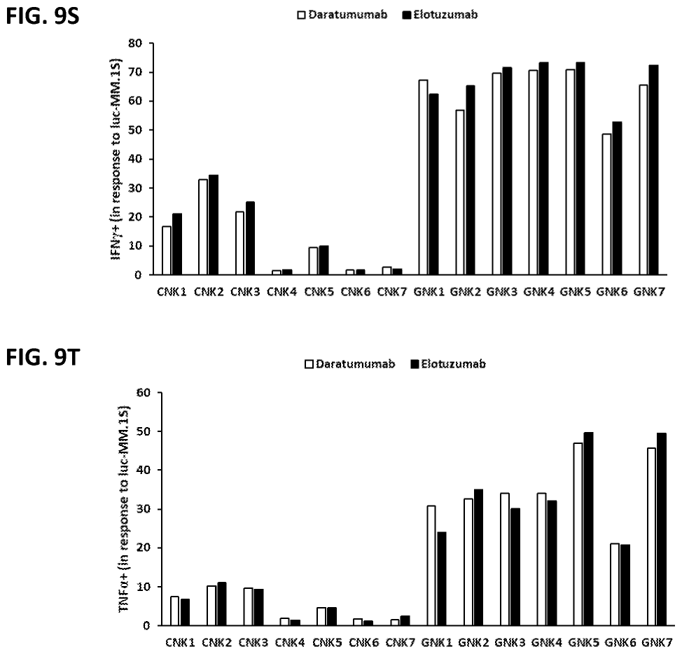

Cells were counted at media change and the percentage of g-NK cells was assessed by flow cytometry at day 0 and day 14. As shown in FIG. 9A and FIG. 9B, NK cells that has been initially enriched for CD3^{neg}/CD57^{pos} cells prior to expansion and then expanded in the presence of IL-21 had higher g-NK cell expansion rates than the similar conditions but without IL-21. As measured using intracellular staining of FcRγ and flow cytometry, higher g-NK cell expansion rates were observed when measuring both the percentage (FIG. 9A) and count (FIG. 9B) of g-NK cells.

Prior to expansion, the proportion of g-NK cells in the CMV seropositive donors was 30.8±3.1% (% of total NK-cells), while the proportion of g-NK cell was only 1.8±0.3% (% of total NK-cells) in the CMV seronegative donors. Following expansion after initial enrichment for CD3^{neg}/CD57^{pos} cells, the proportion of g-NK cells was increased to 84.0±1.4% for CMV-seropositive donors, but was unchanged for CMV-seronegative donors (1.5±0.4%) (FIG. 9C). Representative flow cytometry dot plots and histograms depicting the proportion of g-NK cells in CMV seropositive and seronegative donors are shown in FIGS. 9E and 9F. The percentage of NKG2Cpos/NKG2Aneg NK-cells within the g-NK subset ranged from 1.7 to 51% (26.8±13.9%). Thus, there is a phenotypic overlap between g-NK and NKG2C^{pos}/NKG2C^{neg} NK-cells but they are not identical.

A representative expansion of g-NK cells is shown in FIG. 9D, in which it is shown that the expansion method increased the proportion of g-NK cells from a CMV-seropositive donor with a detectable g-NK population with at least a 400-fold increase in overall NK-cell number.

Together, these results show that the presence of IL-21 improves g-NK cell expansion.

### B. Cell Mediated Cytoxicity

As shown in FIG. 9G and FIG. 9H, NK cells expanded in the presence of IL-21 had greater cell-mediated cytotoxicity against the CD38^{high} MM cell line LP1 (FIG. 9G) and the SLAMF7^{high} MM cell line MM.1S (FIG. 9H) than did g-NK cells expanded without IL-21. Greater cell-mediated cytotoxicity for IL-21 expanded g-NK cells was observed in the absence of antibody as well as in the presence of either daratumumab or elotuzumab.

Together, these results show that g-NK cells expanded in the presence of IL-21 have enhanced cell-mediated cytotoxicity against tumor cells compared to g-NK cells expanded without IL-21.

### C. Degranulation

As shown in FIG. 9I and FIG. 9J, g-NK cells expanded in the presence of IL-21 degranulated more against the CD38^{high} MM cell line LP1 (FIG. 9I) and the SLAMF7^{high} MM cell line MM.1S (FIG. 9J) than did g-NK cells expanded without IL-21. Greater degranulation for IL-21 expanded g-NK cells was observed in the absence of antibody as well as in the presence of either daratumumab or elotuzumab.

Together, these results show that g-NK cells expanded in the presence of IL-21 have enhanced degranulation against tumor cells compared to g-NK cells expanded without IL-21.

### D. Perforin and Granzyme B Expression

As shown in FIG. 9K and FIG. 9L, g-NK cells expanded in the presence of IL-21 expressed more of the cytolytic protein perforin than did g-NK cells expanded without IL-21, as measured by the total perforin expression (GMFI) (FIG. 9L), but not the percentage of perforin positive cells (FIG. 9K). In addition, g-NK cells expanded in the presence of IL-21 expressed more of the pro-apoptotic protein granzyme B than did g-NK cells expanded without IL-21, as measured by both the percentage of granzyme B positive cells (FIG. 9K) and the total granzyme B expression (GMFI) (FIG. 9L).

Baseline expression of perforin (FIG. 9M, left) and granzyme B (FIG. 9M, right) also was significantly higher in expanded g-NK cells than cNK cells (n=5).Representative histograms of perforin and granzyme B expression for NK and cNK cells is shown in FIG. 9N.

Together, these results show that g-NK cells expanded in the presence of IL-21 have enhanced expression of perforin and granzyme B against tumor cells compared to g-NK cells expanded without IL-21.

### E. Interferon-γ Expression

As shown in FIG. 9O and FIG. 9P, g-NK cells expanded in the presence of IL-21 expressed more Interferon-γ against the CD38^{high} MM cell line LP1 (FIG. 9O) and the SLAMF7^{high} MM cell line MM.1S (FIG. 9P) than did g-NK cells expanded without IL-21. Greater Interferon-γ expression for IL-21 expanded g-NK cells was observed in the absence of antibody as well as in the presence of either daratumumab or elotuzumab.

Together, these results show that g-NK cells expanded in the presence of IL-21 have enhanced Interferon-γ expression against tumor cells compared to g-NK cells expanded without IL-21.

### F. TNF-α Expression

As shown in FIG. 9Q and FIG. 9R, g-NK cells expanded in the presence of IL-21 expressed more TNF-α against the CD38^{high} MM cell line LP1 (FIG. 9Q) and the SLAMF7^{high} MM cell line MM.1S (FIG. 9R) than did g-NK cells expanded without IL-21. Greater TNF-α expression for IL-21 expanded g-NK cells was observed in the absence of antibody as well as in the presence of either daratumumab or elotuzumab.

Together, these results show that g-NK cells expanded in the presence of IL-21 have enhanced TNF-α expression against tumor cells compared to g-NK cells expanded without IL-21.

G. Comparison of Effector Functions Amongst g-NK Donors

g-NK cells and cNK cells were expanded as described and effector activity was compared amongst the different donors. Assays were performed as described in Example 2 using target cell line MM.1S at a 0.5:1 NK to MM cell ratio and with antibodies daratumumab and elotuzumab. After co-culture, the cells were fixed and permeabilized and analyzed by intracellular cytokine staining for Interferon-gamma (IFNγ) and TNF-alpha (TNFα). Results depicted in FIG. 9S (IFNγ) and FIG. 9T (TNFα) show that donor variability amongst g-NK donors is low, with a standard error of less than 5 for mAb-dependent IFNγ and TNFα response. Similar results were seen for other effector functions. The results showed that effector functions of all g-NK donors were superior to all cNK donors tested.

### Example 6: Expansion of g-NK Cells in the Presence of IL-21/Anti-IL-21 Complexes

Cryopreserved PBMCs were thawed and enriched for CD3^{neg}CD57^{pos} NK cells via magnetic sorting. Prior to expansion of these NK cells, IL-21/anti-IL-21 complexes were formed by combining IL-21 and an anti-IL-21 antibody. IL-21 and anti-IL-21 antibody were co-incubated for 30 minutes at 37° C. and at concentrations of 25 ng/ml and 250 ng/mL, respectively. The complexes, along with 500 IU/mL IL-2 and 10 ng/ml IL-15, were then added to the NK cell expansion media. NK cells were co-cultured with irradiated 221.AEH feeder cells at a 1:1 NK to 221.AEH feeder cell ratio. For comparison, NK cells were also expanded in the presence of IL-2, IL-15, and IL-21 at concentrations of 500 IU/mL, 10 ng/ml, and 25 ng/ml, respectively.

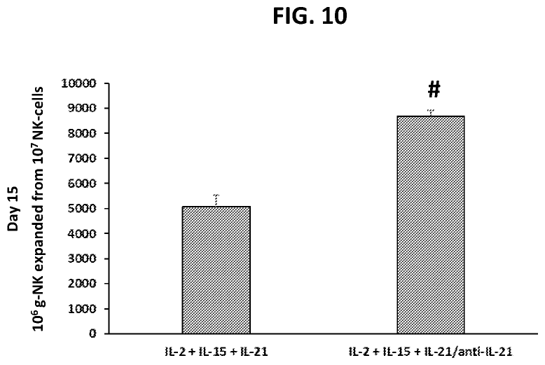

As shown in FIG. 10, g-NK cells expanded in the presence of IL-2, IL-15, and the IL-21/anti-IL-21 complex had a higher expansion rate than did g-NK cells expanded in the presence of IL-2, IL-15, and IL-21.

### Example 7: Maintenance of g-NK Cell Effector Function after Cryopreservation

NK cell effector function of previously cryopreserved g-NK cells was compared to that of freshly enriched (i.e., non-cryopreserved) g-NK cells (n=4). CD3^{neg}/CD57^{pos} enriched NK cells were co-cultured with irradiated 221.AEH feeder cells at a 2:1 221.AEH to NK cell ratio and in the presence of 500 IU/mL of IL-2, 10 ng/ml of IL-15, and 25 ng/ml of IL-21. After expansion, NK cells were functionally assessed fresh or were cryopreserved in 90% FBS with 10% DMSO and at a concentration of 20 million cells per 1.8 ml of cryopreservation media. NK cell effector functions against LP1 and MM.1S cell lines were assessed without antibody as well as in the presence of one μg/mL daratumumab or one μg/mL elotuzumab.

### A. Degranulation

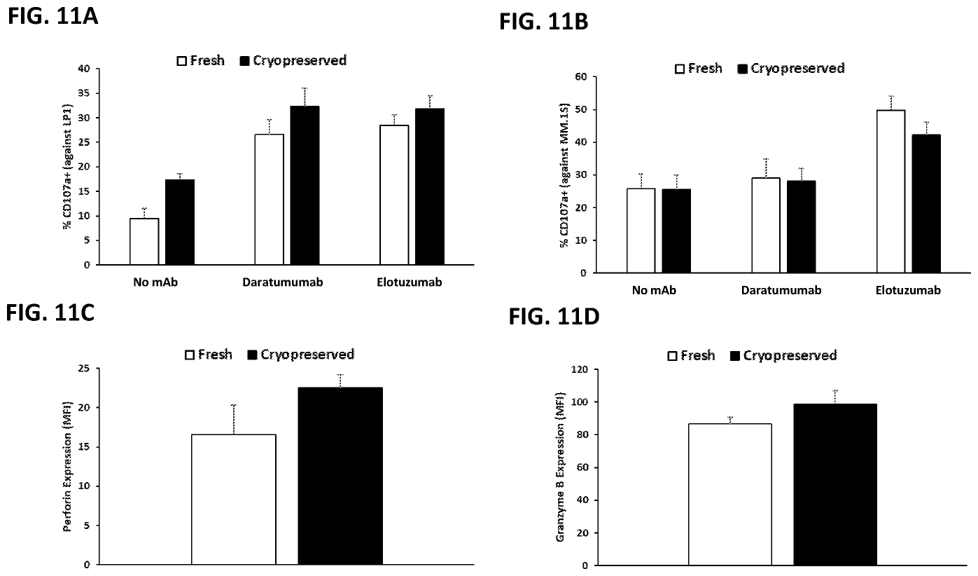

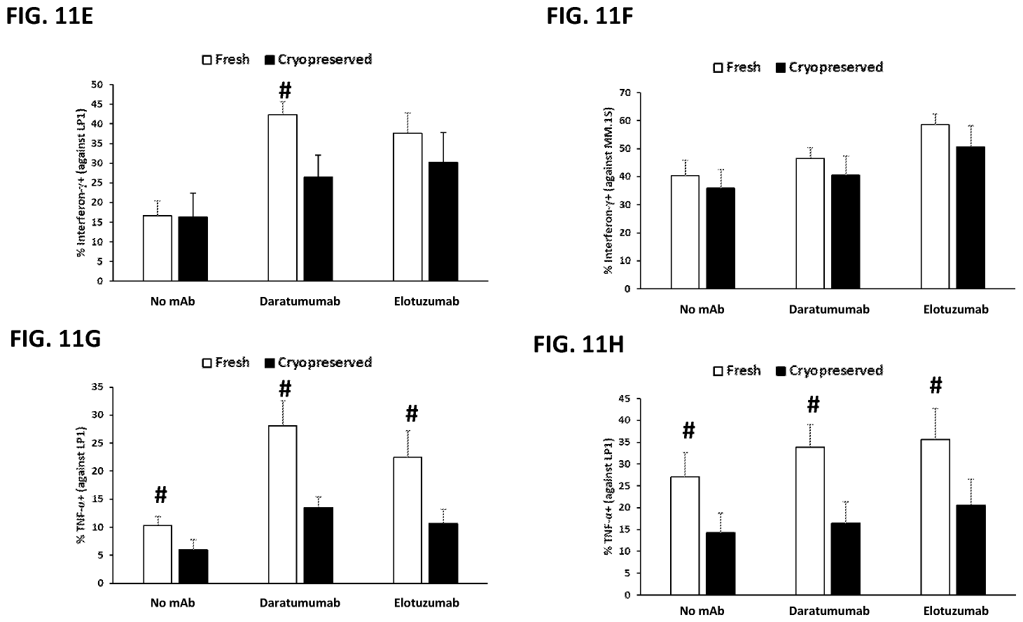

As shown in FIG. 11A and FIG. 11B, previously cryopreserved g-NK cells had degranulation levels comparable to that of fresh g-NK cells against the CD38^{high} MM cell line LP1 (FIG. 11A) and the SLAMF7^{high} MM cell line MM.1S (FIG. 11B). Comparable degranulation levels were observed in the absence of antibody as well as in the presence of either daratumumab or elotuzumab.

Together, these results show that g-NK cell degranulation in response to multiple myeloma target cells is maintained after cryopreservation.

### B. Perforin and Granzyme B Expression

As shown in FIG. 11C and FIG. 11D, previously cryopreserved g-NK cells had perforin (FIG. 11C) and granzyme B expression (FIG. 11D) comparable to that of fresh g-NK cells. Together, these results show that g-NK cell perforin and granzyme B expression is maintained after cryopreservation.

### C. Interferon-γ Expression

As shown in FIG. 11E and FIG. 11F, previously cryopreserved g-NK cells had Interferon-γ expression levels comparable to that of fresh g-NK cells against the CD38^{high} MM cell line LP1 (FIG. 11E) and the SLAMF7^{high} MM cell line MM.1S (FIG. 11F). Comparable Interferon-γ expression was observed in the absence of antibody as well as in the presence of either daratumumab or elotuzumab.

Together, these results show that g-NK cell Interferon-γ expression in response to multiple myeloma target cells is maintained after cryopreservation.

### D. TNF-α Expression

As shown in FIG. 11G and FIG. 11H, previously cryopreserved g-NK cells had decreased TNF-α expression levels compared to that of fresh g-NK cells against the CD38^{high} MM cell line LP1 (FIG. 11G) and the SLAMF7^{high} MM cell line MM.1S (FIG. 11H). Decreased TNF-α expression was observed in the absence of antibody as well as in the presence of either daratumumab or elotuzumab.

Together, these results show that g-NK cell TNF-α expression in response to multiple myeloma target cells is decreased after cryopreservation.

### Example 8: Assessment of Persistence of g-NK Cells In Vivo Compared to cNK Cells

NK cells, expanded substantially as described in Example 5, were injected into mice and biological samples were subjected to analysis using flow cytometry to assess their persistence.

As described in Example 5, g-NK cells were expanded after initially enriching for CD3^{neg}/CD57^{pos} cells from cryopreserved PBMCs, followed by expansion with irradiated 221.AEH feeder cells at a 1:1 221.AEH to NK cell ratio and in the presence of IL-2 (500 IU/mL), IL-15 (10 ng/ml), and IL-21 (25 ng/mL) stimulatory cytokines. The alternative method described in Example 5 was used to expand cNK cells due to insufficient yield of cNK cells from CMV-seronegative donors. cNK cells were expanded for 2 weeks using the transgenic leukemia cell line K562-mb15-41BBL and IL-2. All cells were expanded from cryopreserved PBMCs and cryopreserved feeder cells. Freeze media for the cryopreserved cells was CS-10 (Biolife Solutions, Bothel, WA, USA). Cryopreserved cell products were thawed rapidly in a hot water bath prior to being administered to the mice (37° C.).

A single dose of 1×10⁷ expanded NK cells (fresh g-NK, cryopreserved g-NK, or cryopreserved cNK cells) were intravenously injected via the tail vein into female NOD.Cg-PrkDc^{scid}IL2rg^{tmlWjl}/SzJ (NSG) mice (n=9, 3 per group). To provide NK-cell support, about 2 μg/mouse human recombinant IL-15 was administered via the I.P. route every three days (see Table 2). Blood collected at days 6, 16, 26, and 31 days post-infusion was immediately analyzed by flow cytometry. Mice were sacrificed at day 31, and bone marrow and spleen were harvested for immediate flow cytometry analysis.

TABLE 2
Persistence Study Design

| Group | | Number | Day of | Days of blood |
|---|---|---|---|---|
| Number | Arm | of Mice | NK dose | collection |
| 1 | IL-15 + Fresh | 3 | 1 | 6, 16, 26, |
| | day 14 g-NK | | | 31 (sac) |
| 2 | IL-15 + Cryo | 3 | 1 | 6, 16, 26, |
| | day 14 g-NK | | | 31 (sac) |
| 3 | IL-15 + Cryo | 3 | 1 | 6, 16, 26, |
| | day 14 cNK | | | 31 (sac) |

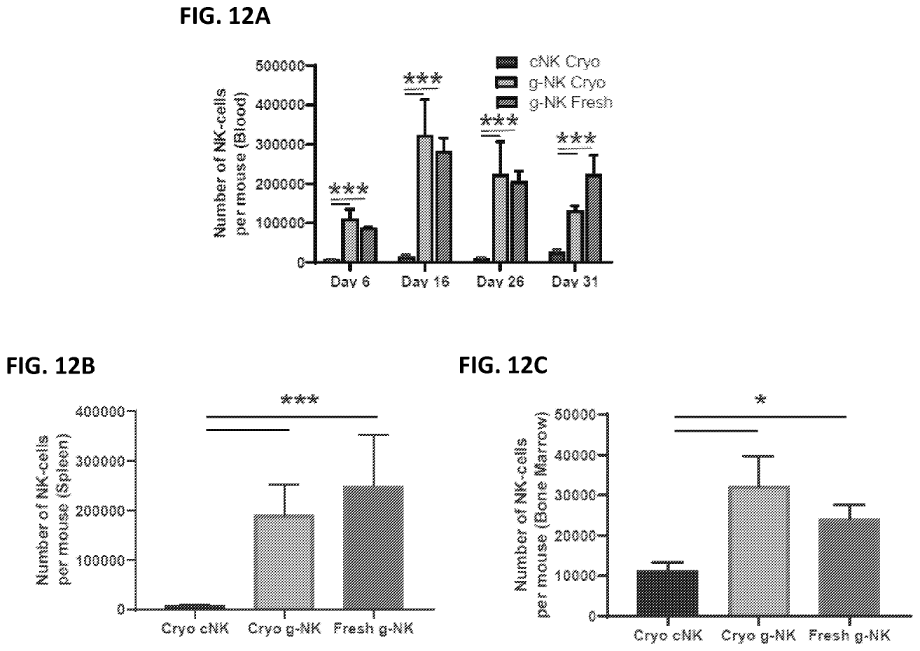

FIG. 12A-C shows enhanced persistence of fresh and cryopreserved g-NK cells relative to cNK cells in peripheral blood (FIG. 12A), spleen (FIG. 12B), and bone marrow (FIG. 12C). Persistence of cryopreserved g-NK cells was >90% greater than that seen with cryopreserved cNK cells in peripheral blood at multiple time points (p<0.001) (FIG. 12A), as well as spleen (p<0.001) (FIG. 12B) and bone marrow (p<0.05) (FIG. 12C) at sacrifice at day 31 (p<0.001). FIG. 12A also shows that levels of fresh and cryopreserved g-NK cells persisted at comparable levels until at least day 26 of the study.

The results are consistent with an observation that g-NK cells exhibit significantly improved persistence. These results demonstrate the utility of fresh or cryopreserved g-NK as a viable, off-the-shelf cellular therapy to enhance mAb ADCC.

### Example 9: Assessment of CD38 and SLAMF7 on g-NK Cells and Fratricide Activity of g-NK Cells

This example demonstrates, in part, the protection of g-NK cells from antibody due to lack of target surface markers.

g-NK cells were expanded substantially by the methods described in Example 5 with certain exceptions: 1) The ratio of 22.AEH target cells to NK cells was 2.5:1 (compared to a 2:1 ratio in Example 5), 2) NK cells were exposed to a lower level of IL-2 (100 IU/ml compared to 500 IU/ml in Example 5) and 3) IL-21 was absent during expansion. Approximately 2.0×10⁵ NK-cells and/or MM.1S or Raji cells were aliquoted into flow tubes and stained with 2 μL of 7-AAD viability dye and 2 μL of anti-CD45, 2 μL of anti-CD20, 2 μL of anti-CD38, 2 μL of anti-CD3, 10 μL of anti-SLAMF7, and 2 μL of anti-CD56 antibodies as described in Table E3. After a 10-minute incubation at 4º C, the cells were washed and intracellular staining was performed using an anti-FceRI antibody (Millipore). After completion of the staining process, the percentages of CD20, CD38, and SLAMF7 expressing g-NK, cNK, and MM.1S or Raji cells were assessed by 8-color flow cytometry (Miltenyi MACSQuant Analyzer 10).

TABLE E3
Flow cytometry panel to determine CD20, CD38, and SLAMF7
expression on NK, MM, and Raji cells.

| Condition | V1 | V2 | B1 | B2 | B3 | B4 | R1 | R2 |
|---|---|---|---|---|---|---|---|---|
| NK Only | CD45 | CD20 | *FcRg | CD38 | 7-AAD | CD3 | SLAMF | CD56 |
| | | | | | | | 7 | |
| NK Only | CD45 | | *FcRg | CD38 | 7-AAD | CD3 | SLAMF | CD56 |
| CD20 | | | | | | | 7 | |
| FMO | | | | | | | | |
| NK Only | CD45 | CD20 | *FcRg | | 7-AAD | CD3 | SLAMF | CD56 |
| CD38 | | | | | | | 7 | |
| FMO | | | | | | | | |
| NK Only | CD45 | CD20 | *FcRg | CD38 | 7-AAD | CD3 | | CD56 |
| SLAMF7 | | | | | | | | |
| FMO | | | | | | | | |
| NK + | CD45 | CD20 | *FcRg | CD38 | 7-AAD | CD3 | SLAMF | CD56 |
| MM | | | | | | | 7 | |
| NK + | CD45 | CD20 | *FcRg | | 7-AAD | CD3 | SLAMF | CD56 |
| MM | | | | | | | 7 | |
| CD38 | | | | | | | | |
| FMO | | | | | | | | |
| NK + | CD45 | CD20 | *FcRg | CD38 | 7-AAD | CD3 | | CD56 |
| MM | | | | | | | | |
| SLAMF7 | | | | | | | | |
| FMO | | | | | | | | |
| MM | CD45 | CD20 | | CD38 | 7-AAD | | SLAMF | |
| Only | | | | | | | 7 | |
| MM | CD45 | CD20 | | | 7-AAD | | SLAMF | |
| Only | | | | | | | | |
| CD38 | | | | | | | 7 | |
| FMO | | | | | | | | |
| MM | CD45 | CD20 | | CD38 | 7-AAD | | | |
| Only | | | | | | | | |
| SLAMF7 | | | | | | | | |
| FMO | | | | | | | | |
| NK + | CD45 | CD20 | *FcRg | CD38 | 7-AAD | CD3 | SLAMF | CD56 |
| Raji | | | | | | | 7 | |
| NK + | CD45 | | *FcRg | CD38 | 7-AAD | CD3 | SLAMF | CD56 |
| Raji | | | | | | | | |
| CD20 | | | | | | | 7 | |
| FMO | | | | | | | | |
| Raji | CD45 | CD20 | | | 7-AAD | | | |
| Only | | | | | | | | |
| Raji | CD45 | | | | 7-AAD | | | |
| Only | | | | | | | | |
| CD20 | | | | | | | | |
| FMO | | | | | | | | |

*FcRg is an intracellular epitope

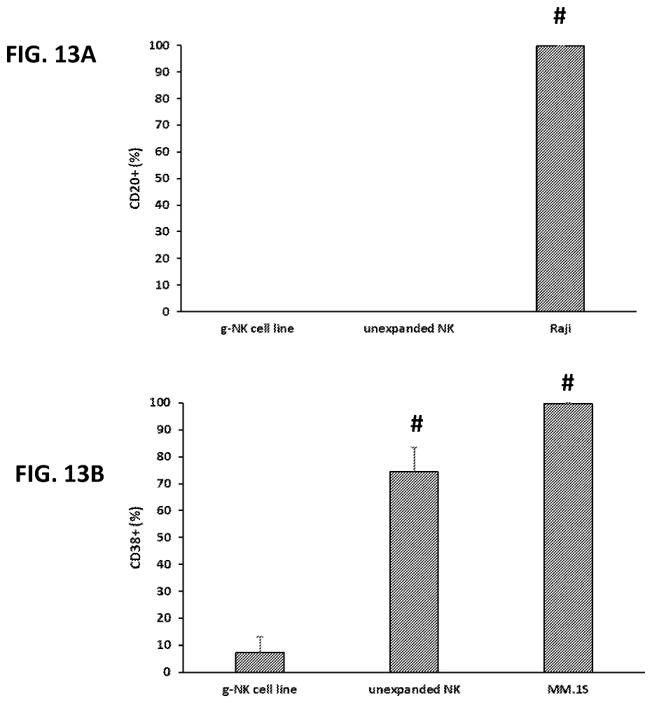

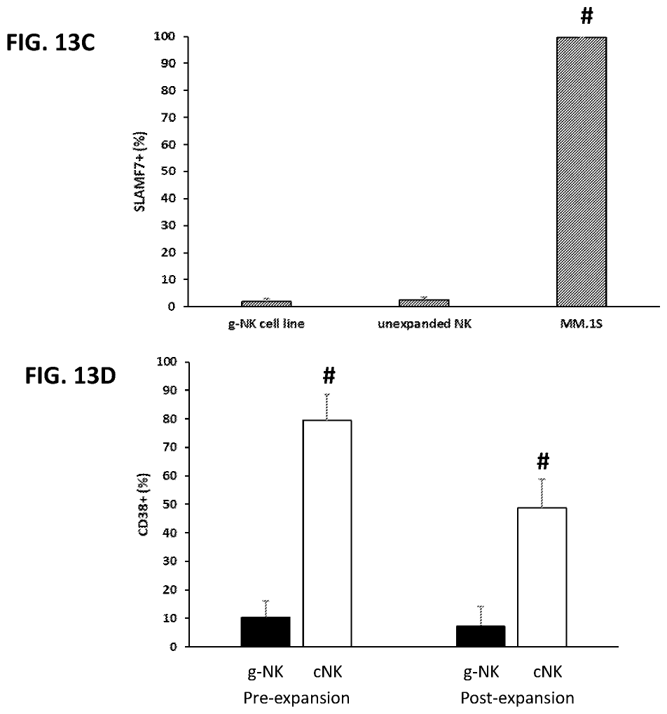

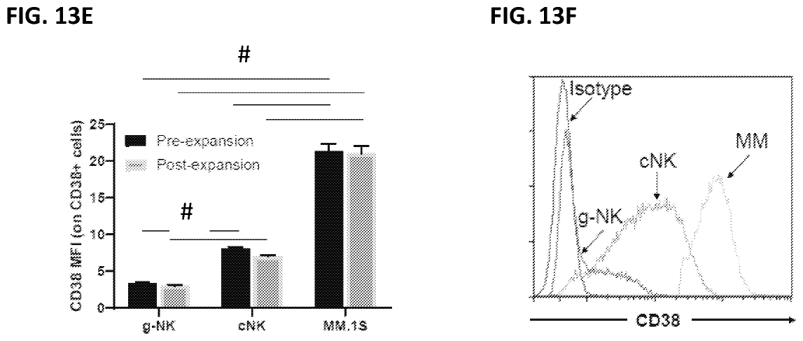

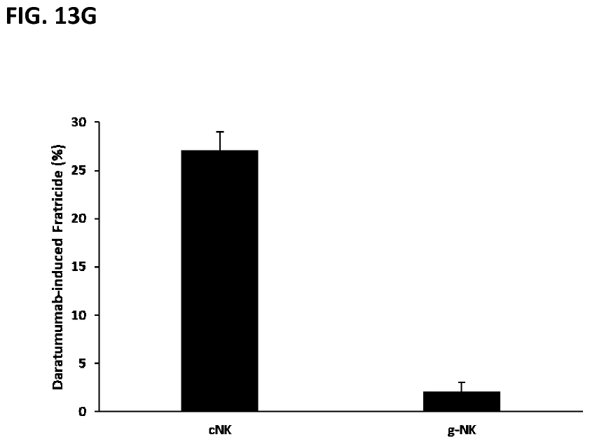

Expression of CD20, CD38, and SLAMF7 on g-NK, cNK, and MM.1S cells is presented in FIG. 13A-13D. Both g-NK and cNK lacked expression of CD20, which was highly expressed on Raji lymphoma cells (FIG. 13A). The expression of CD38 by g-NK was far less than for cNK and MM.1S cells (see FIG. 13B; p<0.001 for both). Expression of SLAMF7 was not different between g-NK and cNK (p=0.9), but both g-NK and cNK exhibited far lower expression of SLAMF7 than MM.1S cells (see FIG. 13C; p<0.001 for both). Reduced percentage of CD38^{pos} NK-cells was also seen on expanded g-NK when compared to expanded cNK (see FIG. 13D, p<0.001). Furthermore, intensity of CD38 expression (MFI) was reduced on CD38^{pos} g-NK cells relative to CD38^{pos} cNK and MM1/S cells (FIG. 13E, p<0.001). A representative histogram depicting the reduced CD38 expression of g-NK cells relative to cNK and MM.1S cells is shown in FIG. 13F.

The lack of CD20, CD38, or SLAMF7 expression by g-NK afforded protection from mAb-induced fratricide by rituximab (anti-CD20), daratumumab (anti-CD38), or elotuzumab (anti-SLAMF7). Overall, this data further illustrates how g-NK have a persistence advantage when compared to cNK, especially when in the presence of therapeutic antibodies such as daratumamab.

Similar results were observed by the expansion method described in Example 5 in the presence of IL-21, indicating that there is no difference in CD38 or SLAMF7 expression between g-NK cells expanded with or without IL-21. In a further assessment, the fratricide rate of expanded g-NK cells was compared to that of expanded cNK cells. As shown in FIGS. 13B and 13D-13F, CD38 expression was markedly lower on g-NK cells than cNK cells, and as shown in FIG. 13C equally low levels of SLAMF7 was present on g-NK and cNK cells. These results indicate the potential for lack of a fratricide effect by g-NK cells against these targets, since if NK cells express a mAb target an ADCC activity may lead to elimination of NK cells by fratricide in addition to the tumor. The finding that cNK cells express high levels of CD38 is consistent with prior results suggesting that >90% of CD38^{high} NK cells are depleted rapidly after daratumumab treatment in patients (Casneuf et al., 2017 Blood Adv, 1(23):2105-2114).

Six (6) unique donors were used to generate the expanded g-NK (6 CMV+, 3 M, 3F, age 39±7 years) and 8 unique donors were used to expand cNK (8 CMV−, 4 M, 4 F, age 38±9 years) using the methods substantially as described in Example 5. The proportion of g-NK was 85±4% for the g-NK donors and 2±1% for the cNK donors.

To assess fratricide, about 1×10⁴ expanded NK cells (g-NK or cNK) were cultured in the presence of 1 μg/mL daratumumab (anti-CD38). After a four-hour incubation at 37° C. in a 5% CO² incubator, the cells were washed and stained with anti-CD3 and anti-CD56 antibodies to quantify the number of NK cells. After a final wash, propidium iodide (PI) was added, and the number of live and dead NK-cells were resolved using 3-color flow cytometry (Bigley et al. (2016), Clin. Exp. Immunol., 185:239-251). As shown in FIG. 13G, g-NK cells have 13 times lower fratricide than cNK. Similar experiments carried out with elotuzumab showed that fratricide was not detected for g-NK or cNK treated with elotuzumab.

Together with the results of g-NK cells expanded in the absence of IL-21, these results are consistent with the ability of g-NK cells to confer enhanced mAb anti-tumor activity in MM without suffering from fratricide-related depletion.

### Example 10: In Vivo Efficacy in a Disseminated Orthotopic Xenograft MM.1S Model of Multiple Myeloma

The in vivo efficacy of NK cells (expanded g-NK cells or cNK cells) in combination with daratumumab was evaluated by measuring tumor inhibition and survival in a murine model of multiple myeloma. g-NK cells were expanded as described in Example 5 after initially enriching for CD3^{neg}/CD57^{pos} cells from cryopreserved PBMCs, followed by expansion with irradiated 221.AEH feeder cells at a 1:1 221.AEH to NK cell ratio and in the presence of IL-2 (500 IU/mL), IL-15 (10 ng/ml), and IL-21 (25 ng/mL) stimulatory cytokines. The alternative method described in Example 5 was used to expand cNK cells due to insufficient yield of cNK cells from CMV-seronegative donors. cNK cells were expanded for 2 weeks using the transgenic leukemia cell line K562-mb15-41BBL and IL-2. All cells were expanded from cryopreserved PBMCs and cryopreserved feeder cells.

Approximately 5×10⁵ luciferase-labeled MM. 1S human myeloma cells were injected intravenously into to tail veins of female NSG mice and allowed to grow for 14 days. The monoclonal antibody daratumumab was administered via the I.P. route in combination with intravenous administration of 6.0×10⁶ expanded g-NK or cNK cells weekly, for a duration of five weeks. Beginning two weeks after tumor administration, 2 μg/mouse human recombinant IL-15 was administered every three days via the I.P. route to provide NK-cell support. Table 4 summarizes the groups of mice treated in the study.

Bioluminescence imaging (BLI) was performed twice per week to monitor tumor burden. Mice were checked daily for signs of discomfort and tolerability, and body weight was measured twice per week beginning one week after tumor inoculation. Mice were imaged after 15 minutes of subcutaneous injection of 150 mg/kg D-luciferin. Total flux (photons/second) of the entire mouse was quantified using Living Image software (PerkinElmer). Tumor bearing mice were sacrificed upon development of symptomatic myeloma, such as hind limb paralysis, grooming, and/or lethargy. Time to sacrifice was used as a proxy for survival. All surviving mice were sacrificed 43 days after initial NK-cell dose for tissue collection. At the completion of the study, flow cytometry was used to quantify g-NK, cNK, and MM.1S (CD138^{pos}/CD45^{neg}) cells from biological samples to determine tumor burden and NK-cell survival.

TABLE E4
MM Efficacy Study Design

| | | | Days of | Days of |
|---|---|---|---|---|
| Group | | Number | Antibody | NK cell |
| Number | Arm | of Mice | Administration | administration |
| 1 | Vehicle control | 8 | N/A | N/A |
| 2 | g-NK I.V. + 10 ug | 7 | 14, 21, 28, | 14, 21, 28, |
| | Daratumumab I.P. + | | 35, 42 | 35, 42 |
| | IL-15 I.P.* | | | |
| 3 | cNK I.V. + 10 ug | 7 | 14, 21, 28, | 14, 21, 28, |
| | Daratumumab I.P. + | | 35, 42 | 35, 42 |
| | IL-15 I.P.* | | | |

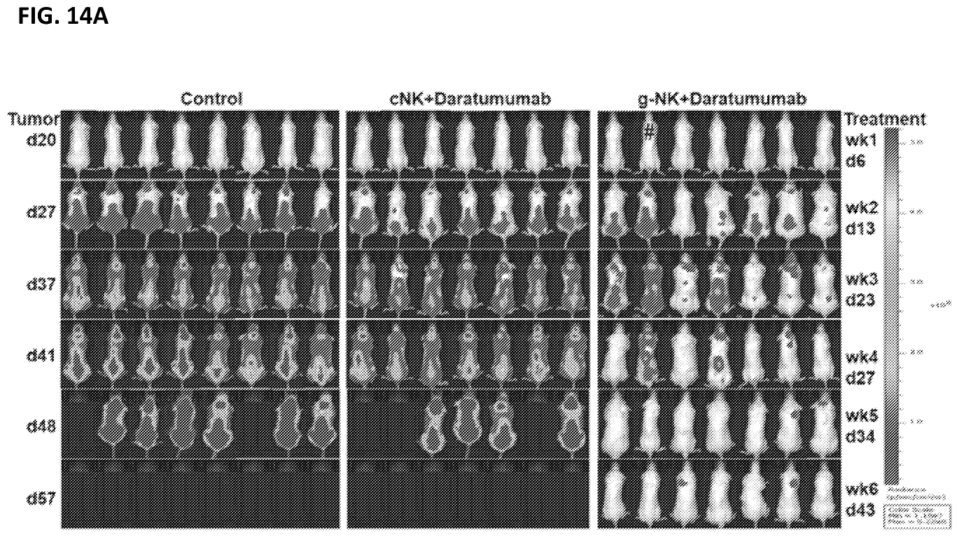

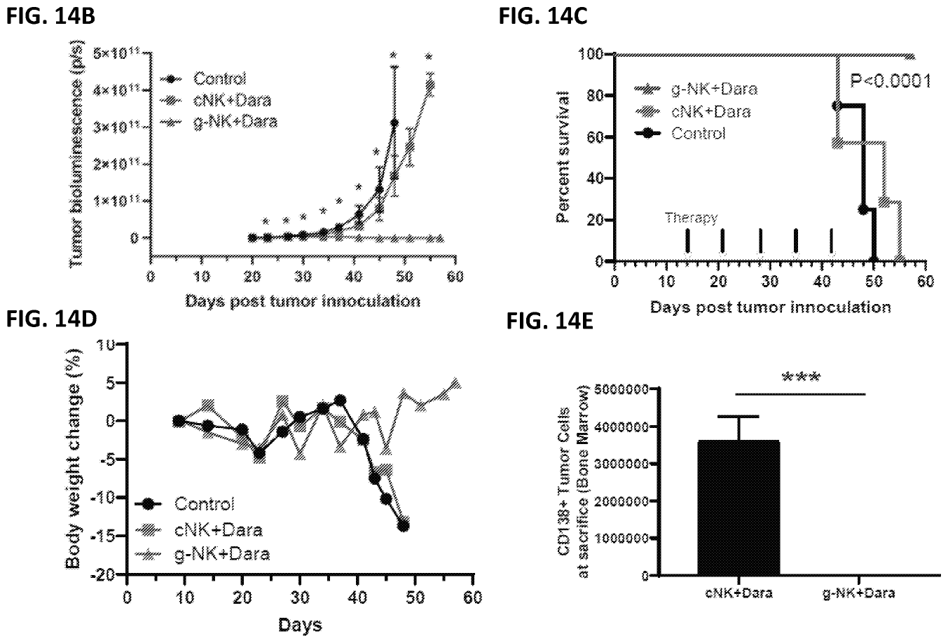

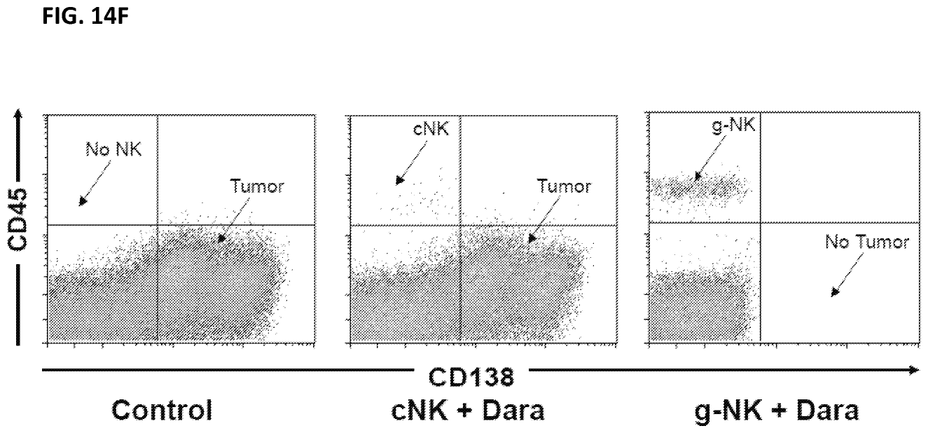

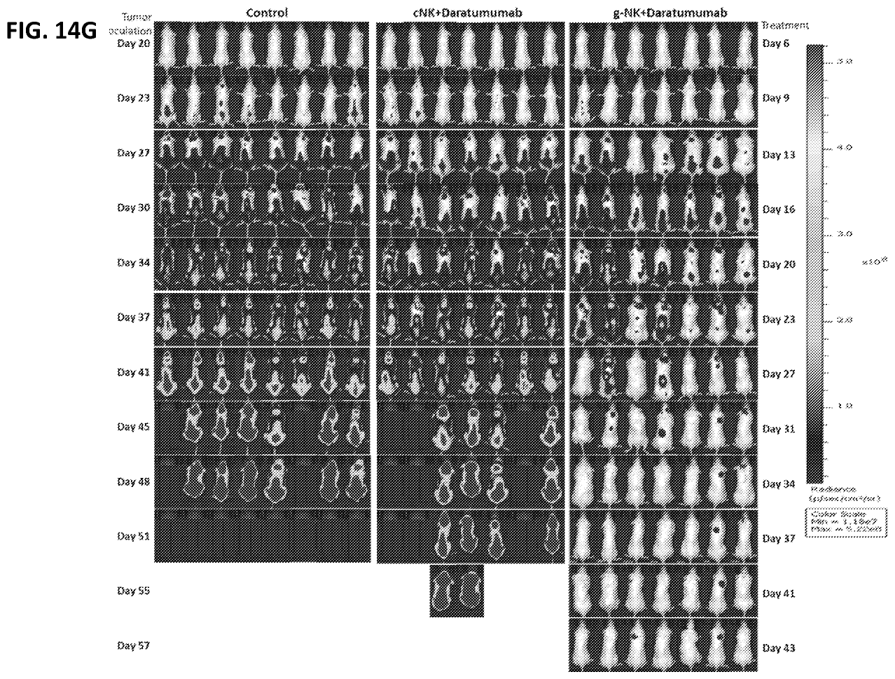

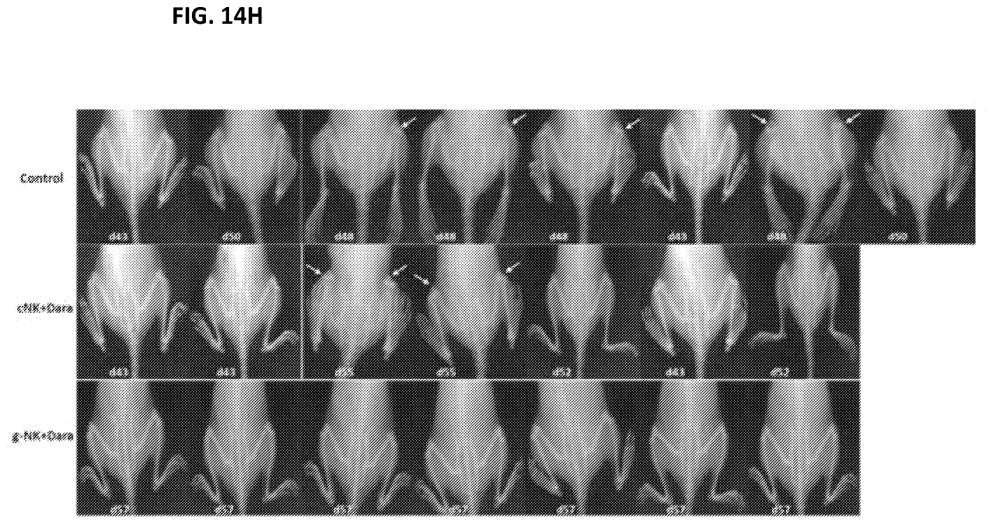

Co-administration of g-NK and daratumumab resulted in significant tumor inhibition and enhanced survival compared to treatment with cNK and daratumumab. As shown in FIG. 14A, g-NK cells plus daratumumab eliminated myeloma tumor burden in 5 of 7 mice evidenced by BLI imaging after 5 weeks of treatment. Quantitative BLI analysis showed g-NK plus daratumumab induced sustained and statistically significant tumor regression (FIG. 14B). The Kaplan-Meier survival analysis showed that the overall survival probability of the g-NK plus daratumumab treated mice was significantly better than those mice treated with vehicle or with cNK and daratumumab (p<0.0001) (FIG. 14C). All mice dosed with g-NK cells were energetic with no weight loss or toxicities observed at the conclusion of the study, while all control mice or mice treated with cNK cells and daratumumab had severe weight loss and succumbed to myeloma before conclusion of the study (FIG. 14D). Interestingly, one of the mice treated with g-NK cells was not dosed until day 21 after tumor inoculation due to anesthesia-induced suffocation of one of the mice, and this mouse had no detectable tumor BLI at the conclusion of the study despite having the highest peak BLI of the g-NK mice (FIG. 14A, mouse labeled as #). Of the 7 mice who were dosed with g-NK cells, only 2 had a minimally detectable amount of residual tumor BLI.

Flow cytometry analysis of the bone marrow confirmed that the 5 g-NK treated mice with no detectable tumor BLI were in fact tumor free (no CD138 pos cell in bone marrow). The average tumor burden for all 7 g-NK treated mice was reduced greater than 99% relative to mice treated with cNK and daratumumab (p<0.001; FIG. 14E). Representative flow cytometry dot plots depicting tumor burden and persistent NK-cells in bone marrow are shown in FIG. 14F. All of the BLI images taken over the course of the study are shown in FIG. 14G. X-ray images were obtained from all of the mice prior to sacrifice and it was determined that control mice or mice treated with cNK cells and daratumumab had fractures and malformations of the hind limb bones, while one of the mice treated with g-NK cells and daratumumab had any bone deformities (FIG. 14H).

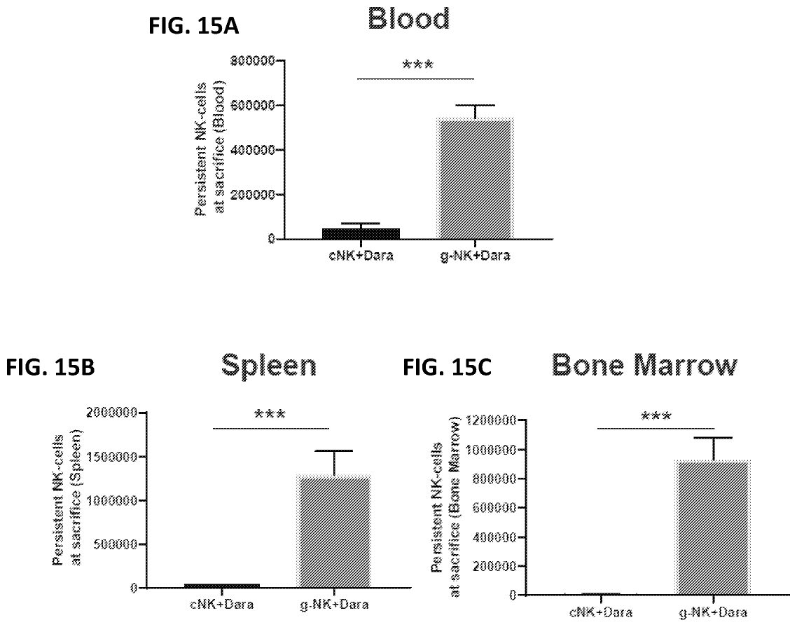

Analysis of NK cells in blood, spleen and bone marrow demonstrated a large increased in persistence of g-NK cells in daratumumab treated mice relative to cNK cells (FIG. 15A-C). Notably, g-NK cell numbers were >90% higher than cNK cells in blood (FIG. 15A), >95% higher in spleen (FIG. 15B), and >99% higher in bone marrow (FIG. 15C).

Taken together, the results further support the superiority of g-NK cells, including compared to cNK cells, for enhancing mAb effects in vivo and suggest that g-NK cells given in combination with daratumumab could be potentially curative for MM. Further, the results support that enhanced survival and resistance to fratricide result in superior anti-tumor effects and persistence of g-NK cells.

### Example 11: In Vivo Efficacy in a Disseminated Orthotopic Xenograft Raji Model of Lymphoma

The in vivo efficacy of NK cells (expanded g-NK cells or cNK cells) in combination with rituximab was evaluated by measuring tumor inhibition and survival in a murine model of lymphoma.

After expansion of the g-NK cells, approximately 5×10⁵ luciferase-labeled Raji human lymphoma cells were injected intravenously into the tail veins of female NSG mice and allowed to grow for 2 days. The monoclonal antibody rituximab (anti-CD20) was administered via I.P. route at 200 μg/mouse in combination with intravenous administration of 15×10⁶ expanded g-NK or cNK cells weekly beginning two weeks after tumor inoculation. Beginning two days after tumor inoculation, IL-15 was administered every three days to provide NK-cell support. Table 5 summarizes the groups of mice treated in the study.

TABLE E5
Lymphoma Efficacy Study Design

| | | | Days of | Days of |
|---|---|---|---|---|
| Group | | Number | Antibody | NK cell |
| Number | Arm | of Mice | Administration | administration |
| 1 | Vehicle control | 8 | N/A | N/A |
| 2 | g-NK I.V. + 200 ug | 7 | 14, 21, 28, | 14, 21, 28, |
| | Rituxumab I.P. + | | 35, 42 | 35, 42 |
| | IL-15 I.P. | | | |
| | (every 3 days) | | | |
| 3 | cNK I.V. + 200 ug | 7 | 14, 21, 28, | 14, 21, 28, |
| | Rituxumab I.P. + | | 35, 42 | 35, 42 |
| | IL-15 I.P | | | |
| | (every 3 days) | | | |

Bioluminescence imaging (BLI) was performed once per week to monitor tumor burden beginning one week after tumor inoculation. Mice were checked daily for signs of discomfort and tolerability, and body weight was measured twice per week beginning one week after tumor inoculation. Mice were imaged after 15 minutes of subcutaneous injection of 150 mg/kg D-luciferin. Total flux (photons/second) of the entire mouse was quantified using Living Image software (PerkinElmer). Tumor bearing mice were sacrificed upon development of symptomatic lymphoma. Time to sacrifice was used as a proxy for survival. At the completion of the study, flow cytometry was used to quantify g-NK, cNK, and Raji.

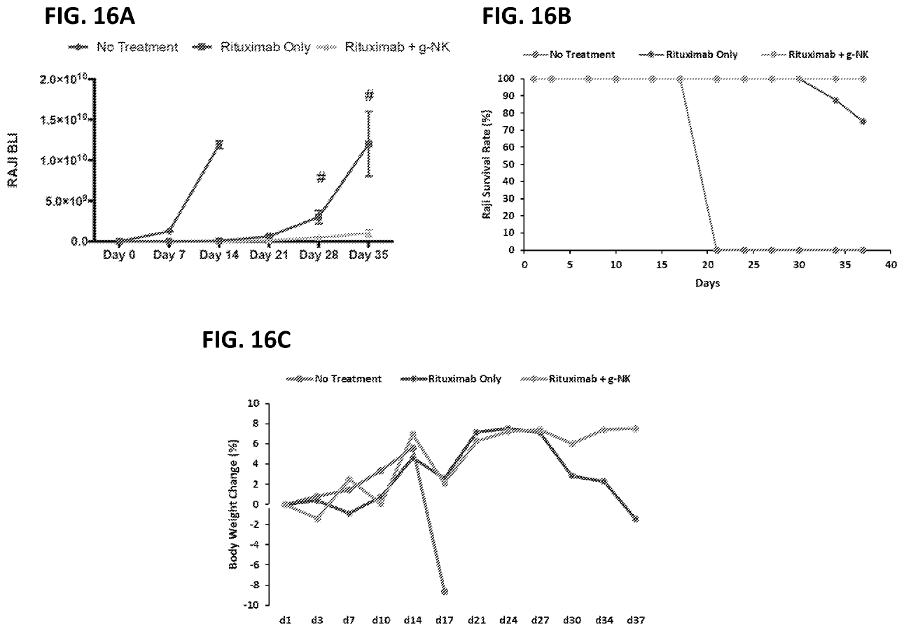

Co-administration of g-NK and rituximab resulted in significant tumor inhibition and enhanced survival compared to treatment with cNK and rituximab. As shown in FIG. 16A, expanded g-NK cells have markedly enhanced antibody-dependent cellular cytotoxicity (ADCC) activity when combined in rituximab in vivo. Qualitative BLI analysis (photons/second) show that g-NK cells plus rituximab resulted in statistically significant decreased presence of Raji lymphoma cells relative to rituximab with cNK cells or no treatment. The Kaplan-Meier survival analysis showed that the overall survival probability of the g-NK plus rituximab treated mice was significantly improved than those mice treated with rituximab and cNK cells or without treatment (FIG. 16B).

All mice dosed with g-NK cells were energetic with no weight loss or toxicities observed at the conclusion of the study, while all mice not receiving any treatment succumbed to lymphoma before the conclusion of the study (FIG. 16B and FIG. 16C). Mice receiving rituximab and cNK cells showed significant weight loss relative to mice receiving g-NK cells plus rituximab (FIG. 16C).

Taken together, the results further support the superiority of g-NK cells, including compared to cNK cells, for enhancing mAb effects in vivo and suggest that g-NK cells given in combination with rituximab could be potentially curative for lymphoma.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

What is claimed:

1. A method of treating multiple myeloma, the method comprising administering a composition of Natural Killer (NK) cells deficient in expression of FcRγ chain (g-NK cells) to a subject having multiple myeloma (MM), wherein the composition of g-NK cells is administered once weekly for a predetermined number of doses.

2. The method of claim 1, wherein the method is a monotherapy without combined administration of an exogenous antibody for treating the multiple myeloma.

3. The method of claim 1, wherein the method further comprises administering to the subject an antibody that is directed against a multiple myeloma antigen.

4. The method of claim 3, wherein the multiple myeloma antigen comprises an antigen selected from the group consisting of CD38, SLAMF7, and BCMA.

5. The method of claim 3 or claim 4, wherein the antibody is a full-length antibody.

6. The method of any one of claims 3-5, wherein the antibody is an anti-SLAMF7 antibody.

7. The method of any one of claims 3-5, wherein the antibody is an anti-BCMA antibody.

8. The method of any one of claims 3-5, wherein the antibody is an anti-CD38 antibody.

9. The method of claim 3, wherein the antibody is a bispecific antibody.

10. The method of claim 9, wherein the bispecific antibody is directed against CD16 and a second multiple myeloma antigen selected from the group consisting of BCMA, SLAMF7, and CD38.

11. The method of claim 9 or claim 10, wherein the bispecific antibody is directed against CD16 and CD38.

12. The method of any one of claims 3-11, wherein the antibody is administered once every four weeks, once every three weeks, once every two weeks, once weekly, or twice weekly.

13. The method of claim 8, wherein at least one dose of anti-CD38 antibody has been administered to the subject prior to administration of a dose of the composition of g-NK cells.

14. A method of treating multiple myeloma, the method comprising administering a composition of Natural Killer (NK) cells deficient in expression of FcRγ chain (g-NK cells) to a subject having multiple myeloma (MM), wherein the composition of g-NK cells is administered once weekly for a predetermined number of doses, and wherein the subject has received prior administration of at least one dose of an anti-CD38 antibody.

15. The method of any of claims 1-14, wherein the g-NK cell composition is administered as two doses in a 14-day cycle, wherein the 14-day cycle is repeated one to three times.

16. The method of any of claims 1-15, wherein the g-NK cell composition is administered as six total doses.

17. The method of any of claims 8 and 13-16, wherein the anti-CD38 antibody is daratumumab.

18. The method of any of claims 13-17, wherein administration of the at least one dose of the anti-CD38 antibody is initiated within one month prior to administration of the composition of g-NK cells.

19. The method of any of claims 13-17, wherein administration of the at least one dose of the anti-CD38 antibody is initiated within three weeks prior to administration of the composition of g-NK cells.

20. The method of any of claims 13-17, wherein administration of the at least one dose of the anti-CD38 antibody is initiated within two weeks prior to administration of the composition of g-NK cells.

21. The method of any of claims 8 and 13-20, wherein the anti-CD38 antibody is administered intravenously.

22. The method of any of claims 8 and 13-21, wherein the anti-CD38 antibody is administered as a once weekly dose, optionally for one or two 28-day cycles.

23. The method of any of claims 8 and 13-22, wherein each dose of the anti-CD38 antibody (e.g. daratumumab) is administered in an amount that is from or from about 8 mg/kg to about 32 mg/kg, optionally at or about 16 mg/kg.

24. The method of any of claims 8 and 13-20, wherein the anti-CD38 antibody is administered subcutaneously.

25. The method of any of claims 8, 13-20, and 24, wherein the anti-CD38 antibody (e.g. daratumumab) is administered in an anti-CD38 antibody composition comprising a hyaluronidase, optionally wherein the anti-CD38 antibody composition comprises daratumumab and recombinant human hyaluronidase PH20 (e.g. hyaluronidase-fihj).

26. The method of claim 25, wherein the anti-CD38 antibody composition is administered as a once weekly dose, optionally for one or two 28-day cycles.

27. The method of claim 25 or claim 26, wherein each dose of the anti-CD38 antibody composition comprises from at or about 1200 mg to about 2400 mg anti-CD38 antibody (e.g. daratumumab) and from at or about 15,000 Units (U) to about 45,000 U hyaluronidase (e.g. hyaluronidase-fihj).

28. The method of any of claims 24-27, wherein each dose of the anti-CD38 antibody composition comprises about 1800 mg anti-CD38 antibody (e.g. daratumumab) and about 30,000 U hyaluronidase (e.g. hyaluronidase-fihj).

29. The method of any of claims 8 and 13-28, wherein the method comprises administering the anti-CD38 antibody, optionally the anti-CD38 antibody composition, once weekly for 8 total doses and administering the g-NK cell composition once weekly for 6 total doses, wherein one dose or two doses of the anti-CD38 antibody is administered prior to administration of the composition comprising g-NK cells.

30. The method of any of claims 1-29, wherein the multiple myeloma is relapsed/refractory multiple myeloma.

31. The method of any of claims 1-30, wherein the g-NK cells have low or no expression of CD38, optionally wherein less than 25% of the cells in the g-NK cell composition are positive for surface CD38.

32. The method of any of claims 1-31, wherein the cells in the g-NK cell composition are not engineered to reduce or eliminate CD38 expression.

33. The method of any of claims 1-32, wherein the g-NK cell composition exhibits minimal anti-CD38-induced fratricide, optionally wherein less than 10% of cells in the g-NK cell composition exhibit anti-CD38 induced fratricide.

34. A method of treating lymphoma, the method comprising administering a composition of Natural Killer (NK) cells deficient in expression of FcRγ chain (g-NK cells) to a subject having lymphoma, wherein the composition of g-NK cells is administered once weekly for a predetermined number of doses.

35. The method of claim 34, wherein the method is a monotherapy without combined administration of an exogenous antibody for treating the lymphoma.

36. The method of claim 34, wherein the method further comprises administering to the subject an antibody that is directed against a lymphoma antigen.

37. The method of claim 36, wherein the lymphoma antigen comprises an antigen selected from the group consisting of CD19, CD20, and CD30.

38. The method of claim 36 or claim 37, wherein the antibody is a full-length antibody.

39. The method of any one of claims 36-38, wherein the antibody is an anti-CD19 antibody.

40. The method of any one of claims 36-38, wherein the antibody is an anti-CD30 antibody.

41. The method of any one of claims 36-38, wherein the antibody is an anti-CD20 antibody.

42. The method of claim 36, wherein the antibody is a bispecific antibody.

43. The method of claim 42, wherein the bispecific antibody is directed against CD16 and a second antigen selected from the group consisting of CD19, CD20, and CD30.

44. The method of claim 43, wherein the bispecific antibody is directed against CD16 and CD20.

45. The method of claim 36-44, wherein the antibody is administered once every four weeks, once every three weeks, once every two weeks, once weekly, or twice weekly.

46. The method of claim 41, wherein at least one dose of anti-CD20 antibody has been administered to the subject prior to administration of a dose of the composition of g-NK cells.

47. A method of treating lymphoma, the method comprising administering a composition of Natural Killer (NK) cells deficient in expression of FcRγ chain (g-NK cells) to a subject having lymphoma, wherein the composition of g-NK cells is administered once weekly for a predetermined number of doses, and wherein the subject has received prior administration of at least one dose of an anti-CD20 antibody.

48. The method of any of claims 34-47, wherein the lymphoma is a Non-Hodgkin's Lymphoma (NHL).

49. The method of any of claims 34-48, wherein the g-NK cell composition is administered as two doses in a 14-day cycle, wherein the 14-day cycle is repeated one to three times.

50. The method of any of claims 34-49, wherein the g-NK cell composition is administered as six total doses.

51. The method of any of claims 41 and 45-50, wherein the anti-CD20 antibody is rituximab.

52. The method of any of claims 41 and 45-51, wherein administration of the at least one dose of the anti-CD20 antibody is initiated within one month prior to administration of the composition of g-NK cells.

53. The method of any of claims 41 and 45-52, wherein administration of the at least one dose of the anti-CD20 antibody is initiated within three weeks prior to administration of the composition of g-NK cells.

54. The method of any of claims 41 and 45-53, wherein administration of the at least one dose of the anti-CD20 antibody is initiated within two weeks prior to administration of the composition of g-NK cells.

55. The method of any of claims 41 and 45-54, wherein the anti-CD20 antibody is administered intravenously.

56. The method of any of claims 41 and 45-55, wherein the anti-CD20 antibody is administered as a once weekly dose, optionally for 4 or 8 doses.

57. The method of any of claims 41 and 45-56, wherein each dose of the anti-CD20 antibody is administered in an amount that is from or from about 250 mg/m² to 500 mg/m², optionally at or about 375 mg/m².

58. The method of any of claims 41 and 45-54, wherein the anti-CD20 antibody is administered subcutaneously.

59. The method of any of claims 41, 45-54, and 58, wherein the anti-CD20 antibody (e.g. rituximab) is administered in an anti-CD20 antibody composition comprising a hyaluronidase, optionally wherein the anti-CD20 antibody composition comprises rituximab and a human recombinant hyaluronidase PH20.

60. The method of claim 59, wherein the anti-CD20 antibody composition is administered as a once weekly dose, optionally for 4 or 8 doses or optionally for 3 or 7 doses following a once weekly dose of the anti-CD20 antibody intravenously.

61. The method of claim 59 or claim 60, wherein each dose of the anti-CD20 antibody composition comprises from at or about 1200 mg to about 2400 mg anti-CD20 antibody (e.g. rituximab) and from at or about 15,000 Units (U) to about 45,000 U hyaluronidase.

62. The method of any of claims 59-61, wherein each dose of the anti-CD20 antibody composition comprises about 1400 mg anti-CD20 antibody (e.g. rituximab) and about 23,400 U hyaluronidase.

63. The method of any of claims 59-61, wherein each dose of the anti-CD20 antibody composition comprises about 1600 mg anti-CD20 antibody (e.g. rituximab) and about 26,800 U hyaluronidase.

64. The method of any of claims 41 and 45-63, wherein the method comprises administering the anti-CD20 antibody, optionally the anti-CD20 antibody composition, once weekly for 8 total doses and administering the g-NK cell composition once weekly for 6 total doses, wherein one dose or two doses of the anti-CD20 antibody is administered prior to administration of the composition comprising g-NK cells.

65. The method of any of claims 1-64, wherein, among cells in the g-NK cell composition, greater than at or about 60% of the cells are g-NK cells, greater than at or about 70% of the cells are g-NK cells, greater than at or about 80% of the cells are g-NK cells, greater than at or about 90% of the cells are g-NK cells, or greater than at or about 95% of the cells are g-NK cells.

66. The method of any of claims 1-64, wherein at least at or about 50% of the cells in the g-NK cell composition are FcRγ-deficient (FcRγ^{neg}) NK cells (g-NK), wherein greater than at or about 70% of the g-NK cells are positive for perforin and greater than at or about 70% of the g-NK cells are positive for granzyme B.

67. The method of claim 65 or claim 66, wherein (i) greater than at or about 80% of the g-NK cells are positive for perforin and greater than at or about 80% of the g-NK cells are positive for granzyme B, (ii) greater than at or about 90% of the g-NK cells are positive for perforin and greater than at or about 90% of the g-NK cells are positive for granzyme B, or (iii) greater than at or about 95% of the g-NK cells are positive for perforin and greater than at or about 95% of the g-NK cells are positive for granzyme B.

68. The method of claim 66 or claim 67, wherein:
among the cells positive for perforin, the cells express a mean level of perforin as measured by intracellular flow cytometry that is, based on mean fluorescence intensity (MFI), at least at or about two times the mean level of perforin expressed by cells that are FcRγ^{pos}; and/or.
among the cells positive for granzyme B, the cells express a mean level of granzyme B as measured by intracellular flow cytometry that is, based on mean fluorescence intensity (MFI), at least at or about two times the mean level of granzyme B expressed by cells that are FcRγ^{pos}.

69. The method of any of claims 1-68, wherein greater than 10% of the cells in the g-NK cell composition are capable of degranulation against tumor target cells, optionally as measured by CD10⁷a expression, optionally wherein the degranulation is measured in the absence of an antibody against the tumor target cells.

70. The method of any of claims 1-69, wherein, among the cells in the g-NK cell composition, greater than at or about 15%, greater than at or about 20%, greater than at or about 30%, greater than at or about 40% or greater than at or about 50% exhibit degranulation, optionally as measured by CD10⁷a expression, in the presence of cells expressing a target antigen (target cells) and an antibody directed against the target antigen (anti-target antibody).

71. The method of any of claims 1-70, wherein greater than 10% of the cells in the g-NK cell composition are capable of producing interferon-gamma or TNF-alpha against tumor target cells, optionally wherein the interferon-gamma or TNF-alpha is measured in the absence of an antibody against the tumor target cells.

72. The method of any of claims 1-71, wherein, among the cells in the g-NK cell composition, greater than at or about 15%, greater than at or about 20%, greater than at or about 30%, greater than at or about 40% or greater than at or about 50% produce an effector cytokine in the presence of cells expressing a target antigen (target cells) and an antibody directed against the target antigen (anti-target antibody).

73. The method of claim 72, wherein the effector cytokine is IFN-gamma or TNF-alpha.

74. The method of claim 72 or claim 73, wherein the effector cytokine is IFN-gamma and TNF-alpha.

75. The method of any of claims 1-74, wherein the g-NK cell composition has been produced by ex vivo expansion of CD3−/CD57+ cells cultured with irradiated HLA-E+ feeder cells, wherein the CD3−/CD57+ cells are enriched from a biological sample from a donor subject.

76. The method of claim 75, wherein the donor subject is CMV-seropositive.

77. The method of claim 75 or claim 76, wherein the donor subject has the CD16 158V/V NK cell genotype or the CD16 158V/F NK cell genotype, optionally wherein the biological sample is from a human subject selected for the CD16 158V/V NK cell genotype or the CD16 158V/F NK cell genotype.

78. The method of any of claims 75-77, wherein at least at or about 20% of natural killer (NK) cells in a peripheral blood sample from the donor subject are positive for NKG2C (NKG2C^{pos}) and at least 70% of NK cells in the peripheral blood sample are negative or low for NKG2A (NKG2A^{neg}).

79. The method of any of claims 75-77, wherein the irradiated feeder cells are deficient in HLA class I and HLA class II.

80. The method of any of claims 75-79, wherein the irradiated feeder cells are 221.AEH cells.

81. The method of any of claims 75-80, wherein the culturing is performed in the presence of two or more recombinant cytokines, wherein at least one recombinant cytokine is interleukin (IL)-2 and at least one recombinant cytokine is IL-21.

82. The method of claim 81, wherein the recombinant cytokines are IL-21 and IL-2.

83. The method of claim 81, wherein the recombinant cytokines are IL-21, IL-2, and IL-15.

84. The method of any of claims 1-83, wherein the g-NK cells in the composition are from a single donor subject that have been expanded from the same biological sample.

85. The method of any of claims 1-84, wherein the g-NK cell composition is formulated in a serum-free cryopreservation medium comprising a cryoprotectant, optionally wherein the cyroprotectant is DMSO and the cryopreservation medium is 5% to 10% DMSO (v/v).

86. The method of any of claims 1-85, wherein the g-NK cells are not engineered with an antigen receptor, optionally wherein the antigen receptor is a chimeric antigen receptor.

87. The method of any of claims 1-86, wherein the g-NK cells are not engineered with a secretable cytokine, optionally a cytokine receptor fusion protein, such as IL-15 receptor fusion (IL-15RF)

88. The method of any of claims 1-87, wherein the method does not include exogenous cytokine administration to the subject to support NK cell survival or expansion, wherein the exogenous cytokine is one or more of IL-2, IL-7, IL-15 or IL-21.

89. The method of any of claims 1-88, each dose of g-NK cells is from at or about from at or about 1×10⁸ cells to at or about 50×10⁹ cells of the g-NK cell composition.

90. The method of any of claims 1-89, wherein each dose of g-NK cells is or is about 5×10⁸ cells of the g-NK cell composition.

91. The method of any of claims 1-89, wherein each dose of g-NK cells is or is about 5×10⁹ cells of the g-NK cell composition.

92. The method of any of claims 1-89, wherein each dose of g-NK cells is or is about 10×10⁹ cells of the g-NK cell composition.

93. The method of any of claims 1-92, wherein prior to the administration of the dose of g-NK cells, the subject has received a lymphodepleting therapy.

94. The method of claim 93, wherein the lymphodepleting therapy comprises fludarabine and/or cyclophosphamide.

95. The method of claim 93 or claim 94, wherein the lymphodepleting comprises the administration of fludarabine at or about 20-40 mg/m² body surface area of the subject, optionally at or about 30 mg/m², daily, for 2-4 days, and/or cyclophosphamide at or about 200-400 mg/m² body surface area of the subject, optionally at or about 300 mg/m², daily, for 2-4 days.

96. The method of claim 94 or claim 95, wherein the lymphodepleting therapy comprises fludarabine and cyclophosphamide.

97. The method of any of claims 1-96, wherein the lymphodepleting therapy comprises the administration of fludarabine at or about 30 mg/m² body surface area of the subject, daily, and cyclophosphamide at or about 300 mg/m² body surface area of the subject, daily, each for 2-4 days, optionally 3 days.

98. The method of any of claims 1-97, wherein administration of a dose of g-NK cells is initiated within two weeks or at or about two weeks after initiation of the lymphodepleting therapy.

99. The method of any of claims 1-97, wherein administration of a dose of g-NK cells is initiated within 7 days or at or about 7 days after initiation of the lymphodepleting therapy.

100. The method of any one of claims 1-99, wherein the individual is a human.

101. The method of any one of claims 1-100, wherein the NK cells in the composition are allogenic to the individual.

102. The method of any one of claims 1-101, further comprising administering exogenous cytokine support to facilitate expansion or persistence of the g-NK cells in vivo in the subject, optionally wherein the exogenous cytokine is or comprises IL-15.
